(19) United States
(12) Patent Application Publication
(10) Pub. No.: US 2011/0039847 A1
(43) Pub. Date: Feb. 17, 2011

(54) AMIDE DERIVATIVES AS SIRTUIN MODULATORS

(75) Inventors: Christopher Oalmann, Watertown, MA (US); Robert B. Perni, Marlborough, MA (US); Jeremy S. Disch, Natick, MA (US); Bruce Szczepankiewicz, Hopkinton, MA (US); Giovanna Gualtieri, Waltham, MA (US); Rebecca L. Casaubon, Cambridge, MA (US); Karsten J. Koppetsch, Woburn, MA (US)

Correspondence Address: ROPES & GRAY LLP;IPRM - Floor 43, PRUDENTIAL TOWER, 800 BOYLSTON STREET, BOSTON, MA, 02199-3600, US

(73) Assignee: Sirtris Pharmaceuticals, Inc, Cambridge, MA (US)

(21) Appl. No.: 12/740,686

(22) PCT Filed: Oct. 31, 2008
(86) PCT No.: PCT/US08/12340

§ 371 Date: Oct. 26, 2010

Related U.S. Application Data
(60) Provisional application No. 61/001,740, filed on Nov. 1, 2007.

(51) Int. Cl. A61K 31/5377; C07D 239/42; C07D 401/12; C07D 403/04; C07D 413/14; A61K 31/497; A61P 3/00; A61P 3/10

(52) U.S. Cl. 514/235.8; 544/329; 544/328; 544/295; 544/122; 514/252.14; 514/256

(57) ABSTRACT

Provided herein are novel sirtuin-modulating compounds represented by Structural Formula (I) and methods of use thereof. The sirtuin-modulating compounds may be used for increasing the lifespan of a cell, and treating and/or preventing a wide variety of diseases and disorders including, for example, diseases or disorders related to aging or stress, diabetes, obesity, neurodegenerative diseases, cardiovascular disease, blood clotting disorders, inflammation, cancer, and/or flushing as well as diseases or disorders that would benefit from increased mitochondrial activity. Also provided are compositions comprising a sirtuin-modulating compound in combination with another therapeutic agent.

### RELATED APPLICATION

This application claims the benefit of U.S. Provisional Application No. 61/001,740, filed Nov. 1, 2007, the contents of which are incorporated by reference in their entirety.

### BACKGROUND

The Silent Information Regulator (SIR) family of genes represents a highly conserved group of genes present in the genomes of organisms ranging from archaebacteria to a variety of eukaryotes (Frye, 2000). The encoded SIR proteins are involved in diverse processes from regulation of gene silencing to DNA repair. The proteins encoded by members of the SIR gene family show high sequence conservation in a 250 amino acid core domain. A well-characterized gene in this family is S. cerevisiae SIR2, which is involved in silencing HM loci that contain information specifying yeast mating type, telomere position effects and cell aging (Guarente, 1999; Kaeberlein et al., 1999; Shore, 2000). The yeast Sir2 protein belongs to a family of histone deacetylases (reviewed in Guarente, 2000; Shore, 2000). The Sir2 homolog, CobB, in Salmonella typhimurium, functions as an NAD (nicotinamide adenine dinucleotide)-dependent ADP-ribosyl transferase (Tsang and Escalante-Semerena, 1998).

The Sir2 protein is a class III deacetylase which uses NAD as a cosubstrate (Imai et al., 2000; Moazed, 2001; Smith et al., 2000; Tanner et al., 2000; Tanny and Moazed, 2001). Unlike other deacetylases, many of which are involved in gene silencing, Sir2 is insensitive to class I and II histone deacetylase inhibitors like trichostatin A (TSA) (Imai et al., 2000; Landry et al., 2000a; Smith et al., 2000).

Deacetylation of acetyl-lysine by Sir2 is tightly coupled to NAD hydrolysis, producing nicotinamide and a novel acetyl-ADP ribose compound (Tanner et al., 2000; Landry et al., 2000b; Tanny and Moazed, 2001). The NAD-dependent deacetylase activity of Sir2 is essential for its functions which can connect its biological role with cellular metabolism in yeast (Guarente, 2000; Imai et al., 2000; Lin et al., 2000; Smith et al., 2000). Mammalian Sir2 homologs have NAD-dependent histone deacetylase activity (Imai et al., 2000; Smith et al., 2000). Most information about Sir2 mediated functions comes from the studies in yeast (Gartenberg, 2000; Gottschling, 2000).

Biochemical studies have shown that Sir2 can readily deacetylate the amino-terminal tails of histones H3 and H4, resulting in the formation of 1-O-acetyl-ADP-ribose and nicotinamide. Strains with additional copies of SIR2 display increased rDNA silencing and a 30% longer life span. It has recently been shown that additional copies of the C. elegans SIR2 homolog, sir-2.1, and the D. melanogaster dSir2 gene greatly extend life span in those organisms. This implies that the SIR2-dependent regulatory pathway for aging arose early in evolution and has been well conserved. Today, Sir2 genes are believed to have evolved to enhance an organism's health and stress resistance to increase its chance of surviving adversity.

SIRT3 is a homolog of SIRT1 that is conserved in prokaryotes and eukaryotes (P. Onyango et al., Proc. Natl. Acad. Sci. USA 99: 13653-13658 (2002)). The SIRT3 protein is targeted to the mitochondrial cristae by a unique domain located at the N-terminus. SIRT3 has NAD+-dependent protein deacetylase activity and is upbiquitously expressed, particularly in metabolically active tissues. Upon transfer to the mitochondria, SIRT3 is believed to be cleaved into a smaller, active form by a mitochondrial matrix processing peptidase (MPP) (B. Schwer et al., J. Cell Biol. 158: 647-657 (2002)).

Caloric restriction has been known for over 70 years to improve the health and extend the lifespan of mammals (Masoro, 2000). Yeast life span, like that of metazoans, is also extended by interventions that resemble caloric restriction, such as low glucose. The discovery that both yeast and flies lacking the SIR2 gene do not live longer when calorically restricted provides evidence that SIR2 genes mediate the beneficial health effects of this diet (Anderson et al., 2003; Helfand and Rogina, 2004). Moreover, mutations that reduce the activity of the yeast glucose-responsive cAMP (adenosine 3′,5′-monophosphate)-dependent (PKA) pathway extend life span in wild type cells but not in mutant sir2 strains, demonstrating that SIR2 is likely to be a key downstream component of the caloric restriction pathway (Lin et al., 2001).

### SUMMARY

Provided herein are novel sirtuin-modulating compounds and methods of use thereof.

In one aspect, the invention provides sirtuin-modulating compounds of Structural Formulas (I) and (II) as are described in detail below.

In another aspect, the invention provides methods for using sirtuin-modulating compounds, or compostions comprising sirtuin-modulating compounds. In certain embodiments, sirtuin-modulating compounds that increase the level and/or activity of a sirtuin protein may be used for a variety of therapeutic applications including, for example, increasing the lifespan of a cell, and treating and/or preventing a wide variety of diseases and disorders including, for example, diseases or disorders related to aging or stress, diabetes, obesity, neurodegenerative diseases, chemotherapeutic induced neuropathy, neuropathy associated with an ischemic event, ocular diseases and/or disorders, cardiovascular disease, blood clotting disorders, inflammation, and/or flushing, etc. Sirtuin-modulating compounds that increase the level and/or activity of a sirtuin protein may also be used for treating a disease or disorder in a subject that would benefit from increased mitochondrial activity, for enhancing muscle performance, for increasing muscle ATP levels, or for treating or preventing muscle tissue damage associated with hypoxia or ischernia. In other embodiments, sirtuin-modulating compounds that decrease the level and/or activity of a sirtuin protein may be used for a variety of therapeutic applications including, for example, increasing cellular sensitivity to stress, increasing apoptosis, treatment of cancer, stimulation of appetite, and/or stimulation of weight gain, etc. As described further below, the methods comprise administering to a subject in need thereof a pharmaceutically effective amount of a sirtuin-modulating compound.

In certain aspects, the sirtuin-modulating compounds may be administered alone or in combination with other compounds, including other sirtuin-modulating compounds, or other therapeutic agents.

### DETAILED DESCRIPTION

### 1. Definitions

As used herein, the following terms and phrases shall have the meanings set forth below. Unless defined otherwise, all technical and scientific terms used herein have the same meaning as commonly understood to one of ordinary skill in the art.

The singular forms “a,” “an,” and “the” include plural reference unless the context clearly dictates otherwise.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent” which is a biologically, physiologically, or pharmacologically active substance (or substances) that acts locally or systemically in a subject.

The term “bioavailable” when referring to a compound is art-recognized and refers to a form of a compound that allows for it, or a portion of the amount of compound administered, to be absorbed by, incorporated to, or otherwise physiologically available to a subject or patient to whom it is administered.

“Biologically active portion of a sirtuin” refers to a portion of a sirtuin protein having a biological activity, such as the ability to deacetylate. Biologically active portions of a sirtuin may comprise the core domain of sirtuins. Biologically active portions of SIRT1 having GenBank Accession No. NP_{—}036370 that encompass the NAD+ binding domain and the substrate binding domain, for example, may include without limitation, amino acids 62-293 of GenBank Accession No. NP_{—}036370, which are encoded by nucleotides 237 to 932 of GenBank Accession No. NM_{—}012238. Therefore, this region is sometimes referred to as the core domain. Other biologically active portions of SIRT1, also sometimes referred to as core domains, include about amino acids 261 to 447 of GenBank Accession No. NP_{—}036370, which are encoded by nucleotides 834 to 1394 of GenBank Accession No. NM_{—}012238; about amino acids 242 to 493 of GenBank Accession No. NP_{—}036370, which are encoded by nucleotides 777 to 1532 of GenBank Accession No. NM_{—}012238; or about amino acids 254 to 495 of GenBank Accession No. NP_{—}036370, which are encoded by nucleotides 813 to 1538 of GenBank Accession No. NM_{—}012238.

The term “companion animals” refers to cats and dogs. As used herein, the term “dog(s)” denotes any member of the species Canis familiaris, of which there are a large number of different breeds. The term “cat(s)” refers to a feline animal including domestic cats and other members of the family Felidae, genus Felis.

“Diabetes” refers to high blood sugar or ketoacidosis, as well as chronic, general metabolic abnormalities arising from a prolonged high blood sugar status or a decrease in glucose tolerance. “Diabetes” encompasses both the type I and type II (Non Insulin Dependent Diabetes Mellitus or NIDDM) forms of the disease. The risk factors for diabetes include the following factors: waistline of more than 40 inches for men or 35 inches for women, blood pressure of 130/85 mmHg or higher, triglycerides above 150 mg/dl, fasting blood glucose greater than 100 mg/dl or high-density lipoprotein of less than 40 mg/dl in men or 50 mg/dl in women.

A “direct activator” of a sirtuin is a molecule that activates a sirtuin by binding to it. A “direct inhibitor” of a sirtuin is a molecule inhibits a sirtuin by binding to it.

The term “ED₅₀” is art-recognized. In certain embodiments, ED₅₀ means the dose of a drug which produces 50% of its maximum response or effect, or alternatively, the dose which produces a pre-determined response in 50% of test subjects or preparations. The term “LD₅₀” is art-recognized. In certain embodiments, LD₅₀ means the dose of a drug which is lethal in 50% of test subjects. The term “therapeutic index” is an art-recognized term which refers to the therapeutic index of a drug, defined as LD₅₀/ED₅₀.

The term “hyperinsulinemia” refers to a state in an individual in which the level of insulin in the blood is higher than normal.

The term “insulin resistance” refers to a state in which a normal amount of insulin produces a subnormal biologic response relative to the biological response in a subject that does not have insulin resistance.

An “insulin resistance disorder,” as discussed herein, refers to any disease or condition that is caused by or contributed to by insulin resistance. Examples include: diabetes, obesity, metabolic syndrome, insulin-resistance syndromes, syndrome X, insulin resistance, high blood pressure, hypertension, high blood cholesterol, dyslipidemia, hyperlipidemia, dyslipidemia, atherosclerotic disease including stroke, coronary artery disease or myocardial infarction, hyperglycemia, hyperinsulinemia and/or hyperproinsulinemia, impaired glucose tolerance, delayed insulin release, diabetic complications, including coronary heart disease, angina pectoris, congestive heart failure, stroke, cognitive functions in dementia, retinopathy, peripheral neuropathy, nephropathy, glomerulonephritis, glomerulosclerosis, nephrotic syndrome, hypertensive nephrosclerosis some types of cancer (such as endometrial, breast, prostate, and colon), complications of pregnancy, poor female reproductive health (such as menstrual irregularities, infertility, irregular ovulation, polycystic ovarian syndrome (PCOS)), lipodystrophy, cholesterol related disorders, such as gallstones, cholescystitis and cholelithiasis, gout, obstructive sleep apnea and respiratory problems, osteoarthritis, and prevention and treatment of bone loss, e.g. osteoporosis.

The term “livestock animals” refers to domesticated quadrupeds, which includes those being raised for meat and various byproducts, e.g., a bovine animal including cattle and other members of the genus Bos, a porcine animal including domestic swine and other members of the genus Sus, an ovine animal including sheep and other members of the genus Ovis, domestic goats and other members of the genus Capra; domesticated quadrupeds being raised for specialized tasks such as use as a beast of burden, e.g., an equine animal including domestic horses and other members of the family Equidae, genus Equus.

The term “mammal” is known in the art, and exemplary mammals include humans, primates, livestock animals (including bovines, porcines, etc.), companion animals (e.g., canines, felines, etc.) and rodents (e.g., mice and rats).

“Obese” individuals or individuals suffering from obesity are generally individuals having a body mass index (BMI) of at least 25 or greater. Obesity may or may not be associated with insulin resistance.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and intrasternal injection and infusion.

A “patient”, “subject”, “individual” or “host” refers to either a human or a non-human animal.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “prophylactic” or “therapeutic” treatment is art-recognized and refers to administration of a drug to a host. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The term “pyrogen-free”, with reference to a composition, refers to a composition that does not contain a pyrogen in an amount that would lead to an adverse effect (e.g., irritation, fever, inflammation, diarrhea, respiratory distress, endotoxic shock, etc.) in a subject to which the composition has been administered. For example, the term is meant to encompass compositions that are free of, or substantially free of, an endotoxin such as, for example, a lipopolysaccharide (LPS).

“Replicative lifespan” of a cell refers to the number of daughter cells produced by an individual “mother cell.” “Chronological aging” or “chronological lifespan,” on the other hand, refers to the length of time a population of non-dividing cells remains viable when deprived of nutrients. “Increasing the lifespan of a cell” or “extending the lifespan of a cell,” as applied to cells or organisms, refers to increasing the number of daughter cells produced by one cell; increasing the ability of cells or organisms to cope with stresses and combat damage, e.g., to DNA, proteins; and/or increasing the ability of cells or organisms to survive and exist in a living state for longer under a particular condition, e.g., stress (for example, heatshock, osmotic stress, high energy radiation, chemically-induced stress, DNA damage, inadequate salt level, inadequate nitrogen level, or inadequate nutrient level). Lifespan can be increased by at least about 20%, 30%, 40%, 50%, 60% or between 20% and 70%, 30% and 60%, 40% and 60% or more using methods described herein.

“Sirtuin-activating compound” refers to a compound that increases the level of a sirtuin protein and/or increases at least one activity of a sirtuin protein. In an exemplary embodiment, a sirtuin-activating compound may increase at least one biological activity of a sirtuin protein by at least about 10%, 25%, 50%, 75%, 100%, or more. Exemplary biological activities of sirtuin proteins include deacetylation, e.g., of histones and p53; extending lifespan; increasing genomic stability; silencing transcription; and controlling the segregation of oxidized proteins between mother and daughter cells.

“Sirtuin-inhibiting compound” refers to a compound that decreases the level of a sirtuin protein and/or decreases at least one activity of a sirtuin protein. In an exemplary embodiment, a sirtuin-inhibiting compound may decrease at least one biological activity of a sirtuin protein by at least about 10%, 25%, 50%, 75%, 100%, or more. Exemplary biological activities of sirtuin proteins include deacetylation, e.g., of histones and p53; extending lifespan; increasing genomic stability; silencing transcription; and controlling the segregation of oxidized proteins between mother and daughter cells.

“Sirtuin-modulating compound” refers to a compound of Formulae (I)-(II) as described herein. In exemplary embodiments, a sirtuin-modulating compound may either up regulate (e.g., activate or stimulate), down regulate (e.g., inhibit or suppress) or otherwise change a functional property or biological activity of a sirtuin protein. Sirtuin-modulating compounds may act to modulate a sirtuin protein either directly or indirectly. In certain embodiments, a sirtuin-modulating compound may be a sirtuin-activating compound or a sirtuin-inhibiting compound.

“Sirtuin protein” refers to a member of the sirtuin deacetylase protein family, or preferably to the sir2 family, which include yeast Sir2 (GenBank Accession No. P53685), C. elegans Sir-2.1 (GenBank Accession No. NP_{—}501912), and human SIRT1 (GenBank Accession No. NM_{—}012238 and NP_{—}036370 (or AF083106)) and SIRT2 (GenBank Accession No. NM_{—}012237, NM_{—}030593, NP_{—}036369, NP_{—}085096, and AF083107) proteins. Other family members include the four additional yeast Sir2-like genes termed “HST genes” (homologues of Sir two) HST1, HST2, HST3 and HST4, and the five other human homologues hSIRT3, hSIRT4, hSIRT5, hSIRT6 and hSIRT7 (Brachmann et al. (1995) Genes Dev. 9:2888 and Frye et al. (1999) BBRC 260:273). Preferred sirtuins are those that share more similarities with SIRT1, i.e., hSIRT1, and/or Sir2 than with SIRT2, such as those members having at least part of the N-terminal sequence present in SIRT1 and absent in SIRT2 such as SIRT3 has.

“SIRT1 protein” refers to a member of the sir2 family of sirtuin deacetylases. In one embodiment, a SIRT1 protein includes yeast Sir2 (GenBank Accession No. P53685), C. elegans Sir-2.1 (GenBank Accession No. NP_{—}501912), human SIRT1 (GenBank Accession No. NM_{—}012238 or NP_{—}036370 (or AF083106)), and human SIRT2 (GenBank Accession No. NM_{—}012237, NM_{—}030593, NP 036369, NP_{—}085096, or AF083107) proteins, and equivalents and fragments thereof. In another embodiment, a SIRT1 protein includes a polypeptide comprising a sequence consisting of, or consisting essentially of, the amino acid sequence set forth in GenBank Accession Nos. NP_{—}036370, NP_{—}501912, NP_{—}085096, NP_{—}036369, or P53685. SIRT1 proteins include polypeptides comprising all or a portion of the amino acid sequence set forth in GenBank Accession Nos. NP_{—}036370, NP_{—}501912, NP_{—}085096, NP_{—}036369, or P53685; the amino acid sequence set forth in GenBank Accession Nos. NP_{—}036370, NP_{—}501912, NP_{—}085096, NP_{—}036369, or P53685 with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to GenBank Accession Nos. NP_{—}036370, NP_{—}501912, NP_{—}085096, NP 036369, or P53685, and functional fragments thereof. Polypeptides of the invention also include homologs (e.g., orthologs and paralogs), variants, or fragments, of GenBank Accession Nos. NP_{—}036370, NP_{—}501912, NP_{—}085096, NP_{—}036369, or P53685.

“SIRT3 protein” refers to a member of the sirtuin deacetylase protein family and/or to a homolog of a SIRT1 protein. In one embodiment, a SIRT3 protein includes human SIRT3 (GenBank Accession No. AAH01042, NP_{—}036371, or NP_{—}001017524) and mouse SIRT3 (GenBank Accession No. NP_{—}071878) proteins, and equivalents and fragments thereof. In another embodiment, a SIRT3 protein includes a polypeptide comprising a sequence consisting of, or consisting essentially of, the amino acid sequence set forth in GenBank Accession Nos. AAH01042, NP_{—}036371, NP_{—}001017524, or NP_{—}071878. SIRT3 proteins include polypeptides comprising all or a portion of the amino acid sequence set forth in GenBank Accession AAH01042, NP_{—}036371, NP_{—}001017524, or NP_{—}071878; the amino acid sequence set forth in GenBank Accession Nos. AAH01042, NP_{—}036371, NP_{—}001017524, or NP_{—}071878 with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to GenBank Accession Nos. AAH01042, NP_{—}036371, NP_{—}001017524, or NP_{—}071878, and functional fragments thereof. Polypeptides of the invention also include homologs (e.g., orthologs and paralogs), variants, or fragments, of GenBank Accession Nos. AAH01042, NP_{—}036371, NP_{—}001017524, or NP_{—}071878. In one embodiment, a SIRT3 protein includes a fragment of SIRT3 protein that is produced by cleavage with a mitochondrial matrix processing peptidase (MPP) and/or a mitochondrial intermediate peptidase (MIP).

The terms “systemic administration,” “administered systemically,” “peripheral administration” and “administered peripherally” are art-recognized and refer to the administration of a subject composition, therapeutic or other material other than directly into the central nervous system, such that it enters the patient's system and, thus, is subject to metabolism and other like processes.

The term “therapeutic agent” is art-recognized and refers to any chemical moiety that is a biologically, physiologically, or pharmacologically active substance that acts locally or systemically in a subject. The term also means any substance intended for use in the diagnosis, cure, mitigation, treatment or prevention of disease or in the enhancement of desirable physical or mental development and/or conditions in an animal or human.

The term “therapeutic effect” is art-recognized and refers to a local or systemic effect in animals, particularly mammals, and more particularly humans caused by a pharmacologically active substance. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art. For example, certain compositions described herein may be administered in a sufficient amount to produce a desired effect at a reasonable benefit/risk ratio applicable to such treatment.

“Treating” a condition or disease refers to curing as well as ameliorating at least one symptom of the condition or disease.

The term “vision impairment” refers to diminished vision, which is often only partially reversible or irreversible upon treatment (e.g., surgery). Particularly severe vision impairment is termed “blindness” or “vision loss”, which refers to a complete loss of vision, vision worse than 20/200 that cannot be improved with corrective lenses, or a visual field of less than 20 degrees diameter (10 degrees radius).

### 2. Sirtuin Modulators

In one aspect, the invention provides novel sirtuin-modulating compounds for treating and/or preventing a wide variety of diseases and disorders including, for example, diseases or disorders related to aging or stress, diabetes, obesity, neurodegenerative diseases, ocular diseases and disorders, cardiovascular disease, blood clotting disorders, inflammation, cancer, and/or flushing, etc. Sirtuin-modulating compounds that increase the level and/or activity of a sirtuin protein may also be used for treating a disease or disorder in a subject that would benefit from increased mitochondrial activity, for enhancing muscle performance, for increasing muscle ATP levels, or for treating or preventing muscle tissue damage associated with hypoxia or ischemia. Other compounds disclosed herein may be suitable for use in a pharmaceutical composition and/or one or more methods disclosed herein.

In one embodiment, sirtuin-modulating compounds of the invention are represented by Structural Formula (I):

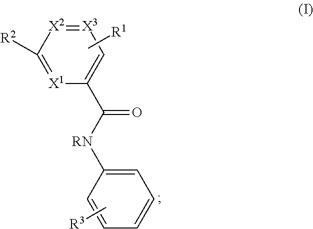

or a salt thereof; wherein:

two of X¹, X² and X³ are independently selected from —CH— and —N—;

the other of X¹, X² and X³ is —CH—;

R¹ is a solubilizing group;

R² is selected from phenyl, fluorophenyl and a 5- to 6-membered heterocycle containing an N heteroatom and, optionally, a second heteroatom selected from N, O or S, wherein said heterocycle is optionally substituted with methyl;

R is —H or —CH₃;

R³ is selected from —H, —C(O)R⁴, —C(O)OR⁴, —OC(O)R⁴, —C(O)NR^{4a}R^{4b}, —NR^{4a}C(O)R^{4b}, —NR^{4a}R^{4b}, —C(═N—OH)R⁴, —OR⁴, —SR⁴, —CH₂R⁴, alkyl, alkenyl, alkynyl, cyano, monocyclyl and halo;

R⁴ in each occurrence is independently selected from hydrogen, lower alkyl and monocyclyl; and

R^{4a} and R^{4b} are independently selected from hydrogen, lower alkyl and monocyclyl; or R^{4a} and R^{4b} taken together with the atom to which they are connected form a heterocycle.

In certain embodiments, R³ is bound to a carbon of the benzene ring in the ortho or para position relative to the amide functionality. In certain such embodiments, sirtuin-modulating compounds of the invention are represented by Structural Formula (II):

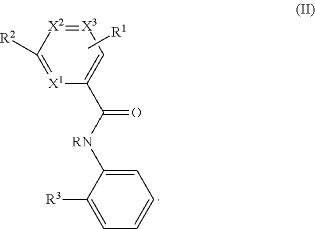

The following values apply to both Structural Formulas (I) and (II).

In certain embodiments, X¹ is —N—. In certain embodiments, X² is —N—. In certain embodiments, X³ is —N—. In certain embodiments, X¹ and X² are —N— and X³ is —CH—.

In certain embodiments, R² is selected from phenyl, fluorophenyl, methylthiazolyl, pyrimidinyl, pyridyl and pyrazolyl. In certain such embodiments, R² is selected from phenyl, fluorophenyl, 2-methylthiazol-4-yl, pyridyl and pyrazol-1-yl. Typically, R² is phenyl or pyridyl.

In certain embodiments, R³ is selected from —C(O)R⁴, —C(O)OR⁴, —OC(O)R⁴, —C(O)NR^{4a}R^{4b}, —NR^{4a}C(O)R^{4b}, —SR⁴, —CH₂R⁴, —NR^{4a}R^{4b}, alkyl, alkenyl, alkynyl, cyano, monocyclo and halo. In certain such embodiments, R⁴, R^{4a} and R^{4b} are independently selected in each occurance from hydrogen or lower alkyl. In other embodiments, R^{4a} and R^{4b} taken together with the atom(s) to which they are attached form a heterocycle. In certain embodiments, R³ is selected from lower alkyl, monocyclo, —C(O)NR^{4a}R^{4b}, —NR^{4a}C(O)R^{4b}, —OC(O)R⁴, —C(O)OR⁴ and cyano. In particular embodiments, R² is selected from phenyl, 3-fluorophenyl and pyridyl, X¹ and X² are —N— and X³ is —CH—, R³ is selected from alkyl, monocyclo, —C(O)NR^{4a}R^{4b}, —NR^{4a}C(O)R^{4b}, —OC(O)R⁴, —C(O)OR⁴ and cyano, and R⁴, R^{4a} and R^{4b} are hydrogen or lower alkyl.

In certain embodiments, R³ is —NR^{4a}R^{4b} and R^{4a} and R^{4b} are independently selected in each occurance from hydrogen or lower alkyl, or R^{4a} and R^{4b} taken together with the atom to which they are attached form a heterocycle. In certain embodiments, R^{4a} and R^{4b} are unsubstituted lower alkyl, amino lower alkyl, alkyl amino lower alkyl, or lower dialkyl amino lower alkyl. In particular embodiments, R² is selected from phenyl, 3-fluorophenyl and pyridyl, X¹ and X² are —N—, X³ is —CH—, and R³ is —NHR⁵ wherein R⁵ is lower alkyl.

In certain embodiments, R³ is —CH₂R⁴ and R⁴ is monocyclyl. In certain embodiments R⁴ is a nitrogen-containing heterocycle such as substituted or unsubstituted thiazolyl, oxazolyl, isoxazolyl, isothiozolyl, triazolyl, tetrazolyl, pyrazolyl, imidazolyl, pyridinyl, pyrrolyl, thiazinyl, oxazinyl, piperidinyl, piperazinyl, pyrimidinyl, morpholinyl, thiomorpholinyl and 1,1-dioxo-1-thiomorpholinyl. In particular embodiments, R² is selected from phenyl, 3-fluorophenyl and pyridyl, X¹ and X² are —N—, X³ is —CH—, and R³ is —CH₂R⁴ wherein R⁴ is a nitrogen-containing heterocycle.

In certain embodiments, R³ is monocyclyl. In certain embodiments R³ is selected from 5-7 membered heterocyclyl and 5-7 membered carbocyclyl. In certain such embodiments, R³ is 5-7 membered heterocyclyl comprising at least one nitrogen, such as substituted or unsubstituted thiazolyl, oxazolyl, isoxazolyl, isothiozolyl, thiadiazolyl, oxadiazolyl, triazolyl, tetrazolyl, pyrazolyl, imidazolyl, isoimidazolyl, pyridinyl, pyrrolyl, thiazinyl, oxazinyl, piperidinyl, piperazinyl, pyrimidinyl, morpholinyl, and thiomorpholinyl, particularly thiadiazolyl, all of which are generally attached to the remainder of the molecule via a carbon atom. Other suitable heterocyclyls include furanyl and tetrahydrofuranyl. Typical substituents for monocyclyl groups, particularly heterocyclyl groups, are lower alkyl, cycloalkyl, lower alkoxy, lower alkylthio, lower alkylsulfinyl, lower alkylsulfonyl, lower alkoxycarbonyl, aminocarbonyl, lower alkyl-aminocarbonyl, di(lower alkyl)-aminocarbonyl, aminoalkylaminocarbonyl, lower alkyl-aminoalkylaminocarbonyl, di(lower alkyl)-aminoalkylaminocarbonyl, sulfonamido, lower alkyl sulfonamido, cyclic amino (including monocyclic and fused bicyclic amino, e.g., morpholino, pyrrolidinyl, piperadinyl, piperazinyl, octahydropyrrolo[1,2-a]pyrazin-2-yl), cyclic aminocarbonyl (e.g, morpholinocarbonyl, pyrrolidinylcarbonyl, piperadinylcarbonyl, piperazinylcarbonyl), cyclic aminocarbonylamino (e.g, morpholinocarbonylamino, pyrrolidinylcarbonylamino, piperadinylcarbonylamino, piperazinylcarbonylamino), cyclic ethers (e.g., tetrahydrofuranyl, tetrahydropyranyl), halo(tetrahydropyranylidene) lower alkyl (e.g., fluoro(4-tetrahydropyranylidene)methyl), pyridyl and phenyl, along with solubilizing groups other than those specifically named above, particularly cyclic solubilizing groups. In particular embodiments, R² is selected from phenyl, 3-fluorophenyl and pyridyl, X¹ and X² are —N—, X³ is —CH—, and R³ is 5-7 membered heterocyclyl comprising at least one nitrogen, such as where R³ is substituted with a solubilizing group (e.g., a cyclic solubilizing group).

In certain embodiments, R³ is selected from —C(O)R⁴, —C(O)OR⁴, —OC(O)R⁴, —C(O)NR^{4a}R^{4b} and —NR^{4a}C(O)R^{4b}. In certain such embodiments, R³ is selected from —C(O)OR⁴ and —C(O)NR^{4a}R^{4b}. In these embodiments, R⁴, R^{4a} and R^{4b} are typically selected from hydrogen and lower alkyl, or R^{4a} and R^{4b} taken together with the atom(s) to which they are attached form a heterocycle. Suitable values of R⁴, R^{4a} and R^{4b} include lower alkyl optionally substituted with the substituents indicated in this paragraph or in the definition of “alkyl,” such as amino lower alkyl, lower alkyl amino lower alkyl, lower dialkyl amino lower alkyl, acetyl amino lower alkyl, carboxy lower alkyl, lower alkyl carboxy lower alkyl, alkyloxycarbonyl alkyl, hydroxy lower alkyl, alkoxy lower alkyl, lower alkyl thio lower alkyl, monocyclyl, monocyclyl lower alkyl, lower alkyl sulfinyl lower alkyl or lower alkyl sulfonyl lower alkyl. In other embodiments, one or more of R⁴, R^{4a} and R^{4b} is monocyclyl, such as cycloalkyl, phenyl, thiazolyl, pyrrolidinyl, piperidinyl or pyridyl. In particular embodiments, R² is selected from phenyl, 3-fluorophenyl and pyridyl, X¹ and X² are —N—, X³ is —CH—, and R³ is selected from —C(O)OR⁴ and —C(O)NR^{4a}R^{4b}.

In certain embodiments, R¹ is —NHR⁵ and R⁵ is lower alkyl optionally substituted with the substituents indicated in this paragraph or in the definitions below. In certain embodiments, R⁵ is amino lower alkyl, lower alkyl amino lower alkyl, lower dialkyl amino lower alkyl, acetyl amino lower alkyl, lower alkyl carboxy lower alkyl, alkyloxycarbonyl alkyl, hydroxy lower alkyl, alkoxy lower alkyl, lower alkyl thio lower alkyl, monocyclyl, monocyclyl lower alkyl, or lower alkyl sulfonylalkyl. In particular embodiments, R² is selected from phenyl, 3-fluorophenyl and pyridyl, X¹ and X² are —N—, X³ is —CH—, and R¹ is —NHR⁵ wherein R⁵ is lower alkyl. In certain of these embodiments, R¹ is —NHR⁵ and R⁵ is lower alkyl, R² is selected from phenyl, 3-fluorophenyl and pyridyl and X¹ and X² are —N— and X³ is —CH— and R³ is selected from alkyl, monocyclyl, —C(O)NR^{4a}R^{4b}, —NR^{4a}C(O)R^{4b}, —OC(O)R⁴, —C(O)OR⁴ and cyano, and R⁴, R^{4a} and R^{4b} are hydrogen or lower alkyl.

In certain embodiments, R¹ is —NR⁷R⁸, wherein R⁷ and R⁸ together with the nitrogen atom to which they are attached form a 4, 5, 6, 7, or 8-membered heterocycle. In certain embodiments, the heterocycle is a 5, 6 or 7-membered heterocycle. In certain embodiments the heterocycle includes one or more of the following substituted or unsubstituted atoms in the ring —N(R⁹)—, —S(O₂)—, —C(R⁹R⁹)—, —N(CO₂R⁹)—, —O— or —S—, where R⁹ in each occurance is independently selected from hydrogen or lower alkyl. In certain embodiments, the heterocycle is substituted with one or more lower alkyl groups. Examples of such R¹ groups include piperazin-1-yl, 3,5-dimethylpiperazin-1-yl, morpholin-4-yl, piperidin-1-yl, and pyrrolidin-1-yl. In particular embodiments, R² is selected from phenyl, 3-fluorophenyl and pyridyl, X¹ and X² are —N—, X³ is —CH—, and R¹ is —NR⁷R⁸, wherein R⁷ and R⁸ together with the nitrogen atom to which they are attached form a 5, 6 or 7-membered heterocycle. In certain of these embodiments, R¹ is —NR⁷R⁸, wherein R⁷ and R⁸ together with the nitrogen atom to which they are attached form a 5, 6 or 7-membered heterocycle and R² is selected from phenyl, 3-fluorophenyl and pyridyl and X¹ and X² are —N— and X³ is —CH— and R³ is selected from alkyl, monocyclyl, —C(O)NR^{4a}R^{4b}, —NR^{4a}C(O)R^{4b}, —OC(O)R⁴, —C(O)OR⁴ and cyano, particularly monocyclyl, and R⁴, R^{4a} and R^{4b} are hydrogen or lower alkyl.

In certain embodiments, R¹ is —CH₂R⁶ and R⁶ is monocyclic, such as a nitrogen-containing heterocycle (e.g., piperazin-1-yl, 4-(methoxyethyl-piperazin-1-yl, 3,5-dimethylpiperazin-1-yl, morpholin-4-yl, piperidin-1-yl, 4-aminopiperidin-1-yl, pyrrolidin-1-yl, 3-fluoropyrrolidin-1-yl, —NH-(pyrrolidin-3-yl), or 1,4-diaza-bicyclo[2.2.1]heptan-1-yl). In particular embodiments, R² is selected from phenyl, 3-fluorophenyl and pyridyl, X¹ and X² are —N—, X³ is —CH—, and R¹ is —CH₂R⁶ wherein R⁶ is a nitrogen-containing heterocycle. In certain of these embodiments, R¹ is —CH₂R⁶ and R⁶ is monocyclic, R² is selected from phenyl, 3-fluorophenyl and pyridyl, X¹ and X² are —N— and X³ is —CH— and R³ is selected from alkyl, monocyclo, —C(O)NR^{4a}R^{4b}, —NR^{4a}C(O)R^{4b}, —OC(O)R⁴, —C(O)OR⁴ and cyano, and R⁴, R^{4a} and R^{4b} are hydrogen or lower alkyl.

In certain embodiments, sirtuin-modulating compounds of the invention can be prepared as represented in Scheme I:

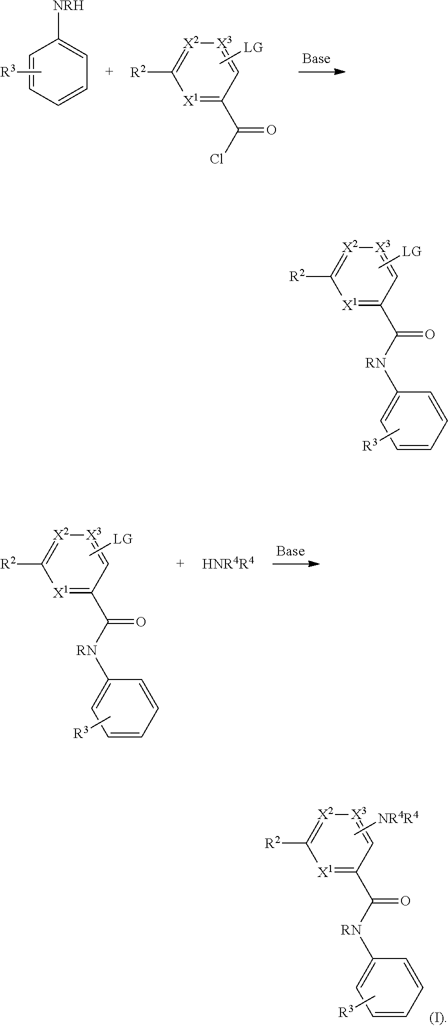

R, R²-R⁴ and X¹-X³ have the values given above, although HNR⁴R⁴ could also represent a nitrogen-containing heterocycle.

The Base is an inorganic or organic base such as Na₂CO₃, K₂CO₃, Cs₂CO₃, N-methyl-morpholine, triethylamine, pyridine, N,N-dimethylethylenediamine or N,N-diisopropylethylamine; and

LG is a leaving group such as halo or tosyl.

Compounds of the invention, including novel compounds of the invention, can also be used in the methods described herein.

The compounds and salts thereof described herein also include their corresponding hydrates (e.g., hemihydrate, monohydrate, dihydrate, trihydrate, tetrahydrate) and solvates. Suitable solvents for preparation of solvates and hydrates can generally be selected by a skilled artisan.

The compounds and salts thereof can be present in amorphous or crystalline (including co-crystalline and polymorph) forms.

Sirtuin-modulating compounds of the invention advantageously modulate the level and/or activity of a sirtuin protein, particularly the deacetylase activity of the sirtuin protein.

Separately or in addition to the above properties, certain sirtuin-modulating compounds of the invention do not substantially have one or more of the following activities: inhibition of PI3-kinase, inhibition of aldoreductase, inhibition of tyrosine kinase, transactivation of EGFR tyrosine kinase, coronary dilation, or spasmolytic activity, at concentrations of the compound that are effective for modulating the deacetylation activity of a sirtuin protein (e.g., such as a SIRT1 and/or a SIRT3 protein).

An alkyl group is a straight chained or branched non-aromatic hydrocarbon which is completely saturated. Typically, a straight chained or branched alkyl group has from 1 to about 20 carbon atoms, preferably from 1 to about 10, and a cyclic alkyl group has from 3 to about 10 carbon atoms, preferably from 3 to about 8. Examples of straight chained and branched alkyl groups include methyl, ethyl, n-propyl, iso-propyl, n-butyl, sec-butyl, tert-butyl, pentyl, hexyl, pentyl and octyl.

Lower alkyl is a straight or branched alkyl group containing from 1-8 carbon atoms, such as methyl, ethyl, n-propyl, iso-propyl, n-butyl, sec-butyl, tert-butyl, pentyl, hexyl, pentyl, octyl and the like. Optionally, lower alkyl is substituted with one or more subtituents selected from halo, cyano, lower alkoxy, hydroxyl, amino, lower alkylamino and lower dialkylamino.

A cycloalkyl group is a cyclic alkyl group.

Alkenyl and alkynyl groups are analogous to alkyl, but contain one or more double or triple bonds, respectively.

Monocyclyl includes 5-7 membered aryl or heteroaryl, 3-7 membered cycloalkyl, and 5-7 membered non-aromatic heterocyclyl. Monocyclyl is optionally substituted with one or more substituents selected from halo, cyano, lower alkoxy, lower alkyl thio, lower alkylsulfinyl, lower alkylsulfonyl, lower alkoxycarbonyl, lower alkyl, monocyclyl (e.g cycloalkyl, pyridyl, phenyl), hydroxyl, aminocarbonyl, lower alkyl-aminocarbonyl, di(lower alkyl)-aminocarbonyl, aminoalkylaminocarbonyl, lower alkyl-aminoalkylaminocarbonyl, di(lower alkyl)-aminoalkylaminocarbonyl, amino, lower alkylamino, lower dialkylamino, sulfonamido, lower alkyl sulfonamido, cyclic amino (including monocyclic and fused bicyclic amino, e.g., morpholino, pyrrolidinyl, piperadinyl, piperazinyl, octahydropyrrolo[1,2-a]pyrazin-2-yl), cyclic aminocarbonyl (e.g, morpholinocarbonyl, pyrrolidinylcarbonyl, piperadinylcarbonyl, piperazinylcarbonyl), cyclic amino-carbonylamino (e.g, morpholinocarbonylamino, pyrrolidinylcarbonylamino, piperadinylcarbonylamino, piperazinylcarbonylamino), cyclic ethers (e.g., tetrahydropyranyl, tetrahydropyranyl), and halo(tetrahydropyranylidene) lower alkyl (e.g., fluoro(4-tetrahydropyranylidene)methyl), along with solubilizing groups other than those specifically named above, particularly cyclic solubilizing groups. Exemplary monocyclyl groups include substituted or unsubstituted heterocycles such as thiazolyl, oxazolyl, oxazinyl, thiazinyl, dithianyl, dioxanyl, isoxazolyl, isothiozolyl, triazolyl, furanyl, tetrahydrofuranyl, dihydrofuranyl, pyranyl, tetrazolyl, pyrazolyl, pyrazinyl, pyridazinyl, imidazolyl, pyridinyl, pyrrolyl, dihydropyrrolyl, pyrrolidinyl, thiazinyl, thiadiazolyl, oxazinyl, piperidinyl, piperazinyl, pyrimidinyl, morpholinyl, tetrahydrothiophenyl, thiophenyl, cyclohexyl, cyclopentyl, cyclopropyl, cyclobutyl, cycloheptanyl, azetidinyl, oxetanyl, thiiranyl, oxiranyl, aziridinyl, and thiomorpholinyl.

Aromatic (aryl) groups include carbocyclic aromatic groups such as phenyl, naphthyl, and anthracyl, and heteroaryl groups such as imidazolyl, thienyl, furyl, pyridyl, pyrimidyl, pyranyl, pyrazolyl, pyrroyl, pyrazinyl, thiazolyl, oxazolyl, and tetrazolyl.

Aromatic groups also include fused polycyclic aromatic ring systems in which a carbocyclic aromatic ring or heteroaryl ring is fused to one or more other heteroaryl rings. Examples include benzothienyl, benzofuryl, indolyl, quinolinyl, benzothiazole, benzoxazole, benzimidazole, quinolinyl, isoquinolinyl and isoindolyl.

Suitable substituents on an alkyl, alkenyl, alkynyl, monocyclyl or aryl group (carbocyclic and heteroaryl) are those which do not substantially interfere with the ability of the disclosed compounds to have one or more of the properties disclosed herein. A substituent substantially interferes with the properties of a compound when the magnitude of the property is reduced by more than about 50% in a compound with the substituent compared with a compound without the substituent. Examples of generally suitable substituents include —OH, halogen (—Br, —Cl, —I and —F), —OR^{a}, —O—COR^{a}, —COR^{a}, —C(O)R^{a}, —CN, —NO², —COOH, —COOR^{a}, —OCO₂R^{a}, —C(O)NR^{a}R^{b}, —OC(O)NR^{a}R^{b}, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}, —NRCOR^{a}, —NHCONH₂, —NHCONR^{a}H, —NHCON(R^{a}R^{b}), —NR^{c}CONH₂, —NR^{c}CONR^{a}H, —NR^{c}CON(R^{a}R^{b}), —C(═NH)—NH₂, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(═NR^{c})—N(R^{a}R^{b}), —NR^{d}H—C(═NH)—NH₂, —NR^{d}—C(═NH)—NHR^{a}, —NR^{d}—C(═NH)—N(R^{a}R^{b}), —NR^{d}—C(═NR^{c})—NH₂, —NR^{d}—C(═NR^{c})—NHR^{a}, —NR^{d}C(═NR^{c})—N(R^{a}R^{b}), —NHNH₂, —NHNHR^{a}, —NHR^{a}R^{b}, —SO₂NH₂, —SO₂NHRₐ, —SO₂NR^{a}R^{b}, —CH═CHR^{a}, —CH═CR^{a}R^{b}, —CR^{c}═CR^{a}R^{b}, CR^{c}═CHR^{a}, —CR^{c}═CR^{a}R^{b}, —CCR^{a}, —SH, —SOₖR^{a} (k is 0, 1 or 2), —S(O)ₖOR^{a} (k is 0, 1 or 2) and —NH—C(═NH)—NH₂. R^{a}-R^{d} are each independently an optionally substituted group selected from an aliphatic, benzyl, or aromatic group, preferably an alkyl, benzylic or aryl group. Optional substituents on R^{a}-R^{d} are selected from NH₂, NH(C₁₋₄aliphatic), N(C₁₋₄aliphatic)₂, halogen, C₁₋₄aliphatic, OH, O(C₁₋₄aliphatic), NO₂, CN, CO₂H, CO₂(C₁₋₄aliphatic), O(haloC₁₋₄ aliphatic), or haloC₁₋₄aliphatic, wherein each of the foregoing C₁₋₄aliphatic groups of is unsubstituted. In addition, —NR^{a}R^{b}, taken together, can also form a substituted or unsubstituted non-aromatic heterocyclic group. A non-aromatic heterocyclic group, or aryl group can also have an aliphatic or substituted aliphatic group as a substituent. A substituted aliphatic group can also have a non-aromatic heterocyclic ring, a substituted a non-aromatic heterocyclic ring, aryl or substituted aryl group as a substituent. A substituted aliphatic, non-aromatic heterocyclic group, substituted aryl, or substituted benzyl group can have more than one substituent.

Generally suitable substituents on an aryl ring are selected from a solubilizing group, halogen; —R^{o}; —OR^{o}; —SR^{o}; 1,2-methylenedioxy; 1,2-ethylenedioxy; phenyl (Ph) optionally substituted with R^{o}; —O(Ph) optionally substituted with R^{o}; —(CH₂)₁₋₂(Ph), optionally substituted with R^{o}; —CH═CH(Ph), optionally substituted with R^{o}; —NO₂; —CN; —N(R^{o})₂; —C(O)C(O)R^{o}; —C(O)CH₂C(O)R^{o}; —CO₂R^{o}; —C(O)R^{o}; —S(O)₂R^{o}; —SO₂N(R^{o})₂; —S(O)R^{o}; —NR^{o}SO₂N(R^{o})₂; —NR^{o}SO₂R^{o}; —C(═S)N(R^{o})₂; or —C(═NH)—N(R^{o})₂; or wherein each independent occurrence of R^{o} is selected from hydrogen, optionally substituted C₁₋₆ aliphatic, an unsubstituted 5-6 membered heteroaryl or heterocyclic ring, phenyl, —O(Ph), or —CH₂(Ph), or, notwithstanding the definition above, two independent occurrences of R^{o}, on the same substituent or different substituents, taken together with the atom(s) to which each R^{o} group is bound, form a 3-8-membered cycloalkyl, heterocyclyl, aryl, or heteroaryl ring having 0-3 heteroatoms independently selected from nitrogen, oxygen, or sulfur. Optional substituents on the aliphatic group of R^{o} are selected from NH₂, NH(C₁₋₄aliphatic), N(C₁₋₄aliphatic)₂, halogen, C₁₋₄aliphatic, OH, O(C₁₋₄aliphatic), NO₂, CN, CO₂H, CO₂(C₁₋₄aliphatic), O(haloC₁₋₄ aliphatic), or haloC₁₋₄aliphatic, wherein each of the foregoing C₁₋₄aliphatic groups of R^{o} is unsubstituted

Combinations of substituents and variables envisioned by this invention are only those that result in the formation of stable compounds. As used herein, the term “stable” refers to compounds that possess stability sufficient to allow manufacture and that maintain the integrity of the compound for a sufficient period of time to be useful for the purposes detailed herein.

As used herein, a “solubilizing group” is a moiety that has hydrophilic character sufficient to improve or increase the water-solubility of the compound in which it is included, as compared to an analog compound that does not include the group. The hydrophilic character can be achieved by any means, such as by the inclusion of functional groups that ionize under the conditions of use to form charged moieties (e.g., carboxylic acids, sulfonic acids, phosphoric acids, amines, etc.); groups that include permanent charges (e.g., quaternary ammonium groups); and/or heteroatoms or heteroatomic groups (e.g., O, S, N, NH, N—(CH₂)_{y}—R^{a}, N—(CH₂)_{y}—C(O)R^{a}, N—(CH₂)_{y}—C(O)OR^{a}, N—(CH₂)_{y}—S(O)₂R^{a}—, N—(CH₂)_{y}—S(O)₂OR^{a}, N—(CH₂)_{y}—C(O)NR^{a}R^{a}, etc., wherein R^{a} is selected from hydrogen, lower alkyl, lower cycloalkyl, (C6-C14) aryl, phenyl, naphthyl, (C7-C20) arylalkyl and benzyl, wherein R^{a} is optionally substituted; and y is an integer ranging from 0 to 6), optionally substituted heterocyclic groups (e.g., —(CH₂)ₙ—R^{b}, —(CH₂)ₙ—C(O)—R^{b}, —(CH₂)ₙ—O—(CH₂)ₙ—R^{b}, wherein R^{b} is selected from an optionally substituted saturated monocyclic heterocycle, an optionally substituted saturated bicyclic fused heterocycle, an optionally substituted saturated bicyclic spiro heterocycle, an optionally substituted heteroaryl and an optionally substituted partially substituted non-aryl heterocycle; and n is an integer ranging from 0 to 2). It should be understood that substituents present on R^{a} or R^{b} need not improve or increase water solubility over their unsubstituted counterparts to be within the scope of this definition. All that is required is that such substituents do not significantly reverse the improvement in water-solubility afforded by the unsubstituted R^{a} or R^{b} moiety.

In one embodiment, the solubilizing group increases the water-solubility of the corresponding compound lacking the solubilizing group at least 5-fold, preferably at least 10-fold, more preferably at least 20-fold and most preferably at least 50-fold.

In one preferred embodiment, the solubilizing group is a moiety of the formula:

—(CH₂)ₙ—R¹⁰⁰—N(R¹⁰¹)(R¹⁰¹), where:

n is selected from 0, 1 or 2;
R¹⁰⁰ is selected from a bond, —C(O)—, or —O(CH₂)ₙ; and
each R¹⁰¹ is independently selected from:
a. hydrogen;
b. C₁-C₄ straight or branched alkyl, wherein said alkyl is optionally substituted with halo, CN, OH, O—(C₁-C₄ straight or branched alkyl), N(R₁′)(R₁′), or ═O;

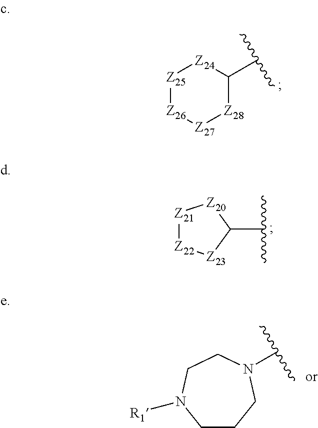

f. both R¹⁰¹ moieties are taken together with the nitrogen atom to which they are bound to form a ring of the structure

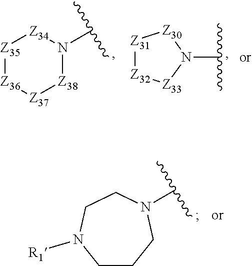

g. both R¹⁰¹ moieties are taken together with the nitrogen atom to which they are bound to form a 5-membered heteroaryl ring containing 1 to 3 additional N atoms, wherein said heteroaryl ring is optionally substituted with R₁′;
   wherein:

each Z is independently selected from —O—, —S—, —NR₁′—, or —C(R⁵⁰)(R⁵⁰)—, wherein:
at least three of Z₂₀, Z₂₁, Z₂₂, and Z₂₃ are —C(R⁵⁰)(R⁵⁰)—;
at least three of Z₂₄, Z₂₅, Z₂₆, Z₂₇, and Z₂₈ are —C(R⁵⁰)(R⁵⁰)—;
at least four of Z₃₀, Z₃₁, Z₃₂, and Z₃₃ are —C(R⁵⁰)(R⁵⁰)—; and
at least four of Z₃₄, Z₃₅, Z₃₆, Z₃₇, and Z₃₈ are —C(R⁵⁰)(R⁵⁰)—;

each R₁′ is independently selected from hydrogen or a C₁-C₃ straight or branched alkyl optionally substituted with one or more substituent independently selected from halo, —CN, —OH, —OCH₃, —NH₂, —NH(CH₃), —N(CH₃)₂, or ═O;

each R⁵⁰ is independently selected from R₁′, halo, CN, OH, O—(C₁-C₄ straight or branched alkyl), N(R₁′)(R₁′), ═CR₁′, SR₁′, ═NR₁′, ═NOR₁′, or ═O;

any two suitable non-cyclic R⁵⁰ are optionally bound to one another directly or via a C₁ to C₂ alkylene, alkenylene or alkanediylidene bridge to produce a bicyclic fused or Spiro ring; and

any

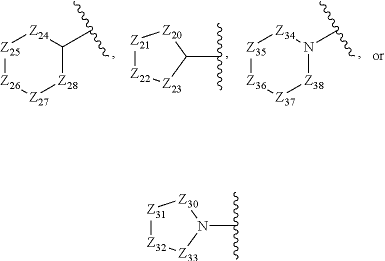

ring structure is optionally benzofused or fused to a monocyclic heteroaryl to produce a bicyclic ring.

For clarity, the term “C₁ to C₂ alkylene, alkenylene or alkanediylidene bridge” means the multivalent structures —CH₂—, —CH₂—CH₂—, —CH═, ═CH—, —CH═CH—, or ═CH—CH═. The two R⁵⁰ moieties that are optionally bound to one another can be either on the same carbon atom or different carbon atoms. The former produces a Spiro bicyclic ring, while the latter produces a fused bicyclic ring. It will be obvious to those of skill in the art that when two R⁵⁰ are bound to one another to form a ring (whether directly or through one of the recited bridges), one or more terminal hydrogen atoms on each R⁵⁰ will be lost. Accordingly, a “suitable non-cyclic R⁵⁰” moiety available for forming a ring is a non-cyclic R⁵⁰ that comprises at least one terminal hydrogen atom.

In another embodiment, the solubilizing group is a moiety of the formula: —(CH₂)ₙ—O—R¹⁰¹, wherein n and R¹⁰¹ are as defined above.

In yet another embodiment, the solubilizing group is a moiety of the formula: —(CH₂)ₙ—C(O)—R₁′, wherein n and R₁′ are as defined above.

In certain embodiments, a solubilizing group is selected from —(CH₂)ₙ—R¹⁰², wherein n is 0, 1 or 2, preferably 2; and R¹⁰² is selected from

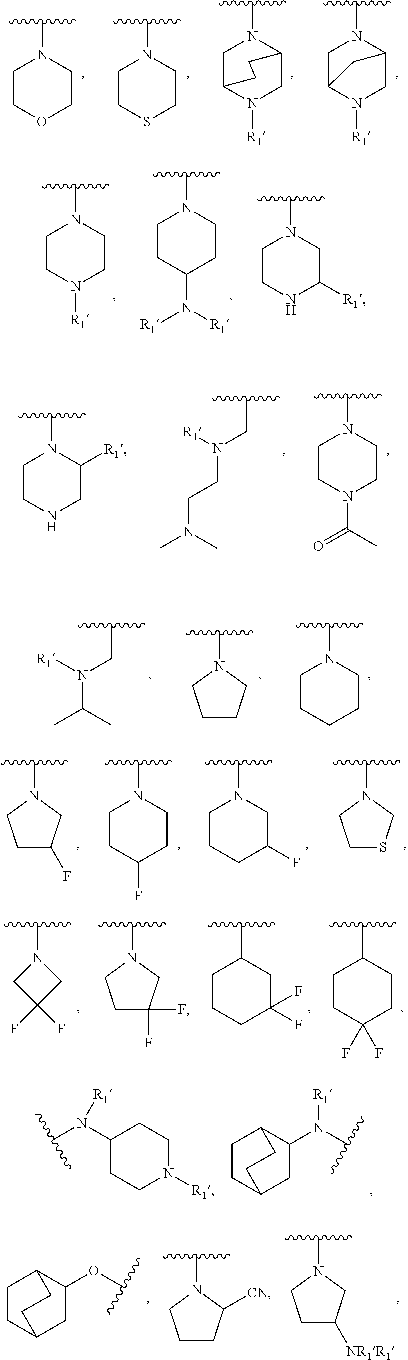

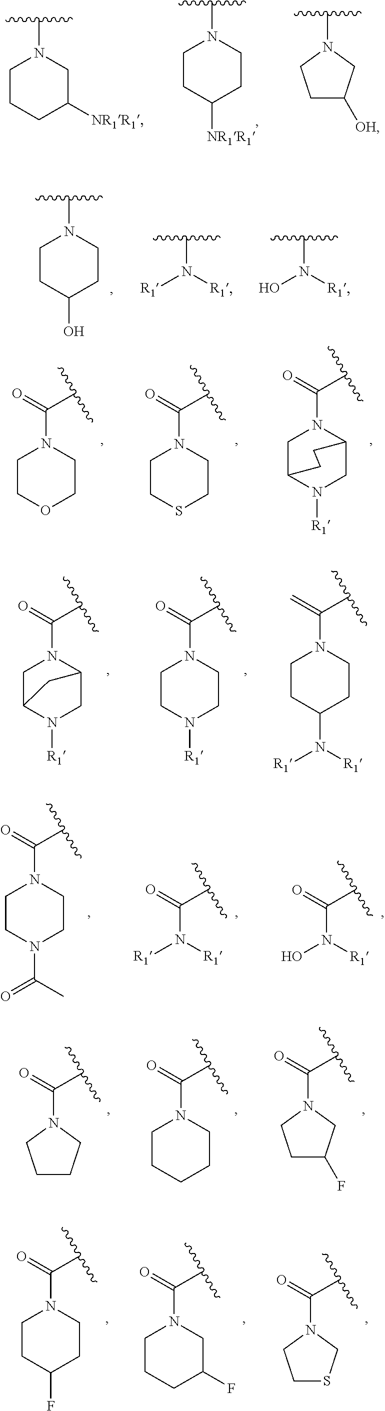

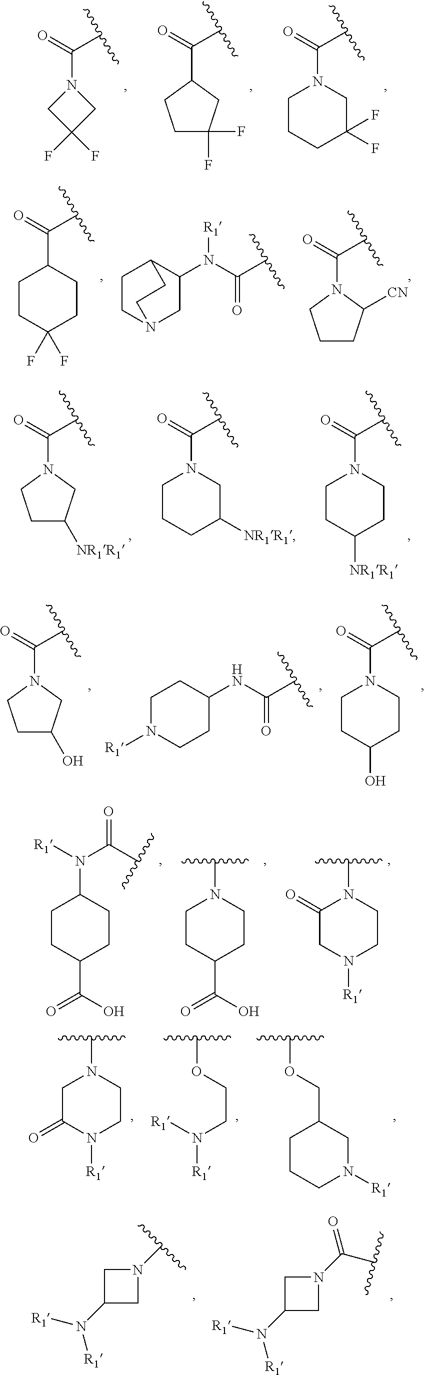

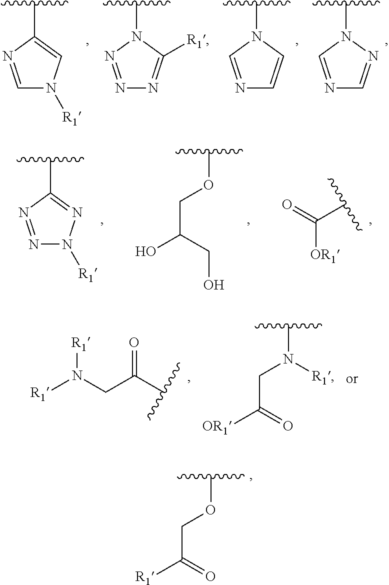

wherein R₁′ groups are as defined above.

In certain particular embodiments, a solubilizing group is selected from 2-dimethylaminoethylcarbamoyl, piperazin-1-ylcarbonyl, piperazinylmethyl, dimethylaminomethyl, 4-methylpiperazin-1-ylmethyl, 4-aminopiperidin-1-yl-methyl, 4-fluoropiperidin-1-yl-methyl, morpholinomethyl, pyrrolidin-1-ylmethyl, 2-oxo-4-benzylpiperazin-1-ylmethyl, 4-benzylpiperazin-1-ylmethyl, 3-oxopiperazin-1-ylmethyl, piperidin-1-ylmethyl, piperazin-1-ylethyl, 2,3-dioxopropylaminomethyl, thiazolidin-3-ylmethyl, 4-acetylpiperazin-1-ylmethyl, 4-acetylpiperazin-1-yl, morpholino, 3,3-difluoroazetidin-1-ylmethyl, 2H-tetrazol-5-ylmethyl, thiomorpholin-4-ylmethyl, 1-oxothiomorpholin-4-ylmethyl, 1,1-dioxothiomorpholin-4-ylmethyl, 1H-imidazol-1-ylmethyl, 3,5-dimethylpiperazin-1 ylmethyl, 4-hydroxypiperidin-1-ylmethyl, N-methyl(1-acetylpiperidin-4-yl)-aminomethyl, N-methylquinuclidin-3-ylaminomethyl, 1H-1,2,4-triazol-1-ylmethyl, 1-methylpiperidin-3-yl-oxymethyl, or 4-fluoropiperidin-1-yl.

To the extent not included within any of the definitions set forth above, the term “solubilizing group” also includes moieties disclosed as being attached to the 7-position of 1-cyclopropyl-6-fluoro-1,4-dihydro-4-oxoquinoline-3-carboxylic acid (ciprofloxacin) and its derivatives, as disclosed in PCT publications WO 2005/026165, WO 2005/049602, and WO 2005/033108, and European Patent publications EP 0343524, EP 0688772, EP 0153163, EP 0159174; as well as “water-solubilizing groups” described in United States patent publication 2006/0035891. The disclosure of each of these patent publications is incorporated herein by reference.

The compounds disclosed herein also include partially and fully deuterated variants. In certain embodiments, one or more deuterium atoms are present for kinetic studies. One of ordinary skill in the art can select the sites at which such deuterium atoms are present.

Also included in the present invention are salts, particularly pharmaceutically acceptable salts, of the sirtuin-modulating compounds described herein. The compounds of the present invention that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion (e.g., a halide such as bromide, chloride, or fluoride, particularly bromide).

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

According to another embodiment, the present invention provides methods of producing the above-defined sirtuin-modulating compounds. The compounds may be synthesized using conventional techniques. Advantageously, these compounds are conveniently synthesized from readily available starting materials.

Synthetic chemistry transformations and methodologies useful in synthesizing the sirtuin-modulating compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

In an exemplary embodiment, a sirtuin-modulating compound may traverse the cytoplasmic membrane of a cell. For example, a compound may have a cell-permeability of at least about 20%, 50%, 75%, 80%, 90% or 95%.

Sirtuin-modulating compounds described herein may also have one or more of the following characteristics: the compound may be essentially non-toxic to a cell or subject; the sirtuin-modulating compound may be an organic molecule or a small molecule of 2000 amu or less, 1000 amu or less; a compound may have a half-life under normal atmospheric conditions of at least about 30 days, 60 days, 120 days, 6 months or 1 year; the compound may have a half-life in solution of at least about 30 days, 60 days, 120 days, 6 months or 1 year; a sirtuin-modulating compound may be more stable in solution than resveratrol by at least a factor of about 50%, 2 fold, 5 fold, 10 fold, 30 fold, 50 fold or 100 fold; a sirtuin-modulating compound may promote deacetylation of the DNA repair factor Ku70; a sirtuin-modulating compound may promote deacetylation of RelA/p65; a compound may increase general turnover rates and enhance the sensitivity of cells to TNF-induced apoptosis.

In certain embodiments, a sirtuin-modulating compound does not have any substantial ability to inhibit a histone deacetylase (HDACs) class I, a HDAC class II, or HDACs I and II, at concentrations (e.g., in vivo) effective for modulating the deacetylase activity of the sirtuin. For instance, in preferred embodiments the sirtuin-modulating compound is a sirtuin-activating compound and is chosen to have an EC₅₀ for activating sirtuin deacetylase activity that is at least 5 fold less than the EC₅₀ for inhibition of an HDAC I and/or HDAC II, and even more preferably at least 10 fold, 100 fold or even 1000 fold less. Methods for assaying HDAC I and/or HDAC II activity are well known in the art and kits to perform such assays may be purchased commercially. See e.g., BioVision, Inc. (Mountain View, Calif.; world wide web at biovision.com) and Thomas Scientific (Swedesboro, N.J.; world wide web at tomassci.com).

In certain embodiments, a sirtuin-modulating compound does not have any substantial ability to modulate sirtuin homologs. In one embodiment, an activator of a human sirtuin protein may not have any substantial ability to activate a sirtuin protein from lower eukaryotes, particularly yeast or human pathogens, at concentrations (e.g., in vivo) effective for activating the deacetylase activity of human sirtuin. For example, a sirtuin-activating compound may be chosen to have an EC₅₀ for activating a human sirtuin, such as SIRT1 and/or SIRT3, deacetylase activity that is at least 5 fold less than the EC₅₀ for activating a yeast sirtuin, such as Sir2 (such as Candida, S. cerevisiae, etc.), and even more preferably at least 10 fold, 100 fold or even 1000 fold less. In another embodiment, an inhibitor of a sirtuin protein from lower eukaryotes, particularly yeast or human pathogens, does not have any substantial ability to inhibit a sirtuin protein from humans at concentrations (e.g., in vivo) effective for inhibiting the deacetylase activity of a sirtuin protein from a lower eukaryote. For example, a sirtuin-inhibiting compound may be chosen to have an IC₅₀ for inhibiting a human sirtuin, such as SIRT1 and/or SIRT3, deacetylase activity that is at least 5 fold less than the IC₅₀ for inhibiting a yeast sirtuin, such as Sir2 (such as Candida, S. cerevisiae, etc.), and even more preferably at least 10 fold, 100 fold or even 1000 fold less.

In certain embodiments, a sirtuin-modulating compound may have the ability to modulate one or more sirtuin protein homologs, such as, for example, one or more of human SIRT1, SIRT2, SIRT3, SIRT4, SIRT5, SIRT6, or SIRT7. In one embodiment, a sirtuin-modulating compound has the ability to modulate both a SIRT1 and a SIRT3 protein.

In other embodiments, a SIRT1 modulator does not have any substantial ability to modulate other sirtuin protein homologs, such as, for example, one or more of human SIRT2, SIRT3, SIRT4, SIRT5, SIRT6, or SIRT7, at concentrations (e.g., in vivo) effective for modulating the deacetylase activity of human SIRT1. For example, a sirtuin-modulating compound may be chosen to have an ED₅₀ for modulating human SIRT1 deacetylase activity that is at least 5 fold less than the ED₅₀ for modulating one or more of human SIRT2, SIRT3, SIRT4, SIRT5, SIRT6, or SIRT7, and even more preferably at least 10 fold, 100 fold or even 1000 fold less. In one embodiment, a SIRT1 modulator does not have any substantial ability to modulate a SIRT3 protein.

In other embodiments, a SIRT3 modulator does not have any substantial ability to modulate other sirtuin protein homologs, such as, for example, one or more of human SIRT1, SIRT2, SIRT4, SIRT5, SIRT6, or SIRT7, at concentrations (e.g., in vivo) effective for modulating the deacetylase activity of human SIRT3. For example, a sirtuin-modulating compound may be chosen to have an ED₅₀ for modulating human SIRT3 deacetylase activity that is at least 5 fold less than the ED₅₀ for modulating one or more of human SIRT1, SIRT2, SIRT4, SIRT5, SIRT6, or SIRT7, and even more preferably at least 10 fold, 100 fold or even 1000 fold less. In one embodiment, a SIRT3 modulator does not have any substantial ability to modulate a SIRT1 protein.

In certain embodiments, a sirtuin-modulating compound may have a binding affinity for a sirtuin protein of about 10⁻⁹M, 10⁻¹⁰M, 10⁻¹¹M, 10⁻¹²M or less. A sirtuin-modulating compound may reduce (activator) or increase (inhibitor) the apparent Km of a sirtuin protein for its substrate or NAD+ (or other cofactor) by a factor of at least about 2, 3, 4, 5, 10, 20, 30, 50 or 100. In certain embodiments, Km values are determined using the mass spectrometry assay described herein. Preferred activating compounds reduce the Km of a sirtuin for its substrate or cofactor to a greater extent than caused by resveratrol at a similar concentration or reduce the Km of a sirtuin for its substrate or cofactor similar to that caused by resveratrol at a lower concentration. A sirtuin-modulating compound may increase the Vmax of a sirtuin protein by a factor of at least about 2, 3, 4, 5, 10, 20, 30, 50 or 100. A sirtuin-modulating compound may have an ED50 for modulating the deacetylase activity of a SIRT1 and/or SIRT3 protein of less than about 1 nM, less than about 10 nM, less than about 100 nM, less than about 1 μM, less than about 10 μM, less than about 100 μM, or from about 1-10 nM, from about 10-100 nM, from about 0.1-1 μM, from about 1-10 μM or from about 10-100 μM. A sirtuin-modulating compound may modulate the deacetylase activity of a SIRT1 and/or SIRT3 protein by a factor of at least about 5, 10, 20, 30, 50, or 100, as measured in a cellular assay or in a cell based assay. A sirtuin-activating compound may cause at least about 10%, 30%, 50%, 80%, 2 fold, 5 fold, 10 fold, 50 fold or 100 fold greater induction of the deacetylase activity of a sirtuin protein relative to the same concentration of resveratrol. A sirtuin-modulating compound may have an ED50 for modulating SIRT5 that is at least about 10 fold, 20 fold, 30 fold, 50 fold greater than that for modulating SIRT1 and/or SIRT3.

### 3. Exemplary Uses

In certain aspects, the invention provides methods for modulating the level and/or activity of a sirtuin protein and methods of use thereof.

In certain embodiments, the invention provides methods for using sirtuin-modulating compounds wherein the sirtuin-modulating compounds activate a sirtuin protein, e.g., increase the level and/or activity of a sirtuin protein. Sirtuin-modulating compounds that increase the level and/or activity of a sirtuin protein may be useful for a variety of therapeutic applications including, for example, increasing the lifespan of a cell, and treating and/or preventing a wide variety of diseases and disorders including, for example, diseases or disorders related to aging or stress, diabetes, obesity, neurodegenerative diseases, cardiovascular disease, blood clotting disorders, inflammation, cancer, and/or flushing, etc. The methods comprise administering to a subject in need thereof a pharmaceutically effective amount of a sirtuin-modulating compound, e.g., a sirtuin-activating compound.

While Applicants do not wish to be bound by theory, it is believed that activators of the instant invention may interact with a sirtuin at the same location within the sirtuin protein (e.g., active site or site affecting the Km or Vmax of the active site). It is believed that this is the reason why certain classes of sirtuin activators and inhibitors can have substantial structural similarity.

In certain embodiments, the sirtuin-modulating compounds described herein may be taken alone or in combination with other compounds. In one embodiment, a mixture of two or more sirtuin-modulating compounds may be administered to a subject in need thereof. In another embodiment, a sirtuin-modulating compound that increases the level and/or activity of a sirtuin protein may be administered with one or more of the following compounds: resveratrol, butein, fisetin, piceatannol, or quercetin. In an exemplary embodiment, a sirtuin-modulating compound that increases the level and/or activity of a sirtuin protein may be administered in combination with nicotinic acid. In another embodiment, a sirtuin-modulating compound that decreases the level and/or activity of a sirtuin protein may be administered with one or more of the following compounds: nicotinamide (NAM), suranim; NF023 (a G-protein antagonist); NF279 (a purinergic receptor antagonist); Trolox (6-hydroxy-2,5,7,8,tetramethylchroman-2-carboxylic acid); (−)-epigallocatechin (hydroxy on sites 3,5,7,3′,4′, 5′); (−)-epigallocatechin gallate (Hydroxy sites 5,7,3′,4′,5′ and gallate ester on 3); cyanidin choloride (3,5,7,3′,4′-pentahydroxyflavylium chloride); delphinidin chloride (3,5,7,3′,4′,5′-hexahydroxyflavylium chloride); myricetin (cannabiscetin; 3,5,7,3′,4′,5′-hexahydroxyflavone); 3,7,3′,4′,5′-pentahydroxyflavone; gossypetin (3,5,7,8,3′,4′-hexahydroxyflavone), sirtinol; and splitomicin. In yet another embodiment, one or more sirtuin-modulating compounds may be administered with one or more therapeutic agents for the treatment or prevention of various diseases, including, for example, cancer, diabetes, neurodegenerative diseases, cardiovascular disease, blood clotting, inflammation, flushing, obesity, ageing, stress, etc. In various embodiments, combination therapies comprising a sirtuin-modulating compound may refer to (1) pharmaceutical compositions that comprise one or more sirtuin-modulating compounds in combination with one or more therapeutic agents (e.g., one or more therapeutic agents described herein); and (2) co-administration of one or more sirtuin-modulating compounds with one or more therapeutic agents wherein the sirtuin-modulating compound and therapeutic agent have not been formulated in the same compositions (but may be present within the same kit or package, such as a blister pack or other multi-chamber package; connected, separately sealed containers (e.g., foil pouches) that can be separated by the user; or a kit where the sirtuin modulating compound(s) and other therapeutic agent(s) are in separate vessels). When using separate formulations, the sirtuin-modulating compound may be administered at the same, intermittent, staggered, prior to, subsequent to, or combinations thereof, with the administration of another therapeutic agent.

In certain embodiments, methods for reducing, preventing or treating diseases or disorders using a sirtuin-modulating compound may also comprise increasing the protein level of a sirtuin, such as human SIRT1, SIRT2 and/or SIRT3, or homologs thereof. Increasing protein levels can be achieved by introducing into a cell one or more copies of a nucleic acid that encodes a sirtuin. For example, the level of a sirtuin can be increased in a mammalian cell by introducing into the mammalian cell a nucleic acid encoding the sirtuin, e.g., increasing the level of SIRT1 by introducing a nucleic acid encoding the amino acid sequence set forth in GenBank Accession No. NP_{—}036370 and/or increasing the level of SIRT3 by introducing a nucleic acid encoding the amino acid sequence set forth in GenBank Accession No. AAH01042.

A nucleic acid that is introduced into a cell to increase the protein level of a sirtuin may encode a protein that is at least about 80%, 85%, 90%, 95%, 98%, or 99% identical to the sequence of a sirtuin, e.g., SIRT1 and/or SIRT3 protein. For example, the nucleic acid encoding the protein may be at least about 80%, 85%, 90%, 95%, 98%, or 99% identical to a nucleic acid encoding a SIRT1 (e.g. GenBank Accession No. NM_{—}012238) and/or SIRT3 (e.g., GenBank Accession No. BC001042) protein. The nucleic acid may also be a nucleic acid that hybridizes, preferably under stringent hybridization conditions, to a nucleic acid encoding a wild-type sirtuin, e.g., SIRT1 and/or SIRT3 protein. Stringent hybridization conditions may include hybridization and a wash in 0.2×SSC at 65° C. When using a nucleic acid that encodes a protein that is different from a wild-type sirtuin protein, such as a protein that is a fragment of a wild-type sirtuin, the protein is preferably biologically active, e.g., is capable of deacetylation. It is only necessary to express in a cell a portion of the sirtuin that is biologically active. For example, a protein that differs from wild-type SIRT1 having GenBank Accession No. NP_{—}036370, preferably contains the core structure thereof. The core structure sometimes refers to amino acids 62-293 of GenBank Accession No. NP_{—}036370, which are encoded by nucleotides 237 to 932 of GenBank Accession No. NM_{—}012238, which encompasses the NAD binding as well as the substrate binding domains. The core domain of SIRT1 may also refer to about amino acids 261 to 447 of GenBank Accession No. NP_{—}036370, which are encoded by nucleotides 834 to 1394 of GenBank Accession No. NM_{—}012238; to about amino acids 242 to 493 of GenBank Accession No. NP_{—}036370, which are encoded by nucleotides 777 to 1532 of GenBank Accession No. NM_{—}012238; or to about amino acids 254 to 495 of GenBank Accession No. NP_{—}036370, which are encoded by nucleotides 813 to 1538 of GenBank Accession No. NM_{—}012238. Whether a protein retains a biological function, e.g., deacetylation capabilities, can be determined according to methods known in the art.

In certain embodiments, methods for reducing, preventing or treating diseases or disorders using a sirtuin-modulating compound may also comprise decreasing the protein level of a sirtuin, such as human SIRT1, SIRT2 and/or SIRT3, or homologs thereof. Decreasing a sirtuin protein level can be achieved according to methods known in the art. For example, an siRNA, an antisense nucleic acid, or a ribozyme targeted to the sirtuin can be expressed in the cell. A dominant negative sirtuin mutant, e.g., a mutant that is not capable of deacetylating, may also be used. For example, mutant H363Y of SIRT1, described, e.g., in Luo et al. (2001) Cell 107:137 can be used. Alternatively, agents that inhibit transcription can be used.

Methods for modulating sirtuin protein levels also include methods for modulating the transcription of genes encoding sirtuins, methods for stabilizing/destabilizing the corresponding mRNAs, and other methods known in the art.

### Aging/Stress

In one embodiment, the invention provides a method extending the lifespan of a cell, extending the proliferative capacity of a cell, slowing aging of a cell, promoting the survival of a cell, delaying cellular senescence in a cell, mimicking the effects of calorie restriction, increasing the resistance of a cell to stress, or preventing apoptosis of a cell, by contacting the cell with a sirtuin-modulating compound of the invention that increases the level and/or activity of a sirtuin protein. In an exemplary embodiment, the methods comprise contacting the cell with a sirtuin-activating compound.

The methods described herein may be used to increase the amount of time that cells, particularly primary cells (i.e., cells obtained from an organism, e.g., a human), may be kept alive in a cell culture. Embryonic stem (ES) cells and pluripotent cells, and cells differentiated therefrom, may also be treated with a sirtuin-modulating compound that increases the level and/or activity of a sirtuin protein to keep the cells, or progeny thereof, in culture for longer periods of time. Such cells can also be used for transplantation into a subject, e.g., after ex vivo modification.

In one embodiment, cells that are intended to be preserved for long periods of time may be treated with a sirtuin-modulating compound that increases the level and/or activity of a sirtuin protein. The cells may be in suspension (e.g., blood cells, serum, biological growth media, etc.) or in tissues or organs. For example, blood collected from an individual for purposes of transfusion may be treated with a sirtuin-modulating compound that increases the level and/or activity of a sirtuin protein to preserve the blood cells for longer periods of time. Additionally, blood to be used for forensic purposes may also be preserved using a sirtuin-modulating compound that increases the level and/or activity of a sirtuin protein. Other cells that may be treated to extend their lifespan or protect against apoptosis include cells for consumption, e.g., cells from non-human mammals (such as meat) or plant cells (such as vegetables).

Sirtuin-modulating compounds that increase the level and/or activity of a sirtuin protein may also be applied during developmental and growth phases in mammals, plants, insects or microorganisms, in order to, e.g., alter, retard or accelerate the developmental and/or growth process.

In another embodiment, sirtuin-modulating compounds that increase the level and/or activity of a sirtuin protein may be used to treat cells useful for transplantation or cell therapy, including, for example, solid tissue grafts, organ transplants, cell suspensions, stem cells, bone marrow cells, etc. The cells or tissue may be an autograft, an allograft, a syngraft or a xenograft. The cells or tissue may be treated with the sirtuin-modulating compound prior to administration/implantation, concurrently with administration/implantation, and/or post administration/implantation into a subject. The cells or tissue may be treated prior to removal of the cells from the donor individual, ex vivo after removal of the cells or tissue from the donor individual, or post implantation into the recipient. For example, the donor or recipient individual may be treated systemically with a sirtuin-modulating compound or may have a subset of cells/tissue treated locally with a sirtuin-modulating compound that increases the level and/or activity of a sirtuin protein. In certain embodiments, the cells or tissue (or donor/recipient individuals) may additionally be treated with another therapeutic agent useful for prolonging graft survival, such as, for example, an immunosuppressive agent, a cytokine, an angiogenic factor, etc.

In yet other embodiments, cells may be treated with a sirtuin-modulating compound that increases the level and/or activity of a sirtuin protein in vivo, e.g., to increase their lifespan or prevent apoptosis. For example, skin can be protected from aging (e.g., developing wrinkles, loss of elasticity, etc.) by treating skin or epithelial cells with a sirtuin-modulating compound that increases the level and/or activity of a sirtuin protein. In an exemplary embodiment, skin is contacted with a pharmaceutical or cosmetic composition comprising a sirtuin-modulating compound that increases the level and/or activity of a sirtuin protein. Exemplary skin afflictions or skin conditions that may be treated in accordance with the methods described herein include disorders or diseases associated with or caused by inflammation, sun damage or natural aging. For example, the compositions find utility in the prevention or treatment of contact dermatitis (including irritant contact dermatitis and allergic contact dermatitis), atopic dermatitis (also known as allergic eczema), actinic keratosis, keratinization disorders (including eczema), epidermolysis bullosa diseases (including penfigus), exfoliative dermatitis, seborrheic dermatitis, erythemas (including erythema multiforme and erythema nodosum), damage caused by the sun or other light sources, discoid lupus erythematosus, dermatomyositis, psoriasis, skin cancer and the effects of natural aging. In another embodiment, sirtuin-modulating compounds that increase the level and/or activity of a sirtuin protein may be used for the treatment of wounds and/or burns to promote healing, including, for example, first-, second- or third-degree burns and/or a thermal, chemical or electrical burns. The formulations may be administered topically, to the skin or mucosal tissue.

Topical formulations comprising one or more sirtuin-modulating compounds that increase the level and/or activity of a sirtuin protein may also be used as preventive, e.g., chemopreventive, compositions. When used in a chemopreventive method, susceptible skin is treated prior to any visible condition in a particular individual.

Sirtuin-modulating compounds may be delivered locally or systemically to a subject. In one embodiment, a sirtuin-modulating compound is delivered locally to a tissue or organ of a subject by injection, topical formulation, etc.

In another embodiment, a sirtuin-modulating compound that increases the level and/or activity of a sirtuin protein may be used for treating or preventing a disease or condition induced or exacerbated by cellular senescence in a subject; methods for decreasing the rate of senescence of a subject, e.g., after onset of senescence; methods for extending the lifespan of a subject; methods for treating or preventing a disease or condition relating to lifespan; methods for treating or preventing a disease or condition relating to the proliferative capacity of cells; and methods for treating or preventing a disease or condition resulting from cell damage or death. In certain embodiments, the method does not act by decreasing the rate of occurrence of diseases that shorten the lifespan of a subject. In certain embodiments, a method does not act by reducing the lethality caused by a disease, such as cancer.

In yet another embodiment, a sirtuin-modulating compound that increases the level and/or activity of a sirtuin protein may be administered to a subject in order to generally increase the lifespan of its cells and to protect its cells against stress and/or against apoptosis. It is believed that treating a subject with a compound described herein is similar to subjecting the subject to hormesis, i.e., mild stress that is beneficial to organisms and may extend their lifespan.

Sirtuin-modulating compounds that increase the level and/or activity of a sirtuin protein may be administered to a subject to prevent aging and aging-related consequences or diseases, such as stroke, heart disease, heart failure, arthritis, high blood pressure, and Alzheimer's disease. Other conditions that can be treated include ocular disorders, e.g., associated with the aging of the eye, such as cataracts, glaucoma, and macular degeneration. Sirtuin-modulating compounds that increase the level and/or activity of a sirtuin protein can also be administered to subjects for treatment of diseases, e.g., chronic diseases, associated with cell death, in order to protect the cells from cell death. Exemplary diseases include those associated with neural cell death, neuronal dysfunction, or muscular cell death or dysfunction, such as Parkinson's disease, Alzheimer's disease, multiple sclerosis, amniotropic lateral sclerosis, and muscular dystrophy; AIDS; fulminant hepatitis; diseases linked to degeneration of the brain, such as Creutzfeld-Jakob disease, retinitis pigmentosa and cerebellar degeneration; myelodysplasis such as aplastic anemia; ischemic diseases such as myocardial infarction and stroke; hepatic diseases such as alcoholic hepatitis, hepatitis B and hepatitis C; joint-diseases such as osteoarthritis; atherosclerosis; alopecia; damage to the skin due to UV light; lichen planus; atrophy of the skin; cataract; and graft rejections. Cell death can also be caused by surgery, drug therapy, chemical exposure or radiation exposure.

Sirtuin-modulating compounds that increase the level and/or activity of a sirtuin protein can also be administered to a subject suffering from an acute disease, e.g., damage to an organ or tissue, e.g., a subject suffering from stroke or myocardial infarction or a subject suffering from a spinal cord injury. Sirtuin-modulating compounds that increase the level and/or activity of a sirtuin protein may also be used to repair an alcoholic's liver.

### Cardiovascular Disease

In another embodiment, the invention provides a method for treating and/or preventing a cardiovascular disease by administering to a subject in need thereof a sirtuin-modulating compound that increases the level and/or activity of a sirtuin protein.

Cardiovascular diseases that can be treated or prevented using the sirtuin-modulating compounds that increase the level and/or activity of a sirtuin protein include cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable or preventable using compounds and methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Other vascular diseases that can be treated or prevented include those related to platelet aggregation, the retinal arterioles, the glomerular arterioles, the vasa nervorum, cardiac arterioles, and associated capillary beds of the eye, the kidney, the heart, and the central and peripheral nervous systems. The sirtuin-modulating compounds that increase the level and/or activity of a sirtuin protein may also be used for increasing HDL levels in plasma of an individual.

Yet other disorders that may be treated with sirtuin-modulating compounds that increase the level and/or activity of a sirtuin protein include restenosis, e.g., following coronary intervention, and disorders relating to an abnormal level of high density and low density cholesterol.

In one embodiment, a sirtuin-modulating compound that increases the level and/or activity of a sirtuin protein may be administered as part of a combination therapeutic with another cardiovascular agent. In one embodiment, a sirtuin-modulating compound that increases the level and/or activity of a sirtuin protein may be administered as part of a combination therapeutic with an anti-arrhythmia agent. In another embodiment, a sirtuin-modulating compound that increases the level and/or activity of a sirtuin protein may be administered as part of a combination therapeutic with another cardiovascular agent.

### Cell Death/Cancer

Sirtuin-modulating compounds that increase the level and/or activity of a sirtuin protein may be administered to subjects who have recently received or are likely to receive a dose of radiation or toxin. In one embodiment, the dose of radiation or toxin is received as part of a work-related or medical procedure, e.g., administered as a prophylactic measure. In another embodiment, the radiation or toxin exposure is received unintentionally. In such a case, the compound is preferably administered as soon as possible after the exposure to inhibit apoptosis and the subsequent development of acute radiation syndrome.

Sirtuin-modulating compounds may also be used for treating and/or preventing cancer. In certain embodiments, sirtuin-modulating compounds that increase the level and/or activity of a sirtuin protein may be used for treating and/or preventing cancer. Calorie restriction has been linked to a reduction in the incidence of age-related disorders including cancer. Accordingly, an increase in the level and/or activity of a sirtuin protein may be useful for treating and/or preventing the incidence of age-related disorders, such as, for example, cancer. Exemplary cancers that may be treated using a sirtuin-modulating compound are those of the brain and kidney; hormone-dependent cancers including breast, prostate, testicular, and ovarian cancers; lymphomas, and leukemias. In cancers associated with solid tumors, a modulating compound may be administered directly into the tumor. Cancer of blood cells, e.g., leukemia, can be treated by administering a modulating compound into the blood stream or into the bone marrow. Benign cell growth, e.g., warts, can also be treated. Other diseases that can be treated include autoimmune diseases, e.g., systemic lupus erythematosus, scleroderma, and arthritis, in which autoimmune cells should be removed. Viral infections such as herpes, HIV, adenovirus, and HTLV-1 associated malignant and benign disorders can also be treated by administration of sirtuin-modulating compound. Alternatively, cells can be obtained from a subject, treated ex vivo to remove certain undesirable cells, e.g., cancer cells, and administered back to the same or a different subject.

Chemotherapeutic agents may be co-administered with modulating compounds described herein as having anti-cancer activity, e.g., compounds that induce apoptosis, compounds that reduce lifespan or compounds that render cells sensitive to stress. Chemotherapeutic agents may be used by themselves with a sirtuin-modulating compound described herein as inducing cell death or reducing lifespan or increasing sensitivity to stress and/or in combination with other chemotherapeutics agents. In addition to conventional chemotherapeutics, the sirtuin-modulating compounds described herein may also be used with antisense RNA, RNAi or other polynucleotides to inhibit the expression of the cellular components that contribute to unwanted cellular proliferation.

Combination therapies comprising sirtuin-modulating compounds and a conventional chemotherapeutic agent may be advantageous over combination therapies known in the art because the combination allows the conventional chemotherapeutic agent to exert greater effect at lower dosage. In a preferred embodiment, the effective dose (ED₅₀) for a chemotherapeutic agent, or combination of conventional chemotherapeutic agents, when used in combination with a sirtuin-modulating compound is at least 2 fold less than the ED₅₀ for the chemotherapeutic agent alone, and even more preferably at 5 fold, 10 fold or even 25 fold less. Conversely, the therapeutic index (TI) for such chemotherapeutic agent or combination of such chemotherapeutic agent when used in combination with a sirtuin-modulating compound described herein can be at least 2 fold greater than the TI for conventional chemotherapeutic regimen alone, and even more preferably at 5 fold, 10 fold or even 25 fold greater.

### Neuronal Diseases/Disorders

In certain aspects, sirtuin-modulating compounds that increase the level and/or activity of a sirtuin protein can be used to treat patients suffering from neurodegenerative diseases, and traumatic or mechanical injury to the central nervous system (CNS), spinal cord or peripheral nervous system (PNS). Neurodegenerative disease typically involves reductions in the mass and volume of the human brain, which may be due to the atrophy and/or death of brain cells, which are far more profound than those in a healthy person that are attributable to aging. Neurodegenerative diseases can evolve gradually, after a long period of normal brain function, due to progressive degeneration (e.g., nerve cell dysfunction and death) of specific brain regions. Alternatively, neurodegenerative diseases can have a quick onset, such as those associated with trauma or toxins. The actual onset of brain degeneration may precede clinical expression by many years. Examples of neurodegenerative diseases include, but are not limited to, Alzheimer's disease (AD), Parkinson's disease (PD), Huntington's disease (HD), amyotrophic lateral sclerosis (ALS; Lou Gehrig's disease), diffuse Lewy body disease, chorea-acanthocytosis, primary lateral sclerosis, ocular diseases (ocular neuritis), chemotherapy-induced neuropathies (e.g., from vincristine, paclitaxel, bortezomib), diabetes-induced neuropathies and Friedreich's ataxia. Sirtuin-modulating compounds that increase the level and/or activity of a sirtuin protein can be used to treat these disorders and others as described below.

AD is a CNS disorder that results in memory loss, unusual behavior, personality changes, and a decline in thinking abilities. These losses are related to the death of specific types of brain cells and the breakdown of connections and their supporting network (e.g. glial cells) between them. The earliest symptoms include loss of recent memory, faulty judgment, and changes in personality. PD is a CNS disorder that results in uncontrolled body movements, rigidity, tremor, and dyskinesia, and is associated with the death of brain cells in an area of the brain that produces dopamine. ALS (motor neuron disease) is a CNS disorder that attacks the motor neurons, components of the CNS that connect the brain to the skeletal muscles.

HD is another neurodegenerative disease that causes uncontrolled movements, loss of intellectual faculties, and emotional disturbance. Tay-Sachs disease and Sandhoff disease are glycolipid storage diseases where GM2 ganglioside and related glycolipidssubstrates for β-hexosaminidase accumulate in the nervous system and trigger acute neurodegeneration.

It is well-known that apoptosis plays a role in AIDS pathogenesis in the immune system. However, HIV-1 also induces neurological disease, which can be treated with sirtuin-modulating compounds of the invention.

Neuronal loss is also a salient feature of prion diseases, such as Creutzfeldt-Jakob disease in human, BSE in cattle (mad cow disease), Scrapie Disease in sheep and goats, and feline spongiform encephalopathy (FSE) in cats. Sirtuin-modulating compounds that increase the level and/or activity of a sirtuin protein may be useful for treating or preventing neuronal loss due to these prior diseases.

In another embodiment, a sirtuin-modulating compound that increases the level and/or activity of a sirtuin protein may be used to treat or prevent any disease or disorder involving axonopathy. Distal axonopathy is a type of peripheral neuropathy that results from some metabolic or toxic derangement of peripheral nervous system (PNS) neurons. It is the most common response of nerves to metabolic or toxic disturbances, and as such may be caused by metabolic diseases such as diabetes, renal failure, deficiency syndromes such as malnutrition and alcoholism, or the effects of toxins or drugs. Those with distal axonopathies usually present with symmetrical glove-stocking sensori-motor disturbances. Deep tendon reflexes and autonomic nervous system (ANS) functions are also lost or diminished in affected areas.

Diabetic neuropathies are neuropathic disorders that are associated with diabetes mellitus. Relatively common conditions which may be associated with diabetic neuropathy include third nerve palsy; mononeuropathy; mononeuritis multiplex; diabetic amyotrophy; a painful polyneuropathy; autonomic neuropathy; and thoracoabdominal neuropathy.

Peripheral neuropathy is the medical term for damage to nerves of the peripheral nervous system, which may be caused either by diseases of the nerve or from the side-effects of systemic illness. Major causes of peripheral neuropathy include seizures, nutritional deficiencies, and HIV, though diabetes is the most likely cause.

In an exemplary embodiment, a sirtuin-modulating compound that increases the level and/or activity of a sirtuin protein may be used to treat or prevent multiple sclerosis (MS), including relapsing MS and monosymptomatic MS, and other demyelinating conditions, such as, for example, chromic inflammatory demyelinating polyneuropathy (CIDP), or symptoms associated therewith.

In yet another embodiment, a sirtuin-modulating compound that increases the level and/or activity of a sirtuin protein may be used to treat trauma to the nerves, including, trauma due to disease, injury (including surgical intervention), or environmental trauma (e.g., neurotoxins, alcoholism, etc.).

Sirtuin-modulating compounds that increase the level and/or activity of a sirtuin protein may also be useful to prevent, treat, and alleviate symptoms of various PNS disorders. The term “peripheral neuropathy” encompasses a wide range of disorders in which the nerves outside of the brain and spinal cord—peripheral nerves—have been damaged. Peripheral neuropathy may also be referred to as peripheral neuritis, or if many nerves are involved, the terms polyneuropathy or polyneuritis may be used.

PNS diseases treatable with sirtuin-modulating compounds that increase the level and/or activity of a sirtuin protein include: diabetes, leprosy, Charcot-Marie-Tooth disease, Guillain-Barré syndrome and Brachial Plexus Neuropathies (diseases of the cervical and first thoracic roots, nerve trunks, cords, and peripheral nerve components of the brachial plexus.

In another embodiment, a sirtuin activating compound may be used to treat or prevent a polyglutamine disease. Exemplary polyglutamine diseases include Spinobulbar muscular atrophy (Kennedy disease), Huntington's Disease (HD), Dentatorubral-pallidoluysian atrophy (Haw River syndrome), Spinocerebellar ataxia type 1, Spinocerebellar ataxia type 2, Spinocerebellar ataxia type 3 (Machado-Joseph disease), Spinocerebellar ataxia type 6, Spinocerebellar ataxia type 7, and Spinocerebellar ataxia type 17.

In certain embodiments, the invention provides a method to treat a central nervous system cell to prevent damage in response to a decrease in blood flow to the cell. Typically the severity of damage that may be prevented will depend in large part on the degree of reduction in blood flow to the cell and the duration of the reduction. In one embodiment, apoptotic or necrotic cell death may be prevented. In still a further embodiment, ischemic-mediated damage, such as cytoxic edema or central nervous system tissue anoxemia, may be prevented. In each embodiment, the central nervous system cell may be a spinal cell or a brain cell.

Another aspect encompasses administrating a sirtuin activating compound to a subject to treat a central nervous system ischemic condition. A number of central nervous system ischemic conditions may be treated by the sirtuin activating compounds described herein. In one embodiment, the ischemic condition is a stroke that results in any type of ischemic central nervous system damage, such as apoptotic or necrotic cell death, cytoxic edema or central nervous system tissue anoxia. The stroke may impact any area of the brain or be caused by any etiology commonly known to result in the occurrence of a stroke. In one alternative of this embodiment, the stroke is a brain stem stroke. In another alternative of this embodiment, the stroke is a cerebellar stroke. In still another embodiment, the stroke is an embolic stroke. In yet another alternative, the stroke may be a hemorrhagic stroke. In a further embodiment, the stroke is a thrombotic stroke.

In yet another aspect, a sirtuin activating compound may be administered to reduce infarct size of the ischemic core following a central nervous system ischemic condition. Moreover, a sirtuin activating compound may also be beneficially administered to reduce the size of the ischemic penumbra or transitional zone following a central nervous system ischemic condition.

In one embodiment, a combination drug regimen may include drugs or compounds for the treatment or prevention of neurodegenerative disorders or secondary conditions associated with these conditions. Thus, a combination drug regimen may include one or more sirtuin activators and one or more anti-neurodegeneration agents.

### Blood Coagulation Disorders

In other aspects, sirtuin-modulating compounds that increase the level and/or activity of a sirtuin protein can be used to treat or prevent blood coagulation disorders (or hemostatic disorders). As used interchangeably herein, the terms “hemostasis”, “blood coagulation,” and “blood clotting” refer to the control of bleeding, including the physiological properties of vasoconstriction and coagulation. Blood coagulation assists in maintaining the integrity of mammalian circulation after injury, inflammation, disease, congenital defect, dysfunction or other disruption. Further, the formation of blood clots does not only limit bleeding in case of an injury (hemostasis), but may lead to serious organ damage and death in the context of atherosclerotic diseases by occlusion of an important artery or vein. Thrombosis is thus blood clot formation at the wrong time and place.

Accordingly, the present invention provides anticoagulation and antithrombotic treatments aiming at inhibiting the formation of blood clots in order to prevent or treat blood coagulation disorders, such as myocardial infarction, stroke, loss of a limb by peripheral artery disease or pulmonary embolism.

As used interchangeably herein, “modulating or modulation of hemostasis” and “regulating or regulation of hemostasis” includes the induction (e.g., stimulation or increase) of hemostasis, as well as the inhibition (e.g., reduction or decrease) of hemostasis.

In one aspect, the invention provides a method for reducing or inhibiting hemostasis in a subject by administering a sirtuin-modulating compound that increases the level and/or activity of a sirtuin protein. The compositions and methods disclosed herein are useful for the treatment or prevention of thrombotic disorders. As used herein, the term “thrombotic disorder” includes any disorder or condition characterized by excessive or unwanted coagulation or hemostatic activity, or a hypercoagulable state. Thrombotic disorders include diseases or disorders involving platelet adhesion and thrombus formation, and may manifest as an increased propensity to form thromboses, e.g., an increased number of thromboses, thrombosis at an early age, a familial tendency towards thrombosis, and thrombosis at unusual sites.

In another embodiment, a combination drug regimen may include drugs or compounds for the treatment or prevention of blood coagulation disorders or secondary conditions associated with these conditions. Thus, a combination drug regimen may include one or more sirtuin-modulating compounds that increase the level and/or activity of a sirtuin protein and one or more anti-coagulation or anti-thrombosis agents.

### Weight Control

In another aspect, sirtuin-modulating compounds that increase the level and/or activity of a sirtuin protein may be used for treating or preventing weight gain or obesity in a subject. For example, sirtuin-modulating compounds that increase the level and/or activity of a sirtuin protein may be used, for example, to treat or prevent hereditary obesity, dietary obesity, hormone related obesity, obesity related to the administration of medication, to reduce the weight of a subject, or to reduce or prevent weight gain in a subject. A subject in need of such a treatment may be a subject who is obese, likely to become obese, overweight, or likely to become overweight. Subjects who are likely to become obese or overweight can be identified, for example, based on family history, genetics, diet, activity level, medication intake, or various combinations thereof.

In yet other embodiments, sirtuin-modulating compounds that increase the level and/or activity of a sirtuin protein may be administered to subjects suffering from a variety of other diseases and conditions that may be treated or prevented by promoting weight loss in the subject. Such diseases include, for example, high blood pressure, hypertension, high blood cholesterol, dyslipidemia, type 2 diabetes, insulin resistance, glucose intolerance, hyperinsulinemia, coronary heart disease, angina pectoris, congestive heart failure, stroke, gallstones, cholescystitis and cholelithiasis, gout, osteoarthritis, obstructive sleep apnea and respiratory problems, some types of cancer (such as endometrial, breast, prostate, and colon), complications of pregnancy, poor female reproductive health (such as menstrual irregularities, infertility, irregular ovulation), bladder control problems (such as stress incontinence); uric acid nephrolithiasis; psychological disorders (such as depression, eating disorders, distorted body image, and low self esteem). Finally, patients with AIDS can develop lipodystrophy or insulin resistance in response to combination therapies for AIDS.

In another embodiment, sirtuin-modulating compounds that increase the level and/or activity of a sirtuin protein may be used for inhibiting adipogenesis or fat cell differentiation, whether in vitro or in vivo. Such methods may be used for treating or preventing obesity.

In other embodiments, sirtuin-modulating compounds that increase the level and/or activity of a sirtuin protein may be used for reducing appetite and/or increasing satiety, thereby causing weight loss or avoidance of weight gain. A subject in need of such a treatment may be a subject who is overweight, obese or a subject likely to become overweight or obese. The method may comprise administering daily or, every other day, or once a week, a dose, e.g., in the form of a pill, to a subject. The dose may be an “appetite reducing dose.”

In an exemplary embodiment, sirtuin-modulating compounds that increase the level and/or activity of a sirtuin protein may be administered as a combination therapy for treating or preventing weight gain or obesity. For example, one or more sirtuin-modulating compounds that increase the level and/or activity of a sirtuin protein may be administered in combination with one or more anti-obesity agents.

In another embodiment, sirtuin-modulating compounds that increase the level and/or activity of a sirtuin protein may be administered to reduce drug-induced weight gain. For example, a sirtuin-modulating compound that increases the level and/or activity of a sirtuin protein may be administered as a combination therapy with medications that may stimulate appetite or cause weight gain, in particular, weight gain due to factors other than water retention.

### Metabolic Disorders/Diabetes

In another aspect, sirtuin-modulating compounds that increase the level and/or activity of a sirtuin protein may be used for treating or preventing a metabolic disorder, such as insulin-resistance, a pre-diabetic state, type II diabetes, and/or complications thereof. Administration of a sirtuin-modulating compounds that increases the level and/or activity of a sirtuin protein may increase insulin sensitivity and/or decrease insulin levels in a subject. A subject in need of such a treatment may be a subject who has insulin resistance or other precursor symptom of type II diabetes, who has type II diabetes, or who is likely to develop any of these conditions. For example, the subject may be a subject having insulin resistance, e.g., having high circulating levels of insulin and/or associated conditions, such as hyperlipidemia, dyslipogenesis, hypercholesterolemia, impaired glucose tolerance, high blood glucose sugar level, other manifestations of syndrome X, hypertension, atherosclerosis and lipodystrophy.

In an exemplary embodiment, sirtuin-modulating compounds that increase the level and/or activity of a sirtuin protein may be administered as a combination therapy for treating or preventing a metabolic disorder. For example, one or more sirtuin-modulating compounds that increase the level and/or activity of a sirtuin protein may be administered in combination with one or more anti-diabetic agents.

### Inflammatory Diseases

In other aspects, sirtuin-modulating compounds that increase the level and/or activity of a sirtuin protein can be used to treat or prevent a disease or disorder associated with inflammation. Sirtuin-modulating compounds that increase the level and/or activity of a sirtuin protein may be administered prior to the onset of, at, or after the initiation of inflammation. When used prophylactically, the compounds are preferably provided in advance of any inflammatory response or symptom. Administration of the compounds may prevent or attenuate inflammatory responses or symptoms.

In another embodiment, sirtuin-modulating compounds that increase the level and/or activity of a sirtuin protein may be used to treat or prevent allergies and respiratory conditions, including asthma, bronchitis, pulmonary fibrosis, allergic rhinitis, oxygen toxicity, emphysema, chronic bronchitis, acute respiratory distress syndrome, and any chronic obstructive pulmonary disease (COPD). The compounds may be used to treat chronic hepatitis infection, including hepatitis B and hepatitis C.

Additionally, sirtuin-modulating compounds that increase the level and/or activity of a sirtuin protein may be used to treat autoimmune diseases and/or inflammation associated with autoimmune diseases such as organ-tissue autoimmune diseases (e.g., Raynaud's syndrome), scleroderma, myasthenia gravis, transplant rejection, endotoxin shock, sepsis, psoriasis, eczema, dermatitis, multiple sclerosis, autoimmune thyroiditis, uveitis, systemic lupus erythematosis, Addison's disease, autoimmune polyglandular disease (also known as autoimmune polyglandular syndrome), and Grave's disease.

In certain embodiments, one or more sirtuin-modulating compounds that increase the level and/or activity of a sirtuin protein may be taken alone or in combination with other compounds useful for treating or preventing inflammation.

### Flushing

In another aspect, sirtuin-modulating compounds that increase the level and/or activity of a sirtuin protein may be used for reducing the incidence or severity of flushing and/or hot flashes which are symptoms of a disorder. For instance, the subject method includes the use of sirtuin-modulating compounds that increase the level and/or activity of a sirtuin protein, alone or in combination with other agents, for reducing incidence or severity of flushing and/or hot flashes in cancer patients. In other embodiments, the method provides for the use of sirtuin-modulating compounds that increase the level and/or activity of a sirtuin protein to reduce the incidence or severity of flushing and/or hot flashes in menopausal and post-menopausal woman.

In another aspect, sirtuin-modulating compounds that increase the level and/or activity of a sirtuin protein may be used as a therapy for reducing the incidence or severity of flushing and/or hot flashes which are side-effects of another drug therapy, e.g., drug-induced flushing. In certain embodiments, a method for treating and/or preventing drug-induced flushing comprises administering to a patient in need thereof a formulation comprising at least one flushing inducing compound and at least one sirtuin-modulating compound that increases the level and/or activity of a sirtuin protein. In other embodiments, a method for treating drug induced flushing comprises separately administering one or more compounds that induce flushing and one or more sirtuin-modulating compounds, e.g., wherein the sirtuin-modulating compound and flushing inducing agent have not been formulated in the same compositions. When using separate formulations, the sirtuin-modulating compound may be administered (1) at the same as administration of the flushing inducing agent, (2) intermittently with the flushing inducing agent, (3) staggered relative to administration of the flushing inducing agent, (4) prior to administration of the flushing inducing agent, (5) subsequent to administration of the flushing inducing agent, and (6) various combination thereof. Exemplary flushing inducing agents include, for example, niacin, faloxifene, antidepressants, anti-psychotics, chemotherapeutics, calcium channel blockers, and antibiotics.

In one embodiment, sirtuin-modulating compounds that increase the level and/or activity of a sirtuin protein may be used to reduce flushing side effects of a vasodilator or an antilipemic agent (including anticholesteremic agents and lipotropic agents). In an exemplary embodiment, a sirtuin-modulating compound that increases the level and/or activity of a sirtuin protein may be used to reduce flushing associated with the administration of niacin.

In another embodiment, the invention provides a method for treating and/or preventing hyperlipidemia with reduced flushing side effects. In another representative embodiment, the method involves the use of sirtuin-modulating compounds that increase the level and/or activity of a sirtuin protein to reduce flushing side effects of raloxifene. In another representative embodiment, the method involves the use of sirtuin-modulating compounds that increase the level and/or activity of a sirtuin protein to reduce flushing side effects of antidepressants or anti-psychotic agent. For instance, sirtuin-modulating compounds that increase the level and/or activity of a sirtuin protein can be used in conjunction (administered separately or together) with a serotonin reuptake inhibitor, or a 5HT2 receptor antagonist.

In certain embodiments, sirtuin-modulating compounds that increase the level and/or activity of a sirtuin protein may be used as part of a treatment with a serotonin reuptake inhibitor (SRI) to reduce flushing. In still another representative embodiment, sirtuin-modulating compounds that increase the level and/or activity of a sirtuin protein may be used to reduce flushing side effects of chemotherapeutic agents, such as cyclophosphamide and tamoxifen.

In another embodiment, sirtuin-modulating compounds that increase the level and/or activity of a sirtuin protein may be used to reduce flushing side effects of calcium channel blockers, such as amlodipine.

In another embodiment, sirtuin-modulating compounds that increase the level and/or activity of a sirtuin protein may be used to reduce flushing side effects of antibiotics. For example, sirtuin-modulating compounds that increase the level and/or activity of a sirtuin protein can be used in combination with levofloxacin.

### Ocular Disorders

One aspect of the present invention is a method for inhibiting, reducing or otherwise treating vision impairment by administering to a patient a therapeutic dosage of sirtuin modulator selected from a compound disclosed herein, or a pharmaceutically acceptable salt, prodrug or a metabolic derivative thereof.

In certain aspects of the invention, the vision impairment is caused by damage to the optic nerve or central nervous system. In particular embodiments, optic nerve damage is caused by high intraocular pressure, such as that created by glaucoma. In other particular embodiments, optic nerve damage is caused by swelling of the nerve, which is often associated with an infection or an immune (e.g., autoimmune) response such as in optic neuritis.

In certain aspects of the invention, the vision impairment is caused by retinal damage. In particular embodiments, retinal damage is caused by disturbances in blood flow to the eye (e.g., arteriosclerosis, vasculitis). In particular embodiments, retinal damage is caused by disrupton of the macula (e.g., exudative or non-exudative macular degeneration).

Exemplary retinal diseases include Exudative Age Related Macular Degeneration, Nonexudative Age Related Macular Degeneration, Retinal Electronic Prosthesis and RPE Transplantation Age Related Macular Degeneration, Acute Multifocal Placoid Pigment Epitheliopathy, Acute Retinal Necrosis, Best Disease, Branch Retinal Artery Occlusion, Branch Retinal Vein Occlusion, Cancer Associated and Related Autoimmune Retinopathies, Central Retinal Artery Occlusion, Central Retinal Vein Occlusion, Central Serous Chorioretinopathy, Eales Disease, Epimacular Membrane, Lattice Degeneration, Macroaneurysm, Diabetic Macular Edema, Irvine-Gass Macular Edema, Macular Hole, Subretinal Neovascular Membranes, Diffuse Unilateral Subacute Neuroretinitis, Nonpseudophakic Cystoid Macular Edema, Presumed Ocular Histoplasmosis Syndrome, Exudative Retinal Detachment, Postoperative Retinal Detachment, Proliferative Retinal Detachment, Rhegmatogenous Retinal Detachment, Tractional Retinal Detachment, Retinitis Pigmentosa, CMV Retinitis, Retinoblastoma, Retinopathy of Prematurity, Birdshot Retinopathy, Background Diabetic Retinopathy, Proliferative Diabetic Retinopathy, Hemoglobinopathies Retinopathy, Purtscher Retinopathy, Valsalva Retinopathy, Juvenile Retinoschisis, Senile Retinoschisis, Terson Syndrome and White Dot Syndromes.

Other exemplary diseases include ocular bacterial infections (e.g. conjunctivitis, keratitis, tuberculosis, syphilis, gonorrhea), viral infections (e.g. Ocular Herpes Simplex Virus, Varicella Zoster Virus, Cytomegalovirus retinitis, Human Immunodeficiency Virus (HIV)) as well as progressive outer retinal necrosis secondary to HIV or other HIV-associated and other immunodeficiency-associated ocular diseases. In addition, ocular diseases include fungal infections (e.g. Candida choroiditis, histoplasmosis), protozoal infections (e.g. toxoplasmosis) and others such as ocular toxocariasis and sarcoidosis.

One aspect of the invention is a method for inhibiting, reducing or treating vision impairment in a subject undergoing treatment with a chemotherapeutic drug (e.g., a neurotoxic drug, a drug that raises intraocular pressure such as a steroid), by administering to the subject in need of such treatment a therapeutic dosage of a sirtuin modulator disclosed herein.

Another aspect of the invention is a method for inhibiting, reducing or treating vision impairment in a subject undergoing surgery, including ocular or other surgeries performed in the prone position such as spinal cord surgery, by administering to the subject in need of such treatment a therapeutic dosage of a sirtuin modulator disclosed herein. Ocular surgeries include cataract, iridotomy and lens replacements.

Another aspect of the invention is the treatment, including inhibition and prophylactic treatment, of age related ocular diseases include cataracts, dry eye, age-related macular degeneration (AMD), retinal damage and the like, by administering to the subject in need of such treatment a therapeutic dosage of a sirtuin modulator disclosed herein.

Another aspect of the invention is the prevention or treatment of damage to the eye caused by stress, chemical insult or radiation, by administering to the subject in need of such treatment a therapeutic dosage of a sirtuin modulator disclosed herein. Radiation or electromagnetic damage to the eye can include that caused by CRT's or exposure to sunlight or UV.

In one embodiment, a combination drug regimen may include drugs or compounds for the treatment or prevention of ocular disorders or secondary conditions associated with these conditions. Thus, a combination drug regimen may include one or more sirtuin activators and one or more therapeutic agents for the treatment of an ocular disorder.

In one embodiment, a sirtuin modulator can be administered in conjunction with a therapy for reducing intraocular pressure. In another embodiment, a sirtuin modulator can be administered in conjunction with a therapy for treating and/or preventing glaucoma. In yet another embodiment, a sirtuin modulator can be administered in conjunction with a therapy for treating and/or preventing optic neuritis. In one embodiment, a sirtuin modulator can be administered in conjunction with a therapy for treating and/or preventing CMV Retinopathy. In another embodiment, a sirtuin modulator can be administered in conjunction with a therapy for treating and/or preventing multiple sclerosis.

### Mitochondrial-Associated Diseases and Disorders

In certain embodiments, the invention provides methods for treating diseases or disorders that would benefit from increased mitochondrial activity. The methods involve administering to a subject in need thereof a therapeutically effective amount of a sirtuin activating compound. Increased mitochondrial activity refers to increasing activity of the mitochondria while maintaining the overall numbers of mitochondria (e.g., mitochondrial mass), increasing the numbers of mitochondria thereby increasing mitochondrial activity (e.g., by stimulating mitochondrial biogenesis), or combinations thereof. In certain embodiments, diseases and disorders that would benefit from increased mitochondrial activity include diseases or disorders associated with mitochondrial dysfunction.

In certain embodiments, methods for treating diseases or disorders that would benefit from increased mitochondrial activity may comprise identifying a subject suffering from a mitochondrial dysfunction. Methods for diagnosing a mitochondrial dysfunction may involve molecular genetic, pathologic and/or biochemical analyses. Diseases and disorders associated with mitochondrial dysfunction include diseases and disorders in which deficits in mitochondrial respiratory chain activity contribute to the development of pathophysiology of such diseases or disorders in a mammal. Diseases or disorders that would benefit from increased mitochondrial activity generally include for example, diseases in which free radical mediated oxidative injury leads to tissue degeneration, diseases in which cells inappropriately undergo apoptosis, and diseases in which cells fail to undergo apoptosis.

In certain embodiments, the invention provides methods for treating a disease or disorder that would benefit from increased mitochondrial activity that involves administering to a subject in need thereof one or more sirtuin activating compounds in combination with another therapeutic agent such as, for example, an agent useful for treating mitochondrial dysfunction or an agent useful for reducing a symptom associated with a disease or disorder involving mitochondrial dysfunction.

In exemplary embodiments, the invention provides methods for treating diseases or disorders that would benefit from increased mitochondrial activity by administering to a subject a therapeutically effective amount of a sirtuin activating compound. Exemplary diseases or disorders include, for example, neuromuscular disorders (e.g., Friedreich's Ataxia, muscular dystrophy, multiple sclerosis, etc.), disorders of neuronal instability (e.g., seizure disorders, migrane, etc.), developmental delay, neurodegenerative disorders (e.g., Alzheimer's Disease, Parkinson's Disease, amyotrophic lateral sclerosis, etc.), ischemia, renal tubular acidosis, age-related neurodegeneration and cognitive decline, chemotherapy fatigue, age-related or chemotherapy-induced menopause or irregularities of menstrual cycling or ovulation, mitochondrial myopathies, mitochondrial damage (e.g., calcium accumulation, excitotoxicity, nitric oxide exposure, hypoxia, etc.), and mitochondrial deregulation.

Muscular dystrophy refers to a family of diseases involving deterioration of neuromuscular structure and function, often resulting in atrophy of skeletal muscle and myocardial dysfunction, such as Duchenne muscular dystrophy. In certain embodiments, sirtuin activating compounds may be used for reducing the rate of decline in muscular functional capacities and for improving muscular functional status in patients with muscular dystrophy.

In certain embodiments, sirtuin modulating compounds may be useful for treatment mitochondrial myopathies. Mitochondrial myopathies range from mild, slowly progressive weakness of the extraocular muscles to severe, fatal infantile myopathies and multisystem encephalomyopathies. Some syndromes have been defined, with some overlap between them. Established syndromes affecting muscle include progressive external ophthalmoplegia, the Kearns-Sayre syndrome (with ophthalmoplegia, pigmentary retinopathy, cardiac conduction defects, cerebellar ataxia, and sensorineural deafness), the MELAS syndrome (mitochondrial encephalomyopathy, lactic acidosis, and stroke-like episodes), the MERFF syndrome (myoclonic epilepsy and ragged red fibers), limb-girdle distribution weakness, and infantile myopathy (benign or severe and fatal).

In certain embodiments, sirtuin activating compounds may be useful for treating patients suffering from toxic damage to mitochondria, such as, toxic damage due to calcium accumulation, excitotoxicity, nitric oxide exposure, drug induced toxic damage, or hypoxia.

In certain embodiments, sirtuin activating compounds may be useful for treating diseases or disorders associated with mitochondrial deregulation.

### Muscle Performance

In other embodiments, the invention provides methods for enhancing muscle performance by administering a therapeutically effective amount of a sirtuin activating compound. For example, sirtuin activating compounds may be useful for improving physical endurance (e.g., ability to perform a physical task such as exercise, physical labor, sports activities, etc.), inhibiting or retarding physical fatigues, enhancing blood oxygen levels, enhancing energy in healthy individuals, enhance working capacity and endurance, reducing muscle fatigue, reducing stress, enhancing cardiac and cardiovascular function, improving sexual ability, increasing muscle ATP levels, and/or reducing lactic acid in blood. In certain embodiments, the methods involve administering an amount of a sirtuin activating compound that increase mitochondrial activity, increase mitochondrial biogenesis, and/or increase mitochondrial mass.

Sports performance refers to the ability of the athlete's muscles to perform when participating in sports activities. Enhanced sports performance, strength, speed and endurance are measured by an increase in muscular contraction strength, increase in amplitude of muscle contraction, shortening of muscle reaction time between stimulation and contraction. Athlete refers to an individual who participates in sports at any level and who seeks to achieve an improved level of strength, speed and endurance in their performance, such as, for example, body builders, bicyclists, long distance runners, short distance runners, etc. Enhanced sports performance in manifested by the ability to overcome muscle fatigue, ability to maintain activity for longer periods of time, and have a more effective workout.

In the arena of athlete muscle performance, it is desirable to create conditions that permit competition or training at higher levels of resistance for a prolonged period of time.

It is contemplated that the methods of the present invention will also be effective in the treatment of muscle related pathological conditions, including acute sarcopenia, for example, muscle atrophy and/or cachexia associated with burns, bed rest, limb immobilization, or major thoracic, abdominal, and/or orthopedic surgery.

In certain embodiments, the invention provides novel dietary compositions comprising sirtuin modulators, a method for their preparation, and a method of using the compositions for improvement of sports performance. Accordingly, provided are therapeutic compositions, foods and beverages that have actions of improving physical endurance and/or inhibiting physical fatigues for those people involved in broadly-defined exercises including sports requiring endurance and labors requiring repeated muscle exertions. Such dietary compositions may additional comprise electrolytes, caffeine, vitamins, carbohydrates, etc.

### Other Uses

Sirtuin-modulating compounds that increase the level and/or activity of a sirtuin protein may be used for treating or preventing viral infections (such as infections by influenza, herpes or papilloma virus) or as antifungal agents. In certain embodiments, sirtuin-modulating compounds that increase the level and/or activity of a sirtuin protein may be administered as part of a combination drug therapy with another therapeutic agent for the treatment of viral diseases. In another embodiment, sirtuin-modulating compounds that increase the level and/or activity of a sirtuin protein may be administered as part of a combination drug therapy with another anti-fungal agent.

Subjects that may be treated as described herein include eukaryotes, such as mammals, e.g., humans, ovines, bovines, equines, porcines, canines, felines, non-human primate, mice, and rats. Cells that may be treated include eukaryotic cells, e.g., from a subject described above, or plant cells, yeast cells and prokaryotic cells, e.g., bacterial cells. For example, modulating compounds may be administered to farm animals to improve their ability to withstand farming conditions longer.

Sirtuin-modulating compounds that increase the level and/or activity of a sirtuin protein may also be used to increase lifespan, stress resistance, and resistance to apoptosis in plants. In one embodiment, a compound is applied to plants, e.g., on a periodic basis, or to fungi. In another embodiment, plants are genetically modified to produce a compound. In another embodiment, plants and fruits are treated with a compound prior to picking and shipping to increase resistance to damage during shipping. Plant seeds may also be contacted with compounds described herein, e.g., to preserve them.

In other embodiments, sirtuin-modulating compounds that increase the level and/or activity of a sirtuin protein may be used for modulating lifespan in yeast cells. Situations in which it may be desirable to extend the lifespan of yeast cells include any process in which yeast is used, e.g., the making of beer, yoghurt, and bakery items, e.g., bread. Use of yeast having an extended lifespan can result in using less yeast or in having the yeast be active for longer periods of time. Yeast or other mammalian cells used for recombinantly producing proteins may also be treated as described herein.

Sirtuin-modulating compounds that increase the level and/or activity of a sirtuin protein may also be used to increase lifespan, stress resistance and resistance to apoptosis in insects. In this embodiment, compounds would be applied to useful insects, e.g., bees and other insects that are involved in pollination of plants. In a specific embodiment, a compound would be applied to bees involved in the production of honey. Generally, the methods described herein may be applied to any organism, e.g., eukaryote, that may have commercial importance. For example, they can be applied to fish (aquaculture) and birds (e.g., chicken and fowl).

Higher doses of sirtuin-modulating compounds that increase the level and/or activity of a sirtuin protein may also be used as a pesticide by interfering with the regulation of silenced genes and the regulation of apoptosis during development. In this embodiment, a compound may be applied to plants using a method known in the art that ensures the compound is bio-available to insect larvae, and not to plants.

At least in view of the link between reproduction and longevity, sirtuin-modulating compounds that increase the level and/or activity of a sirtuin protein can be applied to affect the reproduction of organisms such as insects, animals and microorganisms.

### 4. Assays

Various types of assays to determine sirtuin activity have been described. For example, sirtuin activity may be determined using a fluorescence based assay such as the assay commercially available from Biomol, e.g., the SIRT1 Fluorimetric Drug Discovery Kit (AK-555), SIRT2 Fluorimetric Drug Discovery Kit (AK-556), or SIRT3 Fluorimetric Drug Discovery Kit (AK-557) (Biomol International, Plymouth Meeting, Pa.). Other suitable sirtuin assays include a nicotinamide release assay (Kaeberlein et al., J. Biol. Chem. 280(17): 17038 (2005)), a FRET assay (Marcotte et al., Anal. Biochem. 332: 90 (2004)), and a C¹⁴ NAD boron resin binding assay (McDonagh et al., Methods 36: 346 (2005)). Yet other suitable sirtuin assays include radioimmunoassays (RIA), scintillation proximity assays, HPLC based assays, and reporter gene assays (e.g., for transcription factor targets).

An exemplary assay for determining sirtuin activity is a fluorescence polarization assay. Fluorescence polarization assays are described herein and are also described in PCT Publication No. WO 2006/094239. In other embodiments, sirtuin activity may be determined using a mass spectrometry based assays. Examples of mass spectrometry based assays are described herein and are also described in PCT Publication No. WO 2007/064902. Cell based assays may also be used to determine sirtuin activity. Examples of cell based assays for determining sirtuin activity are described in PCT Publication Nos. WO 2007/064902 and WO 2008/060400.

Yet other methods contemplated herein include screening methods for identifying compounds or agents that modulate sirtuins. An agent may be a nucleic acid, such as an aptamer. Assays may be conducted in a cell based or cell free format. For example, an assay may comprise incubating (or contacting) a sirtuin with a test agent under conditions in which a sirtuin can be modulated by an agent known to modulate the sirtuin, and monitoring or determining the level of modulation of the sirtuin in the presence of the test agent relative to the absence of the test agent. The level of modulation of a sirtuin can be determined by determining its ability to deacetylate a substrate. Exemplary substrates are acetylated peptides which can be obtained from BIOMOL (Plymouth Meeting, Pa.). Preferred substrates include peptides of p53, such as those comprising an acetylated K382. A particularly preferred substrate is the Fluor de Lys-SIRT1 (BIOMOL), i.e., the acetylated peptide Arg-His-Lys-Lys. Other substrates are peptides from human histones H3 and H4 or an acetylated amino acid. Substrates may be fluorogenic. The sirtuin may be SIRT1, Sir2, SIRT3, or a portion thereof. For example, recombinant SIRT1 can be obtained from BIOMOL. The reaction may be conducted for about 30 minutes and stopped, e.g., with nicotinamide. The HDAC fluorescent activity assay/drug discovery kit (AK-500, BIOMOL Research Laboratories) may be used to determine the level of acetylation. Similar assays are described in Bitterman et al. (2002) J. Biol. Chem. 277:45099. The level of modulation of the sirtuin in an assay may be compared to the level of modulation of the sirtuin in the presence of one or more (separately or simultaneously) compounds described herein, which may serve as positive or negative controls. Sirtuins for use in the assays may be full length sirtuin proteins or portions thereof. Since it has been shown herein that activating compounds appear to interact with the N-terminus of SIRT1, proteins for use in the assays include N-terminal portions of sirtuins, e.g., about amino acids 1-176 or 1-255 of SIRT1; about amino acids 1-174 or 1-252 of Sir2.

In one embodiment, a screening assay comprises (i) contacting a sirtuin with a test agent and an acetylated substrate under conditions appropriate for the sirtuin to deacetylate the substrate in the absence of the test agent; and (ii) determining the level of acetylation of the substrate, wherein a lower level of acetylation of the substrate in the presence of the test agent relative to the absence of the test agent indicates that the test agent stimulates deacetylation by the sirtuin, whereas a higher level of acetylation of the substrate in the presence of the test agent relative to the absence of the test agent indicates that the test agent inhibits deacetylation by the sirtuin.

Methods for identifying an agent that modulates, e.g., stimulates, sirtuins in vivo may comprise (i) contacting a cell with a test agent and a substrate that is capable of entering a cell in the presence of an inhibitor of class I and class II HDACs under conditions appropriate for the sirtuin to deacetylate the substrate in the absence of the test agent; and (ii) determining the level of acetylation of the substrate, wherein a lower level of acetylation of the substrate in the presence of the test agent relative to the absence of the test agent indicates that the test agent stimulates deacetylation by the sirtuin, whereas a higher level of acetylation of the substrate in the presence of the test agent relative to the absence of the test agent indicates that the test agent inhibits deacetylation by the sirtuin. A preferred substrate is an acetylated peptide, which is also preferably fluorogenic, as further described herein. The method may further comprise lysing the cells to determine the level of acetylation of the substrate. Substrates may be added to cells at a concentration ranging from about 1 μM to about 10 mM, preferably from about 10 μM to 1 mM, even more preferably from about 100 μM to 1 mM, such as about 200 μM. A preferred substrate is an acetylated lysine, e.g., ε-acetyl lysine (Fluor de Lys, FdL) or Fluor de Lys-SIRT1. A preferred inhibitor of class I and class II HDACs is trichostatin A (TSA), which may be used at concentrations ranging from about 0.01 to 100 μM, preferably from about 0.1 to 10 μM, such as 1 μM. Incubation of cells with the test compound and the substrate may be conducted for about 10 minutes to 5 hours, preferably for about 1-3 hours. Since TSA inhibits all class I and class II HDACs, and that certain substrates, e.g., Fluor de Lys, is a poor substrate for SIRT2 and even less a substrate for SIRT3-7, such an assay may be used to identify modulators of SIRT1 in vivo.

### 5. Pharmaceutical Compositions

The sirtuin-modulating compounds described herein may be formulated in a conventional manner using one or more physiologically acceptable carriers or excipients. For example, sirtuin-modulating compounds and their physiologically acceptable salts and solvates may be formulated for administration by, for example, injection (e.g. SubQ, IM, IP), inhalation or insufflation (either through the mouth or the nose) or oral, buccal, sublingual, transdermal, nasal, parenteral or rectal administration. In one embodiment, a sirtuin-modulating compound may be administered locally, at the site where the target cells are present, i.e., in a specific tissue, organ, or fluid (e.g., blood, cerebrospinal fluid, etc.).

Sirtuin-modulating compounds can be formulated for a variety of modes of administration, including systemic and topical or localized administration. Techniques and formulations generally may be found in Remington's Pharmaceutical Sciences, Meade Publishing Co., Easton, Pa. For parenteral administration, injection is preferred, including intramuscular, intravenous, intraperitoneal, and subcutaneous. For injection, the compounds can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets, lozenges, or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For administration by inhalation (e.g., pulmonary delivery), sirtuin-modulating compounds may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin, for use in an inhaler or insufflator may be formulated containing a powder mix of the compound and a suitable powder base such as lactose or starch.

Sirtuin-modulating compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

Sirtuin-modulating compounds may also be formulated in rectal compositions such as suppositories or retention enemas, e.g., containing conventional suppository bases such as cocoa butter or other glycerides.

In addition to the formulations described previously, sirtuin-modulating compounds may also be formulated as a depot preparation. Such long acting formulations may be administered by implantation (for example subcutaneously or intramuscularly) or by intramuscular injection. Thus, for example, sirtuin-modulating compounds may be formulated with suitable polymeric or hydrophobic materials (for example as an emulsion in an acceptable oil) or ion exchange resins, or as sparingly soluble derivatives, for example, as a sparingly soluble salt. Controlled release formula also includes patches.

In certain embodiments, the compounds described herein can be formulated for delivery to the central nervous system (CNS) (reviewed in Begley, Pharmacology & Therapeutics 104: 29-45 (2004)). Conventional approaches for drug delivery to the CNS include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide).

Liposomes are a further drug delivery system which is easily injectable. Accordingly, in the method of invention the active compounds can also be administered in the form of a liposome delivery system. Liposomes are well-known by a person skilled in the art. Liposomes can be formed from a variety of phospholipids, such as cholesterol, stearylamine of phosphatidylcholines. Liposomes being usable for the method of invention encompass all types of liposomes including, but not limited to, small unilamellar vesicles, large unilamellar vesicles and multilamellar vesicles.

Another way to produce a formulation, particularly a solution, of a sirtuin modulator such as resveratrol or a derivative thereof, is through the use of cyclodextrin. By cyclodextrin is meant α-, β-, or γ-cyclodextrin. Cyclodextrins are described in detail in Pitha et al., U.S. Pat. No. 4,727,064, which is incorporated herein by reference. Cyclodextrins are cyclic oligomers of glucose; these compounds form inclusion complexes with any drug whose molecule can fit into the lipophile-seeking cavities of the cyclodextrin molecule.

Rapidly disintegrating or dissolving dosage forms are useful for the rapid absorption, particularly buccal and sublingual absorption, of pharmaceutically active agents. Fast melt dosage forms are beneficial to patients, such as aged and pediatric patients, who have difficulty in swallowing typical solid dosage forms, such as caplets and tablets. Additionally, fast melt dosage forms circumvent drawbacks associated with, for example, chewable dosage forms, wherein the length of time an active agent remains in a patient's mouth plays an important role in determining the amount of taste masking and the extent to which a patient may object to throat grittiness of the active agent.

Pharmaceutical compositions (including cosmetic preparations) may comprise from about 0.00001 to 100% such as from 0.001 to 10% or from 0.1% to 5% by weight of one or more sirtuin-modulating compounds described herein.

In one embodiment, a sirtuin-modulating compound described herein, is incorporated into a topical formulation containing a topical carrier that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. It is preferable that the selected carrier not adversely affect the active agent or other components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like.

Formulations may be colorless, odorless ointments, lotions, creams, microemulsions and gels.

Sirtuin-modulating compounds may be incorporated into ointments, which generally are semisolid preparations which are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing.

Sirtuin-modulating compounds may be incorporated into lotions, which generally are preparations to be applied to the skin surface without friction, and are typically liquid or semiliquid preparations in which solid particles, including the active agent, are present in a water or alcohol base. Lotions are usually suspensions of solids, and may comprise a liquid oily emulsion of the oil-in-water type.

Sirtuin-modulating compounds may be incorporated into creams, which generally are viscous liquid or semisolid emulsions, either oil-in-water or water-in-oil. Cream bases are water-washable, and contain an oil phase, an emulsifier and an aqueous phase. The oil phase is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington's, supra, is generally a nonionic, anionic, cationic or amphoteric surfactant.

Sirtuin-modulating compounds may be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9).

Sirtuin-modulating compounds may be incorporated into gel formulations, which generally are semisolid systems consisting of either suspensions made up of small inorganic particles (two-phase systems) or large organic molecules distributed substantially uniformly throughout a carrier liquid (single phase gels). Although gels commonly employ aqueous carrier liquid, alcohols and oils can be used as the carrier liquid as well.

Other active agents may also be included in formulations, e.g., other anti-inflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate).

In certain topical formulations, the active agent is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation.

Conditions of the eye can be treated or prevented by, e.g., systemic, topical, intraocular injection of a sirtuin-modulating compound, or by insertion of a sustained release device that releases a sirtuin-modulating compound. A sirtuin-modulating compound that increases the level and/or activity of a sirtuin protein may be delivered in a pharmaceutically acceptable ophthalmic vehicle, such that the compound is maintained in contact with the ocular surface for a sufficient time period to allow the compound to penetrate the corneal and internal regions of the eye, as for example the anterior chamber, posterior chamber, vitreous body, aqueous humor, vitreous humor, cornea, iris/ciliary, lens, choroid/retina and sclera. The pharmaceutically-acceptable ophthalmic vehicle may, for example, be an ointment, vegetable oil or an encapsulating material. Alternatively, the compounds of the invention may be injected directly into the vitreous and aqueous humour. In a further alternative, the compounds may be administered systemically, such as by intravenous infusion or injection, for treatment of the eye.

Sirtuin-modulating compounds described herein may be stored in oxygen free environment. For example, resveratrol or analog thereof can be prepared in an airtight capsule for oral administration, such as Capsugel from Pfizer, Inc.

Cells, e.g., treated ex vivo with a sirtuin-modulating compound, can be administered according to methods for administering a graft to a subject, which may be accompanied, e.g., by administration of an immunosuppressant drug, e.g., cyclosporin A. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000.

Toxicity and therapeutic efficacy of sirtuin-modulating compounds can be determined by standard pharmaceutical procedures in cell cultures or experimental animals. The LD₅₀ is the dose lethal to 50% of the population. The ED50 is the dose therapeutically effective in 50% of the population. The dose ratio between toxic and therapeutic effects (LD₅₀/ED₅₀) is the therapeutic index. Sirtuin-modulating compounds that exhibit large therapeutic indexes are preferred. While sirtuin-modulating compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may lie within a range of circulating concentrations that include the ED₅₀ with little or no toxicity. The dosage may vary within this range depending upon the dosage form employed and the route of administration utilized. For any compound, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

### 6. Kits

Also provided herein are kits, e.g., kits for therapeutic purposes or kits for modulating the lifespan of cells or modulating apoptosis. A kit may comprise one or more sirtuin-modulating compounds, e.g., in premeasured doses. A kit may optionally comprise devices for contacting cells with the compounds and instructions for use. Devices include syringes, stents and other devices for introducing a sirtuin-modulating compound into a subject (e.g., the blood vessel of a subject) or applying it to the skin of a subject.

In yet another embodiment, the invention provides a composition of matter comprising a sirtruin modulator of this invention and another therapeutic agent (the same ones used in combination therapies and combination compositions) in separate dosage forms, but associated with one another. The term “associated with one another” as used herein means that the separate dosage forms are packaged together or otherwise attached to one another such that it is readily apparent that the separate dosage forms are intended to be sold and administered as part of the same regimen. The agent and the sirtruin modulator are preferably packaged together in a blister pack or other multi-chamber package, or as connected, separately sealed containers (such as foil pouches or the like) that can be separated by the user (e.g., by tearing on score lines between the two containers).

In still another embodiment, the invention provides a kit comprising in separate vessels, a) a sirtruin modulator of this invention; and b) another therapeutic agent such as those described elsewhere in the specification.

The practice of the present methods will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXEMPLIFICATION

The invention now being generally described, it will be more readily understood by reference to the following examples which are included merely for purposes of illustration of certain aspects and embodiments of the present invention, and are not intended to limit the invention in any way.

### Example 1

### Preparation of 2-(2-thiazolyl)aniline

To a solution of 157 mg (1.0 mmol) of 50% chloroacetaldehyde in water was added 1 mL DMF, then 152 mg of 2-amino-benzothioamide. The reaction was stirred at 70° C. for 20 min, then 10 mL of water and 1 mL of saturated NaHCO₃ solution were added. The mixture was extracted with ethyl acetate (3×5 mL), then the combined ethyl acetate layers were back extracted with water (2×5 mL), and brine (1×5 mL), dried over MgSO₄, filtered, and concentrated to an oil. This was purified via silica gel chromatography, eluting with a 5-20% ethyl acetate/pentane gradient to give 41 mg (23%) of an oil.

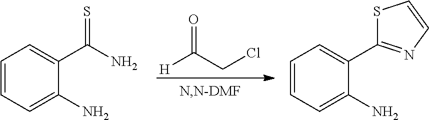

### General Method A:

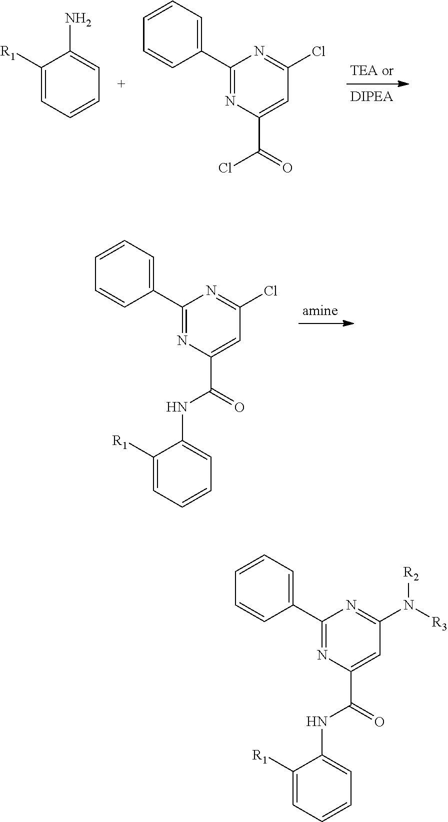

A mixture of aniline (1 eq), acid chloride (1.1 eq), and base (TEA or DIPEA, 1.2 eq) is stirred at room temperature in a suitable solvent (CH₂Cl₂ or CH₃CN) for 2 h. The resulting precipatate is collected by filtration, washed and dried under vacuum to afford the amide. If the reaction mixture is heterogeneous, MeOH can be added to induce precipitation.

A solution of chloropyrimidine (1 eq) and amine (5 eq) in THF is heated at reflux for several hours. The reaction mixture is poured into H₂O, and the resulting solid is collected by filtration, washed and dried under vacuum. The crude product could be further purified by recrystallization from an appropriate solvent (EtOH or CH₃CN) or chromatography.

### General Method A′

### Preparation of 6-(2-(dimethylamino)ethylamino)-2-phenyl-N-(2-(thiazol-2-yl)phenyl)pyrimidine-4-carboxamide

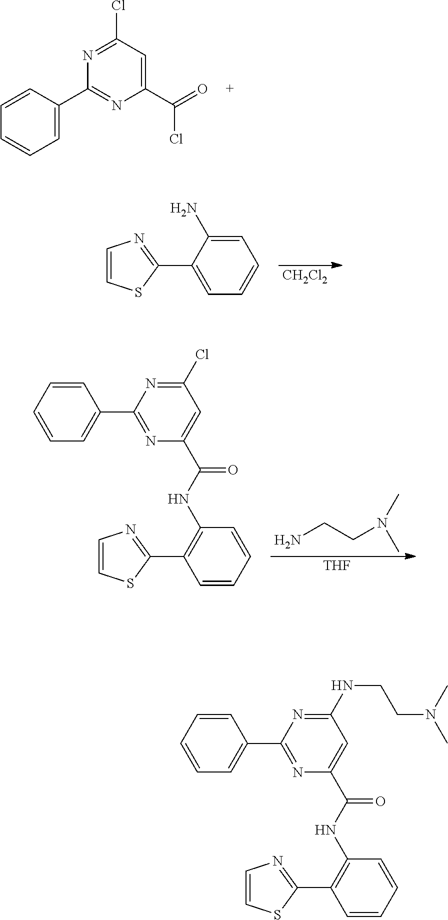

To a solution of 35 mg (0.199 mmol) of 2-(2-thiazolyl)aniline in 1 mL of CH₂Cl₂ was added 3 drops of triethylamine, then 50 mg (0.20 mmol) of 6-chloro-2-phenylpyrimidine-4-carbonyl chloride. The mixture was stirred to dissolve the acid chloride, then a precipitate formed. After 10 min, the reaction was diluted with 5 mL of methanol, then the precipitate was filtered, washed with additional methanol, and dried on the filter to give 64 mg (82%) of the chloropyrimidine amide as a pale yellow solid.

To a solution of 50 mg (0.127 mmol) of the chloropyrimidine amide in 1 mL of THF was added 160 μL (1.50 mmol) of N,N-dimethylethylenediamine. The reaction was stirred at reflux for 30 min, then diluted with 10 mL of water. The suspension was stirred for 10 min, then filtered to give 33 mg (58%) of a white solid. (MS, M⁺+H=445).

### Preparation of 6-(2-(dimethylamino)ethylamino)-N,2-diphenylpyrimidine-4-carboxamide

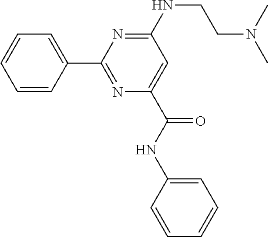

The title compound was prepared according to General Method A′, utilizing the appropriate amine. Yield 239 mg (20%, 2 steps). (MS, M⁺+H=362).

### Preparation of N-(biphenyl-2-yl)-6-(2-(dimethylamino)ethylamino)-2-phenylpyrimidine-4-carboxamide

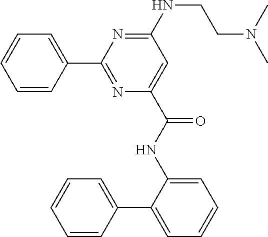

The title compound was prepared according to General Method A′, utilizing the appropriate amine. Yield 188 mg (36%, 2 steps). (MS, M⁺+H=438).

### Preparation of N-(2-carbamoylphenyl)-6-(2-(dimethylamino)ethylamino)-2-phenylpyrimidine-4-carboxamide

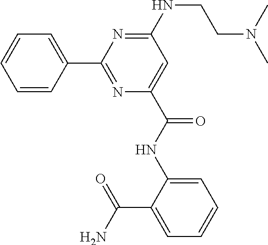

The title compound was prepared according to General Method A′, utilizing the appropriate amine. Yield 157 mg (26%, 2 steps). (MS, M⁺+H=405).

### Preparation of N-(2-cyanophenyl)-6-(2-(dimethylamino)ethylamino)-2-phenylpyrimidine-4-carboxamide

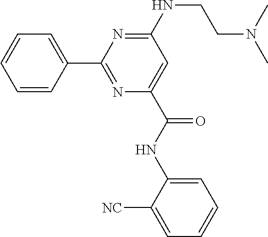

The title compound was prepared according to General Method A′, utilizing the appropriate amine. Yield 91 mg (14%, 2 steps). (MS, M⁺+H=387).

### Preparation of methyl 2-(6-(2-(dimethylamino)ethylamino)-2-phenylpyrimidine-4-carboxamido)benzoate

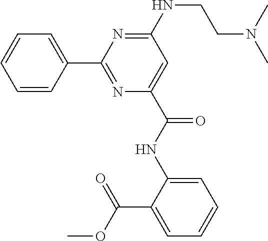

The title compound was prepared according to General Method A′, utilizing the appropriate amine. Yield 292 mg (35%, 2 steps). (MS, M⁺+H=420).

### Preparation of 6-(2-(dimethylamino)ethylamino)-N-(2-ethylphenyl)-2-phenylpyrimidine-4-carboxamide

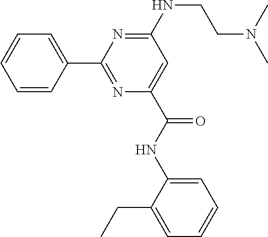

The title compound was prepared according to General Method A′, utilizing the appropriate amine. Yield 245 mg (25%, 2 steps). (MS, M⁺+H=390).

### Preparation of 6-(2-(dimethylamino)ethylamino)-N-(2-(5-methylthiazol-2-yl)phenyl)-2-phenylpyrimidine-4-carboxamide

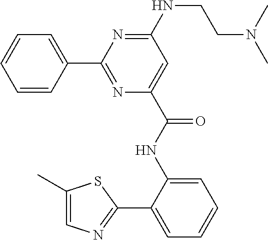

The title compound was prepared according to General Method A′, utilizing the appropriate amine. Yield 40 mg (57%, 2 steps). (MS, M⁺+H=459).

### Preparation of 2-(5-methylthiazol-2-yl)aniline

Prepared according to the procedure for 2-(2-thiazolyl)aniline, substituting chloroacetone for chloroacetaldehyde. Yield 60 mg (32%) of a pale yellow solid.

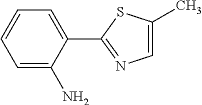

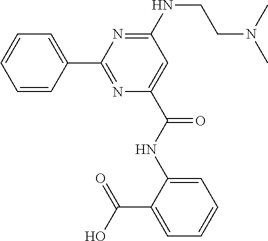

2-(6-(2-(dimethylamino)ethylamino)-2-phenylpyrimidine-4-carboxamido)benzoate. To 120 mg (0.29 mmol) of methyl 2-(6-(2-(dimethylamino)ethylamino)-2-phenylpyrimidine-4-carboxamido)benzoate in THF/H₂O (4 mL/0.5 mL). was added 60 mg (1.43 mmol) of LiOH.in H₂O. The mixture was stirred at ambient temperature overnight and then heated to 50° C. for 30 min. The reaction solution was adjusted to a pH of 3 using 1 M hydrochloric acid. The white precipitate formed was collected by filtration and dried in vacuo to give 77 mg (65%) of the carboxylic acid as a white solid. ESI-MS: 404 [M−1]⁻.

### Preparation of 6-chloro-2-phenylpyrimidine-4-carbonyl chloride

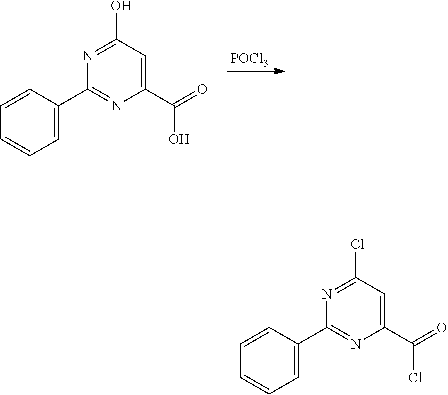

To 1.00 g of 6-hydroxy-2-phenylpyrimidine-4-carboxylic acid was added 10 mL of phosphorus oxychloride. The reaction was heated at reflux for 1 h, then concentrated in vacuo to an oil, removing as much phosphorus oxychloride as possible. The oil was suspended in 30 mL of pentane, then the mixture was extracted with water (3×5 mL) and brine (1×5 mL). The organic layer was dried over MgSO₄, filtered, and concentrated in vacuo to give 1.03 g (88%) of the acid chloride as a white solid.

### Preparation of 4-chloro-6-phenylpicolinoyl chloride

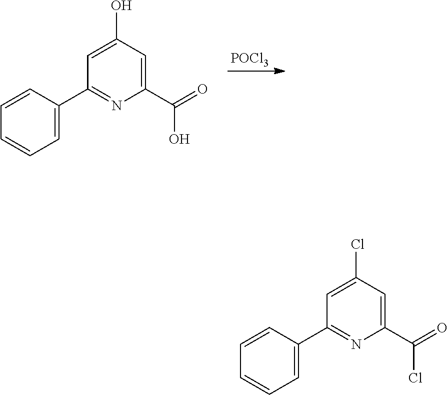

To 1.00 g of 4-hydroxy-6-phenylpicolinic acid was added 5 mL of phosphorus oxychloride. The reaction was heated at reflux for 1 h then concentrated in vacuo to an oil, removing as much phosphorus oxychloride as possible. The oil was suspended in 30 mL of pentane then concentrated in vacuo again. The oil was resuspended in pentane (100 mL) and extracted with water (20 mL) and bicarb (20 mL). The organic layer was dried over sodium sulfate, filtered and concentrated in vacuo to give 0.69 g (55%) of the acid chloride as a white solid.

### Preparation of 2-(2-thiazolyl)aniline

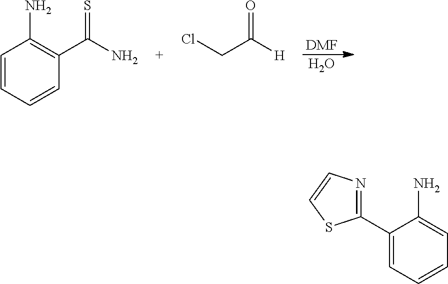

To a solution of 157 mg (1.0 mmol) of 50% chloroacetaldehyde in water was added 1 mL N,N-dimethylformamide (DMF), then 152 mg of 2-amino-benzothioamide (Fontrodona, X.; Diaz, S.; Linden, A.; Villalgordo, J. M. “Copper(I)Bromide-Mediated Synthesis of Novel 2-Arylthiazole-5-carboxylates from α-Diazo-β-Keto Esters and Aromatic Thioamides.” Synthesis, 2001, 13, 2021-2027). The reaction was stirred at 70° C. for 20 min, then 10 mL of water and 1 mL of saturated NaHCO₃ solution were added. The mixture was extracted with ethyl acetate (3×5 mL), then the combined ethyl acetate layers were back extracted with water (2×5 mL), and brine (1×5 mL), dried over MgSO₄, filtered, and concentrated to an oil. This was purified via silica gel chromatography, eluting with a 5-20% ethyl acetate/pentane gradient to give 41 mg (23%) of an oil.

### Preparation of 2-(4-methylthiazol-2-yl)aniline

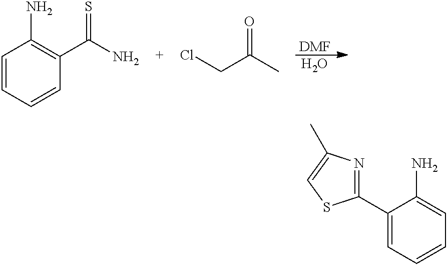

Prepared according to the procedure for 2-(2-thiazolyl)aniline, substituting chloroacetone for chloroacetaldehyde. Yield 60 mg (32%) of a pale yellow solid.

### Preparation of 2-(2-phenylthiazol-4-yl)aniline

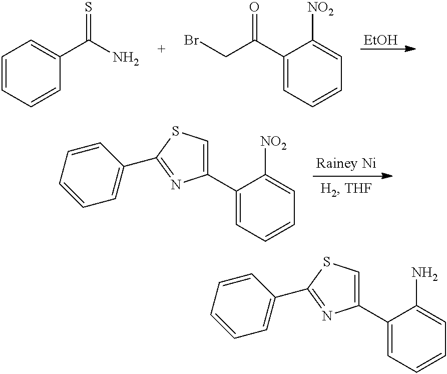

A solution of benzothioamide (2.74 g, 20 mmol) and 2-bromo-1-(2-nitrophenyl)ethanone (2.44 g, 10 mmol) in 95% ethanol (EtOH) (15 mL) was refluxed for 1 h, and cooled to room temperature. Colorless needles were formed and collected by filtration, washed with water and dried to give 4-(2-nitrophenyl)-2-phenylthiazole as a white solid (2.44 g, yield: 86%).

To a solution of 4-(2-nitrophenyl)-2-phenylthiazole (2.44 g, 8.6 mmol) in tetrahydrofuran (THF) (20 mL) was added Raney Nickel (0.24 g). The reaction mixture was stirred under H₂ overnight, and filtered through a celite pad. The filtrate was dried over Na₂SO₄ and concentrated under vacuum to give 2-(2-phenylthiazol-4-yl)aniline as a yellow solid (2.15 g, yield: 98%).

### Preparation of 2-(5-phenylthiazol-2-yl)aniline

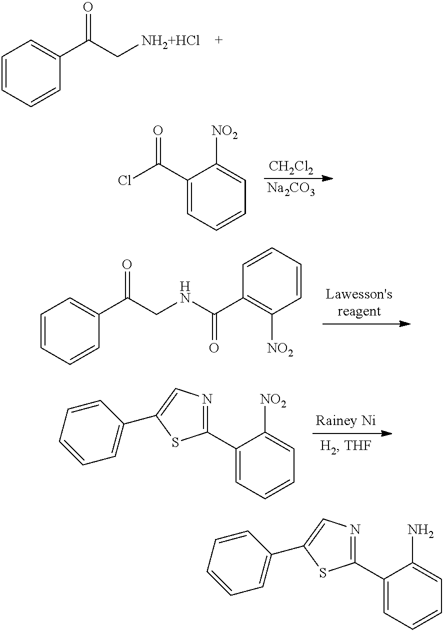

To a suspension of 2-amino-1-phenylethanone hydrochloride (3.43 g, 20 mmol) and Na₂CO₃ (4.24 g, 40 mmol) in CH₂Cl₂ (50 mL) was added 2-nitrobenzoyl chloride (4.45 g, 24 mmol). The mixture was stirred at rt overnight. Water (100 mL) was added and the mixture was stirred for 15 min. The reaction mixture was partitioned between CH₂Cl₂ (100 mL) and Na₂CO₃ solution (100 mL). The organic phase was washed with saturated Na₂CO₃ (100 mL), 10% HCl solution (100 brine (100 mL), dried over Na₂SO₄, and concentrated to give 2-nitro-N-((2-oxo-2-phenylethyl)benzamide as a white solid (5.2 g, yield: 91%)

A solution of 2-nitro-N-((2-oxo-2-phenylethyl)benzamide (2.84 g, 10 mmol) and Lawesson's reagent (2,4-bis(4-methoxyphenyl)-1,3,2,4-dithiadiphosphetane-2,4-disulfide) (4.04 g, 10 mmol) in THF (50 mL) was heated at reflux overnight. The reaction mixture was concentrated in vacuo and purified by chromatography (eluted with pentane:CH₂Cl₂=1:1) to give 2-(2-nitrophenyl)-5-phenylthiazole as a yellow solid (1.7 g, yield: 60%)

2-(2-nitrophenyl)-5-phenylthiazole (1.7 g, 6 mmol) was dissolved in methanol(MeOH) (25 mL) and THF (50 mL). Raney Nickel (1.0 g) was added. The resulting mixture was stirred over H₂ overnight then passed through a celite pad. The solvent was evaporated and the crude product was purified by chromatography (eluted pentane:CH₂Cl₂=2:1) to give 2-(5-phenylthiazol-2-yl)aniline as a yellow solid (1.2 g, yield: 79%)

### Preparation of 2-(5-phenyloxazol-2-yl)aniline

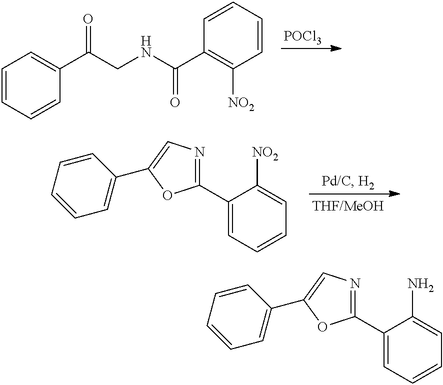

A solution of 2-nitro-N-((2-oxo-2-phenylethyl)benzamide (2.5 g, 8.8 mmol) in POCl₃ (25 mL) was heated at reflux for 16 h. The volatiles were removed in vacuo. The residue was dissolved in ethyl acetate (EtOAc), washed with H2O, 15% NaOH aq, and brine, dried and concentrated to give 2-(2-nitrophenyl)-5-phenyloxazole (2.2 g, 93% yield).

Palladium (Pd) (250 mg, 10 wt % on C) was added to a solution of 2-(2-nitrophenyl)-5-phenyloxazole (2.2 g, 8.3 mmol) in CH₃OH/THF (50/50 mL). The reaction mixture was hydrogenated at balloon pressure for 16 h. The catalyst was removed by filtration through celite and the solvent evaporated to give 2-(5-phenyloxazol-2-yl)aniline (1.7 g, 87% yield).

### Preparation of 2-(4-phenylthiazole-2-yl)aniline

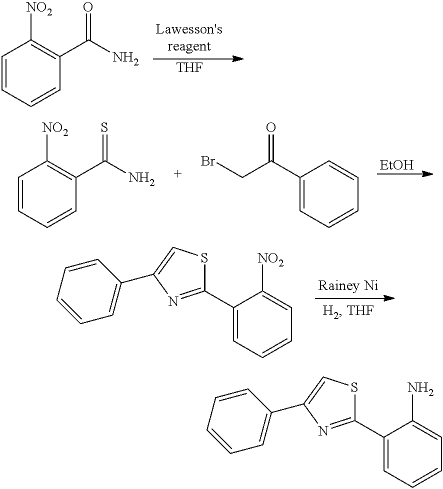

A solution of 2-nitrobenzamide (4.03 g, 24.2 mmol) and Lawesson's reagent (5.92 g, 14.6 mmol) in THF (150 mL) was stirred at room temperature overnight, then concentrated in vacuo. The residue was partitioned between ethyl acetate (100 mL) and H₂O (50 mL). The aqueous layer was extracted by ethyl acetate (50 ml×2). The combined organic layers were washed with brine, dried and concentrated. The crude product was purified by flash chromatography on silica gel (elution: pentane:ethyl acetate=7:1) to give 2-Nitrobenzothioamide as a yellow solid (3.82 g, 86.7% yield).

A solution of nitrobenzothioamide (2.1 g, 11.5 mmol) and 2-bromo-1-phenylethanone (2.3 g, 11.6 mmol) in ethanol was heated at reflux for 6 hours. The reaction mixture was diluted with ethyl acetate, washed with sat. aq NaHCO₃, dried (MgSO₄), and concentrated. The crude product was recrystallized from methanol to give 2-(2-nitrophenyl)-4-phenylthiazole as colorless needles (3 g, 92% yield).

2-(2-nitrophenyl)-4-phenylthiazole (3.0 g, 10.6 mmol) was dissolved in methanol (150 mL). Raney Nickel (1.0 g) was added. The resulting mixture was stirred over H₂ overnight then passed through a celite pad. The solvent was evaporated to give 2-(4-phenylthiazol-2-yl)aniline as a yellow solid (2.3 g, 86% yield).

### Preparation of 2-(2-phenyl-1H-imidazole-4-yl)aniline

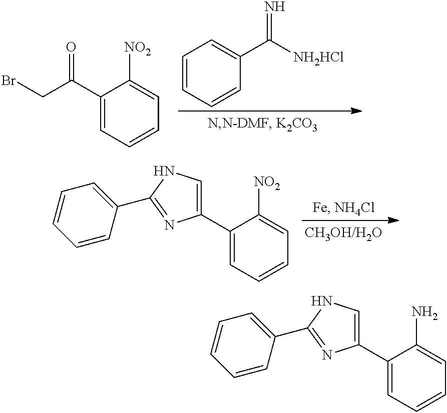

To a solution of 2-bromo-1-(2-nitrophenyl)ethanone (1.23 g, 7 mmol) in N,N-DMF (30 ml) was added K₂CO₃ (1.93 g, 14 mmol) followed by benzamidine hydrochloride (1.71 g, 7 mmol). The reaction mixture was stirred at 50° C. for 5 h. The reaction mixture was poured into H₂O and the resulting precipitate was collected by filtration. Purification by flash chromatography (silica, pentane:ethyl acetate=7:1) gave 2-(2-phenyl-1H-imidazole-4-yl)aniline as a yellow solid (0.5 g, 26.9% yield).

A suspension of 2-(2-phenyl-1H-imidazole-4-yl)aniline (0.5 g, 2 mmol), Fe (0.53 g, 9.4 mmol) and NH₄Cl (0.81 g, 15.1 mmol) in CH₃OH/H₂O (20/5 mL) was heated at reflux overnight. The mixture was filtered through a pad of celite and concentrated. The residue was dissolved in ethyl acetate and washed with H₂O. The organic phase was dried and concentrated to give 2-(2-phenyl-1H-imidazole-4-yl)aniline as a light red solid (0.21 g, 47% yield).

### Preparation of 2-(4-phenyl-1H-pyrazol-1-yl)aniline

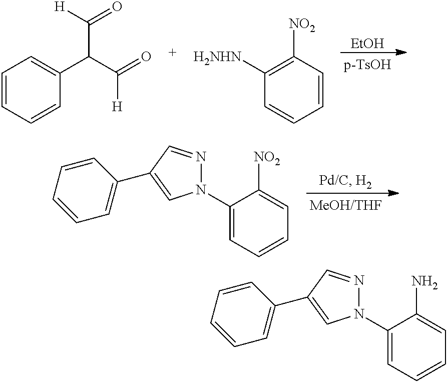

Para-toluene sulfonic acid (p-TsOH) (128 mg, 0.68 mmol) was added to a solution of 2-phenylmalonaldehyde (1.0 g, 6.8 mmol) and (2-nitrophenyl)hydrazine (1.03 g, 6.78 mmol) in EtOH (30 mL). The reaction mixture was heated at reflux for 4 h then cooled to room temperature. The resulting precipitate was collected by filtration, rinsed with EtOH then diethyl ether (Et₂O), and dried under vacuum to give 1-(2-nitrophenyl)-4-phenyl-1H-pyrazole as a white solid (1.02 g, 57% yield).

Pd (150 mg, 10 wt % on C) was added to a solution of 1-(2-nitrophenyl)-4-phenyl-1H-pyrazole (1.02 g, 3.85 mmol) in MeOH/THF (20/20 mL). The reaction mixture was hydrogenated at balloon pressure for 16 h. The catalyst was removed by filtration through celite and the solvent evaporated to give 2-(4-phenyl-1H-pyrazol-1-yl)aniline as a white solid (860 mg, 95% yield).

### Preparation of 2-(5-tert-butylthiazol-2-yl)aniline

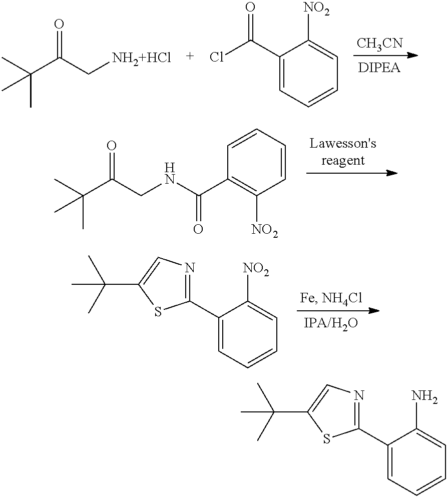

To a suspension of 1-amino-3,3-dimethylbutan-2-one hydrochloride (2.5 g, 16.5 mmol) and 2-nitrobenzoyl chloride (3.4 g, 18.1 mmol) in acetonitrile (CH₃CN) (30 mL) was added diisopropylethylamine (DIPEA) (8.6 mL, 49.5 mmol). The mixture was stirred at room temperature for 16 h. The reaction mixture was diluted with EtOAc (100 mL) and washed with saturated aqueous NaHCO₃. The organic layer was dried and concentrated. The crude product was purified by recrystallization from EtOAc to give N-(3,3-dimethyl-2-oxobutyl)-2-nitrobenzamide as a white solid (4.0 g, yield: 91%)

A solution of N-(3,3-dimethyl-2-oxobutyl)-2-nitrobenzamide (1.5 g, 5.7 mmol) and Lawesson's reagent (4.6 g, 13.4 mmol) in THF (30 mL) was heated at reflux for 16 h. The reaction mixture was concentrated in vacuo and purified by medium pressure liquid chromatography (MPLC) eluting with pentane/EtOAc to give 5-tert-butyl-2-(2-nitrophenyl)thiazole (1.4 g, 94% yield)

A suspension of 5-tert-butyl-2-(2-nitrophenyl)thiazole (1.4 g, 5.5 mmol), Fe (1.5 g, 27.5 mmol) and NH₄Cl (352 mg, 6.6 mmol) in iso-propylalcohol (IPA)/H₂O (30/5 mL) was heated at reflux for 16 h. The mixture was filtered through a pad of celite and concentrated. The residue was suspended in EtOAc, washed with brine, dried and concentrated. The crude product was purified by MPLC eluting with pentane/EtOAc to give 2-(5-tert-butylthiazol-2-yl)aniline (986 mg, 77% yield)

### Preparation of 2-(5-tert-butyloxazol-2-yl)aniline

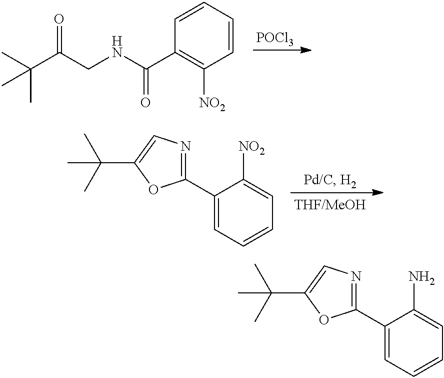

A solution of N-(3,3-dimethyl-2-oxobutyl)-2-nitrobenzamide (1.5 g, 5.7 mmol) in POCl₃ (15 mL) was heated at reflux for 16 h. The volatiles were removed in vacuo. The residue was dissolved in EtOAc, washed with H₂O, dried and concentrated to give 5-tert-butyl-2-(2-nitrophenyl)oxazole (1.4 g, 99% yield).

Pd (250 mg, 10 wt % on C) was added to a solution of 5-tert-butyl-2-(2-nitrophenyl)oxazole (1.4 g, 5.7 mmol) in MeOH/THF (25/25 mL). The reaction mixture was hydrogenated at balloon pressure for 16 h. The catalyst was removed by filtration through celite and the solvent evaporated to give 2-(5-tert-butyloxazol-2-yl)aniline (1.0 g, 83% yield).

### Preparation of 2-(5-(pyridine-2-yl)-1,3,4-thiadiazol-2-yl)aniline

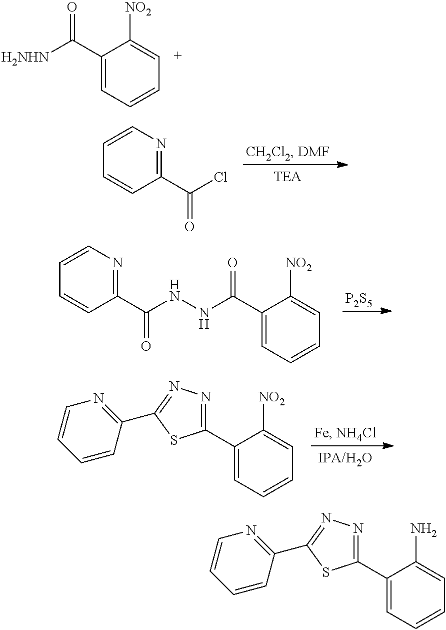

A solution of picolinoyl chloride (5 g, 28 mmol) in DMF (30 mL) was added to a solution of 2-nitrobenzohydrazide (4.6 g, 25.4 mmol) in CH₂Cl₂ (80 mL) at 0° C. Triethylamine (TEA) (5.6 g) was added and the reaction mixture was allowed to warm to room temperature over 16 h. The volatiles were removed in vacuo and H₂O (100 mL) was added to the residue. The resulting precipitate was collected by filtration, rinsed with H₂O and dried under vacuum to give N′-(2-nitrobenzoyl)picolinohydrazide (6.0 g, 83% yield).

N′-(2-nitrobenzoyl)picolinohydrazide (8.0 g, 27.9 mmol) and P₂S₅ (16.7 g, 75 mmol) were suspended in toluene (300 mL) and heated at reflux for 16 h. H₂O (100 mL) and EtOAc (100 mL) were added, and the mixture was vigorously stirred for 20 minutes. The solid was filtered and repeatedly washed with EtOAc. The filtrates were combined and the layers separated. The organic layer was extracted with 2M NaOH (2×100 mL), brine (100 mL), dried and concentrated to give 2-(2-nitrophenyl)-5-(pyridine-2-yl)-1,3,4-thiadiazole (4.0 g, 81% yield).

A suspension of 2-(2-nitrophenyl)-5-(pyridine-2-yl)-1,3,4-thiadiazole (4.0 g, 14 mmol), Fe (1.7 g, 30.7 mmol) and NH₄Cl (1.5 g, 28 mmol) in IPA/H₂O (150/30 mL) was heated at reflux for 6 h. The mixture was filtered through a pad of celite and concentrated. The residue was suspended in EtOAc, washed with 2M NaOH, brine, dried and concentrated to give 2-(5-(pyridine-2-yl)-1,3,4-thiadiazol-2-yl)aniline (2.8 mg, 79% yield).

### Preparation of 2-(5-(pyridine-3-yl)-1,3,4-thiadiazol-2-yl)aniline

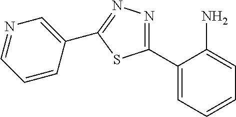

Prepared according to the procedure described for 2-(5-(pyridine-2-yl)-1,3,4-thiadiazol-2-yl)aniline, substituting nicotinyl chloride for picolinyl chloride (9% yield for 3 steps).

### Preparation of 2-(5-(pyridine-4-yl)-1,3,4-thiadiazol-2-yl)aniline

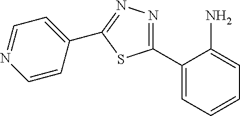

Prepared according to the procedure described for 2-(5-(pyridine-2-yl)-1,3,4-thiadiazol-2-yl)aniline, substituting isonicotinyl chloride for picolinyl chloride (11% yield for 3 steps).

### Preparation of 2-(5-cyclopentyl-1,3,4-thiadiazol-2-yl)aniline

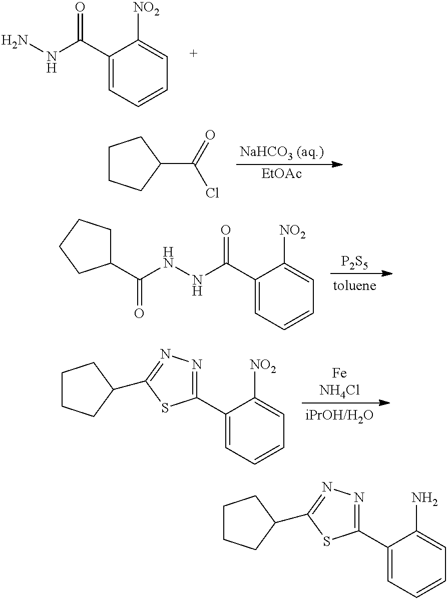

2-nitrobenzhydrazide (0.65 g, 3.59 mmol) was suspended in ethyl acetate (14 mL) and warmed to 45° C. Saturated aqueous sodium bicarbonate (4 mL) was added, followed by cyclopentanecarbonyl chloride (0.44 mL, 3.59 mmol) in 2 mL ethyl acetate. A white precipitate formed immediately. The mixture was allowed to stand for 10 min. at room temperature, then the product was collected by filtration, washed with ethyl acetate (3×10 mL) and water (3×5 mL). The solid was dried overnight in vacuo at 40° C. to give N′-(cyclopentanecarbonyl)-2-nitrobenzohydrazide (0.61 g, 62%).

N′-(cyclopentanecarbonyl)-2-nitrobenzohydrazide (0.61 g, 2.22 mmol) and phosphorus pentasulfide (1.48 g, 3.32 mmol) were premixed. Toluene (30 mL) was added and the mixture was heated to 90° C. for 2 h. The reaction was cooled to room temperature and water was added (10 mL). The mixture was filtered and the solid was washed with ethyl acetate (3×25 mL). The filtrate layers were separated and the organics were washed with brine, dried with sodium sulfate, filtered and concentrated in vacuo. Crude product was purified by silica gel chromatography (0-60% ethyl acetate/pentane) to give 2-cyclopentyl-5-(2-nitrophenyl)-1,3,4-thiadiazole (419 mg, 69%).

2-cyclopentyl-5-(2-nitrophenyl)-1,3,4-thiadiazole (0.42 g, 1.52 mmol) was suspended in isopropanol (28 mL) and water (7 mL). Ammonium chloride (98 mg, 1.82 mmol) and iron powder (0.28 g, 5.02 mmol) were added. The mixture was heated to reflux with vigorous stirring for 4 h. The reaction was cooled to room temperature and filtered through a pad of Celite. The filtrate was concentrated in vacuo and resuspended in ethyl acetate (50 mL). The organic layer was washed with 1 N NaOH (10 mL) and brine (10 mL), dried with sodium sulfate, filtered and concentrated in vacuo to give 2-(5-cyclopentyl-1,3,4-thiadiazol-2-yl)aniline (349 mg, 93%).

### Preparation of 2-(5-butyl-1,3,4-thiadiazol-2-yl)aniline

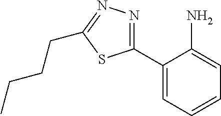

Prepared according to the procedure described for 2-(5-cyclopentyl-1,3,4-thiadiazol-2-yl)aniline, substituting pentanoyl chloride as the acid chloride.

### Preparation of 2-(5-isopropyl-1,3,4-thiadiazol-2-yl)aniline

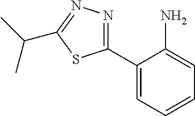

Prepared according to the procedure described for 2-(5-cyclopentyl-1,3,4-thiadiazol-2-yl)aniline, substituting isopropoyl chloride as the acid chloride.

### Preparation of 2-(5-morpholino-1,3,4-thiadiazol-2-yl)aniline

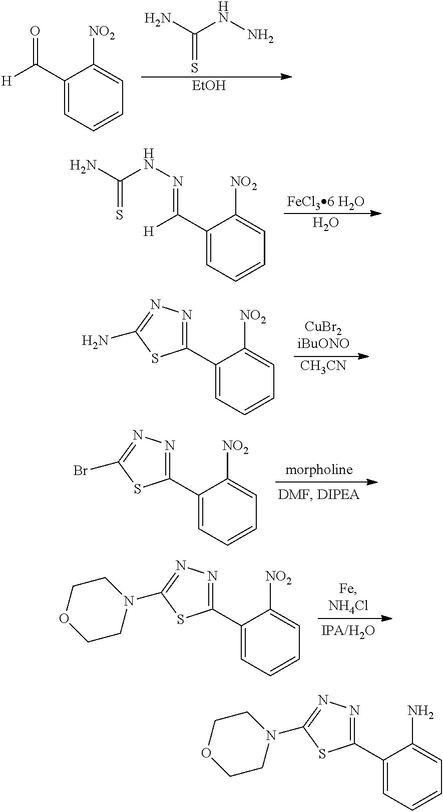

2-nitrobenzaldehyde (6.00 g, 39.7 mmol) and thiosemicarbazide (3.62 g, 39.7 mmol) were suspended in ethanol (120 mL) and heated to reflux for 1 h. The mixture was cooled to room temperature, and the product was collected by filtration. The solid was washed with ethanol (3×10 mL), then ether (3×10 mL) and dried in vacuo to give 2-(2-nitrobenzylidene)hydrazinecarbothioamide as a yellow solid (8.64 g, 97%).

2-(2-nitrobenzylidene)hydrazinecarbothioamide (3.32 g, 14.8 mmol) was suspended in water (330 mL). Iron trichloride hexahydrate (12.00 g, 44.4 mmol) was dissolved in 330 mL water and added to the reaction flask over 5 min. The reaction was heated to 90° C. for 19 h then cooled to room temperature. Citric acid (14.05 g=0.11M) and sodium citrate dihydrate (9.78 g=0.05M) were added. Ammonium hydroxide (5 N) was then added to neutralize (to pH 7). The solution was cooled in an ice bath then filtered. The solid was washed with water and air-dried. Trituration with acetonitrile (10 mL), filtration, and high vacuum gave 5-(2-nitrophenyl)-1,3,4-thiadiazol-2-amine as a yellow-orange solid (2.11 g, 64%).

Copper(II) bromide (0.614 g, 2.75 mmol) and t-butyl nitrite (0.408 mL, 3.44 mmol) were stirred in 4 mL acetonitrile. 5-(2-nitrophenyl)-1,3,4-thiadiazol-2-amine (0.509 g, 2.29 mmol) was suspended in 8 mL acetonitrile and added to the reaction flask. The reaction was warmed to 65° C. for 7 h, then poured into 40 mL of 5 N HCl. Ether (60 mL) was added and the layers separated. The organic layer was washed again with 5 N HCl (20 mL), then dried with sodium sulfate, filtered and concentrated. Purification by silica gel chromatography (0-100% EtOAc/pentane) gave 2-bromo-5-(2-nitrophenyl)-1,3,4-thiadiazole as a tan-colored solid (536 mg, 87%).

DIPEA (2.3 mL, 13.1 mmol) was added to a solution of 2-bromo-5-(2-nitrophenyl)-1,3,4-thiadiazole (1.5 g, 5.2 mmol) and morpholine (1.1 mL, 13.1 mmol) in DMF (20 mL). The reaction mixture was heated at 70 C for 16 h. The mixture was poured into H₂O and extracted with EtOAc. The combined organic layers were washed with brine, dried and concentrated. The crude residue was purified by recrystallization from EtOAc to give 4-(5-(2-nitrophenyl)-1,3,4-thiadiazole-2-yl)morpholine (1.3 g, 85% yield) as a white solid.

A suspension of 4-(5-(2-nitrophenyl)-1,3,4-thiadiazole-2-yl)morpholine (1.3 g, 4.4 mmol), Fe (1.2 g, 22.2 mmol) and NH₄Cl (285 mg, 5.3 mmol) in IPA/H₂O (100/25 ml) was heated at reflux for 6 h. The mixture was filtered through a pad of celite and concentrated. The residue was suspended in EtOAc, washed with saturated aqueous NaHCO₃, brine, dried and concentrated. The crude product was purified by MPLC eluting with pentane/EtOAc to give 2-(5-morpholino-1,3,4-thiadiazol-2-yl)aniline (1.1 g, 94% yield).

### Preparation of (S)-2-(5-(hexahydropyrrolo[1,2-a]pyrazin-2(1H)-yl)-1,3,4-thiadiazol-2-yl)aniline

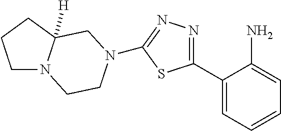

Prepared according to the procedure described for 2-(5-morpholino-1,3,4-thiadiazol-2-yl)aniline, substituting (S)-octahydropyrrolo[1,2-a]pyrazine for morpholine.

### Preparation of 2-(pyridin-2-yl)aniline

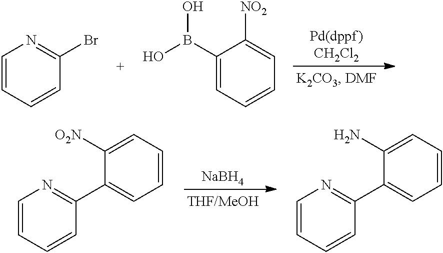

2-bromopyridine (0.25 g, 1.58 mmol) and 2-nitrophenylboronic acid (0.48 g, 2.85 mmol) were suspended in DMF (8.0 mL) in a microwave vial. Nitrogen was bubbled through the solution for 5 min. Pd (1,1′-Bis(diphenylphosphino)ferrocene i.e., dppf) catalyst (0.10 g, 0.13 mmol) and potassium carbonate (0.44 g, 3.16 mmol) were added and the vial was sealed. The mixture was heated in the microwave to 140° C. for 30 min. The vial was cooled to room temperature, and the mixture was neutralized with 1 N sodium hydrogen sulfate (3.16 mL). Ethyl acetate (100 mL) and water (100 mL) were added. The organic layer was separated and washed with brine (30 mL), dried with sodium sulfate, filtered and concentrated in vacuo. The crude product was purified by silica gel chromatography (10-50% ethyl acetate/pentane) to give 2-(2-nitrophenyl)pyridine as a brown oil (58 mg, 18%).

2-(2-nitrophenyl)pyridine (84 mg, 0.42 mmol) was dissolved in THF (13 mL) and methanol (0.65 mL) under nitrogen atmosphere. Sodium borohydride (96 mg, 2.52 mmol) was added and the reaction was stirred at room temperature for 1 h. Water was added (20 mL) and the product was extracted with ethyl acetate (3×50 mL). The combined organics were dried with sodium sulfate, filtered and concentrated in vacuo. The crude product was purified by silica gel chromatography (0-80% ethyl acetate/pentane) to give 2-(pyridin-2-yl)aniline (57 mg, 79%).

### Preparation of (2-aminophenyl)(thiazol-2-yl)methanone

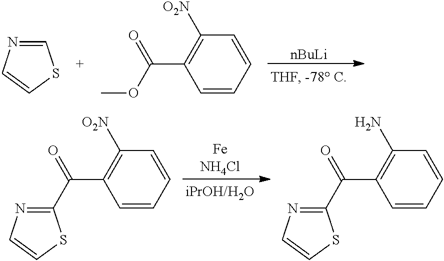

Butyllithium (2.5 M in hexanes, 0.73 mL, 1.82 mmol) was cooled to −78° C. under nitrogen atmosphere. Thiazole (0.13 mL, 0.16 g, 1.82 mmol) was dissolved in THF (2.3 mL) and added to the cooled solution dropwise over 10 min. This was stirred at −78° C. for 1.5 h. Methyl-2-nitrobenzoate was dissolved in THF (6.0 mL) and cooled to −78° C. The thiadiazole solution was quickly poured into this solution, and the mixture was stirred at −78° C. for 1 h. The reaction was warmed to room temperature. Potassium carbonate, 1% (20 mL) and ethyl acetate (40 mL) were added and the layers were separated. The organic layer was washed with brine, dried with sodium sulfate, filtered and concentrated in vacuo. The crude product was purified by silica gel chromatography (0-100% ethyl acetate/pentane) to give (2-nitrophenyl)(thiazol-2-yl)methanone (81 mg, 21%).

(2-nitrophenyl)(thiazol-2-yl)methanone (81 mg, 0.34 mmol) was suspended in isopropanol (6 mL) and water (1.5 mL). Ammonium chloride (22 mg, 0.42 mmol) and iron powder (64 mg, 1.1 mmol) were added and the mixture was heated to reflux, stirring vigorously, for 3 h. The reaction was cooled to room temperature and filtered through a pad of Celite. The filtrate was concentrated in vacuo and the residue was suspended in ethyl acetate (40 mL). The organic layer was washed with 1 N NaOH (10 mL) and brine (10 mL), dried with sodium sulfate, filtered and concentrated in vacuo to give (2-aminophenyl)(thiazol-2-yl)methanone as a yellow oil (72 mg, 100%).

### Preparation of (2-aminophenyl)(pyridin-2-yl)methanone

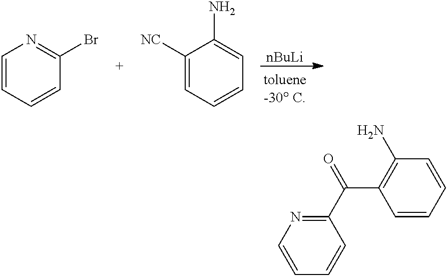

2-aminobenzonitrile (0.50 g, 4.23 mmol) and 2-bromopyridine (0.50 mL, 5.08 mmol) were dissolved in toluene (3.7 mL) and cooled to −30° C. under nitrogen atmosphere. Butyllithium (2.5 M in hexanes, 3.7 mL, 9.31 mmol) was added dropwise over 5 min. The reaction was then warmed to 0° C. and stirred for 1.5 h. The reaction mixture was poured into cooled (0° C.) HCl (3 N, 10.7 mL) and stirred for 30 minutes at room temperature. NaOH (1 N) was added until the solution was basic (pH 9), and the aqueous layer was extracted with dichloromethane (3×30 mL). The combined organic layers were washed with brine (20 mL), dried with sodium sulfate, filtered and concentrated in vacuo. The crude product was purified by silica gel chromatography (0-100% ethyl acetate/pentane) to give (2-aminophenyl)(pyridin-2-yl)methanone (394 mg, 47%).

### Preparation of 2-(5-cyclobutyl-1,3,4-thiadiazol-2-yl)aniline

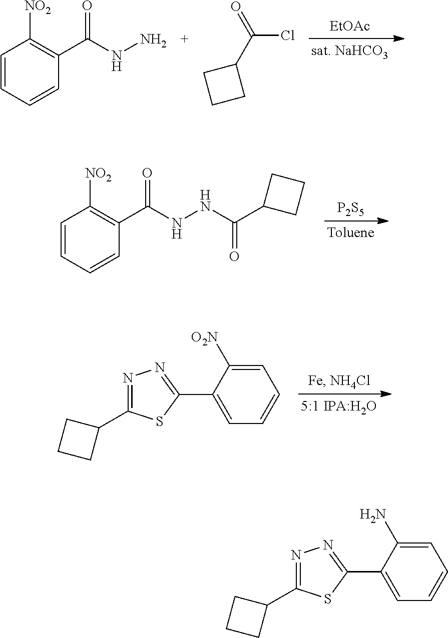

2-Nitrobenzohydrazide (1.53 g, 8.43 mmol) was suspended in ethyl acetate (35 mL) and heated to approximately 50° C. to dissolve completely. The solution was allowed to cool and a saturated aqueous sodium bicarbonate solution (10 mL) was added. A solution of cyclobutanecarbonyl chloride (0.96 mL, 8.43 mmol) in ethyl acetate (4 mL) was prepared and added slowly to the reaction mixture. A precipitate was observed forming almost immediately. After 10 minutes, 1 M aqueous HCl (12 mL) was added, followed by pentane (16 mL). The precipitate was collected by filtration and washed with H₂O. Dried under vacuum to give 1.87 g (84% yield).

N′-(Cyclobutanecarbonyl)-2-nitrobenzohydrazide (1.87 g, 7.10 mmol) was suspended in toluene (70 mL) and heated to reflux. Phosphorous pentasulfide (4.20 g, 18.89 mmol) was added and the reaction mixture was allowed to reflux for 1.75 hrs. The reaction mixture was allowed to cool to room temperature. Water was added to the reaction mixture and this was transferred to a separatory funnel. The organic layer was split off and the aqueous layer was extracted with ethyl acetate. The combined organic layers were washed with water, saturated aqueous sodium bicarbonate, and brine. The organic layer was dried over sodium sulfate and concentrated down to an oil that was used for the next reaction.

2-Cyclobutyl-5-(2-nitrophenyl)-1,3,4-thiadiazole (assumed 1.86 g, 7.10 mmol) was dissolved in a 5:1 2-propanol:water mixture (36 mL). Iron powder (1.98 g, 35.5 mmol) and ammonium chloride (0.46 g, 8.52 mmol) were added and the reaction mixture was heated to reflux for 25 minutes. The reaction mixture was filtered through celite and concentrated down to a brown oil. The oil was dissolved in a 6 M aqueous HCl solution (36 mL) and heated to 80° C. for 30 minutes. The solution was cooled in an ice bath and basified with a 2 M sodium hydroxide solution (150 mL). The mixture was extracted with ethyl acetate (3×, 45 mL). The combined organic layers were washed with 2 M sodium hydroxide (2×, 45 mL) and brine (1×, 45 mL). The organic layer was dried over sodium sulfate and concentrated down to give 0.66 g of the desired compound (40% yield, 2 steps).

### Preparation of 2-(5-ethyl-1,3,4-thiadiazol-2-yl)aniline

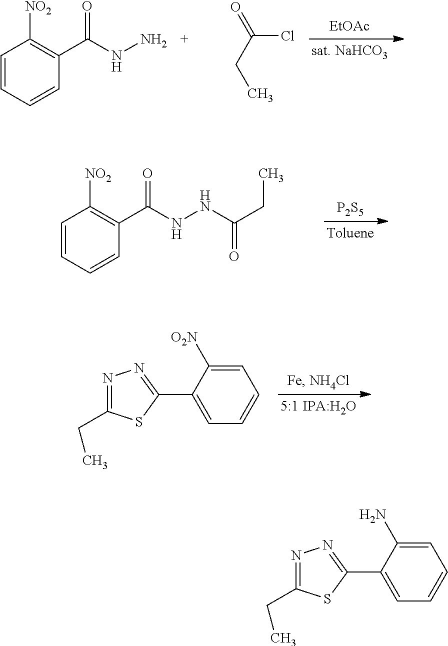

2-Nitrobenzohydrazide (1.81 g, 10.00 mmol) was suspended in ethyl acetate (40 mL) and heated to approximately 50° C. to dissolve completely. The solution was allowed to cool and a saturated aqueous sodium bicarbonate solution (12 mL) was added. A solution of propionyl chloride (0.87 mL, 10.00 mmol) in ethyl acetate (5 mL) was prepared and added slowly to the reaction mixture. A precipitate was observed forming almost immediately. After 10 minutes, 1 M aqueous HCl (15 mL) was added, followed by pentane (20 mL). The precipitate was collected by filtration and washed with H₂O. The compound was dried under vacuum to give 1.89 g (80% yield) of the desired compound.

2-Nitro-N′-propionylbenzohydrazide (1.89 g, 7.97 mmol) was suspended in toluene (70 mL) and heated to reflux. Phosphorous pentasulfide (4.71 g, 21.2 mmol) was added and the reaction mixture was allowed to reflux for 1.75 hrs. The reaction mixture was allowed to cool to room temperature. Water was added to the reaction mixture and this was transferred to a separatory funnel. The organic layer was split off and the aqueous layer was extracted with ethyl acetate. The combined organic layers were washed with water, saturated aqueous sodium bicarbonate, and brine. The organic layer was dried over sodium sulfate and concentrated down to an oil that was used for the next reaction.

2-Ethyl-5-(2-nitrophenyl)-1,3,4-thiadiazole (assumed 1.87 g, 7.97 mmol) was dissolved in a 5:1 2-propanol:water mixture (36 mL). Iron powder (2.22 g, 39.8 mmol) and ammonium chloride (0.51 g, 9.57 mmol) were added and the reaction mixture was heated to reflux for 25 minutes. The reaction mixture was filtered through celite and concentrated down to under vacuum. The residue was dissolved in ethyl acetate. The solution was washed with water, saturated sodium bicarbonate, and brine. Dried over sodium sulfate and concentrated down under vacuum. Obtained 1.04 g (64% yield, 2 steps).

### Preparation of ethyl 5-(2-aminophenyl)-1,3,4-thiadiazole-2-carboxylate

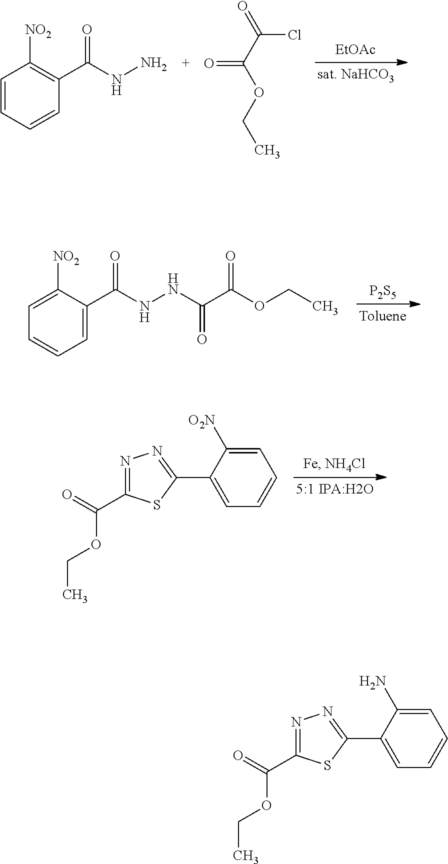

2-Nitrobenzohydrazide (1.81 g, 10.00 mmol) was suspended in ethyl acetate (40 mL) and heated to approximately 50° C. to dissolve completely. The solution was allowed to cool and a saturated aqueous sodium bicarbonate solution (12 mL) was added. A solution of ethyl chlorooxoacetate (1.12 mL, 10.00 mmol) in ethyl acetate (5 mL) was prepared and added slowly to the reaction mixture. After 10 minutes, 1 M aqueous HCl (15 mL) was added. The reaction mixture was split in a separatory funnel. The aqueous layer was extracted with ethyl acetate (2×, 50 mL). The combined organic layers were washed with brine (1×, 50 mL) and dried over sodium sulfate. The solution was concentrated down to give a white solid. The solid was suspended in 1:1 ethyl acetate:pentane and collected via filtration. The solids were washed with pentane and dried under vacuum. Obtained 2.58 g (92% yield).

Ethyl 2-(2-(2-nitrobenzoyl)hydrazinyl)-2-oxoacetate (2.58 g, 9.17 mmol) was suspended in toluene (100 mL) and heated to reflux. Phosphorous pentasulfide (5.42 g, 24.40 mmol) was added and the reaction mixture was allowed to reflux for 1.75 hrs. The reaction mixture was allowed to cool to room temperature. Water was added to the reaction mixture and this was transferred to a separatory funnel. The organic layer was split off and the aqueous layer was extracted with ethyl acetate. The combined organic layers were washed with water, saturated aqueous sodium bicarbonate, and brine. The organic layer was dried over sodium sulfate and concentrated down under vacuum (1.97 g, 77% yield) that was used for the next reaction.

Ethyl 5-(2-nitrophenyl)-1,3,4-thiadiazole-2-carboxylate (1.97 g, 7.97 mmol) was dissolved in a 5:1 2-propanol:water mixture (36 mL). Iron powder (1.97 g, 35.27 mmol) and ammonium chloride (0.45 g, 8.46 mmol) were added and the reaction mixture was heated to reflux for 25 minutes. The reaction mixture was filtered through celite and concentrated down under vacuum. The mixture was dissolved in ethyl acetate. The solution was washed with water, saturated sodium bicarbonate, and brine. Dried over sodium sulfate and concentrated down under vacuum. Obtained 1.55 g (88% yield).

### Preparation of 5-(2-aminophenyl)-1,3,4-thiadiazole-2-carboxamide

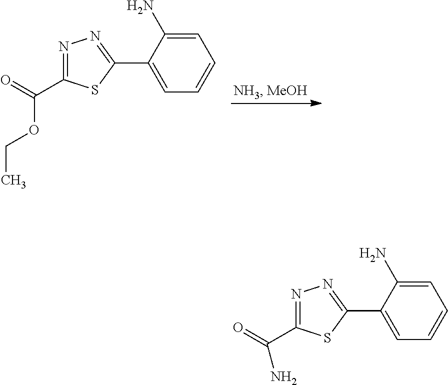

Ethyl 5-(2-nitrophenyl)-1,3,4-thiadiazole-2-carboxylate (300 mg, 1.20 mmol) was dissolved in 5 mL of a 7 M ammonia solution in methanol (35 mmol). The reaction mixture was heated to reflux for 3 hrs. The reaction mixture was concentrated down under vacuum. The residue was dissolved in ethyl acetate and washed with water, saturated sodium bicarbonate, and brine. The organic layer was dried over sodium sulfate and concentrated down under vacuum. Obtained 142 mg (54% yield)

### Preparation of 5-(2-aminophenyl)-N-methyl-1,3,4-thiadiazole-2-carboxamide

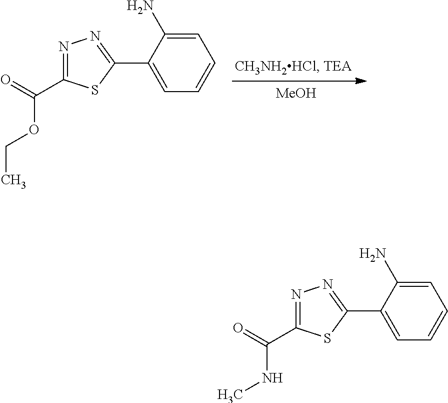

Ethyl 5-(2-nitrophenyl)-1,3,4-thiadiazole-2-carboxylate (400 mg, 1.60 mmol) was dissolved in methanol (10 mL) Methylamine hydrochloride (325 mg, 4.81 mmol) and triethylamine (0.74 mL, 5.30 mmol) were added. The reaction mixture was heated to reflux for 3 hrs. The reaction mixture was concentrated down under vacuum. Dissolved the residue in ethyl acetate and washed with water, saturated sodium bicarbonate, and brine. The organic layer was dried over sodium sulfate and concentrated down under vacuum. Obtained 331 mg (88% yield)

### Preparation of (5-(2-aminophenyl)-1,3,4-thiadiazol-2-yl)(morpholino)methanone

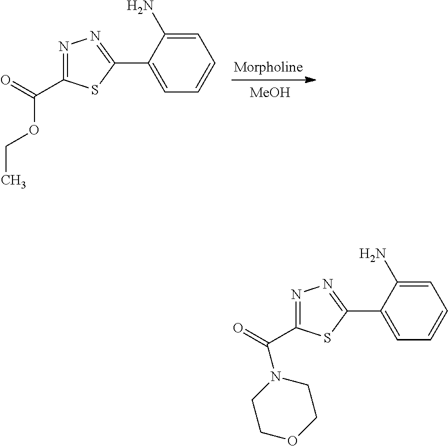

Ethyl 5-(2-nitrophenyl)-1,3,4-thiadiazole-2-carboxylate (1.00 g, 4.01 mmol) was dissolved in methanol (25 mL). The reaction mixture was heated to reflux for 3 hrs. The reaction mixture was concentrated down under vacuum. Solids precipitated out and were filtered and washed with methanol. The precipitate was dried under vacuum. Obtained 0.91 g (78% yield)

### Preparation of N-(5-(2-aminophenyl)-1,3,4-thiadiazol-2-yl)methanesulfonamide

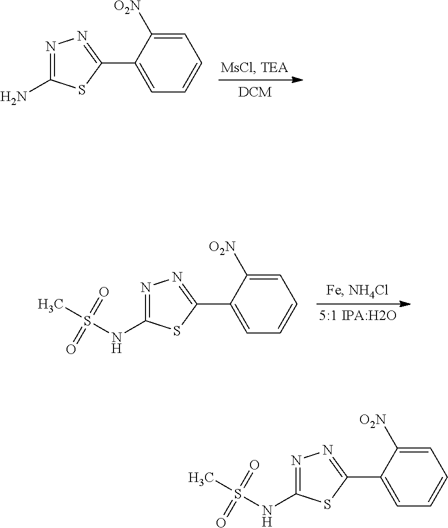

5-(2-Nitrophenyl)-1,3,4-thiadiazol-2-amine (100 mg, 0.45 mmol) was dissolved in dichloromethane (5 mL). Triethylamine was added (0.19 mL, 1.35 mmol). A solution of methanesulfonyl chloride (50 μL, 0.689 mmol) in dichloromethane (5 mL) was added. The reaction mixture was allowed to stir for 30 minutes at room temperature. LC/MS of the reaction mixture showed the formation of a disubstituted product. The reaction mixture was concentrated down under vacuum and suspended in concentrated HCl(aq). The suspension was heated to 80° C. for 30 minutes. The reaction mixture was allowed to cool and added water to the mixture. The aqueous mixture was extracted with ethyl acetate and washed with saturated aqueous sodium bicarbonate and brine. The organic layer was dried over sodium sulfate and concentrated down under vacuum. Obtained 31 mg (23% yield) of the desired compound.

N-(5-(2-Nitrophenyl)-1,3,4-thiadiazol-2-yl)methanesulfonamide (135 mg, 0.45 mmol) was suspended in 5:1 2-propanol:water (12 mL). Iron powder (126 mg, 2.25 mmol) and ammonium chloride (29 mg, 0.54 mmol) were added and the reaction mixture was heated to reflux for 30 minutes. The reaction mixture was filtered through celite and concentrated down under vacuum. The residue was dissolved in ethyl acetate and washed with water, saturated aqueous sodium bicarbonate, and brine. The organic layer was dried over sodium sulfate and concentrated under vacuum. Obtained 87 mg (72% yield) of the desired compound.

### Preparation of ethyl 5-(2-(6-chloro-2-phenylpyrimidine-4-carboxamido)phenyl)-1,3,4-thiadiazole-2-carboxylate

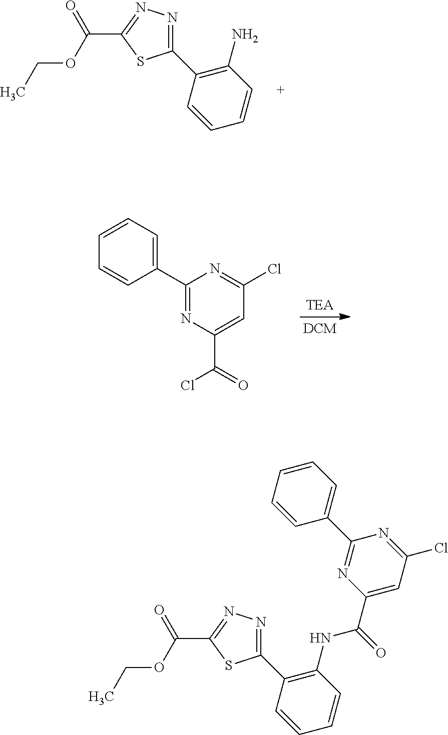

Ethyl 5-(2-aminophenyl)-1,3,4-thiadiazole-2-carboxylate (1.55 g, 5.55 mmol) was suspended in dichloromethane (DCM) (19 mL). Triethylamine (1.88 mL) was added to the suspension. A solution of 6-chloro-2-phenylpyrimidine-4-carbonyl chloride (1.57 g, 6.22 mmol) in dichloromethane (19 mL) was prepared and added slowly to the reaction mixture. The reaction mixture was allowed to stir for 10 minutes at room temperature. Methanol (100 mL) was added to the reaction mixture to precipitate out solids. The solids were filtered and washed with methanol. A second crop was collected from the filtrate. Obtained a total of 2.17 g (84% yield) of the desired compound.

### Preparation of 2-(5-(methylthio)-1,3,4-thiadiazol-2-yl)aniline

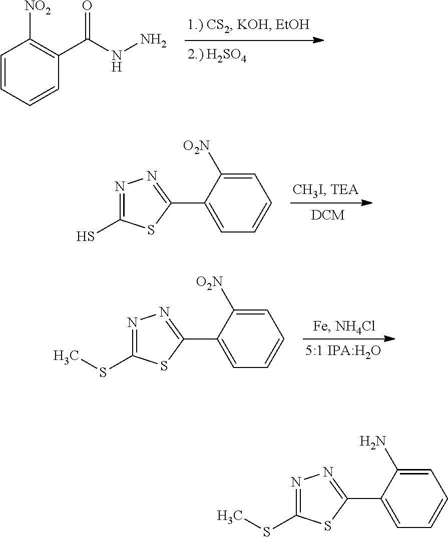

2-Nitrobenzohydrazide (10.00 g, 55.2 mmol) and potassium hydroxide (3.10 g, 55.2 mmol) were dissolved in ethanol (300 mL). Carbon disulfide (5.0 mL, 82.8 mmol) was added and the reaction mixture was allowed to stir at room temperature for 4 hrs. The reaction mixture was concentrated under vacuum. The residue was cooled in an ice bath and concentrated sulfuric acid (50 mL) was added to dissolve the residue. Water (100 mL) was added to the solution and the reaction mixture was basified with 2 M sodium hydroxide (aq.) until a precipitate formed. The precipitate was filtered and washed with water and dried under vacuum. Obtained 7.23 g (55% yield)

5-(2-Nitrophenyl)-1,3,4-thiadiazole-2-thiol (2.00 g, 8.36 mmol) was dissolved in dichloromethane (50 mL). Triethylamine (3.5 mL, 25.08 mmol) was added followed by iodomethane (0.65 mL, 10.45 mmol). The reaction mixture was allowed to stir at room temperature for 1 hour. The reaction mixture was washed with water, saturated sodium bicarbonate (aq), and brine. The organic layer was dried over sodium sulfate and concentrated down under vacuum. Obtained 2.12 g (>99% yield).

2-(Methylthio)-5-(2-nitrophenyl)-1,3,4-thiadiazole (2.12 g, 8.37 mmol) was suspended in 5:1 2-propanol:water (120 mL) Iron powder (2.33 g, 41.85 mmol) and ammonium chloride (0.54 g, 10.04 mmol) were added and the reaction mixture was heated to reflux for 25 minutes. The reaction mixture was filtered through celite and concentrated down under vacuum. The mixture was dissolved in ethyl acetate. The solution was washed with water, saturated sodium bicarbonate, and brine. Dried over sodium sulfate and concentrated down under vacuum. Obtained 1.15 g (62% yield).

### Preparation of N-(5-(2-aminophenyl)-1,3,4-thiadiazol-2-yl)morpholine-4-carboxamide

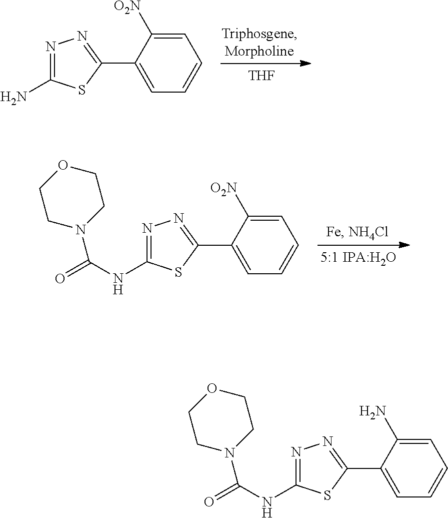

5-(2-Nitrophenyl)-1,3,4-thiadiazol-2-amine (200 mg, 0.90 mmol) was dissolved in tetrahydrofuran (5 mL) and cooled in an ice bath. A solution of triphosgene (89 mg, 0.30 mmol) in tetrahydrofuran (5 mL) was added to the starting material and the reaction mixture was allowed to stir for 1 hr. Morpholine (0.24 mL, 2.70 mmol) was added to the reaction mixture and the reaction was allowed to stir for 1 hr. The reaction mixture was concentrated under vacuum and redissolved in ethyl acetate and washed with water, saturated sodium bicarbonate (aq), and brine. The organic layer was dried over sodium sulfate and concentrated down under vacuum. Obtained 224 mg (74% yield)

N-(5-(2-Nitrophenyl)-1,3,4-thiadiazol-2-yl)morpholine-4-carboxamide (224 mg, 0.668 mmol) was suspended in 5:1 2-propanol:water (24 mL) Iron powder (187 mg, 3.34 mmol) and ammonium chloride (43 mg, 0.802 mmol) were added and the reaction mixture was heated to reflux for 25 minutes. The reaction mixture was filtered through celite and concentrated down under vacuum. The mixture was dissolved in ethyl acetate. The solution was washed with water, saturated sodium bicarbonate, and brine. Dried over sodium sulfate and concentrated down under vacuum. Obtained 167 mg (82% yield).

### 1-(2-aminophenyl)propan-1-one

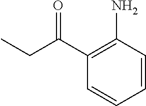

To a solution of 591 mg (5.00 mmol) of anthranilonitrile in 10 mL of THF at −78° C. under N₂ was added 11 mL of 1.0 M ethyl magnesium bromide in THF. The reaction was warmed to reflux for 10 min, then diluted with 5 mL of water, and concentrated in vacuo to remove the THF. The residue was suspended in 20 mL of 0.5 M HCl_{(aq.)}, then the mixture was extracted with ethyl acetate (2×15 mL). The combined ethyl acetate layers were back extracted with brine (1×10 mL), dried over MgSO₄, filtered, and concentrated in vacuo to a yellow oil. This was purified via MPLC, eluting with a 5-15% ethyl acetate/pentane gradient to give 290 mg (39%) of 1-(2-aminophenyl)propan-1-one as a colorless crystalline solid.

### Preparation of 1-(2-aminophenyl)pentan-1-one

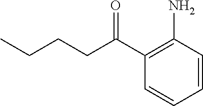

To a solution of 591 mg (5.00 mmol) of anthranilonitrile in 10 mL of THF under N₂ at ambient temperature was added 4.3 mL (10.8 mmol) of 2.5 M n-BuLi in hexanes. The reaction was stirred at ambient temperature for 10 min, then 3 mL of water was added, and the solvents were removed in vacuo. The residue was suspended in 15 mL of 1 M HCl, then 15 mL of saturated NaHCO_{3(aq.)}. The suspension was extracted with ethyl acetate (2×15 mL), then brine (1×10 mL), dried over MgSO₄, filtered, and concentrated to an oil. This was purified via MPLC, eluting with a 5-15% ethyl acetate/pentane gradient to give 550 mg (62%) of a yellow oil.

### Preparation of 1-(2-aminophenyl)-2-methylpropan-1-one

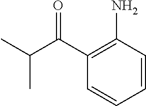

To a solution of 591 mg (5.00 mmol) of anthranilonitrile in 10 mL of THF at ambient temperature was added 6 mL (12 mmol) of 2.0M isopropylmagnesium bromide in THF. The reaction was stirred at ambient temperature for 5 min, then heated at reflux for 1 h. The reaction was cooled, and diluted with 5 mL of water, then concentrated in vacuo. The residue was diluted with 15 mL of 1 M HCl_{(aq.)}, then 15 mL of saturated NaHCO_{3(aq.)} was added, and the mixture was extracted with ethyl acetate (3×10 mL). The combined ethyl acetate layers were back extracted with brine (1×10 mL), dried over MgSO₄, filtered, and concentrated to an oil. This was purified via MPLC, eluting with a 5-15% ethyl acetate/pentane gradient to give 163 mg (20%) of a yellow oil.

### General Method B:

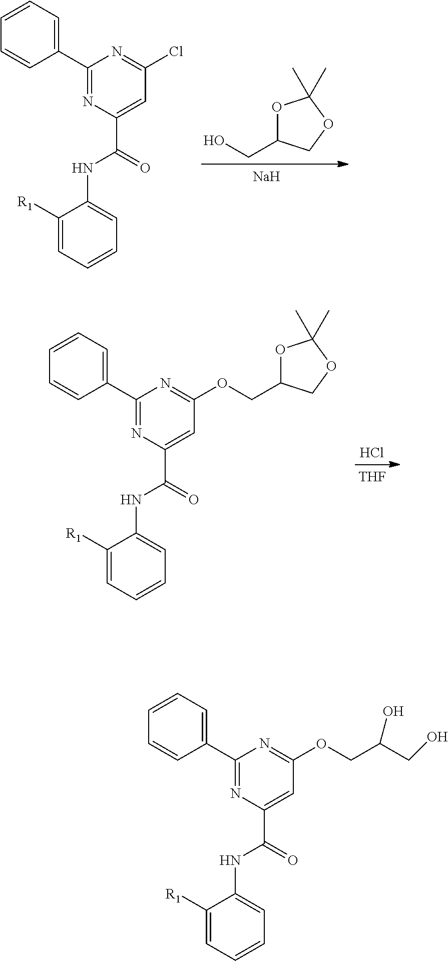

(Chloropyrimidine is prepared as in General Method A.)

Sodium hydride (5.5 eq) is suspended in dimethylsulfoxide (DMSO). (2,2-dimethyl-1,3-dioxolan-4-yl)methanol (5 eq) is dissolved in THF and added to the DMSO solution. The mixture is stirred at room temperature for 5 min. Chloropyrimidine is added in DMSO, and the mixture is stirred at room temperature for 6 h. This is poured into water and 1N HCl (5 eq), extracted with 10% methanol/dichloromethane, dried with sodium sulfate, filtered and concentrated. The crude product can be purified either by silica gel chromatography (0-10% methanol/dichloromethane) or by trituration with ethyl acetate.

Protected glycerol-pyrimidine is suspended in THF. Concentrated HCl (3 eq) is added dropwise. This is stirred at room temperature for 1 day. Acetonitrile is added to induce precipitation, and the product is collected by filtration. This is purified by trituration with 4:1 ether:ethanol.

### General Method C:

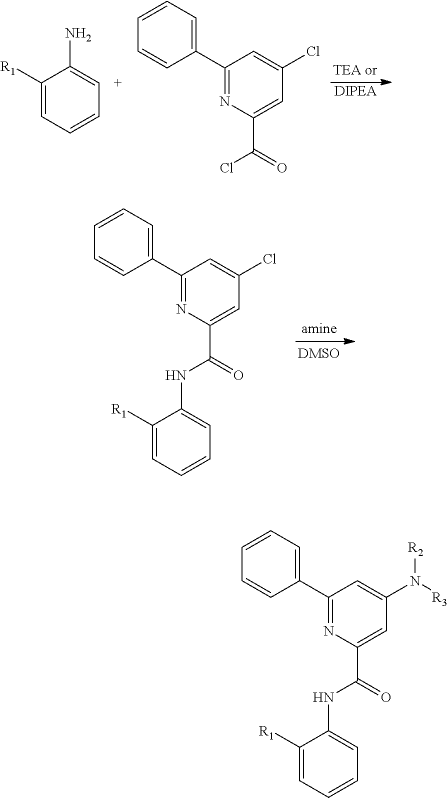

Chloropyridine is prepared in a manner similar to the preparation of chloropyrimidine in general method A, starting with 4-chloro-6-phenylpicolinoyl chloride. The reaction should run 3 h or longer, and heating to reflux may be necessary for complete conversion.

Chloropyrimidine and amine (10 eq) are suspended in DMSO in a sealed tube and heated to 110-120° C. (either conventionally or in a microwave) for 1-3 days until complete conversion. The mixture is poured into water, and the product is collected by filtration. In cases where the product does not precipitate, it is extracted with ethyl acetate, washed with brine, dried with sodium sulfate, filtered and concentrated. The crude product is purified by trituration with acetonitrile, or in some cases by silica gel chromatography (0-10% methanol/dichloromethane).

### General Method D:

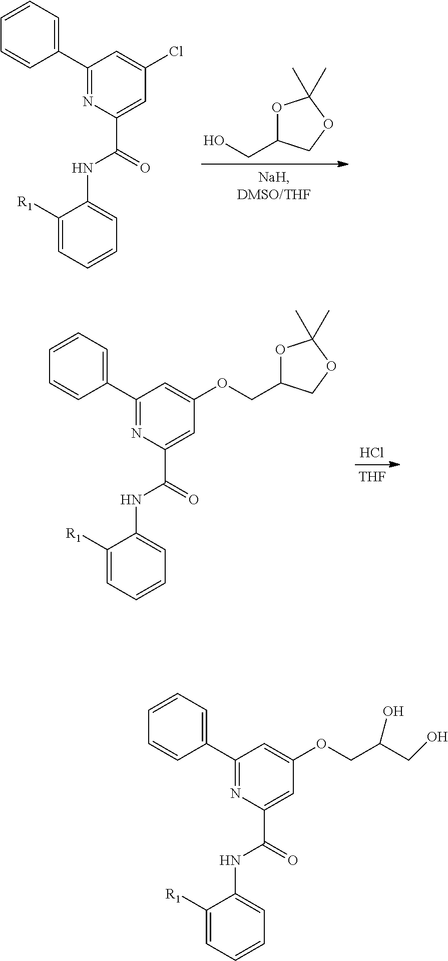

(Chloropyridine is prepared as in General Method C.)

Sodium hydride (5.5 eq) is suspended in DMSO. Glycerol (5 eq) is dissolved in THF and added to the DMSO solution. The mixture is stirred at room temperature for 5 min. Chloropyridine is added in DMSO, and the mixture is stirred at room temperature for 4 days. This is poured into water and 1N HCl (5 eq), extracted with ethyl acetate (3×30 mL), the organic layer washed with saturated aqueous sodium bicarbonate (20 mL) and brine (20 mL), dried with sodium sulfate, filtered and concentrated in vacuo. The crude product is purified by silica gel chromatography (20-50% ethyl acetate/pentane).

Protected glycerol-pyridine is suspended in THF. Concentrated HCl (3 eq) is added dropwise. This is stirred at room temperature for 4 h. Acetonitrile is added to induce precipitation, and the product is collected by filtration.

### Preparation of 6-(2-(dimethylamino)ethylamino)-2-phenyl-N-(2-(thiazol-2-yl)phenyl)pyrimidine-4-carboxamide

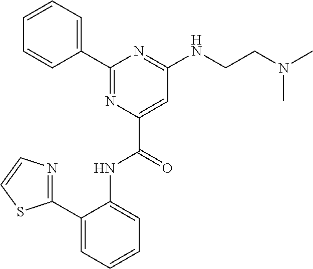

The title compound was prepared according to general method A, utilizing and 2-(2-thiazolyl)aniline and N,N-dimethylethylenediamine. Yield 33 mg (48%, 2 steps). MS Calcd for C₂₄H₂₄N₆OS: 444.17. Found (M+H)⁺ m/z=445.

### Preparation of 6-(2-(dimethylamino)ethylamino)-N,2-diphenylpyrimidine-4-carboxamide

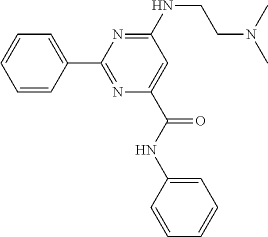

The title compound was prepared according to general method A, utilizing aniline and N,N-dimethylethylenediamine. Yield 239 mg (20%, 2 steps). MS Calcd for C₂₁H₂₃N₅O: 361.19. Found (M+H)⁺ m/z=362.

### Preparation of N-(biphenyl-2-yl)-6-(2-(dimethylamino)ethylamino)-2-phenylpyrimidine-4-carboxamide

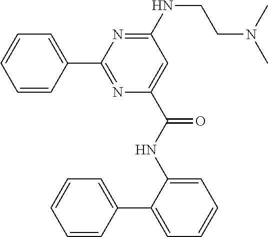

The title compound was prepared according to general method A, utilizing biphenyl-2-amine and N,N-dimethylethylenediamine. Yield 188 mg (36%, 2 steps). MS Calcd for C₂₇H₂₇N₅O: 437.22. Found (M+H)⁺ m/z=438.

### Preparation of N-(2-carbamoylphenyl)-6-(2-(dimethylamino)ethylamino)-2-phenylpyrimidine-4-carboxamide

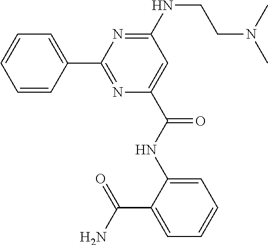

The title compound was prepared according to general method A, utilizing 2-aminobenzamide and N,N-dimethylethylenediamine. Yield 157 mg (26%, 2 steps). MS Calcd for C₂₂H₂₄N₆O₂: 404.20. Found (M+H)⁺ m/z=405.

### Preparation of N-(2-cyanophenyl)-6-(2-(dimethylamino)ethylamino)-2-phenylpyrimidine-4-carboxamide

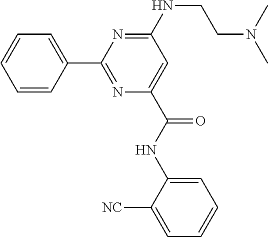

The title compound was prepared according to general method A, utilizing 2-aminobenzonitrile and N,N-dimethylethylenediamine. Yield 91 mg (14%, 2 steps). MS Calcd for C₂₂H₂₂N₆O: 386.19. Found (M+H)⁺ m/z=387.

### Preparation of methyl 2-(6-(2-(dimethylamino)ethylamino)-2-phenylpyrimidine-4-carboxamido)benzoate

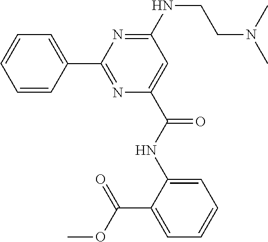

The title compound was prepared according to general method A, utilizing methyl 2-aminobenzoate and N,N-dimethylethylenediamine. Yield 292 mg (35%, 2 steps). MS Calcd for C₂₃H₂₅N₅O₃: 419.20. Found (M+H)⁺ m/z=420.

### Preparation of 2-(6-(2-(dimethylamino)ethylamino)-2-phenylpyrimidine-4-carboxamido)benzoic acid

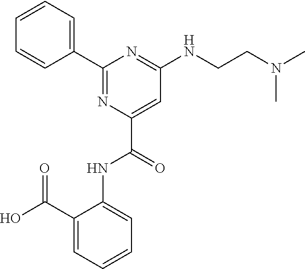

A mixture of 2.0 g (4.8 mmol) of methyl 2-(6-(2-(dimethylamino)ethylamino)-2-phenylpyrimidine-4-carboxamido)benzoate and 0.4 g (9.5 mmol) of lithium hydroxide in 10 mL of THF, 10 mL of methanol, and 20 mL of water was stirred at 45° C. for 18 h. Next, 10% HCl(aq.) was added to pH=4, then most of the solvents were removed in vacuo. A solid precipitated from the remaining volume, and this was filtered. Recrystallization gave 1.1 g (57%) of a white solid.

### Preparation of 6-(2-(dimethylamino)ethylamino)-N-(2-ethylphenyl)-2-phenylpyrimidine-4-carboxamide

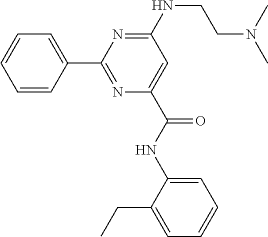

The title compound was prepared according to general method A, utilizing 2-ethylaniline and N,N-dimethylethylenediamine. Yield 245 mg (25%, 2 steps). MS Calcd for C₂₃H₂₇N₅O: 389.22. Found (M+H)⁺ m/z=390.

### Preparation of 6-(2-(dimethylamino)ethylamino)-N-(2-(5-methylthiazol-2-yl)phenyl)-2-phenylpyrimidine-4-carboxamide

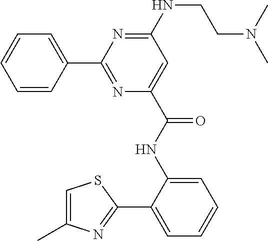

The title compound was prepared according to the general method A using 2-(4-methylthiazol-2-yl)aniline and N,N-dimethylethylenediamine. Yield 40 mg (57%, 2 steps). MS Calcd for C₂₅H₂₆N₆OS: 458.19. Found (M+H)⁺ m/z=459.

### 6-(2-(dimethylamino)ethyl)(ethyl)amino)-2-phenyl-N-(2-(thiazol-2-yl)-phenyl)pyrimidine-4-carboxamide

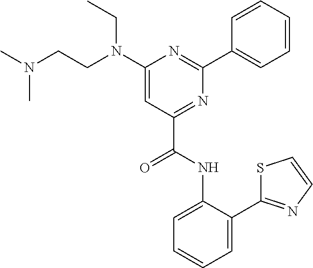

The title compound was prepared according to general method A, from 2-(2-thiazolyl)aniline and N¹-ethyl-N²,N²-dimethylethane-1,2-diamine. Yield 356 mg (62%, 2 steps). MS Calcd. for C₂₆H₂₈N₆OS: 472.20 Found (M+H)⁺ m/z=473.

### Preparation of 6-(2-(dimethylamino)ethylamino)-2-phenyl-N-(2-(2-phenylthiazol-4-yl)phenyl)pyrimidine-4-carboxamide

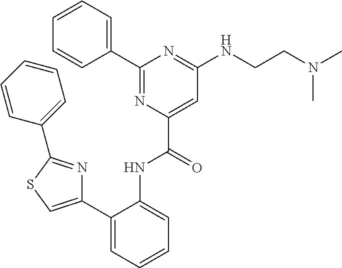

The title compound was prepared according to general method A, utilizing 2-(2-phenylthiazol-4-yl)aniline and N,N-dimethylethylenediamine. Yield 493 mg (68%, 2 steps). MS Calcd for C₃₀H₂₈N₆OS: 520.20. Found (M+H)⁺ m/z=521.

### Preparation of 6-(2-(dimethylamino)ethylamino)-2-phenyl-N-(2-(5-phenylthiazol-2-yl)phenyl)pyrimidine-4-carboxamide

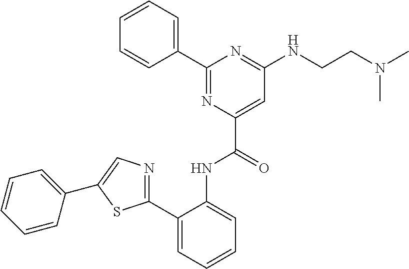

The title compound was prepared according to general method A, utilizing 2-(5-phenylthiazol-2-yl)aniline and N,N-dimethylethylenediamine. Yield 400 mg (85%, 2 steps). MS Calcd for C₃₀H₂₈N₆OS: 520.20. Found (M+H)⁺ m/z=521.

### Preparation of 6-(2-(dimethylamino)ethylamino)-2-phenyl-N-(2-(4-phenylthiazol-2-yl)phenyl)pyrimidine-4-carboxamide

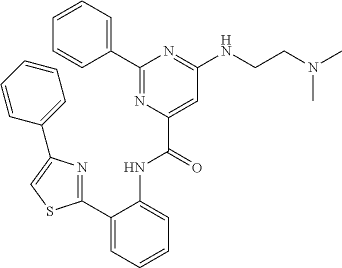

The title compound was prepared according to general method A, utilizing 2-(4-phenylthiazol-2-yl)aniline and N,N-dimethylethylenediamine. Yield 100 mg (40%, 2 steps). MS Calcd for C₃₀H₂₈N₆OS: 520.20. Found (M+H)⁺ m/z=521.

### Preparation of 6-(2-(dimethylamino)ethylamino)-2-phenyl-N-(2-(2-phenyl-1H-imidazol-4-yl)phenyl)pyrimidine-4-carboxamide

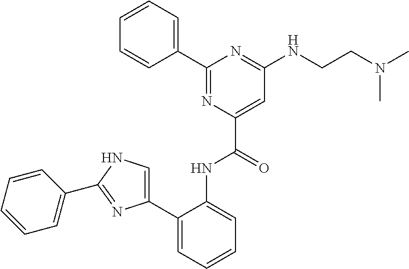

The title compound was prepared according to general method A, utilizing 2-(2-phenyl-1H-imidazole-4-yl)aniline and N,N-dimethylethylenediamine. Yield 190 mg (40%, 2 steps). MS Calcd for C₃₀H₂₉N₇O: 503.24. Found (M+H)⁺ m/z=504.

### Preparation of 2-phenyl-N-(2-(5-phenylthiazol-2-yl)phenyl)-6-(2-(piperidin-1-yl)ethylamino)pyrimidine-4-carboxamide

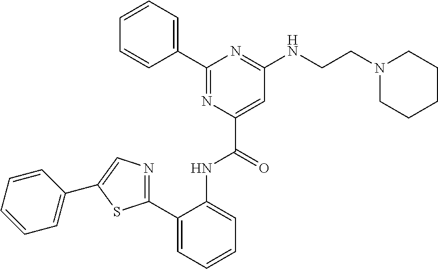

The title compound was prepared according to general method A, utilizing 2-(5-phenylthiazol-2-yl)aniline and 2-(piperidin-1-yl)ethanamine. Yield 205 mg (75%, 2 steps). MS Calcd for C₃₃H₃₂N₆OS: 560.24. Found (M+H)⁺ m/z=561.

### Preparation of 2-phenyl-N-(2-(5-phenylthiazol-2-yl)phenyl)-6-(piperazin-1-yl)pyrimidine-4-carboxamide

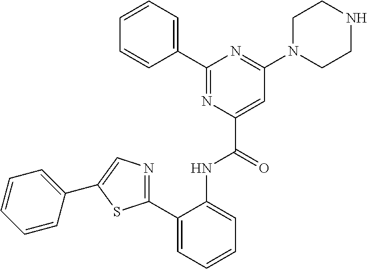

The title compound was prepared according to general method A, utilizing 2-(5-phenylthiazol-2-yl)aniline and Boc-piperazine, followed by treatment with 20% trifluoro acetic acid(TFA) in DCM. The solvent was evaporated, the residue was taken up in acetonitrile/water 1:5, 1N aqueous HCl (6 equiv) was added and the mixture was lyophilized to afford the title compound as the HCl salt. Yield 66 mg (28%, 4 steps). MS Calcd for C₃₀H₂₆N₆OS: 518.19. Found (M+H)⁺ m/z=519.

### Preparation of 2-phenyl-N-(2-(5-phenylthiazol-2-yl)phenyl)-6-(2-(pyrrolidin-1-yl)ethylamino)pyrimidine-4-carboxamide

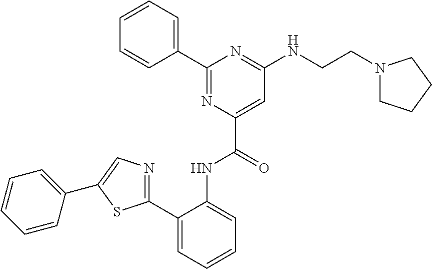

The title compound was prepared according to general method A, utilizing 2-(5-phenylthiazol-2-yl)aniline and 2-(pyrrolidin-1-yl)ethanamine. Yield 219 mg (83%, 2 steps). MS Calcd for C₃₂H₃₀N₆OS: 546.22. Found (M+H)⁺ m/z=547.

### Preparation of 6-(2-(dimethylamino)ethylamino)-2-phenyl-N-(2-(4-phenyl-1H-pyrazol-1-yl)phenyl)pyrimidine-4-carboxamide

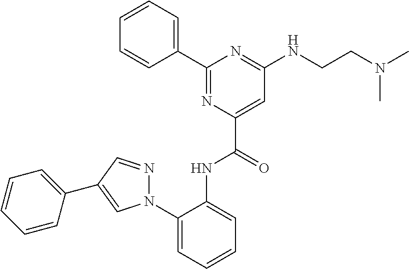

The title compound was prepared according to general method A, utilizing 2-(4-phenyl-1H-pyrazol-1-yl)aniline and N,N-dimethylethylenediamine. Yield 109 mg (43%, 2 steps). MS Calcd for C₃₀H₂₉N₇O: 503.24. Found (M+H)⁺ m/z=504.

### Preparation of N-(2-(5-tert-butylthiazol-2-yl)phenyl)-6-(2-(dimethylamino)ethylamino)-2-phenylpyrimidine-4-carboxamide

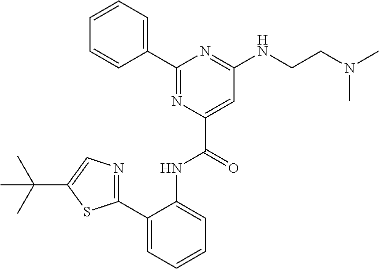

The title compound was prepared according to general method A, utilizing 2-(5-tert-butylthiazol-2-yl)aniline and N,N-dimethylethylenediamine. Yield 156 mg (61%, 2 steps). MS Calcd for C₂₈H₃₂N₆OS: 500.24. Found (M+H)⁺ m/z=501.

### Preparation of 6-(2-(dimethylamino)ethylamino)-2-phenyl-N-(2-(5-phenyloxazol-2-yl)phenyl)pyrimidine-4-carboxamide

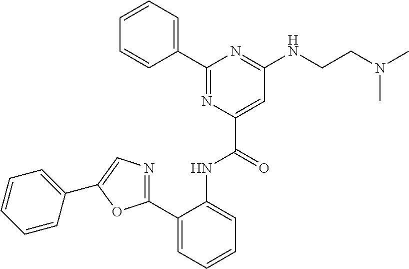

The title compound was prepared according to general method A, utilizing 2-(5-phenyloxazol-2-yl)aniline and N,N-dimethylethylenediamine. Yield 186 mg (73%, 2 steps). MS Calcd for C₃₀H₂₈N₆O₂: 504.23. Found (M+H)⁺ m/z=505.

### Preparation of N-(2-(5-tert-butyloxazol-2-yl)phenyl)-6-(2-(dimethylamino)ethylamino)-2-phenylpyrimidine-4-carboxamide

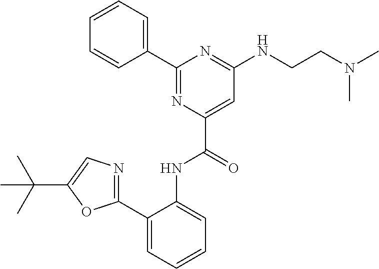

The title compound was prepared according to general method A, utilizing 2-(5-tert-butyloxazol-2-yl)aniline and N,N-dimethylethylenediamine. Yield 166 mg (65%, 2 steps). MS Calcd for C₂₈H₃₂N₆O₂: 484.26. Found (M+H)⁺ m/z=485.

### Preparation of N-(2-(5-tert-butylthiazol-2-yl)phenyl)-6-((2-(dimethylamino)ethyl)(ethyl)amino)-2-phenylpyrimidine-4-carboxamide

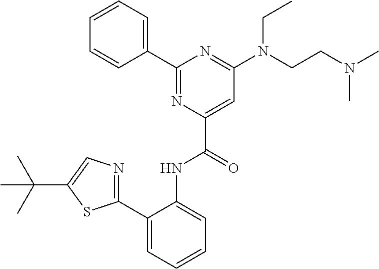

The title compound was prepared according to general method A, utilizing 2-(5-tert-butylthiazol-2-yl)aniline and N,N-dimethyl-N′ ethylethylenediamine. Yield 175 mg (65%, 2 steps). MS Calcd for C₃₀H₃₆N₆OS: 528.27. Found (M+H)⁺ m/z=529.

### Preparation of 6-((2-(dimethylamino)ethyl)(ethyl)amino)-2-phenyl-N-(2-(5-phenyloxazol-2-yl)phenyl)pyrimidine-4-carboxamide

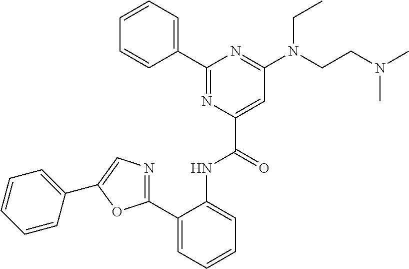

The title compound was prepared according to general method A, utilizing 2-(5-phenyloxazol-2-yl)aniline and N,N-dimethyl-N′ ethylethylenediamine. Yield 170 mg (64%, 2 steps). MS Calcd for C₃₂H₃₂N₆O₂: 532.26. Found (M+H)⁺ m/z=533.

### Preparation of N-(2-(5-tert-butyloxazol-2-yl)phenyl)-6-((2-(dimethylamino)ethyl)(ethyl)amino)-2-phenylpyrimidine-4-carboxamide

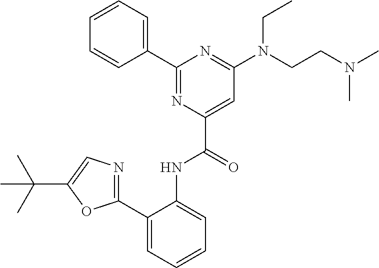

The title compound was prepared according to general method A, utilizing 2-(5-tert-butyloxazol-2-yl)aniline and N,N-dimethyl-N′ ethylethylenediamine. Yield 183 mg (68%, 2 steps). MS Calcd for C₃₀H₃₆N₆O₂: 512.29. Found (M+H)⁺ m/z=513.

### Preparation of 6-((2-(dimethylamino)ethyl)(ethyl)amino)-2-phenyl-N-(2-(2-phenyl-1H-imidazol-4-yl)phenyl)pyrimidine-4-carboxamide

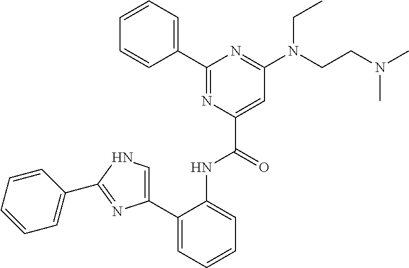

The title compound was prepared according to general method A, utilizing 2-(2-phenyl-1H-imidazole-4-yl)aniline and N,N-dimethyl-N′ ethylethylenediamine. Yield 105 mg (70%, 2 steps). MS Calcd for C₃₂H₃₃N₇O: 531.27. Found (M+H)⁺ m/z=532.

### Preparation of 6-(2-(dimethylamino)ethylamino)-2-phenyl-N-(2-(5-(pyridin-2-yl)-1,3,4-thiadiazol-2-yl)phenyl)pyrimidine-4-carboxamide

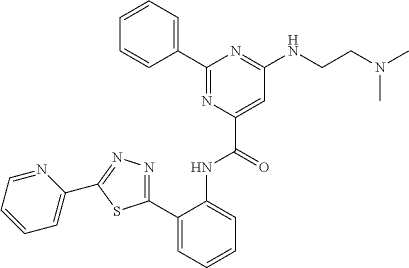

The title compound was prepared according to general method A, utilizing 2-(5-(pyridine-2-yl)-1,3,4-thiadiazol-2-yl)aniline and N,N-dimethylethylenediamine. Yield 100 mg (60%, 2 steps). MS Calcd for C₂₈H₂₆N₈OS: 522.20. Found (M+H)⁺ m/z=523.

### Preparation of 6-((2-(dimethylamino)ethyl)(ethyl)amino)-2-phenyl-N-(2-(5-(pyridin-2-yl)-1,3,4-thiadiazol-2-yl)phenyl)pyrimidine-4-carboxamide

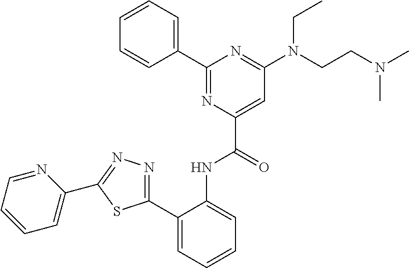

The title compound was prepared according to general method A, utilizing 2-(5-(pyridine-2-yl)-1,3,4-thiadiazol-2-yl)aniline and N,N-dimethyl-N′ ethylethylenediamine. Yield 130 mg (51%, 2 steps). MS Calcd for C₃₀H₃₀N₈OS: 550.23. Found (M+H)⁺ m/z=551.

### Preparation of 6-(2-(dimethylamino)ethylamino)-2-phenyl-N-(2-(5-(pyridin-3-yl)-1,3,4-thiadiazol-2-yl)phenyl)pyrimidine-4-carboxamide

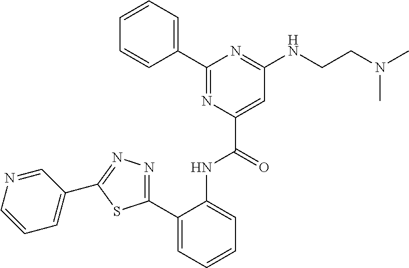

The title compound was prepared according to general method A, utilizing 2-(5-(pyridine-3-yl)-1,3,4-thiadiazol-2-yl)aniline and N,N-dimethylethylenediamine. Yield 80 mg (61%, 2 steps). MS Calcd for C₂₈H₂₆N₈OS: 522.20. Found (M+H)⁺ m/z=523.

### Preparation of 6-(2-(dimethylamino)ethylamino)-2-phenyl-N-(2-(5-(pyridin-4-yl)-1,3,4-thiadiazol-2-yl)phenyl)pyrimidine-4-carboxamide

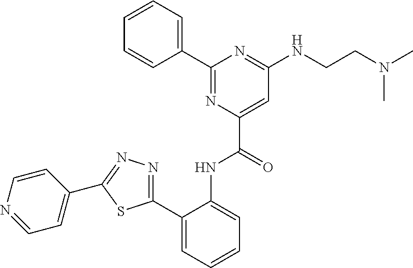

The title compound was prepared according to general method A, utilizing 2-(5-(pyridine-4-yl)-1,3,4-thiadiazol-2-yl)aniline and N,N-dimethylethylenediamine. Yield 88 mg (87%, 2 steps). MS Calcd for C₂₈H₂₆N₈OS: 522.20. Found (M+H)⁺ m/z=523.

### Preparation of 6-((2-(dimethylamino)ethyl)(ethyl)amino)-2-phenyl-N-(2-(5-(pyridin-3-yl)-1,3,4-thiadiazol-2-yl)phenyl)pyrimidine-4-carboxamide

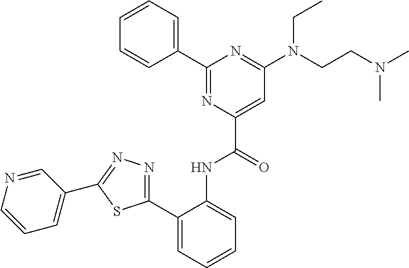

The title compound was prepared according to general method A, utilizing 2-(5-(pyridine-3-yl)-1,3,4-thiadiazol-2-yl)aniline and N,N-dimethyl-N′ ethylethylenediamine. Yield 84 mg (61%, 2 steps). MS Calcd for C₃₀H₃₀N₈OS: 550.23. Found (M+H)⁺ m/z=551.

### Preparation of 6-((2-(dimethylamino)ethyl)(ethyl)amino)-2-phenyl-N-(2-(5-(pyridin-4-yl)-1,3,4-thiadiazol-2-yl)phenyl)pyrimidine-4-carboxamide

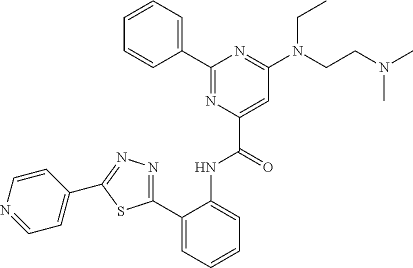

The title compound was prepared according to general method A, utilizing 2-(5-(pyridine-4-yl)-1,3,4-thiadiazol-2-yl)aniline and N,N-dimethyl-N′ ethylethylenediamine. Yield 84 mg (77%, 2 steps). MS Calcd for C₃₀H₃₀N₈OS: 550.23. Found (M+H)⁺ m/z=551.

### Preparation of 6-morpholino-N-(2-(5-morpholino-1,3,4-thiadiazol-2-yl)phenyl)-2-phenylpyrimidine-4-carboxamide

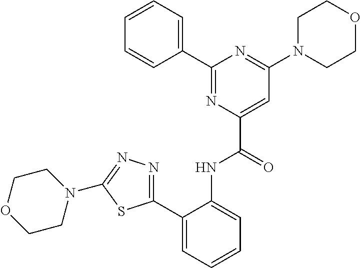

The title compound was prepared according to general method A, utilizing 2-(5-morpholino-1,3,4-thiadiazol-2-yl)aniline and morpholine. Yield 113 mg (60%, 2 steps). MS Calcd for C₂₇H₂₇N₇O₃S: 529.19. Found (M+H)⁺ m/z=530.

### Preparation of 6-(2-(dimethylamino)ethylamino)-N-(2-(5-morpholino-1,3,4-thiadiazol-2-yl)phenyl)-2-phenylpyrimidine-4-carboxamide

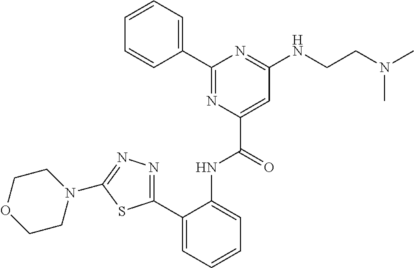

The title compound was prepared according to general method A, utilizing 2-(5-morpholino-1,3,4-thiadiazol-2-yl)aniline and N,N-dimethylethylenediamine. Yield 145 mg (77%, 2 steps). MS Calcd for C₂₇H₃₀N₈O₂S: 530.22. Found (M+H)⁺ m/z=531.

### Preparation of 6-(1,3-dihydroxypropan-2-ylamino)-N-(2-(5-morpholino-1,3,4-thiadiazol-2-yl)phenyl)-2-phenylpyrimidine-4-carboxamide

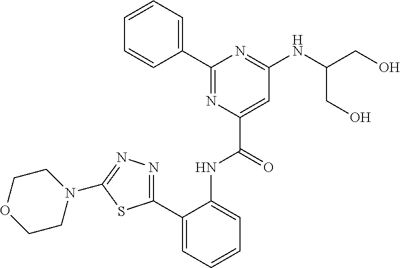

The title compound was prepared according to general method A, utilizing 2-(5-morpholino-1,3,4-thiadiazol-2-yl)aniline and serinol. Yield 95 mg (50%, 2 steps). MS Calcd for C₂₆H₂₇N₇O₄S: 533.18. Found (M+H)⁺ m/z=534.

### Preparation of N-(2-(5-morpholino-1,3,4-thiadiazol-2-yl)phenyl)-6-(3-oxopiperazin-1-yl)-2-phenylpyrimidine-4-carboxamide

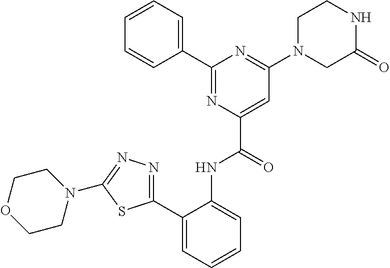

The title compound was prepared according to general method A, utilizing 2-(5-morpholino-1,3,4-thiadiazol-2-yl)aniline and 2-oxopiperazine. Yield 148 mg (77%, 2 steps). MS Calcd for C₂₇H₂₆N₈O₃S: 542.18. Found (M+H)⁺ m/z=543.

### Preparation of 6-(4-ethyl-3-oxopiperazin-1-yl)-N-(2-(5-morpholino-1,3,4-thiadiazol-2-yl)phenyl)-2-phenylpyrimidine-4-carboxamide

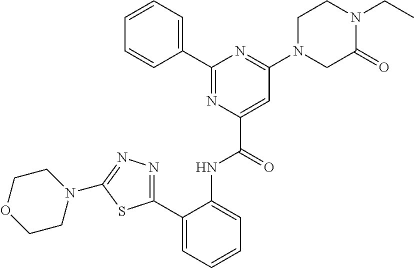

The title compound was prepared according to general method A, utilizing 2-(5-morpholino-1,3,4-thiadiazol-2-yl)aniline and 1-ethylpiperazin-2-one. Yield 139 mg (68%, 2 steps). MS Calcd for C₂₉H₃₀N₈O₃S: 570.22. Found (M+H)⁺ m/z=571.

### Preparation of 6-((2-(dimethylamino)ethyl)(ethyl)amino)-N-(2-(5-morpholino-1,3,4-thiadiazol-2-yl)phenyl)-2-phenylpyrimidine-4-carboxamide

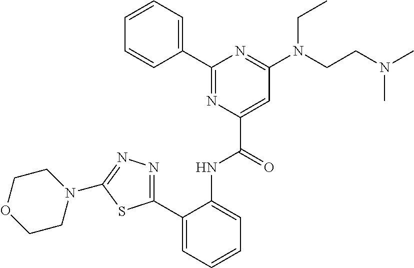

The title compound was prepared according to general method A, utilizing 2-(5-morpholino-1,3,4-thiadiazol-2-yl)aniline and N,N-dimethyl-N′ ethylethylenediamine. Yield 132 mg (66%, 2 steps). MS Calcd for C₂₉H₃₄N₈O₂S: 558.25. Found (M+H)⁺ m/z=559.

### Preparation of (S)-N-(2-(5-(hexahydropyrrolo[1,2-a]pyrazin-2(1H)-yl)-1,3,4-thiadiazol-2-yl)phenyl)-6-(2-methoxyethylamino)-2-phenylpyrimidine-4-carboxamide

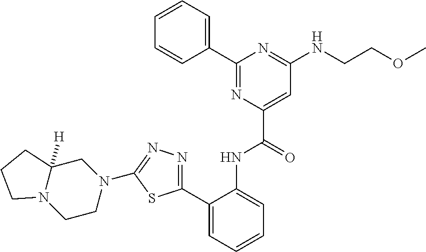

The title compound was prepared according to general method A, utilizing (S)-2-(5-(hexahydropyrrolo[1,2-a]pyrazin-2(1H)-yl)-1,3,4-thiadiazol-2-yl)aniline and 2-methoxyethylamine. Yield 97 mg (79%, 2 steps). MS Calcd for C₂₉H₃₂N₈O₂S: 556.24. Found (M+H)⁺ m/z=557.

### Preparation of (S)-N-(2-(5-(hexahydropyrrolo[1,2-a]pyrazin-2(1H)-yl)-1,3,4-thiadiazol-2-yl)phenyl)-6-(3-oxopiperazin-1-yl)-2-phenylpyrimidine-4-carboxamide

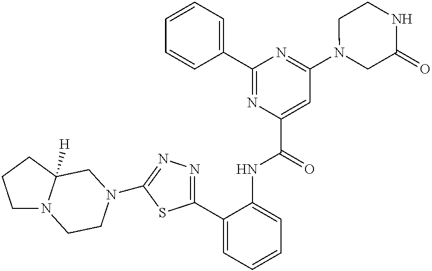

The title compound was prepared according to general method A, utilizing (S)-2-(5-(hexahydropyrrolo[1,2-a]pyrazin-2(1H)-yl)-1,3,4-thiadiazol-2-yl)aniline and 2-oxopiperazine. Yield 53 mg (42%, 2 steps). MS Calcd for C₃₀H₃₁N₉O₂S: 581.23. Found (M+H)⁺ m/z=582.

### Preparation of 6-(2-(dimethylamino)ethylamino)-2-phenyl-N-(2-(pyridin-2-yl)phenyl)pyrimidine-4-carboxamide

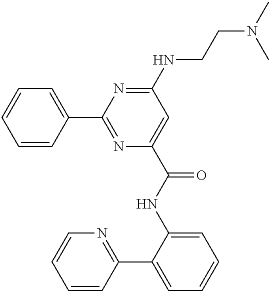

The title compound was prepared according to general method A, utilizing the appropriate aniline and amine, except in the final amine addition both THF and DMSO were used (3:1) and the reaction was heated to 90° C. for 1 h. Yield 77 mg (70%, 2 steps). MS Calcd for C₂₆H₂₆N₆O: 438.22. Found: (M+H)⁺ m/z=439.

### Preparation of N-(2-(5-tert-butylthiazol-2-yl)phenyl)-4-(2-(dimethylamino)ethylamino)-6-phenylpicolinamide

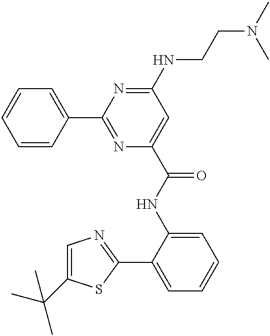

The title compound was prepared according to general method C, utilizing the appropriate aniline and amine. Yield 120 mg (54%, 2 steps). MS Calcd for C₂₉H₃₃N₅OS: 499.24. Found: (M+H)⁺ m/z=500.

### Preparation of N-(2-(5-tert-butyloxazol-2-yl)phenyl)-4-(2-(dimethylamino)ethylamino)-6-phenylpicolinamide

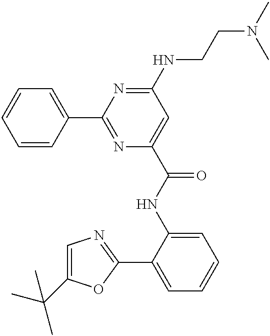

The title compound was prepared according to general method C, utilizing the appropriate aniline and amine. Yield 108 mg (46%, 2 steps). MS Calcd for C₂₉H₃₃N₅O₂: 483.26. Found: (M+H)⁺ m/z=484.

### Preparation of 4-(2-(dimethylamino)ethylamino)-6-phenyl-N-(2-(5-phenyloxazol-2-yl)phenyl)picolinamide

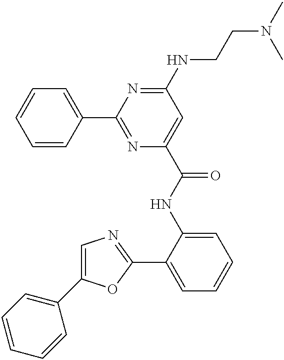

The title compound was prepared according to general method C, utilizing the appropriate aniline and amine. Yield 124 mg (60%, 2 steps). MS Calcd for C₃₁H₂₉N₅O₂: 503.23. Found: (M+H)⁺ m/z=504.

### Preparation of N-(2-(5-tert-butylthiazol-2-yl)phenyl)-4-((2-(dimethylamino)ethyl)(ethyl)amino)-6-phenylpicolinamide

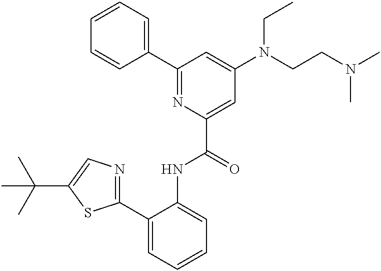

The title compound was prepared according to general method C, utilizing the appropriate amine and aniline. Yield 53 mg (17%, 2 steps). MS Calcd for C₃₁H₃₇N₅OS: 527.72. Found: (M+H)⁺ m/z=528.

### Preparation of N-(2-(5-tert-butyloxazol-2-yl)phenyl)-4-(2-(dimethylamino)ethyl)(ethyl)amino)-6-phenylpicolinamide

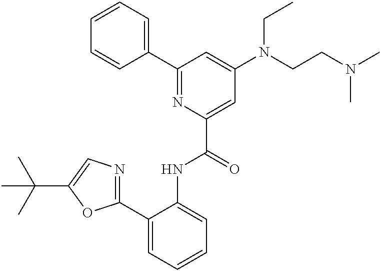

The title compound was prepared according to general method C, utilizing the appropriate amine and aniline. Yield 72 mg (22%, 2 steps). MS Calcd for C₃₁H₃₇N₅O₂: 511.66. Found: (M+H)⁺ m/z=512.

### Preparation of 4-((2-(dimethylamino)ethyl)(ethyl)amino)-6-phenyl-N-(2-(5-phenyloxazol-2-yl)phenyl)picolinamide

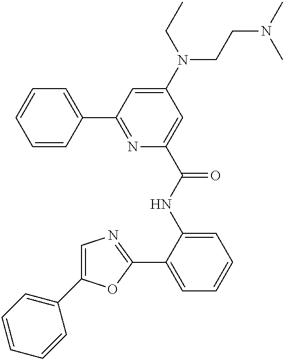

The title compound was prepared according to general method C, utilizing the appropriate amine and aniline. Yield 36 mg (12%, 2 steps). MS Calcd for C₃₃H₃₃N₅O₂: 531.26. Found: (M+H)⁺ m/z=532.

### Preparation of N-(2-(5-cyclopentyl-1,3,4-thiadiazol-2-yl)phenyl)-6-(2-(dimethylamino)ethylamino)-2-phenylpyrimidine-4-carboxamide

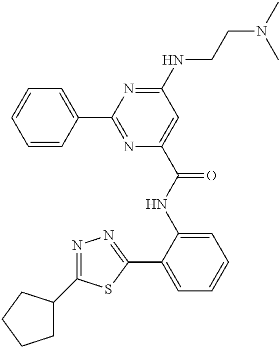

The title compound was prepared according to general method A, utilizing the appropriate aniline and amine. Yield 266 mg (73%, 2 steps). MS Calcd for C₂₈H₃₁N₇OS: 513.23. Found: (M+H)⁺ m/z=514.

### Preparation of N-(2-(5-cyclopentyl-1,3,4-thiadiazol-2-yl)phenyl)-6-((2-(dimethylamino)ethyl)(ethyl)amino)-2-phenylpyrimidine-4-carboxamide

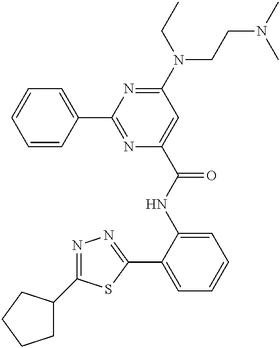

The title compound was prepared according to general method A, utilizing the appropriate aniline and amine. Yield 282 mg (74%, 2 steps). MS Calcd for C₃₀H₃₅N₇OS: 541.26. Found: (M+H)⁺ m/z=542.

### Preparation of N-(2-(5-isopropylyl-1,3,4-thiadiazol-2-yl)phenyl)-6-((2-(dimethylamino)ethyl)(ethyl)amino)-2-phenylpyrimidine-4-carboxamide

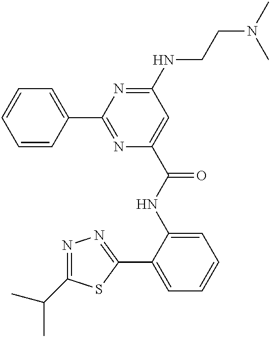

The title compound was prepared according to general method A, utilizing the appropriate aniline and amine. Yield 104 mg (50%, 2 steps). MS Calcd for C₂₆H₂₉N₇OS: 487.22. Found: (M+H)⁺ m/z=488.

### Preparation of 4-(2-(dimethylamino)ethylamino)-6-phenyl-N-(2-(5-phenyl-1,3,4-thiadiazol-2-yl)phenyl)picolinamide

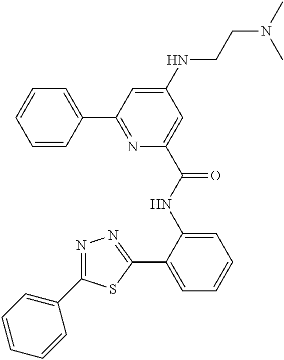

The title compound was prepared according to general method C, utilizing the appropriate aniline and amine. Yield 17 mg (12%, 2 steps). MS Calcd for C₃₀H₂₈N₆OS: 520.20. Found: (M+H)⁺ m/z=521.

### Preparation of N-(2-(5-butyl-1,3,4-thiadiazol-2-yl)phenyl)-6-(2-(dimethylamino)ethylamino)-2-phenylpyrimidine-4-carboxamide

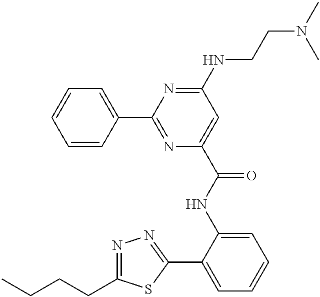

The title compound was prepared according to general method A, utilizing the appropriate aniline and amine. Yield 163 mg (77%, 2 steps). MS Calcd for C₂₇H₃₁N₇OS: 501.23. Found: (M+H)⁺ m/z=502.

### Preparation of N-(2-(5-butyl-1,3,4-thiadiazol-2-yl)phenyl)-6-((2-(dimethylamino)ethyl)(ethyl)amino)-2-phenylpyrimidine-4-carboxamide

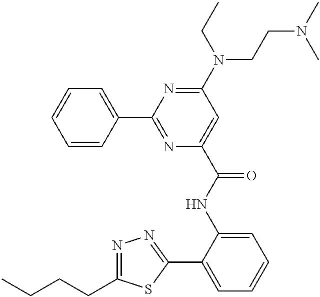

The title compound was prepared according to general method A, utilizing the appropriate aniline and amine. Yield 178 mg (81%, 2 steps). MS Calcd for C₂₉H₃₅N₇OS: 529.26. Found: (M+H)⁺ m/z=530.

### Preparation of N-(2-(5-tert-butyloxazol-2-yl)phenyl)-4-morpholino-6-phenylpicolinamide

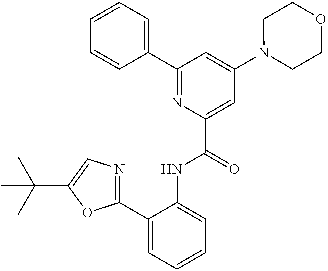

The title compound was prepared according to general method C, utilizing the appropriate aniline and amine. Yield 114 mg (66%, 2 steps). MS Calcd for C₂₉H₃₀N₄O₃: 482.23. Found: (M+H)⁺ m/z=483.

### Preparation of (S)-N-(2-(5-tert-butylthiazol-2-yl)phenyl)-4-(2,3-dihydroxypropoxy)-6-phenylpicolinamide

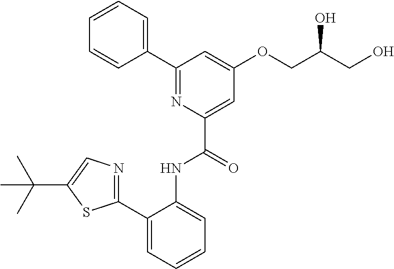

The title compound was prepared according to general method D, utilizing the appropriate aniline and glycerol. Yield 22 mg (21%, 3 steps). MS Calcd for C₂₈H₂₉N₃O₄S: 503.19. Found: (M+H)⁺ m/z=504.

### Preparation of (S)-N-(2-(5-tert-butyloxazol-2-yl)phenyl)-4-(2,3-dihydroxypropoxy)-6-phenylpicolinamide

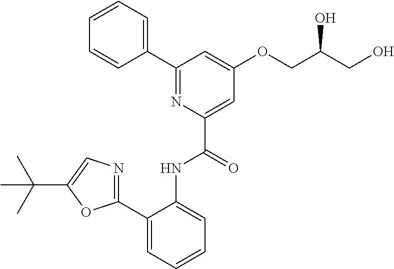

The title compound was prepared according to general method D, utilizing the appropriate aniline and glycerol. Yield 19 mg (11%, 3 steps). MS Calcd for C₂₈H₂₉N₃O₅: 487.21. Found: (M+H)⁺ m/z=488.

### Preparation of N-(2-(5-tert-butylthiazol-2-yl)phenyl)-4-(ethyl(2-methoxyethyl)amino)-6-phenylpicolinamide

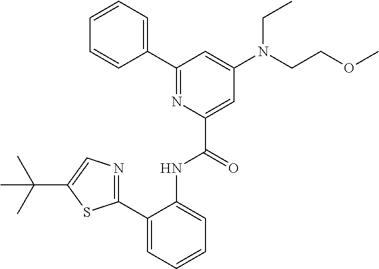

The title compound was prepared according to general method C, utilizing the appropriate aniline and amine. Yield 36 mg (21%, 2 steps). MS Calcd for C₃₀H₃₄N₄O₂S: 514.24. Found: (M+H)⁺ m/z=515.

### Preparation of N-(2-(5-tert-butyloxazol-2-yl)phenyl)-4-(ethyl(2-methoxyethyl)amino)-6-phenylpicolinamide

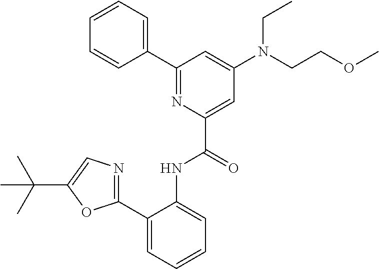

The title compound was prepared according to general method C, utilizing the appropriate aniline and amine. Yield 22 mg (11%, 2 steps). MS Calcd for C₃₀H₃₄N₄O₃: 498.26. Found: (M+H)⁺ m/z=499.

### Preparation of 6-(bis(2-methoxyethyl)amino)-N-(2-(5-morpholino-1,3,4-thiadiazol-2-yl)phenyl)-2-phenylpyrimidine-4-carboxamide

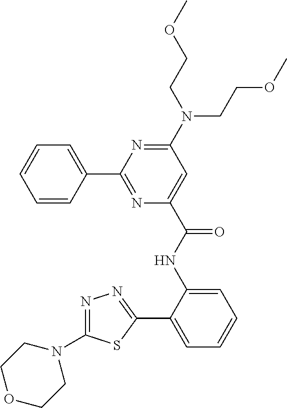

The title compound was prepared according to general method A, utilizing the appropriate aniline and amine. Yield 150 mg (80%, 2 steps). MS Calcd for C₂₉H₃₃N₇O₄S: 575.23. Found: (M+H)⁺ m/z=576.

### Preparation of 6-(ethyl(2-methoxyethyl)amino)-N-(2-(5-morpholino-1,3,4-thiadiazol-2-yl)phenyl)-2-phenylpyrimidine-4-carboxamide

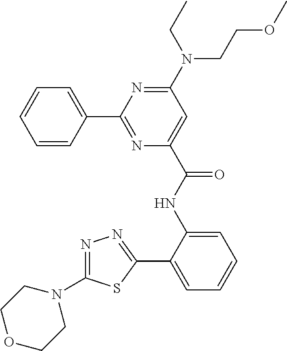

The title compound was prepared according to general method A, utilizing the appropriate aniline and amine. Yield 141 mg (80%, 2 steps). MS Calcd for C₂₈H₃₁N₇O₃S: 545.22. Found: (M+H)⁺ m/z=546.

### Preparation of (S)-6-(2,3-dihydroxypropoxy)-N-(2-(5-morpholino-1,3,4-thiadiazol-2-yl)phenyl)-2-phenylpyrimidine-4-carboxamide

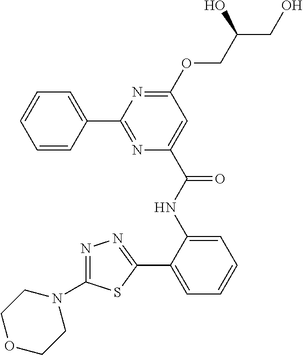

The title compound was prepared according to general method B, utilizing the appropriate aniline and glycerol. Yield 61 mg (36%, 3 steps). MS Calcd for C₂₆H₂₆N₆O₅S: 534.17. Found: (M+H)⁺ m/z=535.

### Preparation of (R)-6-(2,3-dihydroxypropoxy)-N-(2-(5-morpholino-1,3,4-thiadiazol-2-yl)phenyl)-2-phenylpyrimidine-4-carboxamide

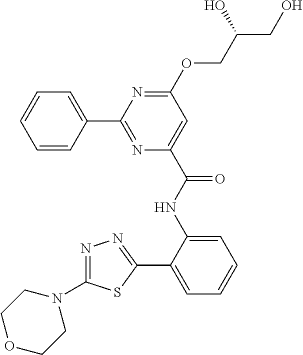

The title compound was prepared according to general method B, utilizing the appropriate aniline and glycerol. Yield 78 mg (45%, 3 steps). MS Calcd for C₂₆H₂₆N₆O₅S: 534.17. Found: (M+H)⁺ m/z=535.

### Preparation of 6-(methyl(2-(methylamino)ethyl)amino)-2-phenyl-N-(2-(5-phenyl-1,3,4-thiadiazol-2-yl)phenyl)pyrimidine-4-carboxamide

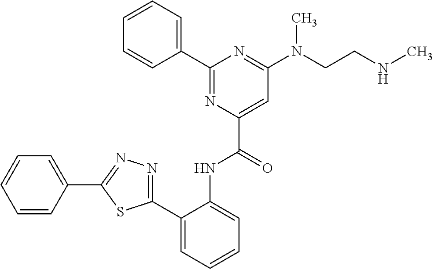

The title compound was prepared according to General Method A using 2-(5-phenyl-1,3,4-thiadiazol-2-yl)aniline and N,N′-dimethylethylenediamine. Yield 65 mg (59%). MS Calcd for C₂₉H₂₇N₇OS: 521.20. Found (M+H)⁺ m/z=522

### Preparation of 6-((2-(dimethylamino)ethyl)(methyl)amino)-2-phenyl-N-(2-(5-phenyl-1,3,4-thiadiazol-2-yl)phenyl)pyrimidine-4-carboxamide

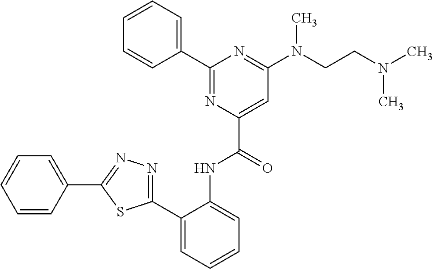

The title compound was prepared according to General Method A using 2-(5-phenyl-1,3,4-thiadiazol-2-yl)aniline and N,N,N′-trimethylethylenediamine. Yield 86 mg (57%). MS Calcd for C₃₀H₂₉N₇OS: 535.22. Found (M+H)⁺ m/z=536

### Preparation of 4-((2-(dimethylamino)ethyl)(methyl)amino)-6-phenyl-N-(2-(5-phenyl-1,3,4-thiadiazol-2-yl)phenyl)picolinamide

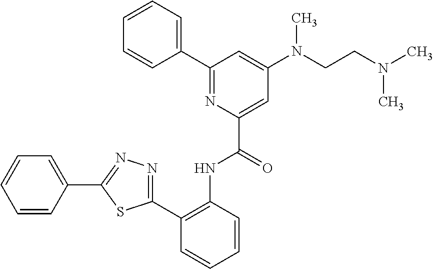

The title compound was prepared according to General Method C using 2-(5-phenyl-1,3,4-thiadiazol-2-yl)aniline and N,N,N′-trimethylethylenediamine. The final compound was purified via preparative high performance liquid chromatography (HPLC). Product containing fractions were concentrated down under vacuum then treated with 1 M aqueous sodium hydroxide to obtain the free base. Yield 33 mg (39%). MS Calcd for C₃₁H₃₀N₆OS: 534.22. Found (M+H)⁺ m/z=535

### Preparation of 6-((2-(dimethylamino)ethyl)(ethyl)amino)-2-phenyl-N-(2-(5-phenyl-1,3,4-thiadiazol-2-yl)phenyl)pyrimidine-4-carboxamide

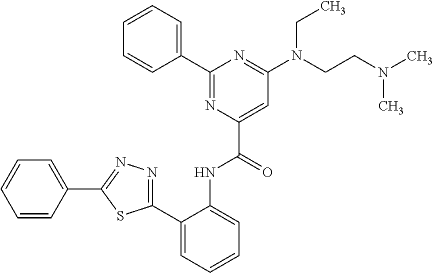

The title compound was prepared according to General Method A using 2-(5-phenyl-1,3,4-thiadiazol-2-yl)aniline and N,N-dimethyl-N′-ethylethylenediamine. The final compound was purified via preparative HPLC. Product containing fractions were concentrated down under vacuum then treated with 1 M aqueous sodium hydroxide to obtain the free base. Yield 76 mg (65%). MS Calcd for C₃₁H₃₁N₇OS: 549.23. Found (M+H)⁺ m/z=550

### Preparation of N-(2-(1,3,4-thiadiazol-2-yl)phenyl)-6-(methyl(2-(methylamino)ethyl)amino)-2-phenylpyrimidine-4-carboxamide

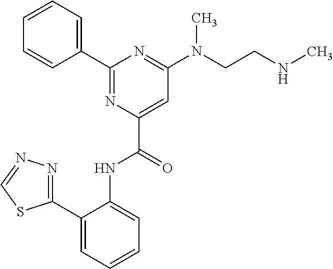

The title compound was prepared according to General Method A using 2-(1,3,4-thiadiazol-2-yl)aniline and N,N′-dimethylethylenediamine. The final compound was purified via preparative HPLC. Product containing fractions were concentrated down under vacuum then treated with 1 M aqueous sodium hydroxide to obtain the free base. Yield 30 mg (27%). MS Calcd for C₂₃H₂₃N₇OS: 445.17. Found (M+H)⁺ m/z=446

### Preparation of N-(2-(1,3,4-thiadiazol-2-yl)phenyl)-6-((2-(dimethylamino)ethyl)(ethyl)amino)-2-phenylpyrimidine-4-carboxamide

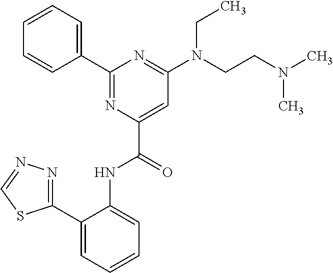

The title compound was prepared according to General Method A using 2-(1,3,4-thiadiazol-2-yl)aniline and N,N-dimethyl-N′-ethylethylenediamine. The final compound was purified via preparative HPLC. Product containing fractions were concentrated down under vacuum then treated with 1 M aqueous sodium hydroxide to obtain the free base. Yield 76 mg (63%). MS Calcd for C₂₅H₂₇N₇OS: 473.20. Found (M+H)⁺ m/z=474

### Preparation of 4-(methyl(2-(methylamino)ethyl)amino)-6-phenyl-N-(2-(5-phenyl-1,3,4-thiadiazol-2-yl)phenyl)picolinamide

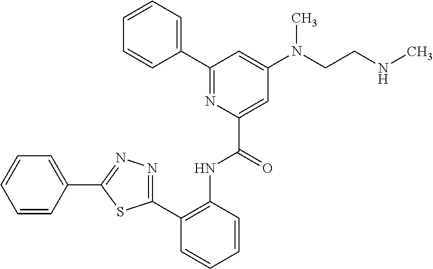

The title compound was prepared according to General Method C using 2-(5-phenyl-1,3,4-thiadiazol-2-yl)aniline and N,N′-dimethylethylenediamine. The final compound was purified via preparative HPLC. Product containing fractions were concentrated down under vacuum then treated with 1 M aqueous sodium hydroxide to obtain the free base. Yield 10 mg (9%). MS Calcd for C₃₀H₂₈N₆OS: 520.20. Found (M+H)⁺ m/z=521

### Preparation of N-(2-(5-cyclobutyl-1,3,4-thiadiazol-2-yl)phenyl)-6-((2-(dimethylamino)ethyl)(methyl)amino)-2-phenylpyrimidine-4-carboxamide

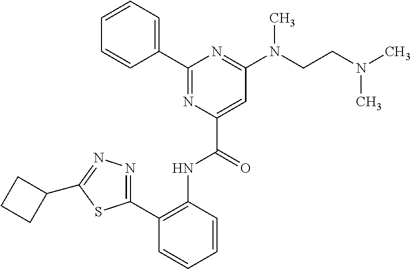

The title compound was prepared according to General Method A using 2-(5-cyclobutyl-1,3,4-thiadiazol-2-yl)aniline and N,N,N′-trimethylethylenediamine. Yield 66 mg (58%). MS Calcd for C₂₈H₃₁N₇OS: 513.23. Found (M+H)⁺ m/z=514

### Preparation of N-(2-(5-cyclobutyl-1,3,4-thiadiazol-2-yl)phenyl)-6-(methyl(2-(methylamino)ethyl)amino)-2-phenylpyrimidine-4-carboxamide

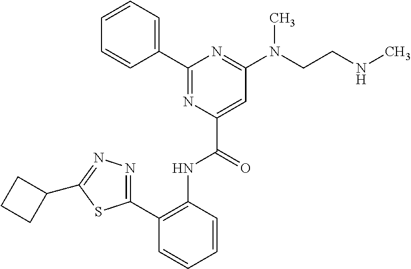

The title compound was prepared according to General Method A using 2-(5-cyclobutyl-1,3,4-thiadiazol-2-yl)aniline and N,N′-dimethylethylenediamine. Yield 126 mg (75%). MS Calcd for C₂₇H₂₉N₇OS: 499.22. Found (M+H)⁺ m/z=500

### Preparation of N-(2-(5-cyclobutyl-1,3,4-thiadiazol-2-yl)phenyl)-6-(2-(dimethylamino)ethylamino)-2-phenylpyrimidine-4-carboxamide

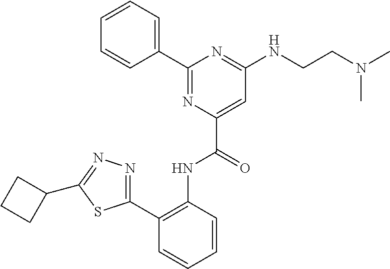

The title compound was prepared according to General Method A using 2-(5-cyclobutyl-1,3,4-thiadiazol-2-yl)aniline and N,N-dimethylethylenediamine. Yield 129 mg (77%). MS Calcd for C₂₇H₂₉N₇OS: 499.22. Found (M+H)⁺ m/z=500

### Preparation of N-(2-(5-cyclobutyl-1,3,4-thiadiazol-2-yl)phenyl)-6-((2-(dimethylamino)ethyl)(ethyl)amino)-2-phenylpyrimidine-4-carboxamide

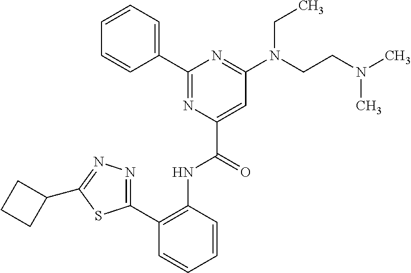

The title compound was prepared according to General Method A using 2-(5-cyclobutyl-1,3,4-thiadiazol-2-yl)aniline and N,N-dimethyl-N′-ethylethylenediamine. Yield 66 mg (37%). MS Calcd for C₂₉H₃₃N₇OS: 527.25. Found (M+H)⁺ m/z=528

### Preparation of N-(2-(5-ethyl-1,3,4-thiadiazol-2-yl)phenyl)-6-(methyl(2-(methylamino)ethyl)amino)-2-phenylpyrimidine-4-carboxamide

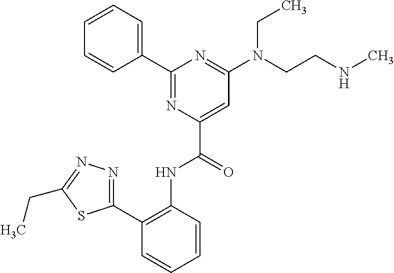

The title compound was prepared according to General Method A using 2-(5-ethyl-1,3,4-thiadiazol-2-yl)aniline and N,N′-dimethylethylenediamine. Yield 94 mg (56%). MS Calcd for C₂₅H₂₇N₇OS: 473.20. Found (M+H)⁺ m/z=474

### Preparation of 6-(2-(dimethylamino)ethylamino)-N-(2-(5-ethyl-1,3,4-thiadiazol-2-yl)phenyl)-2-phenylpyrimidine-4-carboxamide

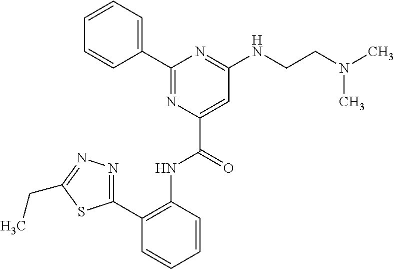

The title compound was prepared according to General Method A using 2-(5-ethyl-1,3,4-thiadiazol-2-yl)aniline and N,N-dimethylethylenediamine. Yield 141 mg (84%). MS Calcd for C₂₅H₂₇N₇OS: 473.20. Found (M+H)⁺ m/z=474

### Preparation of 6-((2-(dimethylamino)ethyl)(ethyl)amino)-N-(2-(5-ethyl-1,3,4-thiadiazol-2-yl)phenyl)-2-phenylpyrimidine-4-carboxamide

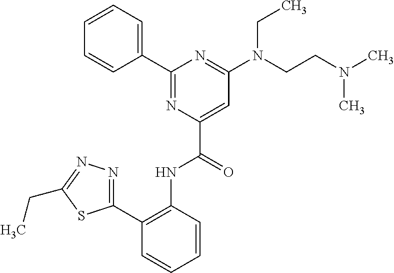

The title compound was prepared according to General Method A using 2-(5-ethyl-1,3,4-thiadiazol-2-yl)aniline and N,N-dimethyl-N′-ethylethylenediamine. The final compound was purified via preparative HPLC. Product containing fractions were concentrated down under vacuum then treated with 1 M aqueous sodium hydroxide to obtain the free base. Yield 80 mg (45%). MS Calcd for C₂₇H₃₁N₇OS: 501.23. Found (M+H)⁺ m/z=502

### Preparation of 6-((2-(dimethylamino)ethyl)(methyl)amino)-N-(2-(5-ethyl-1,3,4-thiadiazol-2-yl)phenyl)-2-phenylpyrimidine-4-carboxamide

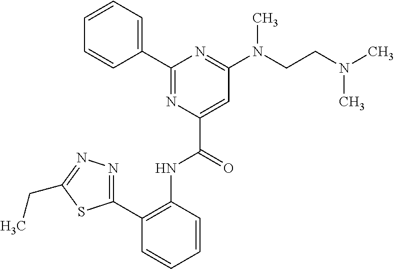

The title compound was prepared according to General Method A using 2-(5-ethyl-1,3,4-thiadiazol-2-yl)aniline and N,N,N′-trimethylethylenediamine. The final compound was purified via preparative HPLC. Product containing fractions were concentrated down under vacuum then treated with 1 M aqueous sodium hydroxide to obtain the free base. Yield 58 mg (33%). MS Calcd for C₂₆H₂₉N₇OS: 487.22. Found (M+H)⁺ m/z=488

### Preparation of N-(2-(1,3,4-thiadiazol-2-yl)phenyl)-6-(3-oxopiperazin-1-yl)-2-phenylpyrimidine-4-carboxamide

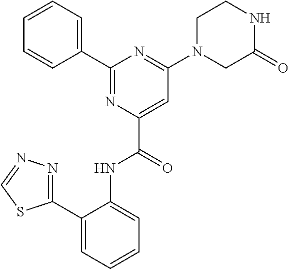

The title compound was prepared according to the same procedure as for N-(2-(1,3,4-thiadiazol-2-yl)phenyl)-6-(2-(dimethylamino)ethylamino)-2-phenylpyrimidine-4-carboxamide, substituting 2-piperazinone for N,N-dimethylethylenediamine. Yield 82 mg (70%). MS Calcd. for C₂₃H₁₉N₇O₂S: 457.13. Found (M+H)⁺ m/z=458.

### N-(2-(1,3,4-thiadiazol-2-yl)phenyl)-6-(4-ethyl-3-oxopiperazin-1-yl)-2-phenylpyrimidine-4-carboxamide

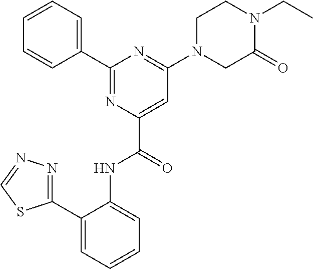

The title compound was prepared according to the same procedure as for N-(2-(1,3,4-thiadiazol-2-yl)phenyl)-6-(2-(dimethylamino)ethylamino)-2-phenylpyrimidine-4-carboxamide, substituting 1-ethyl-2-piperazinone for N,N-dimethylethylenediamine. Yield 112 mg (91%). MS Calcd. for C₂₃H₁₉N₇O₂S: 485.16. Found (M+H)⁺ m/z=486.

### N-(2-(1,3,4-thiadiazol-2-yl)phenyl)-6-(1,3-dihydroxypropan-2-ylamino)-2-phenylpyrimidine-4-carboxamide

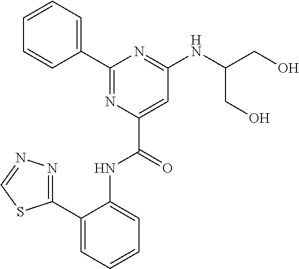

The title compound was prepared according to the same procedure as for N-(2-(1,3,4-thiadiazol-2-yl)phenyl)-6-(2-(dimethylamino)ethylamino)-2-phenylpyrimidine-4-carboxamide, substituting 2-amino-1,3-propanediol for N,N-dimethylethylenediamine. Yield 63 mg (56%). MS Calcd. for C₂₂H₂₀N₆O₃S: 448.13. Found (M+H)⁺ m/z=449.

### N-(2-(1,3,4-thiadiazol-2-yl)phenyl)-6-(4-methyl-3-oxopiperazin-1-yl)-2-phenylpyrimidine-4-carboxamide

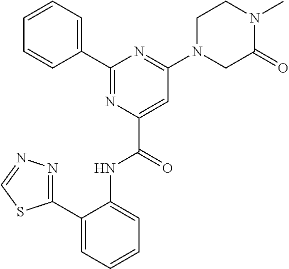

The title compound was prepared according to the same procedure as for N-(2-(1,3,4-thiadiazol-2-yl)phenyl)-6-(2-(dimethylamino)ethylamino)-2-phenylpyrimidine-4-carboxamide, substituting 1-methyl-2-piperazinone for N,N-dimethylethylenediamine. Yield 100 mg (85%). MS Calcd. for C₂₄H₂₁N₇O₂S: 471.15. Found (M+H)⁺ m/z=472.

### (S)-N-(2-(1,3,4-thiadiazol-2-yl)phenyl)-6-(2,3-dihydroxypropoxy)-2-phenylpyrimidine-4-carboxamide

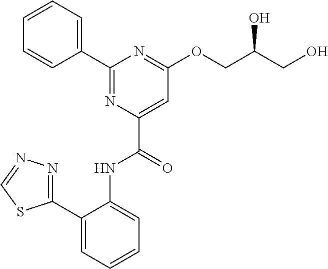

The title compound was prepared according to general method B utilizing 2-(1,3,4-thiadiazol-2-yl)aniline and (R)-(2,2-dimethyl-1,3-dioxolan-4-yl)methanol. (80 mg, 74% yield for 3 steps). MS Calcd. for C₂₂H₁₉N₅O₄S: 449.12. Found (M+H)⁺ m/z=450.

### N-(2-(1,3,4-thiadiazol-2-yl)phenyl)-6-(2,3-dihydroxypropoxy)-2-phenylpyrimidine-4-carboxamide

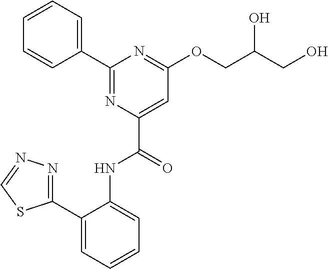

To a mixture of 3 mmol of glycerol in 4 mL of DMSO was added 48 mg (1.2 mmol) of NaH in mineral oil. The mixture was stirred until all of the NaH had reacted, then 197 mg (0.5 mmol) of the N-(2-(1,3,4-thiadiazol-2-yl)phenyl)-6-chloro-2-phenylpyrimidine-4-carboxamide was added. The reaction was stirred at ambient temperature for 30 min, then it was diluted with 15 mL of water and filtered. The precipitate was washed with pentate (2×5 mL), and water (3×5 mL). The product was recrystallized from THF to give 58 mg (52%) of a white solid. MS Calcd. for C₂₂H₁₉N₅O₄S: 449.12. Found (M+H)⁺ m/z=450.

### Preparation of ethyl 5-(2-(6-((2-(dimethylamino)ethyl)(methyl)amino)-2-phenylpyrimidine-4-carboxamido)phenyl)-1,3,4-thiadiazole-2-carboxylate

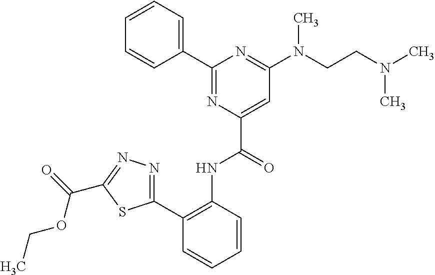

The title compound was prepared according to General Method A using ethyl 5-(2-aminophenyl)-1,3,4-thiadiazole-2-carboxylate and N,N,N′-trimethylethylenediamine. Yield 74 mg (43%) of a white solid. MS Calcd for C₂₇H₂₉N₇O₃S: 531.21. Found (M+H)⁺ m/z=532

### Preparation of N-(2-(dimethylamino)ethyl)-5-(2-(6-(2-(dimethylamino)ethylamino)-2-phenylpyrimidine-4-carboxamido)phenyl)-1,3,4-thiadiazole-2-carboxamide

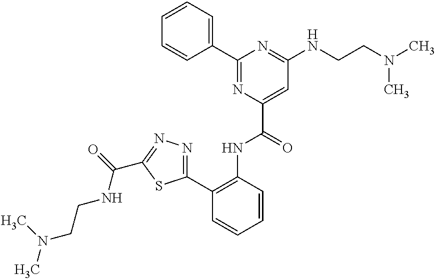

Ethyl 5-(2-(6-chloro-2-phenylpyrimidine-4-carboxamido)phenyl)-1,3,4-thiadiazole-2-carboxylate (150 mg, 0.32 mmol) was suspended in tetrahydrofuran (3 mL). N,N-Dimethylethylenediamine (0.25 mL, 2.29 mmol) was added and the reaction mixture was heated to reflux for 30 minutes. The reaction mixture was allowed to cool, and water was added to precipitate out the compound. The precipitate was filtered and washed with water. The solids were triturated with methanol at reflux. Yield 164 mg (91%). MS Calcd for C₂₈H₃₃N₉O₂S: 559.25. Found (M+H)⁺ m/z=560

### Preparation of ethyl 5-(2-(6-((2-(dimethylamino)ethyl)(ethyl)amino)-2-phenylpyrimidine-4-carboxamido)phenyl)-1,3,4-thiadiazole-2-carboxylate

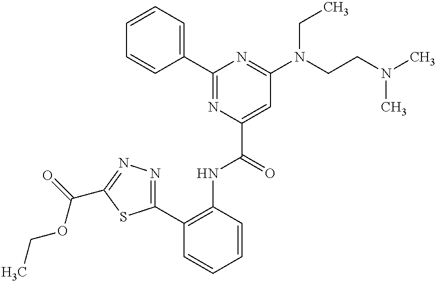

The title compound was prepared according to General Method A using ethyl 5-(2-aminophenyl)-1,3,4-thiadiazole-2-carboxylate and N,N-dimethyl-N′-ethylethylenediamine. Yield 49 mg (28%). MS Calcd for C₂₈H₃₁N₇O₃S: 545.22. Found (M+H)⁺ m/z=546

### Preparation of 5-(2-(6-(2-(dimethylamino)ethylamino)-2-phenylpyrimidine-4-carboxamido)phenyl)-1,3,4-thiadiazole-2-carboxamide

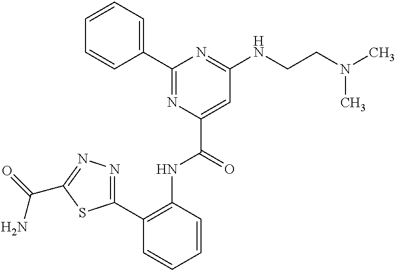

The title compound was prepared according to General Method A using 5-(2-aminophenyl)-1,3,4-thiadiazole-2-carboxamide and N,N-dimethyl ethylenediamine. Yield 16 mg (26%). MS Calcd for C₂₄H₂₄N₈O₂S: 488.17. Found (M+H)⁺ m/z=489

### Preparation of 5-(2-(2-phenyl-6-(piperazin-1-yl)pyrimidine-4-carboxamido)phenyl)-1,3,4-thiadiazole-2-carboxamide

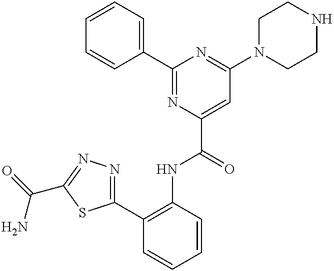

The title compound was prepared according to General Method A using 5-(2-aminophenyl)-1,3,4-thiadiazole-2-carboxamide and piperazine. Yield 33 mg (59%). MS Calcd for C₂₄H₂₂N₈O₂S: 486.16. Found (M+H)⁺ m/z=487

### Preparation of (S)-5-(2-(6-(hexahydropyrrolo[1,2-a]pyrazin-2(1H)-yl)-2-phenylpyrimidine-4-carboxamido)phenyl)-1,3,4-thiadiazole-2-carboxamide

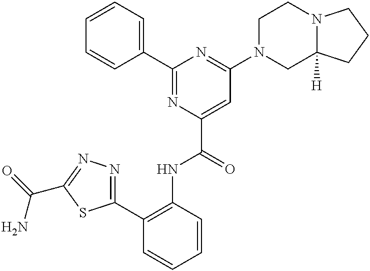

The title compound was prepared according to General Method A using 5-(2-aminophenyl)-1,3,4-thiadiazole-2-carboxamide and (S)-1,4-diazabicyclo[4.3.0]nonane. Yield 21 mg (35%). MS Calcd for C₂₇H₂₆N₈O₂S: 526.19. Found (M+H)⁺ m/z=527

### Preparation of 5-(2-(6-((2-(dimethylamino)ethyl)(ethyl)amino)-2-phenylpyrimidine-4-carboxamido)phenyl)-1,3,4-thiadiazole-2-carboxamide

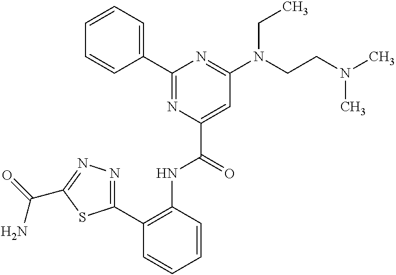

The title compound was prepared according to General Method A using 5-(2-aminophenyl)-1,3,4-thiadiazole-2-carboxamide and N,N-dimethyl-N′-ethylethylenediamine. Yield 24 mg (41%). MS Calcd for C₂₆H₂₈N₈O₂S: 516.21. Found (M+H)⁺ m/z=517

### Preparation of 5-(2-(6-((2-(dimethylamino)ethyl)(ethyl)amino)-2-phenylpyrimidine-4-carboxamido)phenyl)-N-methyl-1,3,4-thiadiazole-2-carboxamide

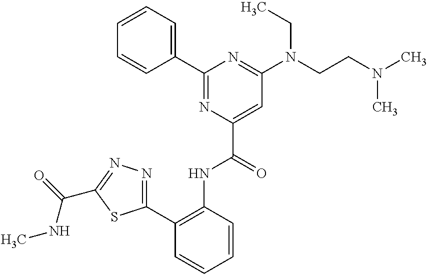

The title compound was prepared according to General Method A using 5-(2-aminophenyl)-N-methyl-1,3,4-thiadiazole-2-carboxamide and N,N-dimethyl-N′-ethylethylenediamine. The final compound was purified via preparative HPLC. Product containing fractions were concentrated down under vacuum then treated with 1 M aqueous sodium hydroxide to obtain the free base. Yield 15 mg (12%). MS Calcd for C₂₇H₃₀N₈O₂S: 530.22. Found (M+H)⁺ m/z=531

### Preparation of 6-((2-(dimethylamino)ethyl)(ethyl)amino)-N-(2-(5-(morpholine-4-carbonyl)-1,3,4-thiadiazol-2-yl)phenyl)-2-phenylpyrimidine-4-carboxamide

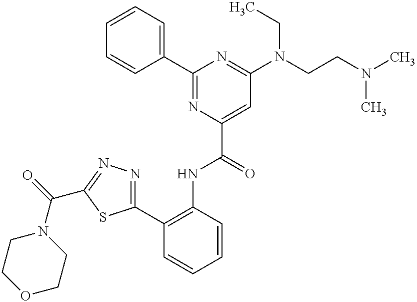

The title compound was prepared according to General Method A using (5-(2-aminophenyl)-1,3,4-thiadiazol-2-yl)(morpholino)methanone and N,N-dimethyl-N′-ethylethylenediamine. The final compound was purified via preparative HPLC. Product containing fractions were concentrated down under vacuum then treated with 1 M aqueous sodium hydroxide to obtain the free base. Yield 21 mg (13%). MS Calcd for C₃₀H₃₄N₈O₃S: 586.25. Found (M+H)⁺ m/z=587

### Preparation of 6-(4-methyl-3-oxopiperazin-1-yl)-N-(2-(5-(morpholine-4-carbonyl)-1,3,4-thiadiazol-2-yl)phenyl)-2-phenylpyrimidine-4-carboxamide

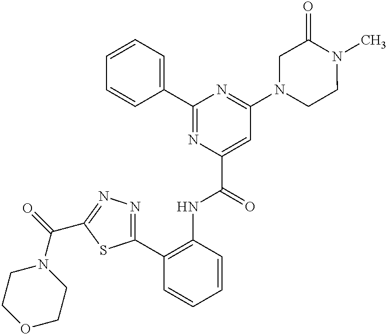

The title compound was prepared according to General Method A using (5-(2-aminophenyl)-1,3,4-thiadiazol-2-yl)(morpholino)methanone and 1-methylpiperazin-2-one. Yield 61 mg (71%). MS Calcd for C₂₉H₂₈N₈O₄S: 584.20. Found (M+H)⁺ m/z 585

### Preparation of N-(2-(5-(morpholine-4-carbonyl)-1,3,4-thiadiazol-2-yl)phenyl)-6-(3-oxopiperazin-1-yl)-2-phenylpyrimidine-4-carboxamide

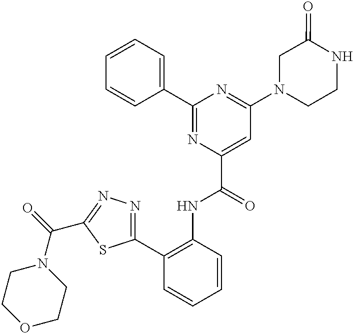

The title compound was prepared according to General Method A using (5-(2-aminophenyl)-1,3,4-thiadiazol-2-yl)(morpholino)methanone and piperazin-2-one. Yield 21 mg (25%). MS Calcd for C₂₈H₂₆N₈O₄S: 570.18. Found (M+H)⁺ m/z=571

### Preparation of 6-(1,3-dihydroxypropan-2-ylamino)-N-(2-(5-(morpholine-4-carbonyl)-1,3,4-thiadiazol-2-yl(phenyl)-2-phenylpyrimidine-4-carboxamide

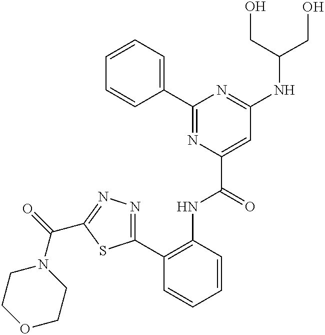

The title compound was prepared according to General Method A using (5-(2-aminophenyl)-1,3,4-thiadiazol-2-yl)(morpholino)methanone and serinol. Yield 62 mg (75%). MS Calcd for C₂₇H₂₇N₇O₅S: 561.18. Found (M+H)⁺ m/z=562

### Preparation of (S)-6-(2,3-dihydroxypropoxy)-N-(2-(5-(morpholine-4-carbonyl)-1,3,4-thiadiazol-2-yl)phenyl)-2-phenylpyrimidine-4-carboxamide

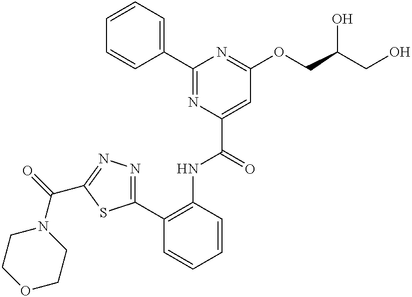

The title compound was prepared according to General Method B using (5-(2-aminophenyl)-1,3,4-thiadiazol-2-yl)(morpholino)methanone and (R)-(−)-2,2-dimethyl-1,3-dioxolane-4-methanol. Yield 30 mg (43%). MS Calcd for C₂₇H₂₆N₆O₆S: 562.16. Found (M+H)⁺ m/z=563

### Preparation of 6-(bis(2-methoxyethyl)amino)-N-(2-(5-(morpholine-4-carbonyl)-1,3,4-thiadiazol-2-yl)phenyl)-2-phenylpyrimidine-4-carboxamide

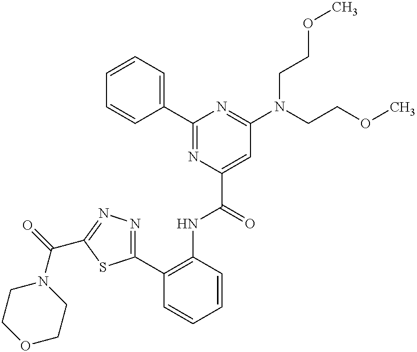

The title compound was prepared according to General Method A using (5-(2-aminophenyl)-1,3,4-thiadiazol-2-yl)(morpholino)methanone and bis(2-methoxyethyl)amine. Yield 32 mg (64%). MS Calcd for C₃₀H₃₃N₇O₅S: 603.23. Found (M+H)⁺ m/z=604

### Preparation of 6-((2-(dimethylamino)ethyl)(ethyl)amino)-N-(2-(5-(methylsulfonamido)-1,3,4-thiadiazol-2-yl)phenyl)-2-phenylpyrimidine-4-carboxamide

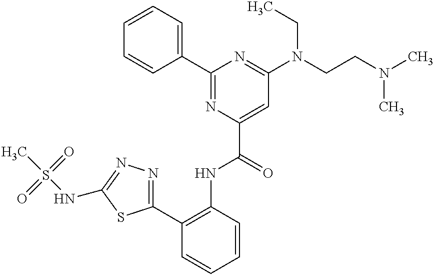

The title compound was prepared according to General Method A using N-(5-(2-aminophenyl)-1,3,4-thiadiazol-2-yl)methanesulfonamide and N,N-dimethyl-N′-ethylethylenediamine. The final compound was purified via preparative HPLC. Product containing fractions were concentrated down under vacuum then treated with 1 M aqueous sodium hydroxide to obtain the free base. Yield 14 mg (23%). MS Calcd for C₂₆H₃₀N₈O₃S₂: 566.19. Found (M+H)⁺ m/z=567

### Preparation of N-(5-(2-(6-((2-(dimethylamino)ethyl)(ethyl)amino)-2-phenylpyrimidine-4-carboxamido)phenyl)-1,3,4-thiadiazol-2-yl)morpholine-4-carboxamide

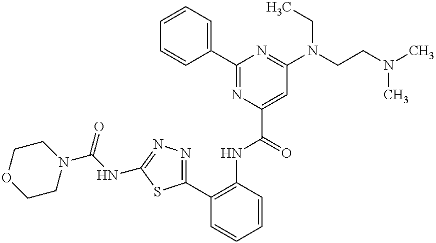

The title compound was prepared according to General Method A using N-(5-(2-aminophenyl)-1,3,4-thiadiazol-2-yl)morpholine-4-carboxamide and N,N-dimethyl-N′-ethylethylenediamine. The final compound was purified via preparative HPLC. Product containing fractions were concentrated down under vacuum then treated with 1 M aqueous sodium hydroxide to obtain the free base. Yield 17 mg (12%). MS Calcd for C₃₀H₃₅N₉O₃S₂: 601.26. Found (M+H)⁺ m/z=602

### Preparation of 6-((2-(dimethylamino)ethyl)(ethyl)amino)-N-(2-(5-(methylthio)-1,3,4-thiadiazol-2-yl)phenyl)-2-phenylpyrimidine-4-carboxamide

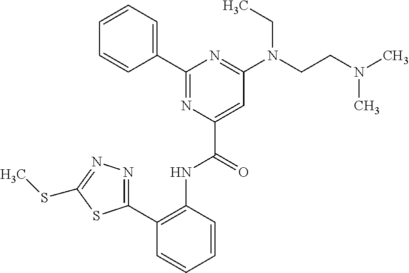

The title compound was prepared according to General Method A using 2-(5-(methylthio)-1,3,4-thiadiazol-2-yl)aniline and N,N-dimethyl-N′-ethylethylenediamine. Yield 745 mg (74%). MS Calcd for C₂₆H₂₉N₇OS₂: 519.19. Found (M+H)⁺ m/z=520

### Preparation of 6-((2-(dimethylamino)ethyl)(ethyl)amino)-N-(2-(5-(methylsulfinyl)-1,3,4-thiadiazol-2-yl)phenyl)-2-phenylpyrimidine-4-carboxamide

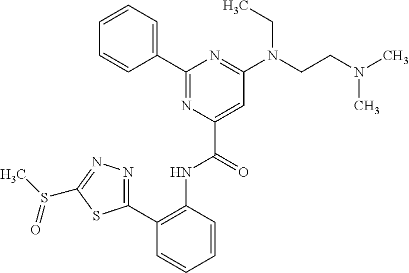

6-((2-(dimethylamino)ethyl)(ethyl)amino)-N-(2-(5-(methylthio)-1,3,4-thiadiazol-2-yl)phenyl)-2-phenylpyrimidine-4-carboxamide (250 mg, 0.481 mmol) was dissolved in glacial acetic acid (10 mL). Hydrogen peroxide (30% solution in water, 40 μL, 0.481 mmol) was added and the reaction mixture was heated to 60° C. for 2 hrs. Both the sulfoxide and sulfone were observed in the LC/MS, so the desired compound was purified via preparative HPLC. Product containing fractions were concentrated down under vacuum then treated with 1 M aqueous sodium hydroxide to obtain the free base. Yield 13 mg (5%). MS Calcd for C₂₆H₂₉N₇O₂S₂: 535.18. Found (M+H)⁺ m/z=536

### Preparation of 6-morpholino-2-phenyl-N-(2-(thiazole-2-carbonyl)phenyl)pyrimidine-4-carboxamide

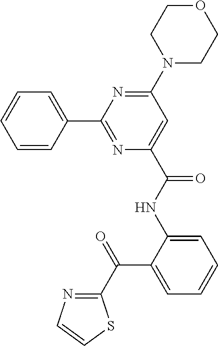

The title compound was prepared according to general method A, utilizing the appropriate aniline and amine. Yield 32 mg (40%, 2 steps). MS Calcd for C₂₅H₂₁N₅O₃S: 471.14. Found: (M+H)⁺ m/z=472.

### Preparation of 6-((2-(dimethylamino)ethyl)(ethyl)amino)-2-phenyl-N-(2-(thiazole-2-carbonyl)phenyl)pyrimidine-4-carboxamide

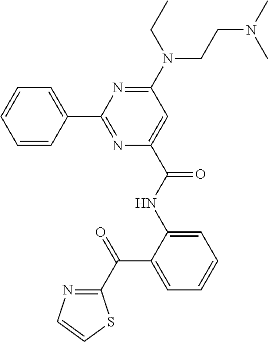

The title compound was prepared according to general method A, utilizing the appropriate aniline and amine. Yield 67 mg (73%, 2 steps). MS Calcd for C₂₇H₂₈N₆O₂S: 500.20. Found: (M+H)⁺ m/z=501.

### Preparation of 6-morpholino-2-phenyl-N-(2-picolinoylphenyl)pyrimidine-4-carboxamide

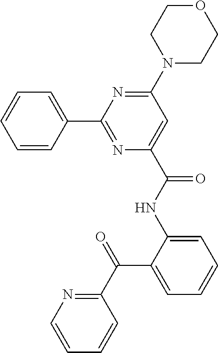

The title compound was prepared according to general method A, utilizing the appropriate aniline and amine. Yield 98 mg (44%, 2 steps). MS Calcd for C₂₇H₂₃N₅O₃: 465.18. Found: (M+H)⁺ m/z=466.

### Preparation of 6-(2-(dimethylamino)ethylamino)-2-phenyl-N-(2-propionylphenyl)pyrimidine-4-carboxamide

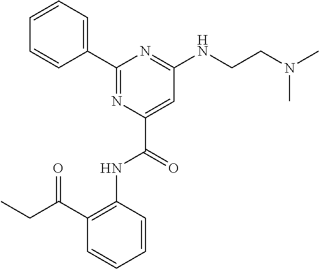

The title compound was prepared from 1-(2-aminophenyl)propan-1-one according to General Procedure A. Yield 82 mg (40%, 2 steps). MS Calcd for C₂₄H₂₇N₅O₂: 417.22. Found (M+H)⁺ m/z=418.

### Preparation of 6-(2-(dimethylamino)ethylamino)-N-(2-pentanoylphenyl)-2-phenylpyrimidine-4-carboxamide

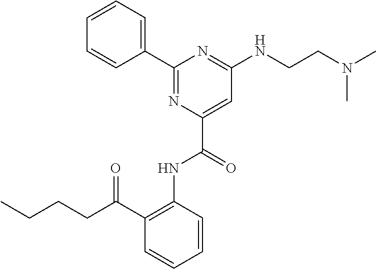

The title compound was prepared from 1-(2-aminophenyl)pentan-1-one according to General Procedure A. Yield 106 mg (51%, 2 steps). MS Calcd for C₂₆H₃₁N₅O₂: 445.58. Found (M+H)⁺ m/z=446.

### Preparation of N-(2-acetylphenyl)-6-(1,3-dihydroxypropan-2-ylamino)-2-phenylpyrimidine-4-carboxamide

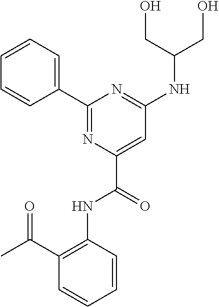

The title compound was prepared from (2-aminophenyl)ethan-1-one and 2-amino-1,3-propandiol according to General Procedure A. Yield 72 mg (53%, 2 steps). MS Calcd for C₂₂H₂₂N₄O₄: 445.58. Found (M+H)⁺ m/z=446.

### Preparation of N-(2-benzoylphenyl)-6-(2-(dimethylamino)ethylamino)-2-phenylpyrimidine-4-carboxamide

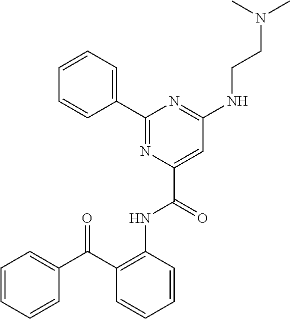

The title compound was prepared from (2-aminophenyl)(phenyl)methanone and N,N-dimethylethylenediamine according to General Procedure A. Yield 51 mg. MS Calcd for C₂₈H₂₇N₅O₂: 465.22. Found (M+H)⁺ m/z=466.

### Preparation of N-(2-acetylphenyl)-6-morpholino-2-phenylpyrimidine-4-carboxamide

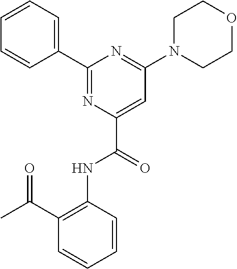

The title compound was prepared from (2-aminophenyl)ethan-1-one and morpholine according to General Procedure A. Yield 210 mg (79%, 2 steps). MS Calcd for C₂₃H₂₂N₄O₃: 402.17. Found (M+H)⁺ m/z=403.

### Preparation of N-(2-acetylphenyl)-6-((2-(dimethylamino)ethyl)(ethyl)amino)-2-phenylpyrimidine-4-carboxamide

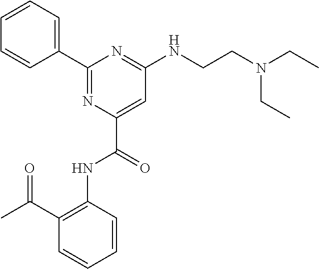

The title compound was prepared from (2-aminophenyl)ethan-1-one and N²,N²-diethylethylenediamine according to General Procedure A. Yield 102 mg. MS Calcd for C₂₃H₂₂N₄O₃: 431.23. Found (M+H)⁺ m/z=432.

### Preparation of N-(2-acetylphenyl)-6-(4-isopropylpiperazin-1-yl)-2-phenylpyrimidine-4-carboxamide

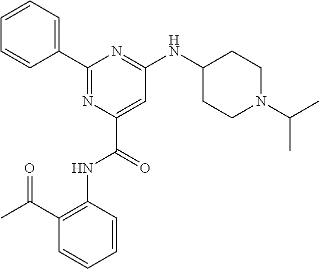

The title compound was prepared from (2-aminophenyl)ethan-1-one and 1-isopropylpiperidin-4-amine according to General Procedure A. Yield 104 mg. MS Calcd for C₂₇H₃₁N₅O₂: 457.25. Found (M+H)⁺ m/z=458.

### Preparation of N-(2-acetylphenyl)-2-phenyl-6-(piperazin-1-yl)pyrimidine-4-carboxamide

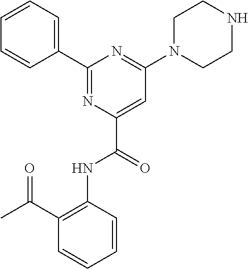

The title compound was prepared from (2-aminophenyl)ethan-1-one and piperazine according to General Procedure A. Yield 123 mg. MS Calcd for C₂₃H₂₅N₅O₂: 401.19. Found (M+H)⁺ m/z=402.

### Preparation of ethyl 3-(6-(2-acetylphenylcarbamoyl)-2-phenylpyrimidin-4-ylamino)propanoate

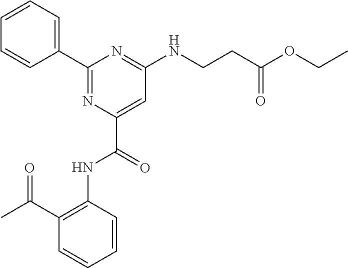

The title compound was prepared from (2-aminophenyl)ethan-1-one and ethyl 3-aminopropanoate according to General Procedure A. Yield 119 mg. MS Calcd for C₂₉H₃₅N₅O₆: 549.26. Found (M+H)⁺ m/z=550.

### Preparation of N-(2-acetylphenyl)-2-phenyl-6-(piperidin-4-ylamino)pyrimidine-4-carboxamide

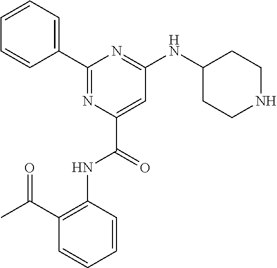

The title compound was prepared from (2-aminophenyl)ethan-1-one and tert-butyl 4-aminopiperidine-1-carboxylate according to General Procedure A. The (t-butoxycarbonyl) Boc group was removed by dissolution in TFA, then extraction with NaHCO₃/ethyl acetate to give the title compound. Yield 130 mg. MS Calcd for C₂₄H₂₅N₅O₂: 415.20. Found (M+H)⁺ m/z=416.

### Preparation of N-(2-acetylphenyl)-6-(bis(2-methoxyethyl)amino)-2-phenylpyrimidine-4-carboxamide

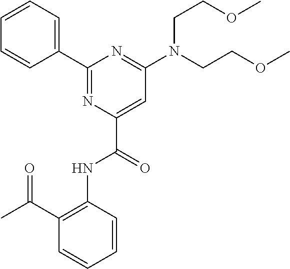

The title compound was prepared from (2-aminophenyl)ethan-1-one and bis(2-methoxyethyl)amine according to General Procedure A. Yield 108 mg. MS Calcd for C₂₅H₂₈N₄O₄: 448.21. Found (M+H)⁺ m/z=449.

### Preparation of 6-(3-aminobutylamino)-N-(2-isobutyrylphenyl)-2-phenylpyrimidine-4-carboxamide

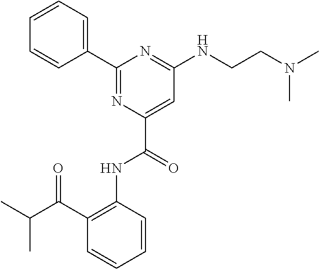

The title compound was prepared from 1-(2-aminophenyl)-2-methylpropan-1-one and N¹,N¹-dimethylethane-1,2-diamine according to General Procedure A. Yield 78 mg (34%, 2 steps). MS Calcd for C₂₅H₂₉N₅O₂: 431.55. Found (M+H)⁺ m/z=432.

### Preparation of N-(2-acetylphenyl)-6-(1-methylpiperidin-4-ylamino)-2-phenylpyrimidine-4-carboxamide

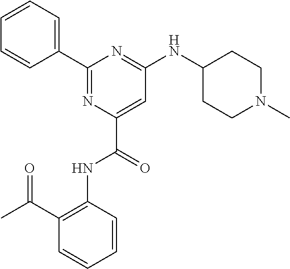

The title compound was prepared from 1-(2-aminophenyl)-ethan-1-one and 1-methylpiperidin-4-amine according to General Procedure A. Yield 99 mg. MS Calcd for C₂₅H₂₇N₅O₂: 429.22. Found (M+H)⁺ m/z=430.

### Preparation of N-(2-acetylphenyl)-6-chloro-2-phenylpyrimidine-4-carboxamide

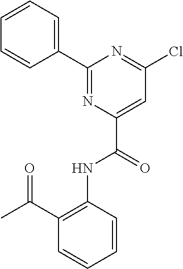

To a solution of 540 mg (4.00 mmol) of 2-aminoacetophenone in 10 mL of dichloromethane was added 0.9 mL of triethylamine, then a solution of 1.01 g (4.0 mmol) of 6-chloro-2-phenylpyrimidine-4-carbonyl chloride in 5 mL of dichloromethane. The reaction was stirred at ambient temperature for 10 min, then the precipitate was filtered, washed with methanol, and dried on the filter to give 1.195 g (85%) of the amine as a white solid.

### Preparation of N-(2-acetylphenyl)-6-(2-(dimethylamino)ethylamino)-2-phenylpyrimidine-4-carboxamide

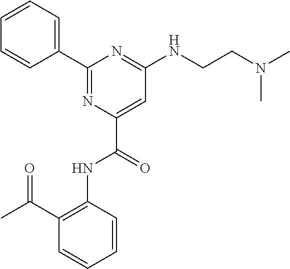

To a suspension of 582 mg (1.65 mmol) of N-(2-acetylphenyl)-6-chloro-2-phenylpyrimidine-4-carboxamide in 10 mL of tetrahydrofuran was added 0.9 mL of N,N-dimethylethylenediamine. The reaction was heated at reflux for 40 min, then diluted with 25 mL of water. The precipitate was filtered, washed with additional water, then dried on the filter to give 598 mg (90%) of a white solid. MS Calcd. for C₂₃H₂₅N₅O₂: 403.20. Found (M+H)⁺ m/z=404.

### Preparation of (E/Z)-6-chloro-N-(2-(1-(hydroxyimino)ethyl)phenyl)-2-phenylpyrimidine-4-carboxamide

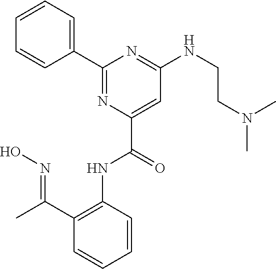

To a solution of 100 mg (0.25 mmol) of N-(2-acetylphenyl)-6-(2-(dimethylamino)ethylamino)-2-phenylpyrimidine-4-carboxamide in 5 mL of ethanol and 1 mL of pyridine at 70° C. was added 35 mg (0.5 mmol) of hydroxylamine hydrochloride. The reaction was stirred at 70° C. for 16 h, then concentrated to a thick oil. The oil was taken up in ethanol and concentrated again to remove more pyridine, giving a solid residue. This was partitioned between 10 mL of ethyl acetate and 3 mL of water, warming as necessary to dissolve the crude product. The layers were separated, then the organic layer was extracted with water (1×3 mL), and brine (1×3 mL), dried over MgSO₄, filtered, and concentrated to a white solid. This was taken up in ethanol and concentrated to give 27 mg (26%) of a white solid. MS Calcd. for C₂₃H₂₆N₆O₂: 418.21. Found (M+H)⁺ m/z=419.

### Preparation of 6-(2-(dimethylamino)ethylamino)-N-(2-(1-hydroxyethyl)phenyl)-2-phenylpyrimidine-4-carboxamide

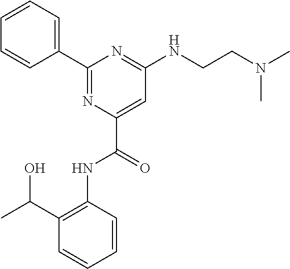

To a solution of 96 mg (0.24 mmol) of N-(2-acetylphenyl)-6-(2-(dimethylamino)ethylamino)-2-phenylpyrimidine-4-carboxamide in 5 mL of ethanol and 2 mL of tetrahydrofuran was added 100 mg (2.64 mmol) of sodium borohydride. The reaction was stirred at ambient temperature, reduction was complete within 10 min. The solvents were removed in vacuo, then the residue was taken up in 10 mL of water. The suspension was extracted with ethyl acetate (2×5 mL), then the combined ethyl acetate layers were back extracted with water (1×3 mL), and brine (1×3 mL), dried over MgSO₄, filtered, and concentrated to a white foam. Trituration with ethyl acetate, followed by drying at 100 C under hi vacuum gave 67 mg (69%) of white crystals. MS Calcd. for C₂₃H₂₇N₅O₂: 405.22. Found (M+H)⁺ m/z=406.

### N-(2-bromophenyl)-6-chloro-2-phenylpyrimidine-4-carboxamide

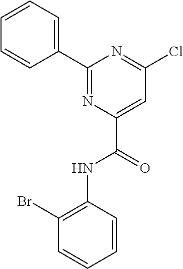

To a solution of 1.72 g (10.0 mmol) of 2-bromoaniline in 10 mL of chloroform was added 2.5 mL (18 mmol) of triethylamine. To the stirred solution was added a solution of 2.52 g (10.0 mmol) of 6-chloro-2-phenylpyrimidine-4-carbonyl chloride in 10 mL of chloroform, with stirring. The reaction was stirred for 10 min, during which time the solution became a thick mixture. The reaction was diluted with 50 mL of methanol, then filtered. The precipitate was washed with additional methanol, then dried on the filter to give 3.39 g (87%) of white crystals.

### N-(2-bromophenyl)-6-(2-(dimethylamino)ethylamino)-2-phenylpyrimidine-4-carboxamide

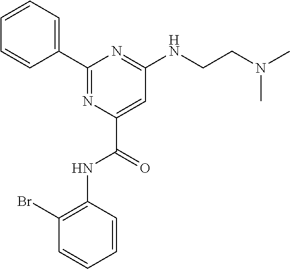

To a suspension of 2.0 g (5.15 mmol) of N-(2-bromophenyl)-6-chloro-2-phenylpyrimidine-4-carboxamide in 30 mL of tetrahydrofuran was added 3 mL (27.5 mmol) of N,N-dimethylethylenediamine. The reaction was heated at reflux for 1.5 h, then cooled. The solution was diluted with 150 mL of water, then the precipitate was filtered, washed with 50 mL of water, and dried on the filter to give 2.16 g (95%) of a white solid. MS Calcd. for C₂₃H₂₇N₅O₂: 439.10. Found (M+H)⁺ m/z=440, 442.

### N-(2-bromophenyl)-6-((2-(dimethylamino)ethyl)(ethyl)amino)-2-phenylpyrimidine-4-carboxamide hydrochloride

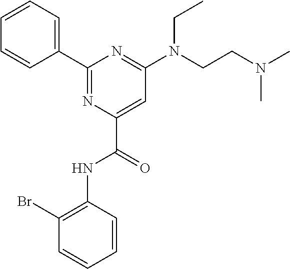

To a suspension of 594 mg (1.53 mmol) of N-(2-bromophenyl)-6-chloro-2-phenylpyrimidine-4-carboxamide in 10 mL of tetrahydrofuran was added 1 mL (6.35 mmol) of (N-ethyl,N′N′-dimethyl)ethylenediamine. The reaction was heated at reflux for 30 min, then diluted with 50 mL of water to give an oily precipitate. The suspension was extracted with ethyl acetate (3×15 mL), then the combined ethyl acetate layers were back extracted with water (2×10 mL), and brine (1×10 mL), dried over MgSO₄, filtered, and concentrated to an oil. This was dried at 100° C. under hi vacuum to give 666 mg (93%) of a thick oil. The hydrochloride salt was prepared by dissolving 84 mg of the oil in 1 mL of tetrahydrofuran, and adding 0.5 mL of 1M HCl_{(aq.)}. The solution was concentrated in vacuo to give 87 mg (97%) of a white foam. MS Calcd. for C₂₃H₂₆BrN₅O: 467.13. Found (M+H)⁺ m/z=468, 470.

### 6-(2-(dimethylamino)ethylamino)-N-(2-(furan-2-yl)phenyl)-2-phenylpyrimidine-4-carboxamide

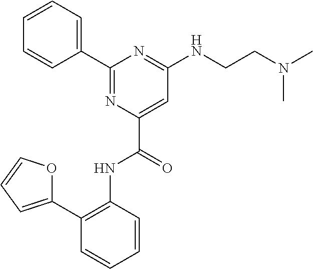

To a sealable tube, suitable for microwave heating, was added a mixture of 55 mg (0.50 mmol) of furan-2-ylboronic acid, 150 mg (0.341 mmol) of N-(2-bromophenyl)-6-(2-(dimethylamino)ethylamino)-2-phenylpyrimidine-4-carboxamide, 4 mg (0.02 mmol) of palladium(11)acetate, 18 mg (0.69 mmol) of triphenylphosphine, and 200 mg (2.0 mmol) of sodium carbonate was added 3 mL of 1,2-dimethoxyethane, and 1 mL of water. The reaction headspace was purged with N₂, then the tube was sealed. The reaction was heated at 150° C. for 30 min, then the solution was diluted with 20 mL of ethyl acetate and extracted with water (2×5 mL), and brine (1×5 mL), dried over MgSO₄, filtered, and concentrated to an oil that slowly crystallized. This was recrystallized from ethyl acetate to give 9 mg (6%) of a white solid. MS Calcd. for C₂₅H₂₅N₅O₂: 427.52. Found (M+H)⁺ m/z=428.

### 6-(2-(dimethylamino)ethylamino)-2-phenyl-N-(2-(pyridin-4-yl)phenyl)pyrimidine-4-carboxamide

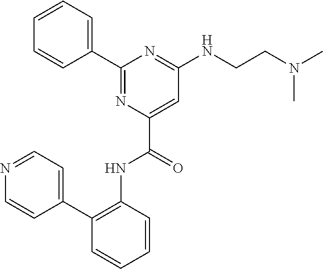

The title compound was prepared according to the procedure for 6-(2-(dimethylamino)ethylamino)-N-(2-(furan-2-yl)phenyl)-2-phenylpyrimidine-4-carboxamide, substituting pyridin-4-ylboronic acid for furan-2-ylboronic acid. (Yield 69 mg, 46%). MS Calcd. for C₂₆H₂₆N₆O: 438.22. Found (M+H)⁺ m/z=439.

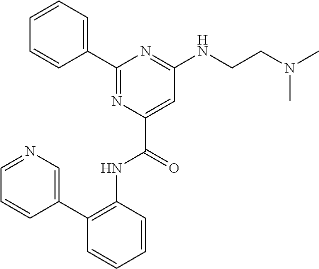

### 6-(2-(dimethylamino)ethylamino)-2-phenyl-N-(2-(pyridin-3-yl)phenyl)pyrimidine-4-carboxamide

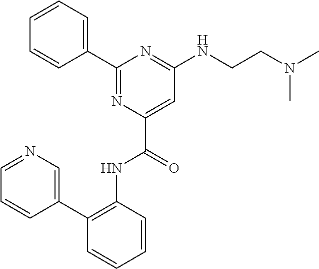

The title compound was prepared according to the procedure for 6-(2-(dimethylamino)ethylamino)-N-(2-(furan-2-yl)phenyl)-2-phenylpyrimidine-4-carboxamide, substituting pyridin-3-ylboronic acid for furan-2-ylboronic acid. (Yield 85 mg, 57%). MS Calcd. for C₂₆H₂₆N₆O: 438.22. Found (M+H)⁺ m/z=439.

### N′-acetyl-2-nitrobenzohydrazide

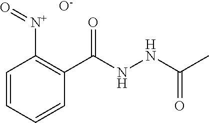

To a solution of 1.81 g (10 mmol) of 2-nitrobenzhydrazide in 10 mL of ethyl acetate was added 1.7 mL of acetic anhydride. The reaction was stirred at ambient temperature for 20 min, during which time a thick mixture formed. The precipitate was filtered, washed with ethyl acetate, and dried on the filter to give 1.69 g (75%) of a white solid.

### 2-methyl-5-(2-nitrophenyl)-1,3,4-oxadiazole

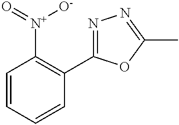

To 100 mg (0.448 mol) of N′-acetyl-2-nitrobenzohydrazide was added 3 mL of phosphorus oxychloride. The solution was heated at 100° C. for 1 h, then the excess phosphorus oxychloride was removed in vacuo. The residue was dissolved in 5 mL of water, and a white precipitate formed. This was extracted with ethyl acetate (1×5 mL), then the ethyl acetate layer was back extracted with saturated NaHCO_{3(aq.)} (1×5 mL), and brine (1×5 mL), dried over MgSO₄, filtered, and concentrated to give 84 mg (91%) of a white crystalline solid.

### 2-(5-methyl-1,3,4-oxadiazol-2-yl)aniline

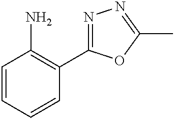

To 81 mg (0.40 mmol) of 2-methyl-5-(2-nitrophenyl)-1,3,4-oxadiazole was added 2 mL of 5:1 (v/v) isopropanol:H₂O, 25 mg (0.47 mmol) of ammonium chloride, and 130 mg (2.37 mmol) of iron powder. The reaction was heated at reflux for 1.5 h with vigorous stirring, then the excess iron and precipitated salts were filtered, and the filtrate was concentrated in vacuo. The residue was taken up in 5 mL of hot ethyl acetate, and the insoluble ammonium chloride was extracted away with water (1×2 mL). The ethyl acetate layer was extracted with brine (1×2 mL), dried over MgSO₄, filtered, and concentrated to 61 mg (88%) of a solid.

### 6-chloro-N-(2-(5-methyl-1,3,4-oxadiazol-2-yl)phenyl)-2-phenylpyrimidine-4-carboxamide

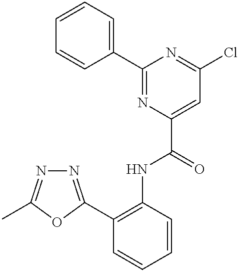

To a solution of 55 mg (0.31 mmol) of 2-(5-methyl-1,3,4-oxadiazol-2-yl)aniline and 0.1 mL (0.72 mmol) of triethylamine in 1 mL of dichloromethane was added a solution of 85 mg (0.34 mmol) of 6-chloro-2-phenylpyrimidine-4-carbonyl chloride in 1 mL of dichloromethane. The mixture was stirred for 10 min at ambient temperature, then it was diluted with 5 mL of methanol. The precipitate was filtered, washed with methanol, and dried in vacuo to give 81 mg (66%) of a white solid. The product is slightly soluble in DMSO and in methanol.

### 6-(2-(dimethylamino)ethylamino)-N-(2-(5-methyl-1,3,4-oxadiazol-2-yl)phenyl)-2-phenylpyrimidine-4-carboxamide

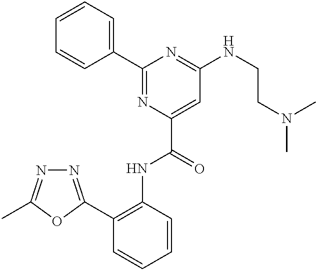

To a suspension of 81 mg (0.21 mmol) of 6-chloro-N-(2-(5-methyl-1,3,4-oxadiazol-2-yl)phenyl)-2-phenylpyrimidine-4-carboxamide in 1 mL of tetrahydrofuran was added 0.1 mL (0.64 mmol) of (N,N-dimethyl)ethylenediamine. The reaction was heated at reflux for 30 min, then cooled and diluted with 5 mL of water. The precipitate was filtered and partially dried on the filter, then taken up in 1 mL of tetrahydrofuran while still damp. The solution was diluted with 5 mL of water, then the precipitate was filtered, washed with water, and dried in vacuo at 100° C. The product was recrystallized from isopropyl alcohol to give 27 mg (29%) of a white solid. MS Calcd. for C₂₄H₂₅N₇O₂: 443.52. Found (M+H)⁺ m/z=444.

### N-(2-hydroxypropyl)-2-nitrobenzamide

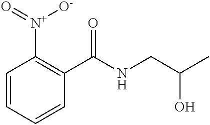

To a solution of 751 mg (10.0 mmol) of 1-amino-2-propanol in 15 mL of saturated NaHCO_{3(aq.)} was added a solution of 1.85 g (10.0 mmol) of 2-nitrobenzoyl chloride in 15 mL of ethyl acetate. The mixture was stirred at ambient temperature for 10 min, then the layers were separated, and the organic layer was extracted with brine (1×15 mL), dried over MgSO₄, filtered, and concentrated to 2.01 g (90%) of the amide as a white, crystalline solid.

### 2-nitro-N-(2-oxopropyl)benzamide

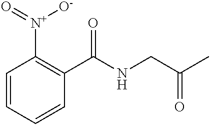

To a solution of 2.16 g (9.6 mmol) of N-(2-hydroxypropyl)-2-nitrobenzamide in 50 mL of acetone was added 7 mL of 2.0M H₂CrO₄(aq.). The solution was stirred at ambient temperature for 10 min, then the excess chromic acid was quenched by the addition of 10 mL of 2M NaHSO_{3(aq.)}, giving a blue lower layer. The reaction was concentrated to remove most of the acetone, then 15 mL of ethyl acetate was added and the biphasic solution was stirred for 5 min. The layers were separated, then the aqueous layer was extracted with additional ethyl acetate (2×15 mL). The combined ethyl acetate layers were back extracted with water (1×5 mL) saturated NaHCO_{3(aq.)} (2×10 mL), and brine (1×10 mL), dried over MgSO₄, filtered, and concentrated in vacuo to a white solid. This was purified via silica gel MPLC, eluting with a 50 to 100% ethyl acetate/pentane gradient to give 1.10 g (52%) of the ketoamide as a white solid. The ketoamide eluted after a significant amount of 2-nitrobenzamide.

### 5-methyl-2-(2-nitrophenyl)oxazole

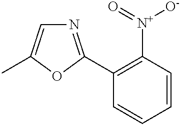

To 202 mg (0.91 mmol) of 2-nitro-N-(2-oxopropyl)benzamide was added 1 mL of phosphorus oxychloride. The reaction was stirred at 90° C. for 30 min, then concentrated in vacuo. The residue was taken up in 5 mL of water, then the suspension was extracted with ethyl acetate (3×5 mL). The combined ethyl acetate layers were back extracted with 2M NaOH_{(aq.)} (2×5 mL), and brine (1×5 mL), dried over MgSO₄, filtered, and concentrated in vacuo to a solid. The product was purified via MPLC, eluting with a 20-50% ethyl acetate/pentane gradient to give 159 mg (86%) of a solid.

### 2-(5-methyloxazol-2-yl)aniline

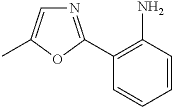

To 147 mg (0.720 mmol) of 5-methyl-2-(2-nitrophenyl)oxazole in 3 mL of isopropanol and 1 mL of water was added 198 mg (3.60 mmol) of iron powder, and 46 mg (0.86 mmol) of ammonium chloride. The reaction was stirred at reflux for 1.5 h, then filtered. The solids were washed with ethyl acetate, then the combined filtrate and washings were concentrated in vacuo. The residue was partitioned between 10 mL of ethyl acetate and 3 mL of water, and the layers were separated. The organic layer was extracted with brine (1×3 mL), dried over MgSO₄, filtered, and concentrated in vacuo to give 116 mg (92%) of a solid.

### 6-chloro-N-(2-(5-methyloxazol-2-yl)phenyl)-2-phenylpyrimidine-4-carboxamide

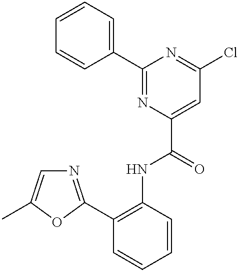

To a solution of 107 mg (0.524 mmol) of 2-(5-methyloxazol-2-yl)aniline in 1 mL of dichloromethane was added 0.2 mL (1.43 mmol) of triethylamine, then a solution of 133 mg (0.524 mmol) of 6-chloro-2-phenylpyrimidine-4-carbonyl chloride in 1 mL of dichloromethane. The reaction was stirred at ambient temperature for 20 min, then diluted with 10 mL of methanol. The precipitate was filtered, washed with methanol, and dried on the filter to give 168 mg (82%) of a light yellow solid.

### 6-(2-(dimethylamino)ethylamino)-N-(2-(5-methyloxazol-2-yl)phenyl)-2-phenylpyrimidine-4-carboxamide

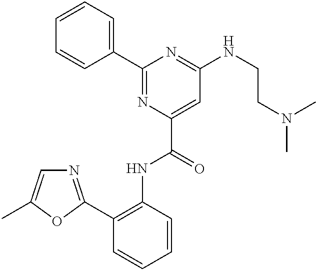

To a suspension of 100 mg (0.256 mmol) of 6-chloro-N-(2-(5-methyloxazol-2-yl)phenyl)-2-phenylpyrimidine-4-carboxamide in 1.5 mL of tetrahydrofuran was added 0.15 mL (0.96 mmol) of (N,N-dimethyl)ethylenediamine. The reaction was heated at reflux for 40 min, then diluted with 10 mL of water and filtered. The damp precipitate was recrystallized from ethanol to give 80 mg (71%) of white needles. MS Calcd. for C₂₅H₂₆N₆O₂: 442.53. Found (M+H)⁺ m/z=443.

### 2-methyl-5-(2-nitrophenyl)-1,3,4-thiadiazole

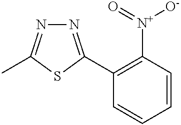

A suspension of 446 mg (2.0 mmol) of N′-acetyl-2-nitrobenzohydrazide in 40 mL of toluene was heated to a gentle reflux. To this was added 1.2 g (5.4 mmol) of phosphorus pentasulfide. After 10 min, the toluene was decanted from any remaining solids, then concentrated in vacuo. The residue was taken up in 20 mL of ethyl acetate and 10 mL of water. The layers were separated, draining any insoluble material with the water layer. The organic layer was extracted with 0.78M NaOCl (1×5 mL), water (1×5 mL), and brine (1×5 mL), dried over MgSO₄, filtered, and concentrated to an oil that slowly crystallized to give 250 mg (56%) of the thiadiazole.

### 2-(5-methyl-1,3,4-thiadiazol-2-yl)aniline

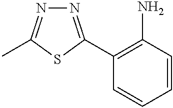

To a mixture of 250 mg (1.13 mmol) of 2-methyl-5-(2-nitrophenyl)-1,3,4-thiadiazole, 373 mg (6.80 mmol) of iron powder, and 73 mg (1.36 mmol) of ammonium chloride was added 6 mL of 5:1 (v/v) isopropanol (i-PrOH): H₂O. The reaction was stirred at reflux for 1 h, then filtered. The filtrate was concentrated in vacuo, then the residue was taken up in 10 mL of ethyl acetate and extracted with water (1×2 mL), then brine (1×2 mL), dried over MgSO₄, filtered, and concentrated to a solid. This was taken up in 1 mL of 12M HCl and heated at 80 C for 15 min to hydrolyze some contaminating oxadiazole. Next, 10 mL of 2M NaOH was added, and the suspension was extracted with ethyl acetate (3×5 mL). The combined ethyl acetate layers were back extracted with water (1×5 mL), dried over MgSO₄, filtered, and concentrated to a solid. This was purified via MPLC, loading in CH₂Cl₂ solution and eluting with a 10-50% ethyl acetate/pentane gradient to give 101 mg of the desired amine as a solid.

### 6-chloro-N-(2-(5-methyl-1,3,4-thiadiazol-2-yl)phenyl)-2-phenylpyrimidine-4-carboxamide

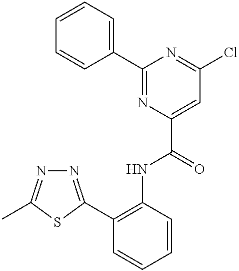

This was prepared according to the procedure for 6-chloro-N-(2-(5-methyloxazol-2-yl)phenyl)-2-phenylpyrimidine-4-carboxamide, substituting 2-(5-methyl-1,3,4-thiadiazol-2-yl)aniline for 2-(5-methyloxazol-2-yl)aniline. Yield 155 mg (73%).

### 6-(2-(dimethylamino)ethylamino)-N-(2-(5-methyl-1,3,4-thiadiazol-2-yl)phenyl)-2-phenylpyrimidine-4-carboxamide

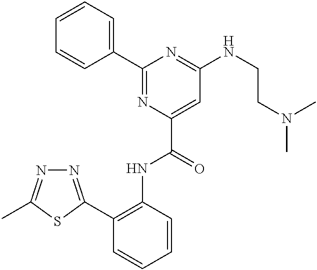

This was prepared according to the procedure for 6-(2-(dimethylamino)ethylamino)-N-(2-(5-methyloxazol-2-yl)phenyl)-2-phenylpyrimidine-4-carboxamide, substituting 6-chloro-N-(2-(5-methyl-1,3,4-thiadiazol-2-yl)phenyl)-2-phenylpyrimidine-4-carboxamide for 6-chloro-N-(2-(5-methyloxazol-2-yl)phenyl)-2-phenylpyrimidine-4-carboxamide. The crude product was triturated with hot ethanol to purify, giving 125 mg (72%) of a white powder. MS Calcd. for C₂₄H₂₅N₇OS: 459.18. Found (M+H)⁺ m/z=460.

### N′-benzoyl-2-nitrobenzohydrazide

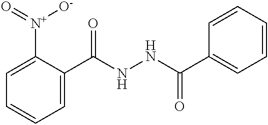

To 1.81 g (10.0 mmol) of 2-nitrobenzohydrazide was added 40 mL of ethyl acetate. The suspension was warmed to dissolve the hydrazide, then 12 mL of saturated NaHCO_{3(aq.)} was added. To the stirred solution was added a solution of 1.40 g (10.0 mmol) of benzoyl chloride in 5 mL of ethyl acetate. A white precipitate formed. The suspension was stirred at ambient temperature for 10 min, then 15 mL of 1M HCl_{(aq.)} was added, slowly, to control gas evolution. Next, 20 mL of pentane was added, then the suspension was filtered. The precipitate was washed with water, and dried in vacuo at 105° C. to give 2.63 g (92%) of a white solid.

### 2-(2-nitrophenyl)-5-phenyl-1,3,4-thiadiazole

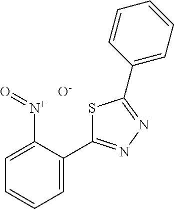

A suspension of 2.65 g (9.29 mmol) of N′-benzoyl-2-nitrobenzohydrazide in 30 mL of toluene was heated to reflux. To the mixture was added 2.41 g (10.9 mmol) of P₂S₅. The mixture was heated at reflux for an additional 2 h, during which time a gummy precipitate formed. The mixture was allowed to stand at ambient temperature for 18 h, then 30 mL of saturated NaHCO_{3(aq.)} was added, and the biphasic mixture was stirred for 30 min. Next, 30 mL of ethyl acetate was added, giving a biphasic solution with some small pieces of undissolved yellow solid. The layers were separated, and the aqueous layer was extracted with ethyl acetate (1×30 mL). The combined organic layers were back extracted with saturated NaHCO_{3(aq.)} (2×20 mL), 0.78 M NaOCl (2×20 mL), and brine (1×20 mL), dried over MgSO₄, filtered, and concentrated in vacuo to 1.2 g (46%) of a red solid.

### 2-(5-phenyl-1,3,4-thiadiazol-2-yl)aniline

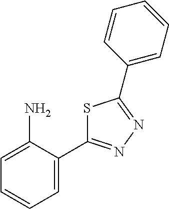

To 1.2 g (4.24 mmol) of 2-(2-nitrophenyl)-5-phenyl-1,3,4-thiadiazole was added 20 mL of 4:1 (v/v) isopropanol: water, 1.16 g (21 mmol) of iron powder, and 272 mg (5.09 mmol) of ammonium chloride. The reaction was stirred at reflux for 1 h, then cooled, and filtered. The insoluble material was washed with ethyl acetate, then the combined filtrate and washings were concentrated in vacuo. The residue was dissolved in 5 mL of 12 M HCl, and the mixture was heated to reflux, giving a homogenous solution. A little ethyl acetate or acetic acid could be added if dissolution was incomplete. After 1 h, 50 mL of 2M NaOH_{(aq.)} was added, then the suspension was extracted with ethyl acetate (3×20 mL). The combined organic layers were back extracted with brine (1×20 mL), dried over MgSO₄, filtered, and concentrated to 794 mg (74%) of a yellow solid. The product fluoresced when illuminated with 254 nm light.

### 4-chloro-6-phenyl-N-(2-(5-phenyl-1,3,4-thiadiazol-2-yl)phenyl)picolinamide

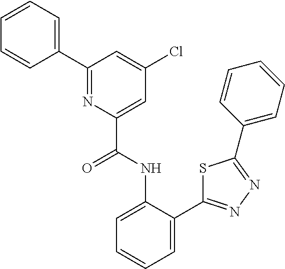

To a solution of 279 mg (1.10 mmol) of 2-(5-phenyl-1,3,4-thiadiazol-2-yl)aniline in 4 mL of CH₂Cl₂ was added 0.3 mL (2.2 mmol) of triethylamine, then 296 mg (1.17 mmol) of 4-chloro-6-phenylpicolinoyl chloride as a solution in 5 mL of CH₂Cl₂. The reaction was stirred at ambient temperature for 20 min, then 50 mL of methanol was added. The yellow precipitate was collected, washed with additional methanol, and dried on the filter to give 413 mg (80%) of a yellow solid.

### 6-chloro-2-phenyl-N-(2-(5-phenyl-1,3,4-thiadiazol-2-yl)phenyl)pyrimidine-4-carboxamide

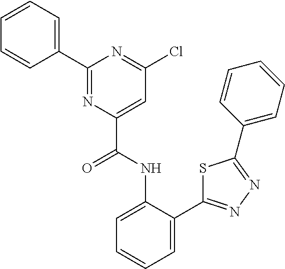

To a solution of 300 mg (1.18 mmol) of 2-(5-phenyl-1,3,4-thiadiazol-2-yl)aniline in 4 mL of CH₂Cl₂ was added 0.3 mL (2.2 mmol) of triethylamine, then a solution of 345 mg (1.36 mmol) of 6-chloro-2-phenylpyrimidine-4-carbonyl chloride in 3 mL of CH₂Cl₂. The reaction was stirred at ambient temperature for 10 min, then 50 mL of methanol was added. The precipitate was filtered, washed with water, and dried on the filter to give 542 mg (97%) of a yellow solid.

### 6-(2-(dimethylamino)ethylamino)-2-phenyl-N-(2-(5-phenyl-1,3,4-thiadiazol-2-yl)phenyl)pyrimidine-4-carboxamide

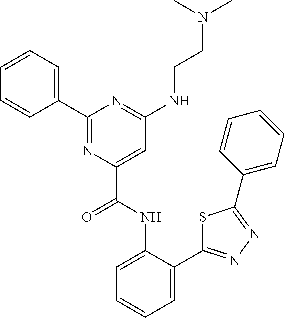

To a suspension of 100 mg (0.213 mmol) of 6-chloro-2-phenyl-N-(2-(5-phenyl-1,3,4-thiadiazol-2-yl)phenyl)pyrimidine-4-carboxamide in 2 mL of THF was added 0.2 mL (1.3 mmol) of N,N-dimethylethylenediamine. The reaction was heated at reflux for 45 min, then it was cooled and diluted with 10 mL of water. The precipitate was filtered, and the damp material was redissolved in 2 mL of THF with gentle warming. Next, 10 mL of water was added, and the precipitate was filtered. The product was dissolved in 8 mL of hot 1,2-dimethoxyethane, and 0.35 mL of 1 M HCl_{(aq.)} was added. The solvent was removed in vacuo, then the solid residue was suspended in water and filtered. The product was dried at 110° C. in vacuo to give 54 mg (45%) of a solid. MS Calcd. for C₂₉H₂₇N₇OS: 521.20. Found (M+H)⁺ m/z=522.

### N′-formyl-2-nitrobenzohydrazide

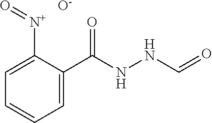

To 6 mL of formic acid was added 5 mL of acetic anhydride. The solution was stirred at ambient temperature for 2 h. A suspension of 4.0 g (22.1 mmol) of 2-nitrobenzohydrazide in 130 mL of ethyl acetate was warmed slightly to dissolve the hydrazide, then the acetic-formic anhydride solution was added. After stirring for 10 min, the reaction was cooled with an ice bath. The precipitate was filtered, washed with ethyl acetate, then dried in vacuo at ambient temperature to give 4.00 g (87%) of a white solid.

### 2-(2-nitrophenyl)-1,3,4-thiadiazole

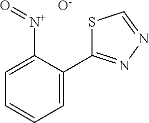

To 4.0 g (19 mmol) of N′-formyl-2-nitrobenzohydrazide was added 9.0 g (40.5 mmol) of phosphorus pentasulfide. The two solids were thoroughly mixed with a metal spatula, then 100 mL of toluene was added. The mixture was heated at reflux for 25 min, then cooled with an ice bath. Next, 50 mL of water was added, slowly at first to control heat evolution. The biphasic mixture was filtered to remove a yellow precipitate, occasionally washing the filter paper with ethyl acetate to improve the rate of filtration. The yellow precipitate was suspended in 25 mL of ethyl acetate, then filtered again. The filtrate and ethyl acetate washings were combined, and the layers were separated. The aqueous layer was extracted with ethyl acetate (1×25 mL), then the combined organic layers were extracted with 2 M NaOH_{(aq.)} (2×25 mL), then brine (1×25 mL), dried over MgSO₄, filtered, and concentrated to 3.50 g (88%) of a yellow solid.

### 2-(1,3,4-thiadiazol-2-yl)aniline

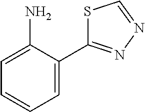

To 3.50 g (16.9 mmol) of 2-(2-nitrophenyl)-1,3,4-thiadiazole was added 4.64 g (84.0 mmol) of iron powder, then 1.08 g (20.3 mmol) of ammonium chloride. The mixture was suspended in 50 mL of isopropanol and 10 mL of water, then heated at reflux with vigorous stirring for 45 min. The hot solution was filtered, and the precipitate was washed with methanol. The combined filtrate and washings were concentrated in vacuo, then the residue was taken up in 40 mL of 6M HCl and heated at 100° C. for 30 min. The mixture was cooled with an ice bath, then 100 mL of 2M NaOH_{(aq.)} was added, followed by NaOH₍ₛ₎ until pH>13. The suspension was extracted with ethyl acetate (1×75 mL, then 2×25 mL), then the combined ethyl acetate layers were back extracted with 1 M NaOH (2×20 mL), and brine (1×20 mL), dried over MgSO₄, filtered, and concentrated to 2.69 g (90%) of a yellow solid.

### N-(2-(1,3,4-thiadiazol-2-yl)phenyl)-6-chloro-2-phenylpyrimidine-4-carboxamide

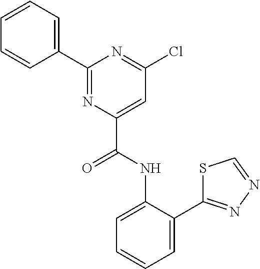

The title compound was prepared according to the procedure for 6-chloro-2-phenyl-N-(2-(5-phenyl-1,3,4-thiadiazol-2-yl)phenyl)pyrimidine-4-carboxamide, substituting 2-(1,3,4-thiadiazol-2-yl)aniline for 2-(5-phenyl-1,3,4-thiadiazol-2-yl)aniline. Yield 627 mg (90%).

### N-(2-(1,3,4-thiadiazol-2-yl)phenyl)-6-(2-(dimethylamino)ethylamino)-2-phenylpyrimidine-4-carboxamide

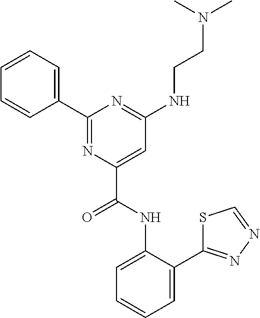

To 100 mg (0.254 mmol) of N-(2-(1,3,4-thiadiazol-2-yl)phenyl)-6-chloro-2-phenylpyrimidine-4-carboxamide was added 2 mL of THF, then 0.2 mL (1.3 mmol) of N,N-dimethylethylenediamine. The reaction was heated at reflux for 20 min, then diluted with 10 mL of water. The precipitate was collected, washed with water, and the damp solid was taken up again in 2 mL of THF. The solution was diluted with 10 mL of water, then the precipitate was filtered, washed with water, and dried in vacuo at 80° C. to give 69 mg (61%) of a pale yellow solid. MS Calcd. for C₂₃H₂₃N₇OS: 445.54. Found (M+H)⁺ m/z=446.

### N-(2-(1,3,4-thiadiazol-2-yl)phenyl)-6-((2-(dimethylamino)ethyl)(methyl)amino)-2-phenylpyrimidine-4-carboxamide

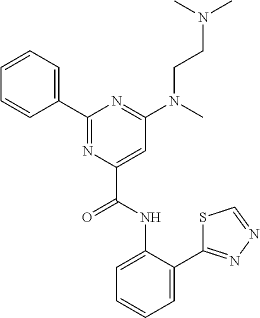

The title compound was prepared according to the same procedure as for N-(2-(1,3,4-thiadiazol-2-yl)phenyl)-6-(2-(dimethylamino)ethylamino)-2-phenylpyrimidine-4-carboxamide, substituting N,N,N′-trimethylethylenediamine for N,N-dimethylethylenediamine. Yield 85 mg (73%). MS Calcd. for C₂₄H₂₅N₇OS: 459.18 Found (M+H)⁺ m/z=460.

### N′-butyryl-2-nitrobenzohydrazide

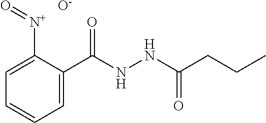

The title compound was prepared according to the same procedure as for N′-benzoyl-2-nitrobenzohydrazide, substituting n-butyryl chloride for benzoyl chloride. Yield 2.68 g (>100%).

### 2-(2-nitrophenyl)-5-propyl-1,3,4-thiadiazole

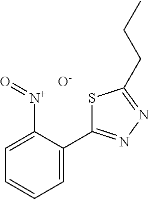

The title compound was prepared according to the same procedure as for 2-(2-nitrophenyl)-1,3,4-thiadiazole, substituting N′-butyryl-2-nitrobenzohydrazide for N′-formyl-2-nitrobenzohydrazide. Yield 2.0 g (80%).

### 2-(5-propyl-1,3,4-thiadiazol-2-yl)aniline

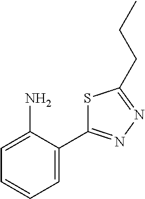

The title compound was prepared according to the same procedure as for 2-(1,3,4-thiadiazol-2-yl)aniline, substituting 2-(2-nitrophenyl)-5-propyl-1,3,4-thiadiazole for 2-(2-nitrophenyl)-1,3,4-thiadiazole. Yield 1.47 g (84%).

### 6-chloro-2-phenyl-N-(2-(5-propyl-1,3,4-thiadiazol-2-yl)phenyl)pyrimidine-4-carboxamide

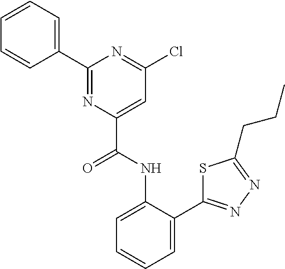

The title compound was prepared according to the same procedure as for 6-chloro-2-phenyl-N-(2-(5-phenyl-1,3,4-thiadiazol-2-yl)phenyl)pyrimidine-4-carboxamide, substituting 2-(5-propyl-1,3,4-thiadiazol-2-yl)aniline for 2-(5-phenyl-1,3,4-thiadiazol-2-yl)aniline. Yield 200 mg (79%).

### 6-(2-(dimethylamino)ethylamino)-2-phenyl-N-(2-(5-propyl-1,3,4-thiadiazol-2-yl)phenyl)pyrimidine-4-carboxamide

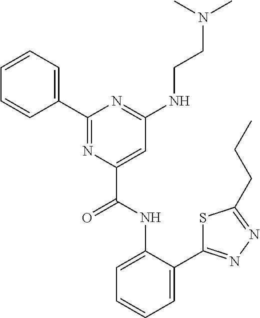

To a suspension of 200 mg, (0.44 mmol) of 6-chloro-2-phenyl-N-(2-(5-propyl-1,3,4-thiadiazol-2-yl)phenyl)pyrimidine-4-carboxamide in 15 mL of THF was added 640 mg (7.3 mmol) of N,N-dimethylethane-1,2-diamine. The reaction was stirred at reflux for 2 hours, then cooled to ambient temperature and diluted with 30 mL water. The precipitate was collected, washed with water, and dried to give 200 mg (93%) of a solid. MS Calcd. for C₂₆H₂₉N₇OS: 487.22 Found (M+H)⁺ m/z=488.

### N′-(3-methylbutanoyl)-2-nitrobenzohydrazide

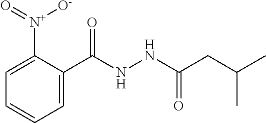

The title compound was prepared according to the same procedure as for N′-benzoyl-2-nitrobenzohydrazide, substituting 3-methylbutyryl chloride for benzoyl chloride. Yield 4.20 g (96%).

### 2-isobutyl-5-(2-nitrophenyl)-1,3,4-thiadiazole

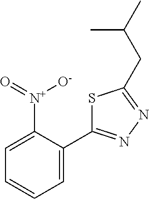

The title compound was prepared according to the same procedure as for 2-(2-nitrophenyl)-1,3,4-thiadiazole, substituting N′-(3-methylbutanoyl)-2-nitrobenzohydrazide for N′-formyl-2-nitrobenzohydrazide. Yield 3.5 g (84%).

### 2-(5-isobutyl-1,3,4-thiadiazol-2-yl)aniline

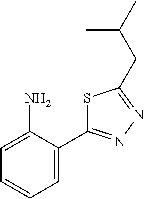

The title compound was prepared according to the same procedure as for 2-(1,3,4-thiadiazol-2-yl)aniline, substituting 2-(2-nitrophenyl)-5-propyl-1,3,4-thiadiazole for 2-(2-nitrophenyl)-1,3,4-thiadiazole. Yield 2.67 g (87%).

### 6-chloro-N-(2-(5-isobutyl-1,3,4-thiadiazol-2-yl)phenyl)-2-phenylpyrimidine-4-carboxamide

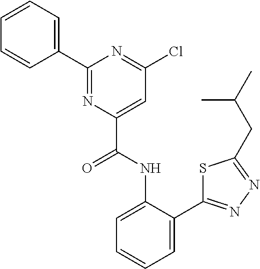

The title compound was prepared according to the same procedure as for 6-chloro-2-phenyl-N-(2-(5-phenyl-1,3,4-thiadiazol-2-yl)phenyl)pyrimidine-4-carboxamide, substituting 2-(5-isobutyl-1,3,4-thiadiazol-2-yl)aniline for 2-(5-phenyl-1,3,4-thiadiazol-2-yl)aniline. Yield 230 mg (91%).

### 6-(2-(dimethylamino)ethylamino)-N-(2-(5-isobutyl-1,3,4-thiadiazol-2-yl)phenyl)-2-phenylpyrimidine-4-carboxamide

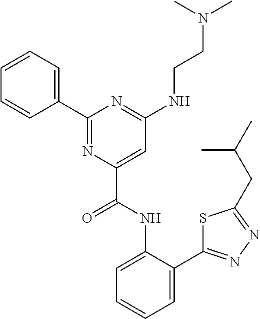

The title compound was prepared according to the same procedure as for 6-(2-(dimethylamino)ethylamino)-2-phenyl-N-(2-(5-propyl-1,3,4-thiadiazol-2-yl)phenyl)pyrimidine-4-carboxamide, substituting 6-chloro-N-(2-(5-isobutyl-1,3,4-thiadiazol-2-yl)phenyl)-2-phenylpyrimidine-4-carboxamide for 6-chloro-2-phenyl-N-(2-(5-propyl-1,3,4-thiadiazol-2-yl)phenyl)pyrimidine-4-carboxamide. Yield 207 mg (81%). MS Calcd. for C₂₇H₃₁N₇OS: 501.23 Found (M+H)⁺ m/z=502.

### N′-(isobutyryl)-2-nitrobenzohydrazide

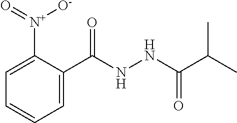

The title compound was prepared according to the same procedure as for N′-benzoyl-2-nitrobenzohydrazide, substituting isobutyryl chloride for benzoyl chloride. Yield 3.6 g (87%).

### 2-isopropyl-5-(2-nitrophenyl)-1,3,4-thiadiazole

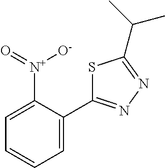

The title compound was prepared according to the same procedure as for 2-(2-nitrophenyl)-1,3,4-thiadiazole, substituting N′-(isobutyryl)-2-nitrobenzohydrazide for N′-formyl-2-nitrobenzohydrazide. Yield 3.17 g (89%).

### 2-(5-isobutyl-1,3,4-thiadiazol-2-yl)aniline

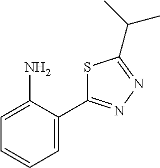

The title compound was prepared according to the same procedure as for 2-(1,3,4-thiadiazol-2-yl)aniline, substituting 2-isopropyl-5-(2-nitrophenyl)-1,3,4-thiadiazole for 2-(2-nitrophenyl)-1,3,4-thiadiazole. Yield 2.0 g (72%).

### 6-chloro-N-(2-(5-isopropyl-1,3,4-thiadiazol-2-yl)phenyl)-2-phenylpyrimidine-4-carboxamide

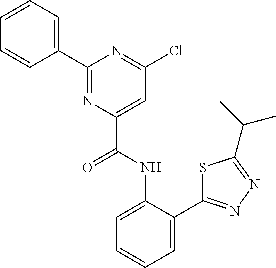

The title compound was prepared according to the same procedure as for 6-chloro-2-phenyl-N-(2-(5-phenyl-1,3,4-thiadiazol-2-yl)phenyl)pyrimidine-4-carboxamide, substituting 2-(5-isobutyl-1,3,4-thiadiazol-2-yl)aniline for 2-(5-phenyl-1,3,4-thiadiazol-2-yl)aniline. Yield 180 mg (96%).

### 6-((2-(dimethylamino)ethyl)(ethyl)amino)-2-phenyl-N-(2-(5-propyl-1,3,4-thiadiazol-2-yl)phenyl)pyrimidine-4-carboxamide

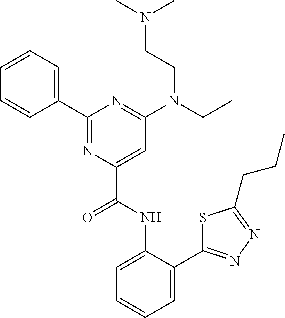

To a mixture of 130 mg (0.28 mmol) of 6-chloro-2-phenyl-N-(2-(5-propyl-1,3,4-thiadiazol-2-yl)phenyl)pyrimidine-4-carboxamide and 15 mL of THF was added 292 mg (2.50 mmol) of N¹-ethyl-N²,N²-dimethylethane-1,2-diamine. The reaction was stirred at reflux for 2 h, then cooled to ambient temperature and diluted with 30 mL of water. The precipitate was collected, washed with water, and dried to give 90 mg (58%) of a white solid.

Hydrochloride salt. To 5 mL of 2M HCl/MeOH was added 90 mg (0.60 mmol) of the free base. The precipitate was filtered and washed with diethyl ether to give 90 mg (96%) of a white solid. MS Calcd. for C₂₈H₃₃N₇OS: 515.25 Found (M+H)⁺ m/z=516.

### 6-((2-(dimethylamino)ethyl)(ethyl)amino)-N-(2-(5-isobutyl-1,3,4-thiadiazol-2-yl)phenyl)-2-phenylpyrimidine-4-carboxamide

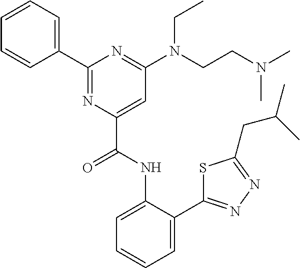

The title compound was prepared according to the same procedure as for 6-((2-(dimethylamino)ethyl)(ethyl)amino)-2-phenyl-N-(2-(5-propyl-1,3,4-thiadiazol-2-yl)phenyl)pyrimidine-4-carboxamide, substituting 6-chloro-N-(2-(5-isobutyl-1,3,4-thiadiazol-2-yl)phenyl)-2-phenylpyrimidine-4-carboxamide for 6-chloro-2-phenyl-N-(2-(5-propyl-1,3,4-thiadiazol-2-yl)phenyl)pyrimidine-4-carboxamide. Yield 180 mg (67%). MS Calcd. for C₂₉H₃₅N₇OS: 529.26 Found (M+H)⁺ m/z=530.

### 6-(2-(dimethylamino)ethyl)(ethyl)amino)-N-(2-(5-isopropyl-1,3,4-thiadiazol-2-yl)phenyl)-2-phenylpyrimidine-4-carboxamide

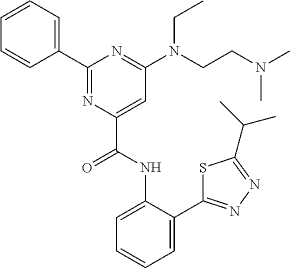

The title compound was prepared according to the same procedure as for 6-((2-(dimethylamino)ethyl)(ethyl)amino)-2-phenyl-N-(2-(5-propyl-1,3,4-thiadiazol-2-yl)phenyl)pyrimidine-4-carboxamide, substituting 6-chloro-N-(2-(5-isopropyl-1,3,4-thiadiazol-2-yl)phenyl)-2-phenylpyrimidine-4-carboxamide for 6-chloro-2-phenyl-N-(2-(5-propyl-1,3,4-thiadiazol-2-yl)phenyl)pyrimidine-4-carboxamide. Yield 100 mg (42%). MS Calcd. for C₂₈H₃₃N₇OS: 515.25 Found (M+H)⁺ m/z=516.

### N′-(2-nitrobenzoyl)tetrahydro-2H-pyran-4-carbohydrazide

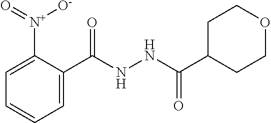

The title compound was prepared according to the same procedure as for N′-benzoyl-2-nitrobenzohydrazide, substituting tetrahydro-2H-pyran-4-carbonyl chloride for benzoyl chloride. Yield 965 mg (82%).

### 2-(2-nitrophenyl)-5-(tetrahydro-2H-pyran-4-yl)-1,3,4-thiadiazole

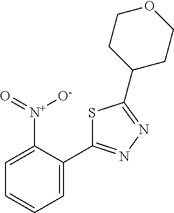

The title compound was prepared according to the same procedure as for 2-(2-nitrophenyl)-1,3,4-thiadiazole, substituting N′-(2-nitrobenzoyl)tetrahydro-2H-pyran-4-carbohydrazide for N′-formyl-2-nitrobenzohydrazide. Yield 438 mg (46%).

### 2-(5-(tetrahydro-2H-pyran-4-yl)-1,3,4-thiadiazol-2-yl)aniline

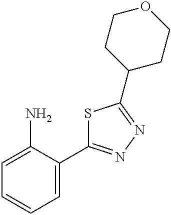

The title compound was prepared according to the same general procedure as for 2-(1,3,4-thiadiazol-2-yl)aniline, substituting 2-(2-nitrophenyl)-5-(tetrahydro-2H-pyran-4-yl)-1,3,4-thiadiazole for 2-(2-nitrophenyl)-1,3,4-thiadiazole. Yield 337 mg (86%).

### 6-chloro-2-phenyl-N-(2-(5-(tetrahydro-2H-pyran-4-yl)-1,3,4-thiadiazol-2-yl)phenyl)pyrimidine-4-carboxamide

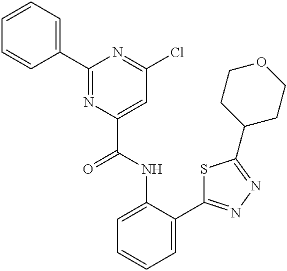

The title compound was prepared according to the same procedure as for 6-chloro-2-phenyl-N-(2-(5-phenyl-1,3,4-thiadiazol-2-yl)phenyl)pyrimidine-4-carboxamide, substituting 2-(5-(tetrahydro-2H-pyran-4-yl)-1,3,4-thiadiazol-2-yl)aniline for 2-(5-phenyl-1,3,4-thiadiazol-2-yl)aniline. Yield 382 mg (88%).

### 6-((2-(dimethylamino)ethyl)(ethyl)amino)-2-phenyl-N-(2-(5-(tetrahydro-2H-pyran-4-yl)-1,3,4-thiadiazol-2-yl)phenyl)pyrimidine-4-carboxamide hydrochloride

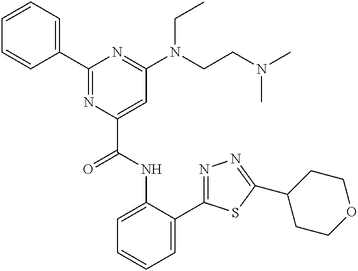

To a suspension of 100 mg (0.21 mmol) of 6-chloro-2-phenyl-N-(2-(5-(tetrahydro-2H-pyran-4-yl)-1,3,4-thiadiazol-2-yl)phenyl)pyrimidine-4-carboxamide in 2 mL of THF was added 0.20 mL (1.3 mmol) of N¹-ethyl-N²,N²-dimethylethane-1,2-diamine. The reaction was heated at reflux for 30 min, then diluted with 15 mL of water. The oily suspension was extracted with ethyl acetate (2×5 mL), then 5 mL of pentane was added and the organic layers were back extracted with water (2×5 mL), and brine (1×5 mL), dried over MgSO₄, filtered, and concentrated to an oil. This was taken up in 3 mL of THF, and 0.1 mL of 12M HCl was added to give a waxy precipitate. The THF was removed via pipette, and the solid was dried in vacuo at 60° C. to give 60 mg (48%) of a white solid. MS Calcd. for C₃₀H₃₅N₇O₂S: 557.26 Found (M+H)⁺ m/z=558.

### 6-(4-ethyl-3-oxopiperazin-1-yl)-2-phenyl-N-(2-(5-(tetrahydro-2H-pyran-4-yl)-1,3,4-thiadiazol-2-yl)phenyl)pyrimidine-4-carboxamide

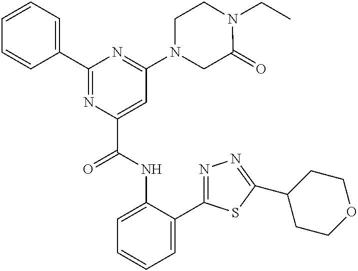

To 100 mg (0.41 mmol) of 1-ethylpiperazin-2-one trifluoroacetate salt was added 2 mL of THF, then 0.30 mL (2.2 mmol) of triethylamine. Next, 100 mg (0.209 mmol) of 6-chloro-2-phenyl-N-(2-(5-(tetrahydro-2H-pyran-4-yl)-1,3,4-thiadiazol-2-yl)phenyl)pyrimidine-4-carboxamide was added, and the suspension was heated at reflux for 2 h. The reaction was diluted with 15 mL of water, and the oily suspension was extracted with ethyl acetate (3×3 mL). The combined ethyl acetate layers were back extracted with 1M HCl (3×3 mL), saturated NaHCO_{3(aq.)} (2×3 mL), and brine (1×3 mL), dried over MgSO₄, filtered, and concentrated in vacuo to 62 mg (52%) of a pale yellow solid. MS Calcd. for C₃₀H₃₁N₇O₃S: 569.22 Found (M+H)⁺ m/z=570.

### N′-(2-nitrobenzoyl)tetrahydrofuran-3-carbohydrazide

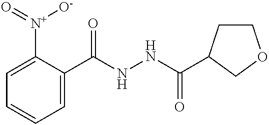

To a solution of 563 mg (4.85 mmol) of tetrahydrofuran-3-carboxylic acid in 3 mL of CH₂Cl₂ was added 423 μL (4.85 mmol) of oxalyl chloride. The mixture was stirred at ambient temperature for 18 h. A suspension of 725 mg (4.00 mmol) of 2-nitrobenzoylhydrazide in 20 mL of ethyl acetate was warmed to dissolve the hydrazide. To the solution was added 6 mL of saturated NaHCO_{3(aq.)}, then the solution of the acid chloride was added. The biphasic mixture was stirred at ambient temperature for 30 min, during which time a white precipitate formed. The precipitate was filtered, washed with water, and dried to give 771 mg (57%) of an off-white solid.

### 2-(2-nitrophenyl)-5-(tetrahydrofuran-3-yl)-1,3,4-thiadiazole

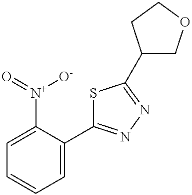

The title compound was prepared according to the same procedure as for 2-(2-nitrophenyl)-1,3,4-thiadiazole, substituting N′-(2-nitrobenzoyl)tetrahydrofuran-3-carbohydrazide for N′-formyl-2-nitrobenzohydrazide. Yield 243 mg (32%).

### 2-(5-(tetrahydrofuran-3-yl)-1,3,4-thiadiazol-2-yl)aniline

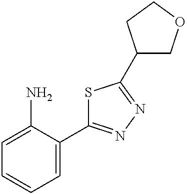

The title compound was prepared according to the same general procedure as for 2-(1,3,4-thiadiazol-2-yl)aniline, substituting 2-(2-nitrophenyl)-5-(tetrahydrofuran-3-yl)-1,3,4-thiadiazole for 2-(2-nitrophenyl)-1,3,4-thiadiazole. The product was purified via MPLC, eluting with a 10-40% ethyl acetate/pentane gradient. Yield 56 mg (26%).

### 6-chloro-2-phenyl-N-(2-(5-(tetrahydrofuran-3-yl)-1,3,4-thiadiazol-2-yl)phenyl)pyrimidine-4-carboxamide

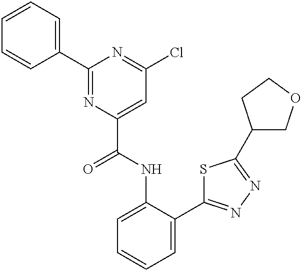

The title compound was prepared according to the same procedure as for 6-chloro-2-phenyl-N-(2-(5-phenyl-1,3,4-thiadiazol-2-yl)phenyl)pyrimidine-4-carboxamide, substituting 2-(5-(tetrahydrofuran-3-yl)-1,3,4-thiadiazol-2-yl)aniline for 2-(5-phenyl-1,3,4-thiadiazol-2-yl)aniline. Yield 65 mg (61%).

### 6-((2-(dimethylamino)ethyl)(ethyl)amino)-2-phenyl-N-(2-(5-(tetrahydrofuran-3-yl)-1,3,4-thiadiazol-2-yl)phenyl)pyrimidine-4-carboxamide

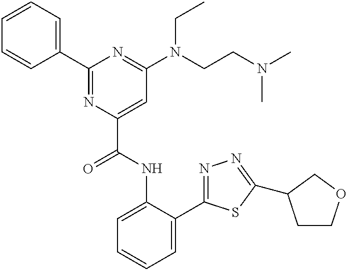

To 65 mg (0.14 mmol) of 6-chloro-2-phenyl-N-(2-(5-(tetrahydrofuran-3-yl)-1,3,4-thiadiazol-2-yl)phenyl)pyrimidine-4-carboxamide in 1 mL of THF was added 0.10 mL (0.65 mmol) of N1-ethyl-N2,N2-dimethylethane-1,2-diamine. The reaction was heated at reflux for 1 h, then concentrated in vacuo. The residue was taken up in 5 mL of ethyl acetate and 10 mL of pentane, then extracted with water (3×3 mL), and brine (1×3 mL), dried over MgSO₄, filtered, and concentrated in vacuo to a foam. This was dissolved in 1 mL of THF, then 1 drop of 12M HCl_{(aq)} was added. The solvents were removed in vacuo, and the solid was dried in vacuo at 120° C. for 3 h to give 38 mg (47%) of a solid. MS Calcd. for C₂₉H₃₃N₇O₂S: 543.24 Found (M+H)⁺ m/z=544.

### 2-fluoro-2-(2H-pyran-4(3H,5H,6H)-ylidene)acetic acid

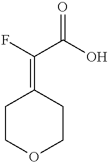

To a solution of 1.055 g (4.36 mmol) of ethyl 2-(diethoxyphosphoryl)-2-fluoroacetate in 15 mL of THF was added 174 mg (4.36 mmol) of 60% NaH in mineral oil. The reaction was stirred at ambient temperature for 45 min, then 436 mg (4.36 mmol) of 4-tetrahydropyranone was added. A gummy precipitate formed, and the reaction was stirred at ambient temperature for 10 min, then concentrated in vacuo. The residue was suspended in 10 mL of water, then the aqueous suspension was extracted with pentane (3×5 mL). The combined pentane layers were back extracted with water (2×3 mL), and brine (1×3 mL), dried over MgSO₄, filtered, and concentrated in vacuo to a colorless oil. This was dissolved in 5 mL of a solution prepared from 1.0 g of NaOH in 3 mL of water and 15 mL of methanol (1.39M NaOH in 5:1 methanol:water). The reaction was stirred for 25 min, then concentrated in vacuo. The residue was taken up in 5 mL of water, then extracted with pentane (3×3 mL) to remove the mineral oil. Next, 1 mL of 12 M HCl_{(aq)} was added to the aqueous layer, then the suspension was extracted with CH₂Cl₂ (3×3 mL). The combined CH₂Cl₂ layers were dried over MgSO₄, filtered, and concentrated in vacuo to 276 mg (40%) of a white solid.

### N′-(2-fluoro-2-(2H-pyran-4(3H,5H,6H)-ylidene)acetyl)-2-nitrobenzohydrazide

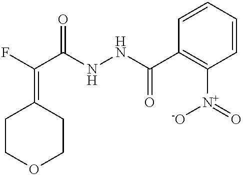

To 247 mg (1.54 mmol) of 2-fluoro-2-(2H-pyran-4(3H,5H,6H)-ylidene)acetic acid was added 2 mL of thionyl chloride. The reaction was heated at reflux for 20 min, then concentrated in vacuo to an oil. The acid chloride was dissolved in 2 mL of ethyl acetate. A suspension of 300 mg (1.66 mmol) of 2-nitrobenzoylhydrazide in 15 mL of ethyl acetate was heated to dissolve the hydrazide, then 10 mL of saturated NaHCO_{3(aq.)} was added. To the stirred, biphasic mixture was added the acid chloride solution, and the reaction was stirred for 30 min at ambient temperature. The aqueous layer was made acidic (pH=1) by addition of 12 M HCl, then the layers were separated. The aqueous layer was extracted with ethyl acetate (1×5 mL), then the combined organic layers were back extracted with brine (1×5 mL), dried over MgSO₄, filtered, and concentrated in vacuo to a white foam. Trituration with ethyl acetate gave 292 mg (59%) of a white crystalline solid.

### 2-(fluoro(2H-pyran-4(3H,5H,6H)-ylidene)methyl)-5-(2-nitrophenyl)-1,3,4-thiadiazole

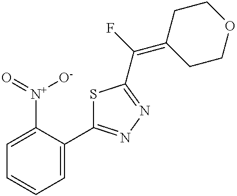

To a mixture of 292 mg (0.903 mmol) of N′-(2-fluoro-2-(2H-pyran-4(3H,5H,6H)-ylidene)acetyl)-2-nitrobenzohydrazide and 700 mg (3.15 mmol) of phosphorus pentasulfide was added 5 mL of toluene. The reaction was heated at reflux for 1.5 h, then cooled and diluted with 10 mL of water and 5 mL of ethyl acetate. The biphasic mixture was stirred for 5 min, then the layers were separated. The aqueous layer and any suspended material were drained, then extracted with ethyl acetate (1×5 mL). The combined organic layers were back extracted with 2M NaOH (1×5 mL), then brine (1×5 mL), dried over MgSO₄, filtered, and concentrated to 170 mg (59%) of a yellow solid.

### 2-(5-(fluoro(2H-pyran-4(3H,5H,6H)-ylidene)methyl)-1,3,4-thiadiazol-2-yl)aniline

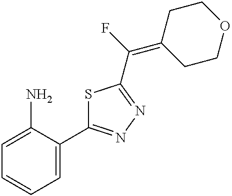

The title compound was prepared according to the same general procedure as for 2-(1,3,4-thiadiazol-2-yl)aniline, substituting 2-(fluoro(2H-pyran-4(3H,5H,6H)-ylidene)methyl)-5-(2-nitrophenyl)-1,3,4-thiadiazole for 2-(2-nitrophenyl)-1,3,4-thiadiazole. The product was purified via MPLC, eluting with a 5-25% ethyl acetate/pentane gradient. Yield 81 mg (53%).

### 6-chloro-N-(2-(5-(fluoro(2H-pyran-4(3H,5H,6H)-ylidene)methyl)-1,3,4-thiadiazol-2-yl)phenyl)-2-phenylpyrimidine-4-carboxamide

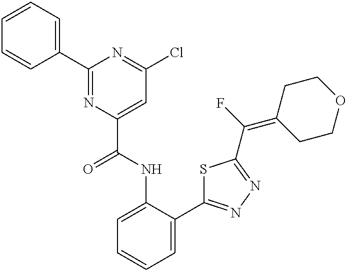

The title compound was prepared according to the same procedure as for 6-chloro-2-phenyl-N-(2-(5-phenyl-1,3,4-thiadiazol-2-yl)phenyl)pyrimidine-4-carboxamide, substituting 2-(5-(fluoro(2H-pyran-4(3H,5H,6H)-ylidene)methyl)-1,3,4-thiadiazol-2-yl)aniline for 2-(5-phenyl-1,3,4-thiadiazol-2-yl)aniline. Yield 89 mg (63%).

### 6-((2-(dimethylamino)ethyl)(ethyl)amino)-N-(2-(5-(fluoro(2H-pyran-4(3H,5H,6H)-ylidene)methyl)-1,3,4-thiadiazol-2-yl)phenyl)-2-phenylpyrimidine-4-carboxamide

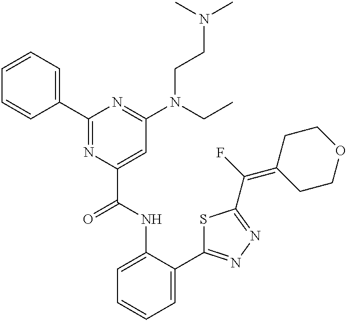

The title compound was prepared according to the same procedure as for 6-((2-(dimethylamino)ethyl)(ethyl)amino)-2-phenyl-N-(2-(5-(tetrahydrofuran-3-yl)-1,3,4-thiadiazol-2-yl)phenyl)pyrimidine-4-carboxamide, substituting 6-chloro-N-(2-(5-(fluoro(2H-pyran-4(3H,5H,6H)-ylidene)methyl)-1,3,4-thiadiazol-2-yl)phenyl)-2-phenylpyrimidine-4-carboxamide for 6-chloro-2-phenyl-N-(2-(5-(tetrahydrofuran-3-yl)-1,3,4-thiadiazol-2-yl)phenyl)pyrimidine-4-carboxamide. Trituration of the free base foam with ethyl acetate or methanol gave pale yellow microcrystals. The free base was dissolved in 2 mL of THF, then 2 drops of 12 M HCl was added. The solvents were removed in vacuo, then the residue was triturated with ethyl acetate to give 49 mg (40%) of a yellow solid. MS Calcd. for C₃₁H₃₄FN₇O₂S: 587.25 Found (M+H)⁺ m/z=588.

### 5-methyl-2-(2-nitrophenyl)thiazole

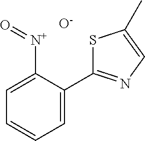

To a solution of 907 mg (4.08 mmol) of 2-nitro-N-(2-oxopropyl)benzamide in 15 mL of toluene at reflux was added 1.62 g (7.30 mmol) of phosphorus pentasulfide. The reaction was stirred at reflux for 15 minutes, then 10 mL of water was added, followed by 20 mL of 2M NaOH_{(aq.)}. The mixture was sonicated to suspend any clumps of P₂S₅, then the mixture was extracted with ethyl acetate (3×10 mL). The combined ethyl acetate layers were back extracted with 2M NaOH_{(aq.)} (2×10 mL), and brine (1×10 mL), dried over MgSO₄, filtered, and concentrated to a yellow oil. The product was purified via MPLC, eluting with a 5-40% ethyl acetate/pentane gradient to give 387 mg (43%) of a pale yellow solid.

### 2-(5-methylthiazol-2-yl)aniline

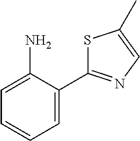

To 378 mg (1.72 mol) of 5-methyl-2-(2-nitrophenyl)thiazole was added 472 mg (8.58 mmol) of iron powder, 110 mg (2.06 mmol) of ammonium chloride, then 10 mL of isopropyl alcohol and 2 mL of water. The reaction was heated at reflux for 15 minutes, then filtered and concentrated in vacuo. The residue was taken up in 15 mL of ethyl acetate, then extracted with water (2×3 mL), and brine (1×3 mL), dried over MgSO₄, filtered, and concentrated to give 304 mg (93%) of a pale yellow solid.

### 6-chloro-N-(2-(5-methylthiazol-2-yl)phenyl)-2-phenylpyrimidine-4-carboxamide

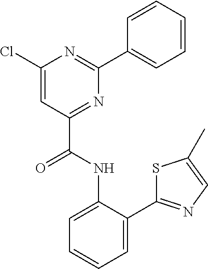

The title compound was prepared according to the same procedure as for 6-chloro-N-(2-(5-methyloxazol-2-yl)phenyl)-2-phenylpyrimidine-4-carboxamide, substituting 2-(5-methylthiazol-2-yl)aniline for 2-(5-methyloxazol-2-yl)aniline. Yield 370 mg (91%).

### 6-(2-(dimethylamino)ethylamino)-N-(2-(5-methylthiazol-2-yl)phenyl)-2-phenylpyrimidine-4-carboxamide

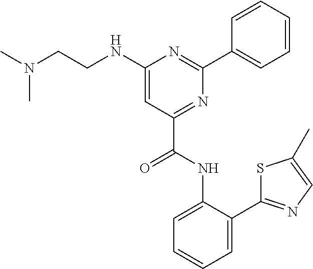

To a suspension of 100 mg (0.25 mmol) of 6-chloro-N-(2-(5-methylthiazol-2-yl)phenyl)-2-phenylpyrimidine-4-carboxamide in 2 mL of THF was added 0.15 mL (1.4 mmol) of N,N-dimethylethylenediamine. The reaction was heated at reflux for 25 min, then diluted with 10 mL of water. The precipitate was filtered, then recrystallized from ethanol to give 79 mg (69%) of white needles. MS Calcd. for C₂₅H₂₆N₆OS: 458.19 Found (M+H)⁺ m/z=459.

### Preparation of 2-(6-((2-(dimethylamino)ethyl)(ethyl)amino)-2-phenylpyrimidine-4-carboxamido)benzoic acid

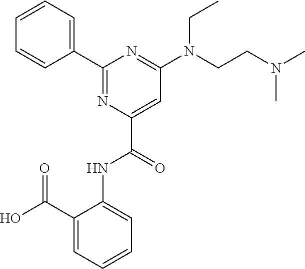

Prepared according to the same procedure as for 2-(6-(2-(dimethylamino)ethylamino)-2-phenylpyrimidine-4-carboxamido)benzoic acid, eliminating the recrystallization procedure. Yield 1.93 g (93%).

### General Procedure E

To a solution of 0.37 mmol, (1 eq.) of 2-(6-(2-(dimethylamino)ethylamino)-2-phenylpyrimidine-4-carboxamido)benzoic acid, or 2-(6-((2-(dimethylamino)ethyl)(ethyl)amino)-2-phenylpyrimidine-4-carboxamido)benzoic acid and 0.44 mmol (1.2 eq.) of the appropriate amine in 3 mL of DMF was added 213 mg (0.56 mmol, 1.5 eq) of 2-(7-Aza-1H-benzotriazole-1-yl)-1,1,3,3-tetramethyluronium hexafluorophosphate (HATU), and 144 mg (1.11 mmol, 3 eq.) of N,N-diisopropyl-N-ethylamine. The reaction was stirred at ambient temperature for 3 h, then diluted with 15 mL of water. The suspension was extracted with ethyl acetate, then the organic layer was dried over Na₂SO₄, filtered and concentrated. If necessary, the crude product was purified via silica gel chromatography, eluting with a 1-5% methanol/CH₂Cl₂ gradient to give the final compound.

### General Method F:

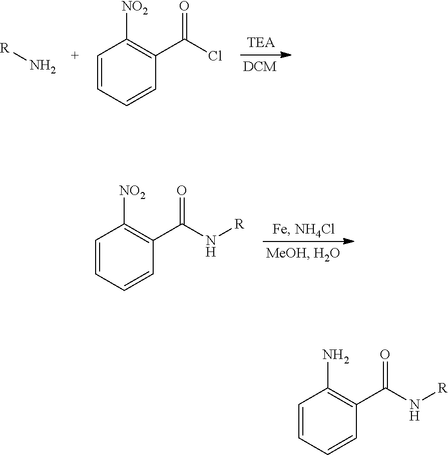

The amine (1 eq) was dissolved in dichloromethane. Triethylamine (1.2 eq) is added and the mixture was stirred at room temperature for 5 minutes. A solution of 2-nitrobenzoyl chloride (1.5 eq.) in dichloromethane was added dropwise to the reaction mixture. The reaction mixture was stirred for 1.5 hrs. The reaction mixture was concentrated down under vacuum and purified via column chromatography (eluted with CH₂Cl₂).

The amide (1 eq) was dissolved in 4:1 methanol:water. Iron powder (5 eq) and ammonium chloride (8 eq) were added and the mixture is heated to reflux for 3 hrs. The reaction mixture was filtered, and the filtrate was diluted with ethyl acetate and washed with water. The organic layer was dried over magnesium sulfate and concentrated under vacuum.

### General Method G:

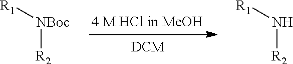

The Boc-protected amine was dissolved in dichloromethane. A 4 M HCl solution in methanol was added and the reaction mixture is allowed to stir overnight. The solution was diluted with diethyl ether to form a precipitate. The precipitate was washed with diethyl ether and is dried under vacuum.

### Preparation of 6-(2-(dimethylamino)ethylamino)-2-phenyl-N-(2-(pyrrolidine-1-carbonyl)phenyl)pyrimidine-4-carboxamide

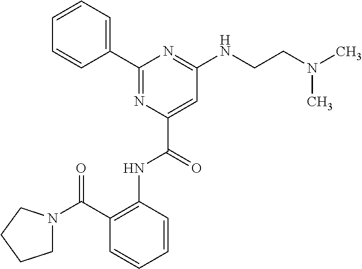

The title compound was prepared according to General Method E using pyrrolidine. Yield 70 mg. MS Calcd for C₂₆H₃₀N₆O₂: 458.24. Found (M+H)⁺ m/z=459

### Preparation of 6-(2-(dimethylamino)ethylamino)-N-(2-(2-(dimethylamino)ethylcarbamoyl)phenyl)-2-phenylpyrimidine-4-carboxamide

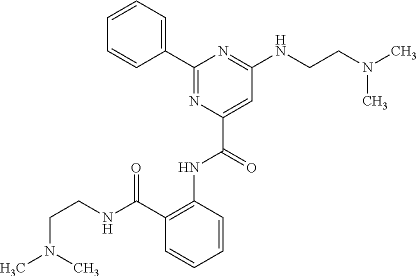

The title compound was prepared according to General Method E using N,N-dimethylethylenediamine. Yield 17 mg. MS Calcd for C₂₆H₃₃N₇O₂: 475.27. Found (M+H)⁺ m/z=476

### Preparation of 6-(2-(dimethylamino)ethylamino)-N-(2-(2-methoxyethylcarbamoyl)phenyl)-2-phenylpyrimidine-4-carboxamide

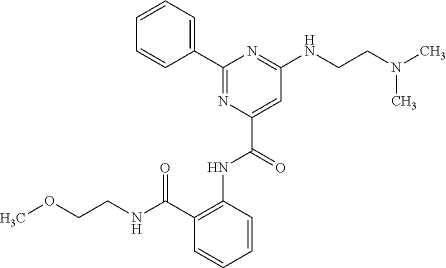

The title compound was prepared according to General Method E using 2-methoxyethanamine. Yield 26 mg. MS Calcd for C₂₅H₃₀N₆O₃: 462.24. Found (M+H)⁺ m/z=463

### Preparation of N-(2-(butylcarbamoyl)phenyl)-6-(2-(dimethylamino)ethylamino)-2-phenylpyrimidine-4-carboxamide

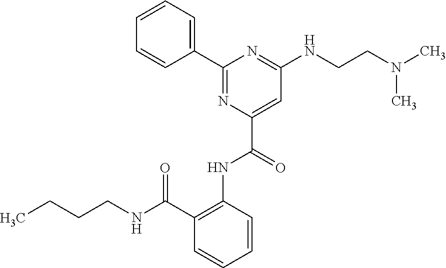

The title compound was prepared according to General Method E using buytlamine. Yield 64 mg. MS Calcd for C₂₆H₃₂N₆O₂: 460.26. Found (M+H)⁺ m/z=461

### Preparation of N-(2-(diethylcarbamoyl)phenyl)-6-(2-(dimethylamino)ethylamino)-2-phenylpyrimidine-4-carboxamide

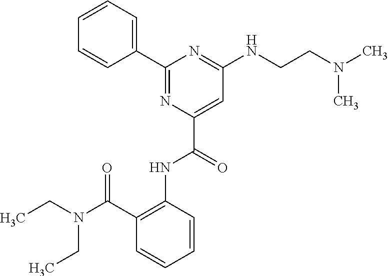

The title compound was prepared according to General Method E using diethylamine. Yield 50 mg. MS Calcd for C₂₆H₃₂N₆O₂: 460.26. Found (M+H)⁺ m/z=461

### Preparation of 6-(2-(dimethylamino)ethylamino)-N-(2-(methylcarbamoyl)phenyl)-2-phenylpyrimidine-4-carboxamide

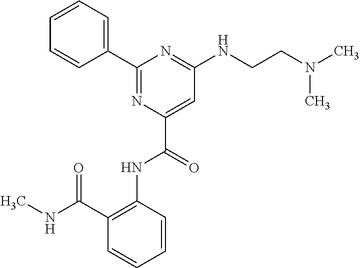

The title compound was prepared according to General Method E using methylamine. Yield 43 mg. MS Calcd for C₂₃H₂₆N₆O₂: 418.21. Found (M+H)⁺ m/z=419

### Preparation of 6-(2-(dimethylamino)ethylamino)-2-phenyl-N-(2-(phenylcarbamoyl)phenyl)pyrimidine-4-carboxamide

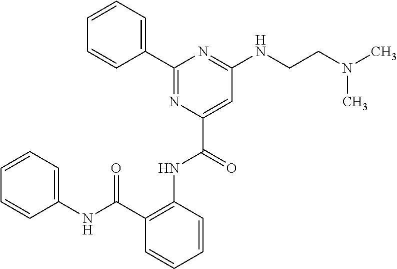

The title compound was prepared according to General Method E. Yield 170 mg. MS Calcd for C₂₈H₂₈N₆O₂: 480.23. Found (M+H)⁺ m/z=481

### Preparation of 3-(2-(6-(2-(dimethylamino)ethylamino)-2-phenylpyrimidine-4-carboxamido)benzamido)propanoic acid

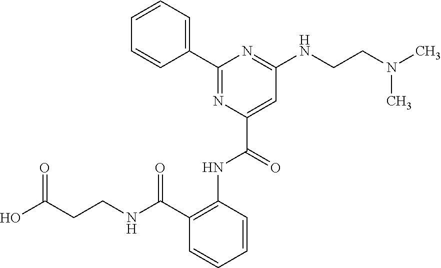

The title compound was prepared according to General Method E, starting with ethyl (3-amino)propionate. To a solution of 95 mg (0.20 mmol) of the intermediate ethyl ester in 3 mL of THF and 3 mL of methanol was added 2 mL of 1.5 M NaOH_{(aq.)}. The mixture was stirred for 18 h, then concentrated in vacuo. The residue was taken up in 10 mL of water, then the mixture was extracted with ethyl acetate (1×20 mL). The aqueous phase was adjusted to pH=4-5 with 10% HCl_{(aq.)}, then the precipitate was filtered, washed with water, and dried in vacuo to give 35 mg (38%) of a white solid. MS Calcd for C₂₅H₂₈N₆O₄: 476.22. Found (M+H)⁺ m/z=477

### Preparation of 6-(2-(dimethylamino)ethylamino)-2-phenyl-N-(2-(p-tolylcarbamoyl)phenyl)pyrimidine-4-carboxamide

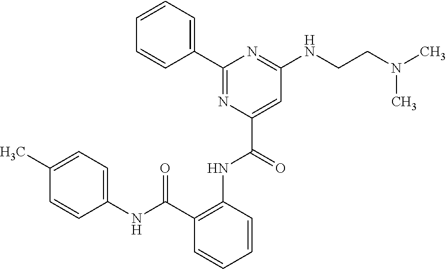

The title compound was prepared according to General Method A. The aniline was prepared according to General Method F using p-toluidine. Yield 25 mg (23%). MS Calcd for C₂₉H₃₀N₆O₂: 494.24. Found (M+H)⁺ m/z=495

### Preparation of N-(2-(3-chlorophenylcarbamoyl)phenyl)-6-(2-(dimethylamino)ethylamino)-2-phenylpyrimidine-4-carboxamide

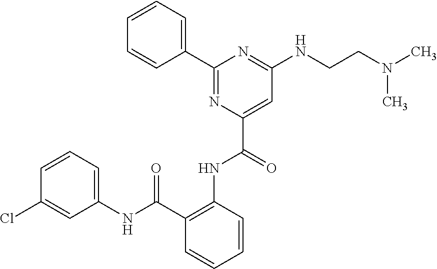

The title compound was prepared according to General Method A. The aniline was prepared according to General Method F using 3-chloroaniline. Yield 210 mg (73%). MS Calcd for C₂₈H₂₇ClN₆O₂: 514.19. Found (M+H)⁺ m/z=515

### Preparation of 6-(2-(dimethylamino)ethylamino)-2-phenyl-N-(2-(m-tolylcarbamoyl)phenyl)pyrimidine-4-carboxamide

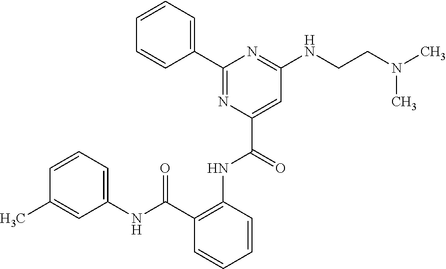

The title compound was prepared according to General Method A. The aniline was prepared according to General Method F using m-toluidine. Yield 220 mg (90%). MS Calcd for C₂₉H₃₀N₆O₂: 494.24. Found (M+H)⁺ m/z=495

### Preparation of N-(2-(3-carbamoylphenylcarbamoyl)phenyl)-6-(2-(dimethylamino)ethylamino)-2-phenylpyrimidine-4-carboxamide

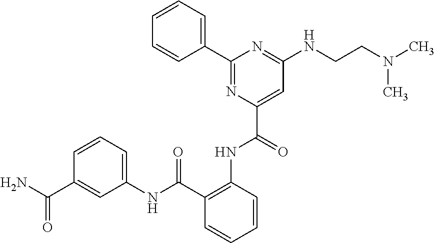

The title compound was prepared according to General Method A. The aniline was prepared according to General Method F using 3-aminobenzamide. Yield 140 mg (85%). MS Calcd for C₂₉H₂₉N₇O₃: 523.23. Found (M+H)⁺ m/z=524

### Preparation of 6-(2-(dimethylamino)ethylamino)-N-(2-(2-(methylthio)ethylcarbamoyl)phenyl)-2-phenylpyrimidine-4-carboxamide

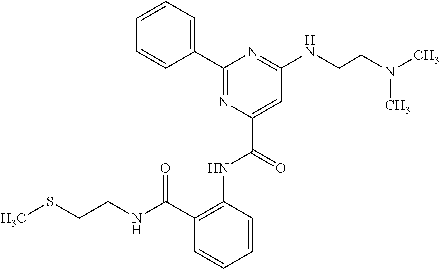

The title compound was prepared according to General Method E using 2-(methylthio)ethanamine. Yield 550 mg (46%). MS Calcd for C₂₅H₃₀N₆O₂S: 478.22. Found (M+H)⁺ m/z=479

### Preparation of 6-(2-(dimethylamino)ethylamino)-N-(2-(2-(methylsulfonyl)ethylcarbamoyl)phenyl)-2-phenylpyrimidine-4-carboxamide

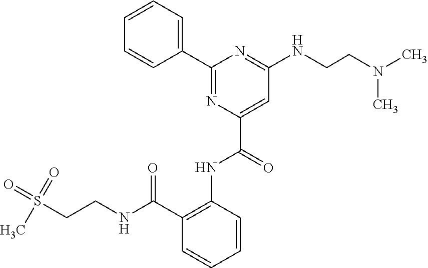

The title compound was prepared according to General Method E using 2-(methylsulfonyl)ethanamine. Yield 140 mg (89%). MS Calcd for C₂₅H₃₀N₆O₄S: 510.20. Found (M+H)⁺ m/z=511

### Preparation of N-(2-(2-carbamoylphenylcarbamoyl)phenyl)-6-(2-(dimethylamino)ethylamino)-2-phenylpyrimidine-4-carboxamide

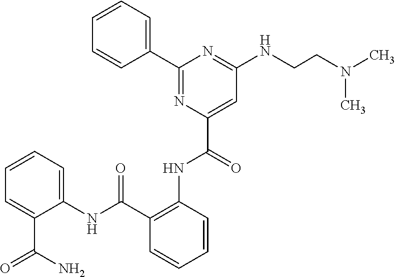

The title compound was prepared according to General Method A. The aniline was prepared according to General Method F using 2-aminobenzamide. Yield 210 mg (94%). MS Calcd for C₂₉H₂₉N₇O₃: 523.23. Found (M+H)⁺ m/z=524

### Preparation of 6-(2-(dimethylamino)ethylamino)-N-(2-(methyl(phenyl)carbamoyl)phenyl)-2-phenylpyrimidine-4-carboxamide

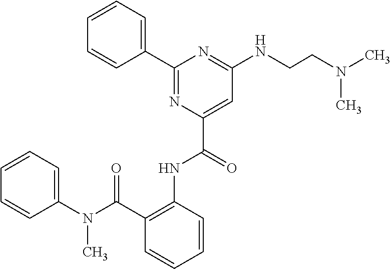

The title compound was prepared according to General Method A. The aniline was prepared according to General Method F using N-methylaniline. Yield 102 mg (75%). MS Calcd for C₂₉H₃₀N₆O₂: 494.24. Found (M+H)⁺ m/z=495

### Preparation of 6-(2-(dimethylamino)ethylamino)-N-(2-(2-(methylsulfinyl)ethylcarbamoyl)phenyl)-2-phenylpyrimidine-4-carboxamide

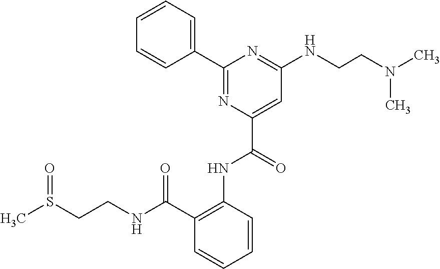

The title compound was prepared according to General Method E using 2-(methylsulfinyl)ethanamine. Yield 100 mg (48%). MS Calcd for C₂₅H₃₀N₆O₃S: 494.21. Found (M+H)⁺ m/z=495

### Preparation of N-(2-(3,4-dichlorophenylcarbamoyl)phenyl)-6-(2-(dimethylamino)ethylamino)-2-phenylpyrimidine-4-carboxamide

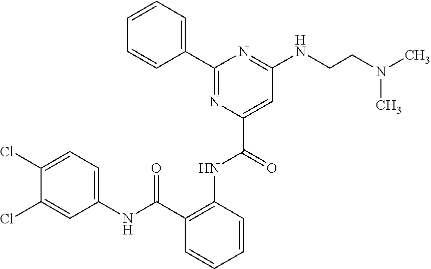

The title compound was prepared according to General Method A. The aniline was prepared according to General Method F using 3,4-dichloroaniline. Yield 200 mg (91%). MS Calcd for C₂₈H₂₆Cl₂N₆O₂: 548.15. Found (M+H)⁺ m/z=549

### Preparation of 6-(2-(dimethylamino)ethylamino)-N-(2-(3-(methylsulfonyl)phenylcarbamoyl)phenyl)-2-phenylpyrimidine-4-carboxamide

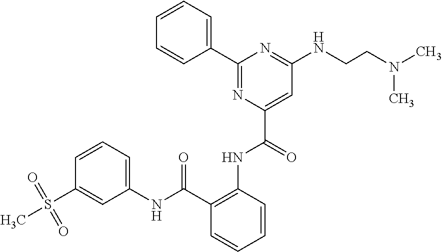

The title compound was prepared according to General Method A. The aniline was prepared according to General Method F using 3-(methylsulfonyl)aniline. Yield 100 mg (94%). MS Calcd for C₂₉H₃₀N₆O₄S: 558.20. Found (M+H)⁺ m/z=559

### Preparation of 6-(2-(dimethylamino)ethylamino)-2-phenyl-N-(2-(pyridin-3-ylcarbamoyl)phenyl)pyrimidine-4-carboxamide

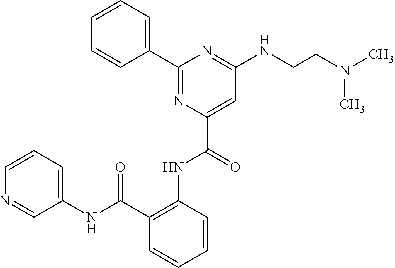

The title compound was prepared according to General Method A. The aniline was prepared according to General Method F using pyridin-3-amine. Yield 209 mg (94%). MS Calcd for C₂₇H₂₇N₇O₂: 481.22. Found (M+H)⁺ m/z=482

### Preparation of 6-(2-(dimethylamino)ethylamino)-2-phenyl-N-(2-(pyridin-4-ylcarbamoyl)phenyl)pyrimidine-4-carboxamide

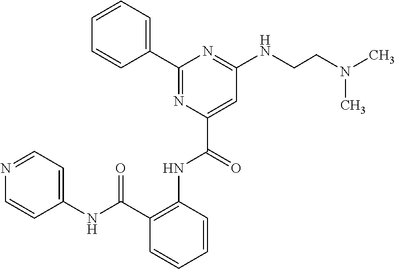

The title compound was prepared according to General Method A. The aniline was prepared according to General Method F using pyridin-4-amine. Yield 50 mg (87%). MS Calcd for C₂₇H₂₇N₇O₂: 481.22. Found (M+H)⁺ m/z=482

### Preparation of N-(2-(4-carbamoylphenylcarbamoyl)phenyl)-6-(2-(dimethylamino)ethylamino)-2-phenylpyrimidine-4-carboxamide

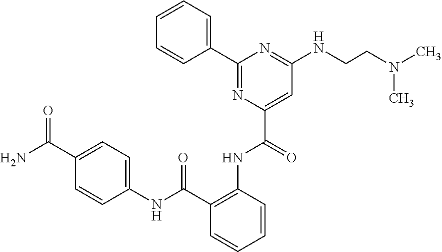

The title compound was prepared according to General Method A. The aniline was prepared according to General Method F using 4-aminobenzamide. Yield 70 mg (67%). MS Calcd for C₂₉H₂₉N₇O₃: 523.23. Found (M+H)⁺ m/z=524

### Preparation of 6-(2-(dimethylamino)ethylamino)-2-phenyl-N-(2-(pyridin-2-ylcarbamoyl)phenyl)pyrimidine-4-carboxamide

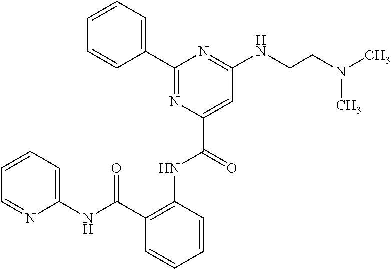

The title compound was prepared according to General Method A. The aniline was prepared according to General Method F using pyridin-2-amine. Yield 70 mg (62%). MS Calcd for C₂₇H₂₇N₇O₂: 481.22. Found (M+H)⁺ m/z=482

### Preparation of N-(2-(2-carbamoylphenylcarbamoyl)phenyl)-6-((2-(dimethylamino)ethyl)(ethyl)amino)-2-phenylpyrimidine-4-carboxamide

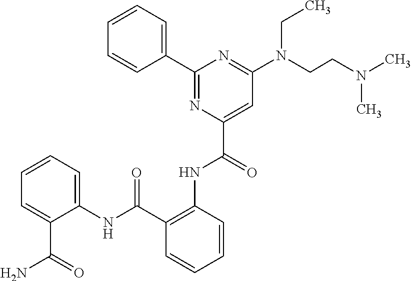

The title compound was prepared according to General Method A. The aniline was prepared according to General Method F using 2-aminobenzamide. Yield 42 mg (36%). MS Calcd for C₃₁H₃₃N₇O₃: 551.26. Found (M+H)⁺ m/z=552

### Preparation of N-(2-(2,5-dichlorophenylcarbamoyl)phenyl)-6-(2-(dimethylamino)ethylamino)-2-phenylpyrimidine-4-carboxamide

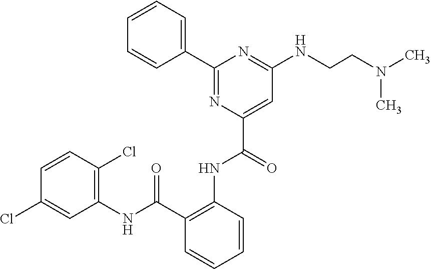

The title compound was prepared according to General Method A. The aniline was prepared according to General Method F using 2,5-dichloroaniline. Yield 130 mg (73%). MS Calcd for C₂₈H₂₆Cl₂N₆O₂: 548.15. Found (M+H)⁺ m/z=549

### Preparation of N-(2-(2,6-dichlorophenylcarbamoyl)phenyl)-6-(2-(dimethylamino)ethylamino)-2-phenylpyrimidine-4-carboxamide

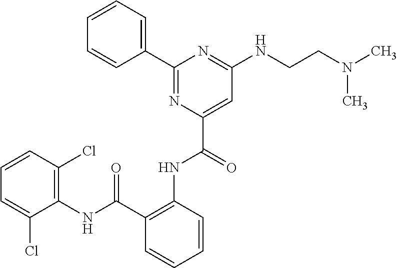

The title compound was prepared according to General Method A. The aniline was prepared according to General Method F using 2,6-dichloroaniline. Yield 70 mg (49%). MS Calcd for C₂₈H₂₆Cl₂N₆O₂: 548.15. Found (M+H)⁺ m/z=549

### Preparation of 6-(2-(dimethylamino)ethylamino)-N-(2-(2-(methylsulfonyl)phenylcarbamoyl)phenyl)-2-phenylpyrimidine-4-carboxamide

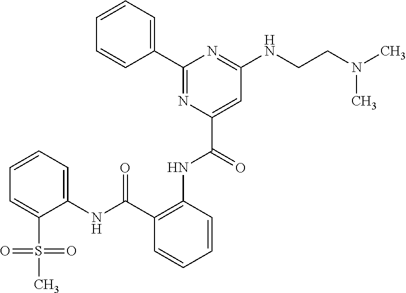

The title compound was prepared according to General Method A. The aniline was prepared according to General Method F using 2-(methylsulfonyl)aniline. Yield 60 mg (28%). MS Calcd for C₂₉H₃₀N₆O₄S: 558.20. Found (M+H)⁺ m/z=559

### Preparation of N-(2-(cyclobutylcarbamoyl)phenyl)-6-((2-(dimethylamino)ethyl)(ethyl)amino)-2-phenylpyrimidine-4-carboxamide

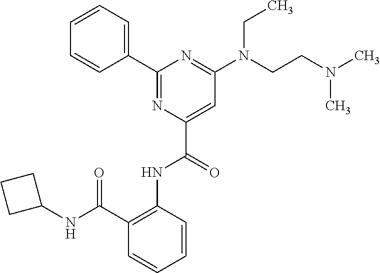

The title compound was prepared according to General Method E using cyclobutylamine. Yield 60 mg (49%). MS Calcd for C₂₈H₃₄N₆O₂: 486.27. Found (M+H)⁺ m/z=487

### Preparation of N-(2-(cyclohexylcarbamoyl)phenyl)-6-((2-(dimethylamino)ethyl)(ethyl)amino)-2-phenylpyrimidine-4-carboxamide

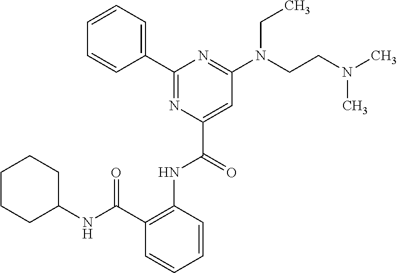

The title compound was prepared according to General Method E using cyclohexylamine. Yield 118 mg (91%). MS Calcd for C₃₀H₃₈N₆O₂: 514.31. Found (M+H)⁺ m/z=515

### Preparation of tert-butyl 4-(2-(6-((2-(dimethylamino)ethyl)(ethyl)amino)-2-phenylpyrimidine-4-carboxamido)benzamido)piperidine-1-carboxylate

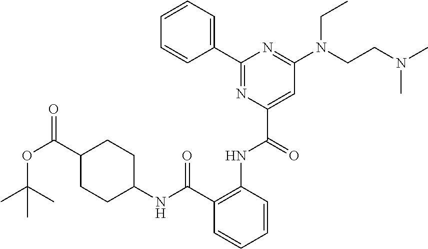

The title compound was prepared according to General Method E using tert-butyl 4-aminopiperidine-1-carboxylate. Yield 190 mg (56%). MS Calcd for C₃₄H₄₅N₇O₄: 615.35. Found (M+H)⁺ m/z=616

### Preparation of 6-((2-(dimethylamino)ethyl)(ethyl)amino)-2-phenyl-N-(2-(piperidin-4-ylcarbamoyl)phenyl)pyrimidine-4-carboxamide

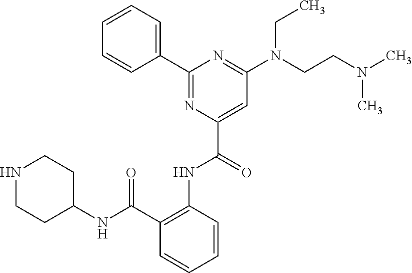

The title compound was prepared from tert-butyl 4-(2-(6-((2-(dimethylamino)ethyl)(ethyl)amino)-2-phenylpyrimidine-4-carboxamido)benzamido)piperidine-1-carboxylate according to General Method G. Yield 72 mg (63%). MS Calcd for C₂₉H₃₇N₇O₂: 515.30. Found (M+H)⁺ m/z=516

### Preparation of N-(2-(cyclopropylcarbamoyl)phenyl)-6-((2-(dimethylamino)ethyl)(ethyl)amino)-2-phenylpyrimidine-4-carboxamide

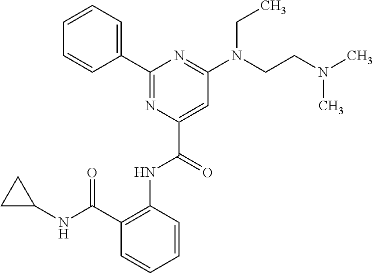

The title compound was prepared according to General Method E using cyclopropylamine. Yield 95 mg (79%). MS Calcd for C₂₇H₃₂N₆O₂: 472.26. Found (M+H)⁺ m/z=473

### Preparation of N-(2-(cyclopentylcarbamoyl)phenyl)-6-((2-(dimethylamino)ethyl)(ethyl)amino)-2-phenylpyrimidine-4-carboxamide

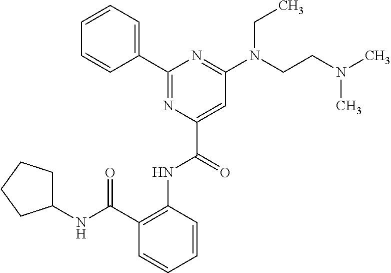

The title compound was prepared according to General Method E using cyclopentylamine. Yield 105 mg (83%). MS Calcd for C₂₉H₃₆N₆O₂: 500.29. Found (M+H)⁺ m/z=501

### Preparation of 6-((2-(dimethylamino)ethyl)(ethyl)amino)-N-(2-((1R,2S)-2-hydroxycyclohexylcarbamoyl)phenyl)-2-phenylpyrimidine-4-carboxamide

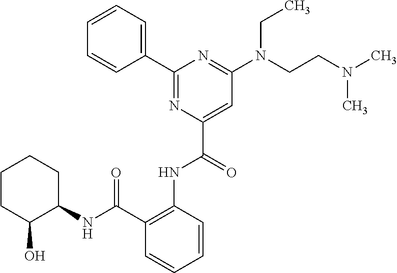

The title compound was prepared according to General Method E using (1S,2R)-2-aminocyclohexanol. Yield 70 mg (52%). MS Calcd for C₃₀H₃₈N₆O₃: 530.30. Found (M+H)⁺ m/z=531

### Preparation of 6-((2-(dimethylamino)ethyl)(ethyl)amino)-N-(2-((1R,2R)-2-hydroxycyclohexylcarbamoyl)phenyl)-2-phenylpyrimidine-4-carboxamide

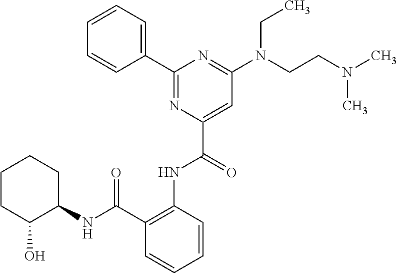

The title compound was prepared according to General Method E using (1R,2R)-2-aminocyclohexanol. Yield 99 mg (73%). MS Calcd for C₃₀H₃₈N₆O₃: 530.30. Found (M+H)⁺ m/z=531

### Preparation of tert-butyl 3-(2-(6-((2-(dimethylamino)ethyl)(ethyl)amino)-2-phenylpyrimidine-4-carboxamido)benzamido)piperidine-1-carboxylate

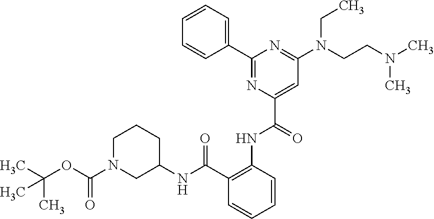

The title compound was prepared according to General Method E using tert-butyl 3-aminopiperidine-1-carboxylate. Yield 270 mg (86%). MS Calcd for C₃₄H₄₅N₇O₄: 615.35. Found (M+H)⁺ m/z=616

### Preparation of tert-butyl 3-(2-(6-((2-(dimethylamino)ethyl)(ethyl)amino)-2-phenylpyrimidine-4-carboxamido)benzamido)pyrrolidine-1-carboxylate

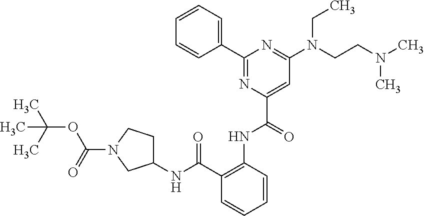

The title compound was prepared according to General Method E using tert-butyl 3-aminopyrrolidine-1-carboxylate. Yield 275 mg (90%). MS Calcd for C₃₃H₄₃N₇O₄: 601.34. Found (M+H)⁺ m/z=602

### Preparation of 6-((2-(dimethylamino)ethyl)(ethyl)amino)-2-phenyl-N-(2-(piperidin-3-ylcarbamoyl)phenyl)pyrimidine-4-carboxamide

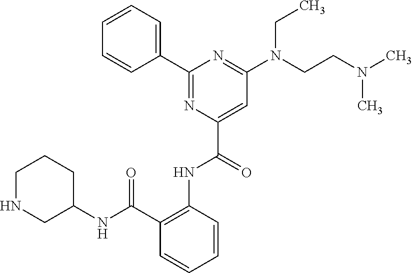

The title compound was prepared from tert-butyl 3-(2-(6-((2-(dimethylamino)ethyl)(ethyl)amino)-2-phenylpyrimidine-4-carboxamido)benzamido)piperidine-1-carboxylate according to General Method G. Yield 90 mg (79%). MS Calcd for C₂₉H₃₇N₇O₂: 515.30. Found (M+H)⁺ m/z=516

### Preparation of 6-((2-(dimethylamino)ethyl)(ethyl)amino)-2-phenyl-N-(2-(pyrrolidin-3-ylcarbamoyl)phenyl)pyrimidine-4-carboxamide

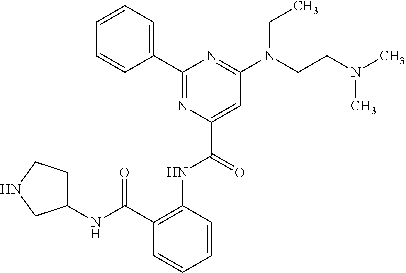

The title compound was prepared from tert-butyl 3-(2-(6-((2-(dimethylamino)ethyl)(ethyl)amino)-2-phenylpyrimidine-4-carboxamido)benzamido)pyrrolidine-1-carboxylate according to General Method G. Yield 80 mg (75%). MS Calcd for C₂₈H₃₅N₇O₂: 501.29. Found (M+H)⁺ m/z=502

### Example 2

### Biological Activity

A mass spectrometry based assay was used to identify modulators of SIRT1 activity. The mass spectrometry based assay utilizes a peptide having 20 amino acid residues as follows: Ac-EE-K(biotin)-GQSTSSHSK(Ac)NleSTEG-K(5TMR)-EE-NH2 (SEQ ID NO: 1) wherein K(Ac) is an acetylated lysine residue and Nle is a norleucine. The peptide is labeled with the fluorophore 5TMR (excitation 540 nm/emission 580 nm) at the C-terminus. The sequence of the peptide substrate is based on p53 with several modifications. In addition, the methionine residue naturally present in the sequence was replaced with the norleucine because the methionine may be susceptible to oxidation during synthesis and purification.

The mass spectrometry assay is conducted as follows: 0.5 μM peptide substrate and 120 μM βNAD⁺ is incubated with 10 nM SIRT1 for 25 minutes at 25° C. in a reaction buffer (50 mM Tris-acetate pH 8, 137 mM NaCl, 2.7 mM KCl, 1 mM MgCl₂, 5 mM DTT, 0.05% BSA). Test compounds may be added to the reaction as described above. The SirT1 gene is cloned into a T7-promoter containing vector and transformed into BL21(DE3). After the 25 minute incubation with SIRT1, 10 μL of 10% formic acid is added to stop the reaction. Reactions are sealed and frozen for later mass spec analysis. Determination of the mass of the substrate peptide allows for precise determination of the degree of acetylation (i.e. starting material) as compared to deacetylated peptide (product).

A control for inhibition of sirtuin activity is conducted by adding 1 μL of 500 mM nicotinamide as a negative control at the start of the reaction (e.g., permits determination of maximum sirtuin inhibition). A control for activation of sirtuin activity is conducted using 10 nM of sirtuin protein, with 1 μL of DMSO in place of compound, to determine the amount of deacetylation of the substrate at a given timepoint within the linear range of the assay. This timepoint is the same as that used for test compounds and, within the linear range, the endpoint represents a change in velocity.

For the above assay, SIRT1 protein was expressed and purified as follows. The SirT1 gene was cloned into a T7-promoter containing vector and transformed into BL21(DE3). The protein was expressed by induction with 1 mM IPTG as an N-terminal His-tag fusion protein at 18° C. overnight and harvested at 30,000×g. Cells were lysed with lysozyme in lysis buffer (50 mM Tris-HCl, 2 mM Tris[2-carboxyethyl]phosphine (TCEP), 10 μM ZnCl₂, 200 mM NaCl) and further treated with sonication for 10 min for complete lysis. The protein was purified over a Ni-NTA column (Amersham) and fractions containing pure protein were pooled, concentrated and run over a sizing column (Sephadex S200 26/60 global). The peak containing soluble protein was collected and run on an Ion-exchange column (MonoQ). Gradient elution (200 mM-500 mM NaCl) yielded pure protein. This protein was concentrated and dialyzed against dialysis buffer (20 mM Tris-HCl, 2 mM TCEP) overnight. The protein was aliquoted and frozen at −80° C. until further use.

Sirtuin modulating compounds that activated SIRT1 were identified using the assay described above and are shown below in Table 1. The EC_{1.5} values for the activating compounds are represented by A (EC_{1.5}≦1 uM), B (EC_{1.5}>1 and ≦10 uM), or C (EC_{1.5}>10 uM). The percent maximum fold activation is represented by A (Fold activation≧300%), B (Fold Activation≧150% and <300%), or C (Fold Activation<150%).

TABLE 1

| Compound | | | | % Fold |
|---|---|---|---|---|
| No | [M + H]+ | Structure | EC_{1.5} | Act. |
| 1 | 390 | 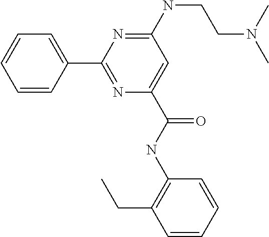 | C | B |
| 2 | 438 | 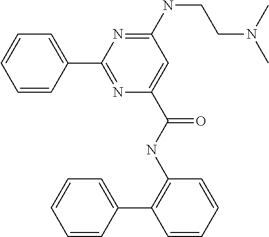 | C | C |
| 3 | 405 | 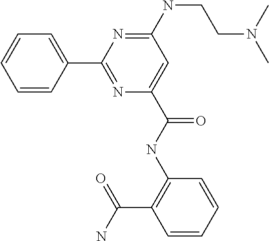 | B | A |
| 4 | 387 | 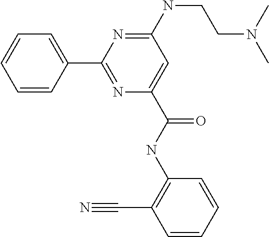 | B | B |
| 5 | 420 | 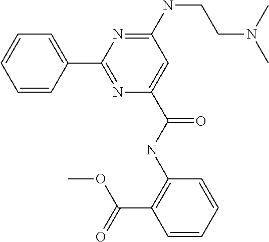 | A | A |
| 6 | 362 | 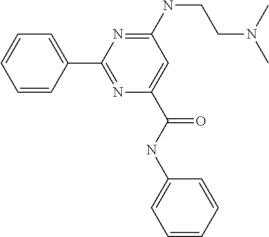 | C | B |
| 7 | 459 | 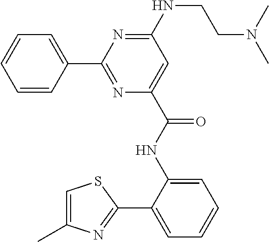 | B | A |
| 8 | 445 | 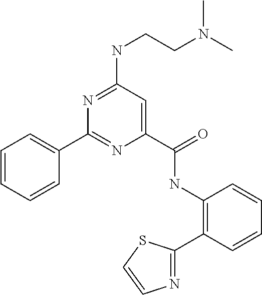 | A | A |
| 9 | 404 | 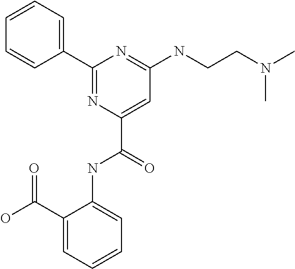 | C | C |
| 10 | 439 | 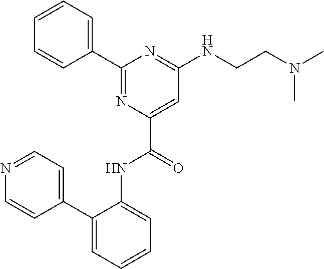 | B | B |
| 11 | 439 | 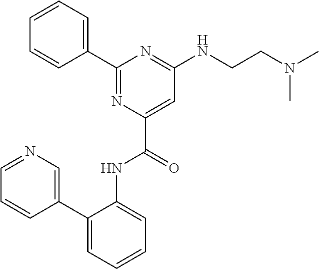 | B | B |
| 12 | 521 | 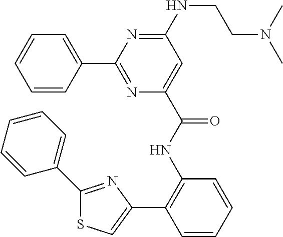 | B | B |
| 13 | 521 | 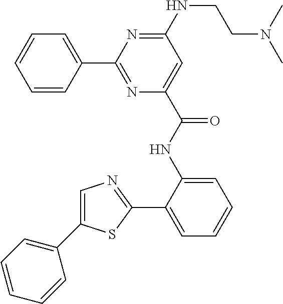 | A | B |
| 14 | 521 | 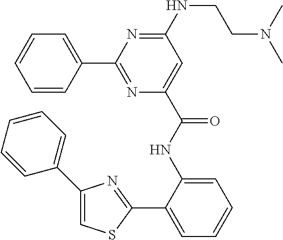 | B | B |
| 15 | 504 | 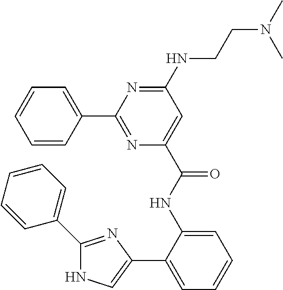 | A | A |
| 16 | 428 | 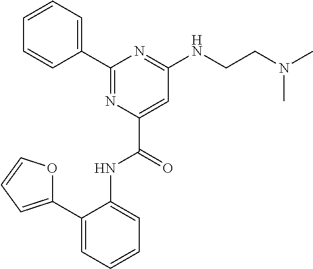 | B | A |
| 17 | 561 | 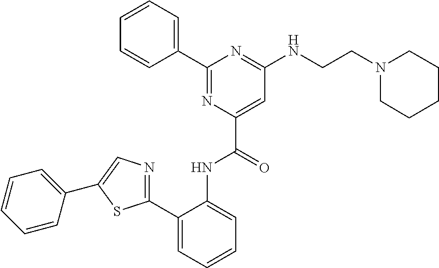 | A | B |
| 18 | 519 | 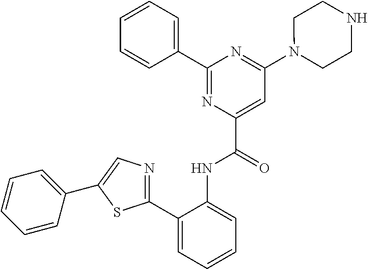 | A | B |
| 19 | 547 | 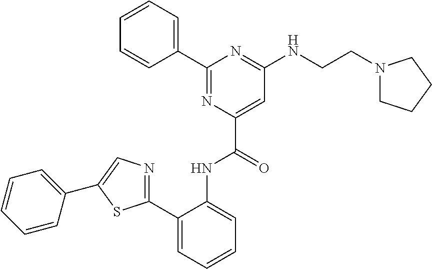 | A | B |
| 20 | 444 | 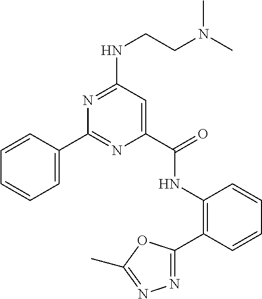 | B | A |
| 21 | 460 | 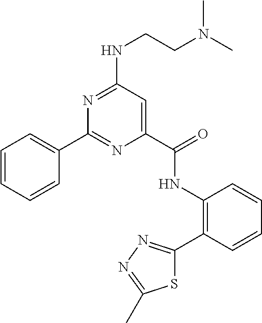 | A | B |
| 22 | 459 | 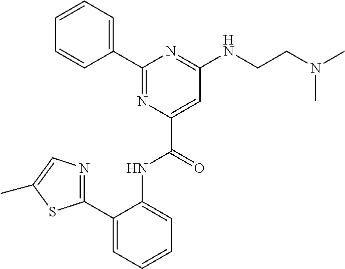 | B | A |
| 23 | 443 | 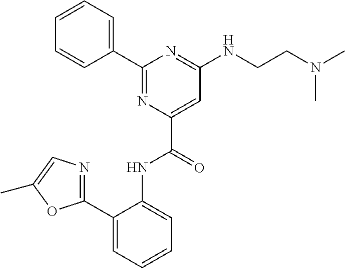 | B | A |
| 24 | 504 | 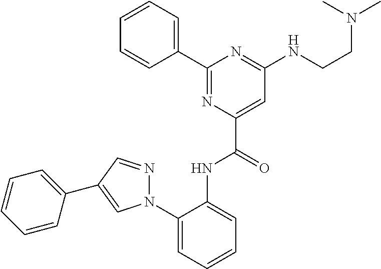 | B | B |
| 25 | 522 | 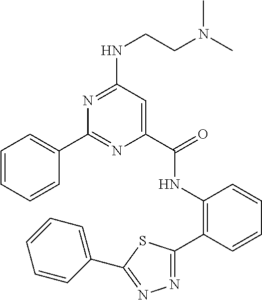 | A | B |
| 26 | 446 | 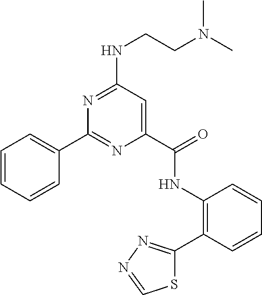 | A | A |
| 27 | 460 | 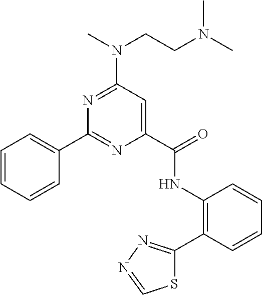 | A | A |
| 28 | 439 | 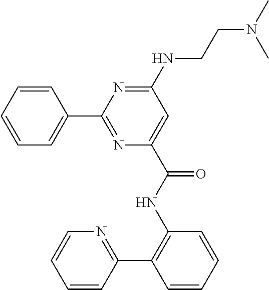 | B | A |
| 29 | 500 | 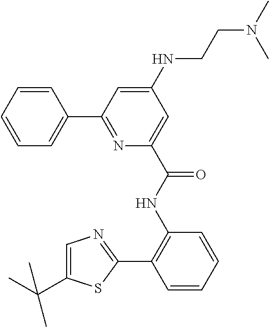 | B | A |
| 30 | 504 | 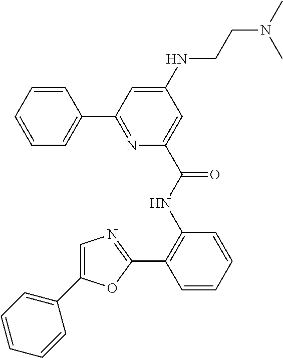 | A | A |
| 31 | 484 | 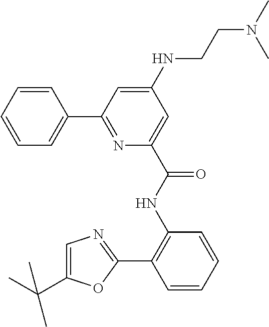 | B | A |
| 32 | 522 | 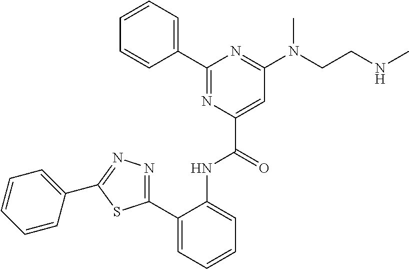 | A | A |
| 33 | 521 | 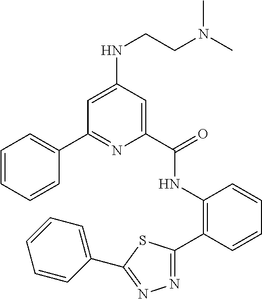 | A | A |
| 34 | 532 | 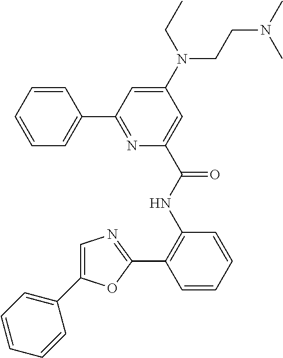 | A | A |
| 35 | 535 | 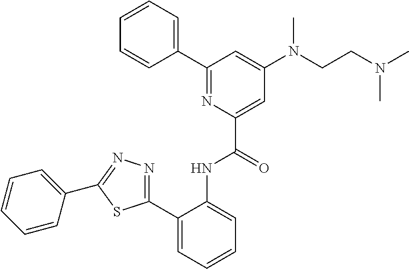 | A | B |
| 36 | 550 | 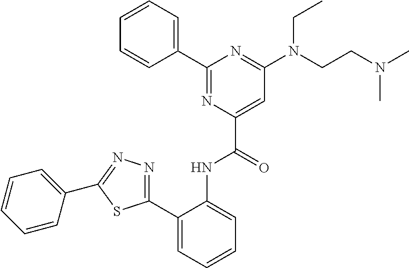 | A | B |
| 37 | 501 | 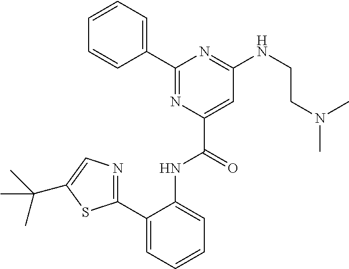 | B | B |
| 38 | 505 | 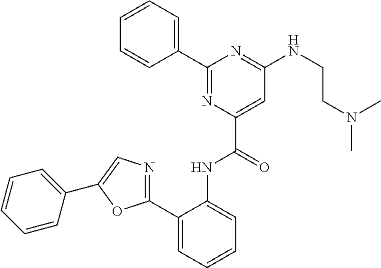 | A | B |
| 39 | 485 | 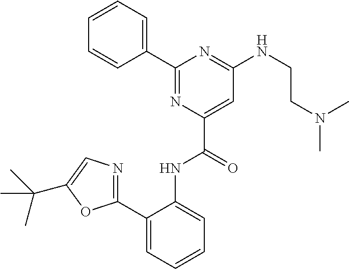 | B | B |
| 40 | 446 | 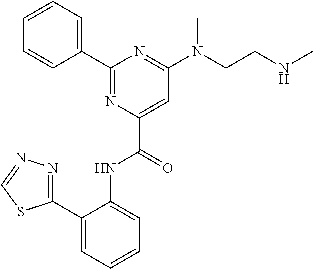 | A | A |
| 41 | 474 | 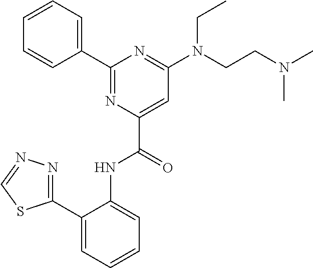 | A | A |
| 42 | 528 | 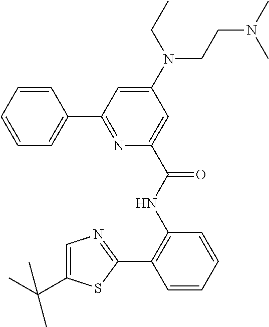 | A | B |
| 43 | 512 | 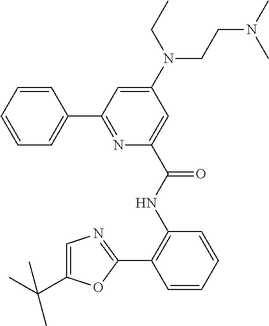 | A | A |
| 44 | 521 | 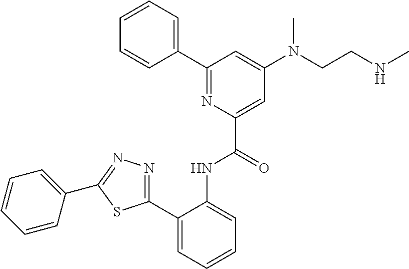 | A | A |
| 45 | 514 | 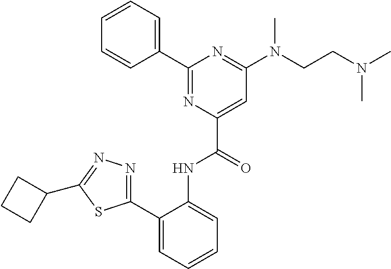 | A | B |
| 46 | 473 | 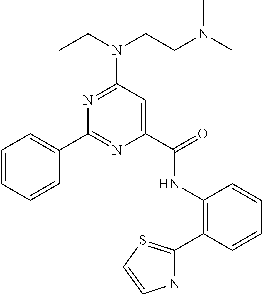 | A | A |
| 47 | 500 | 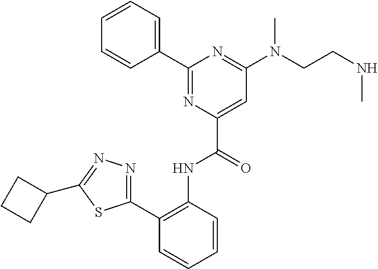 | A | B |
| 48 | 500 | 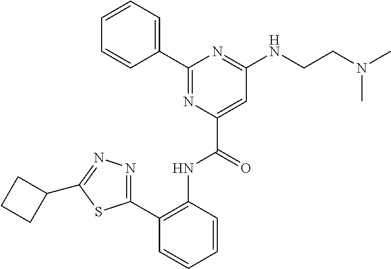 | B | B |
| 49 | 514 | 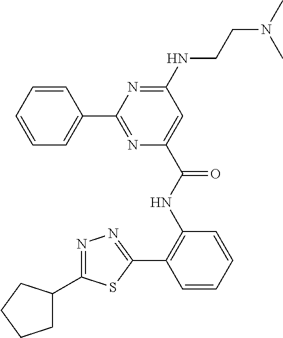 | B | B |
| 50 | 542 | 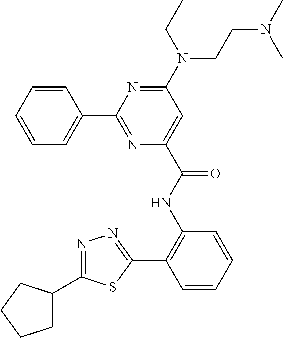 | B | B |
| 51 | 528 | 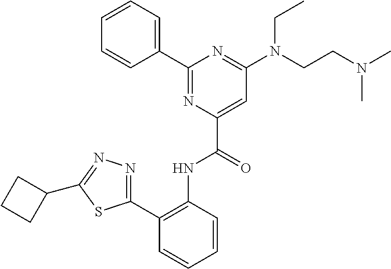 | A | B |
| 52 | 474 | 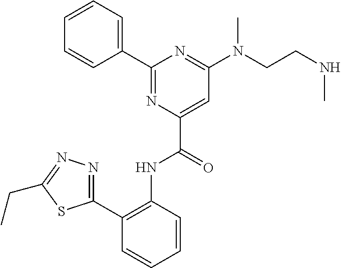 | A | B |
| 53 | 474 | 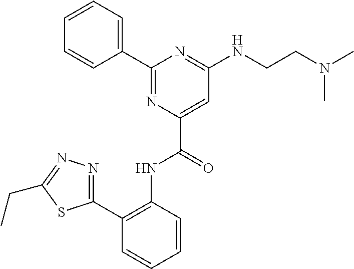 | B | B |
| 54 | 488 | 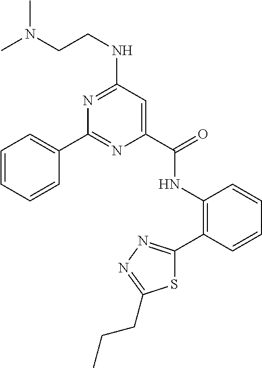 | B | B |
| 55 | 502 | 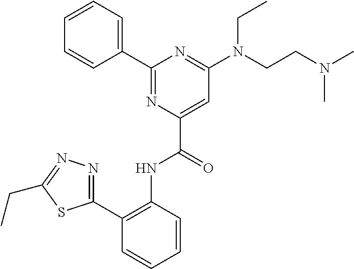 | A | B |
| 56 | 488 | 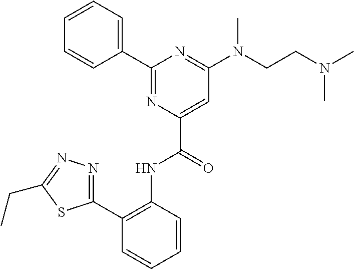 | B | B |
| 57 | 532 | 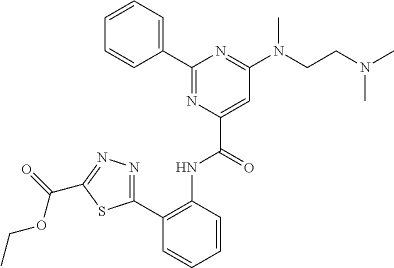 | C | C |
| 58 | 560 | 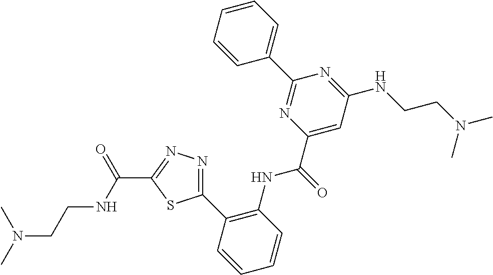 | A | A |
| 59 | 546 | 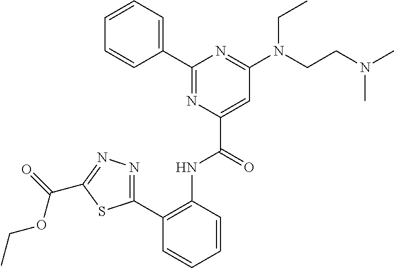 | A | B |
| 60 | 502 | 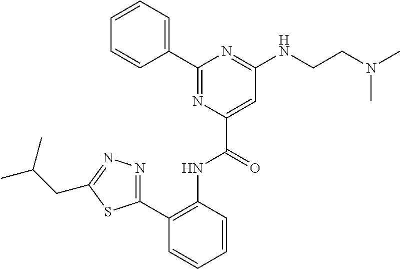 | A | B |
| 61 | 502 | 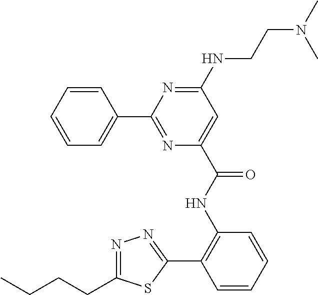 | B | C |
| 62 | 530 | 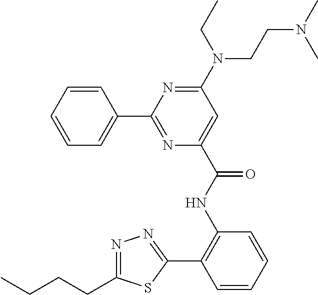 | A | B |
| 63 | 529 | 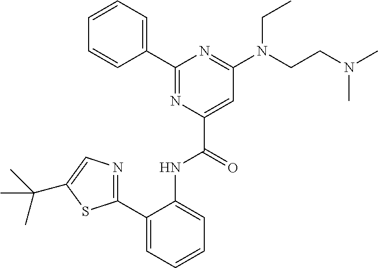 | A | B |
| 64 | 533 | 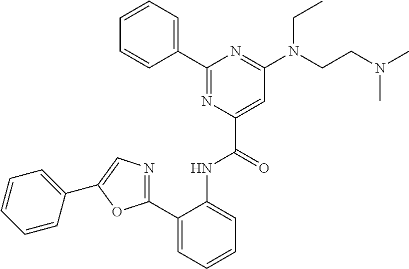 | A | B |
| 65 | 513 | 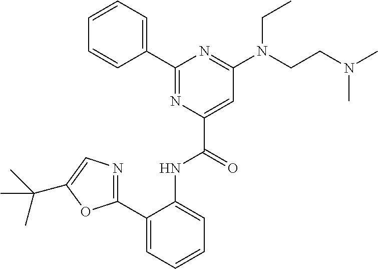 | B | B |
| 66 | 489 | 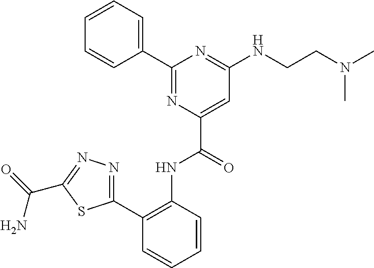 | A | B |
| 67 | 487 | 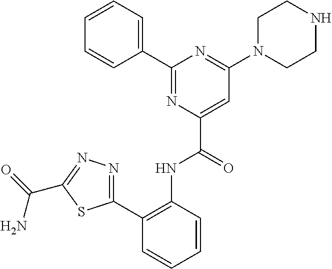 | A | B |
| 68 | 532 | 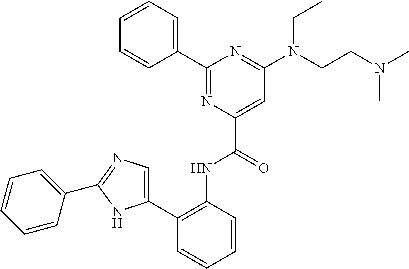 | A | A |
| 69 | 527 | 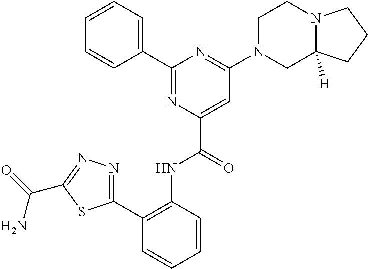 | A | B |
| 70 | 483 | 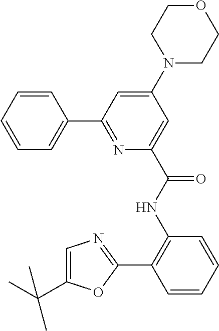 | A | B |
| 71 | 517 | 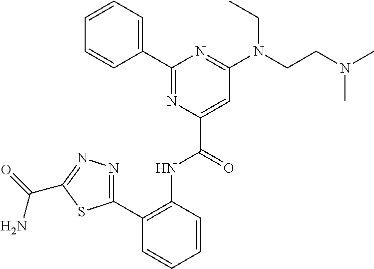 | A | B |
| 72 | 488 | 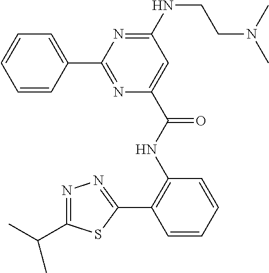 | A | A |
| 73 | 523 | 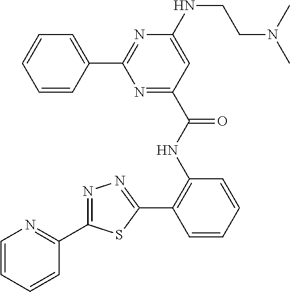 | A | A |
| 74 | 551 | 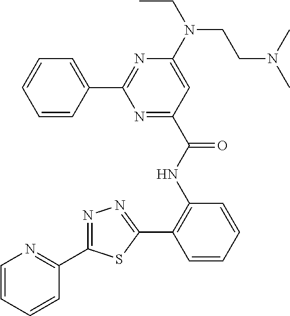 | A | A |
| 75 | 504 | 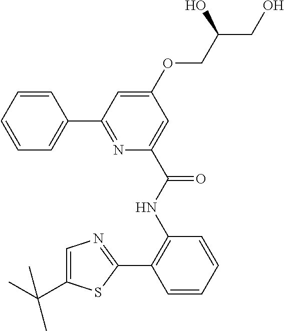 | B | A |
| 76 | 488 | 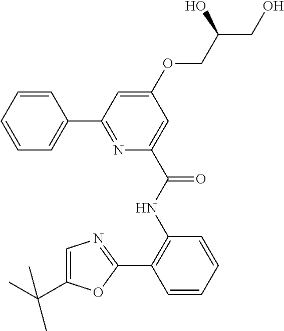 | B | A |
| 77 | 523 | 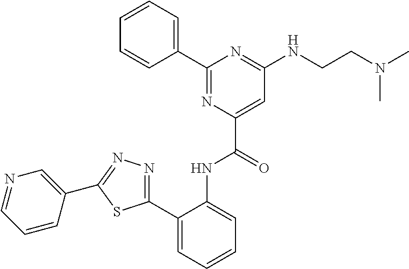 | A | A |
| 78 | 523 | 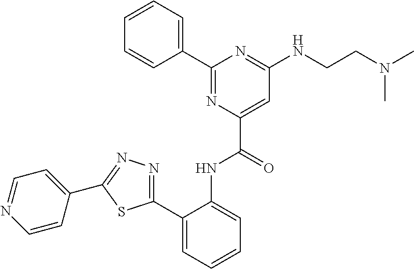 | A | A |
| 79 | 516 | 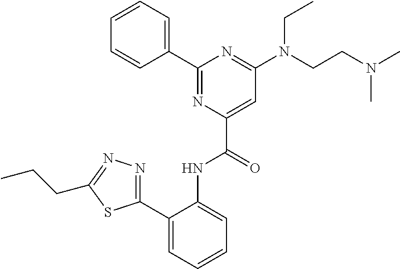 | A | A |
| 80 | 530 | 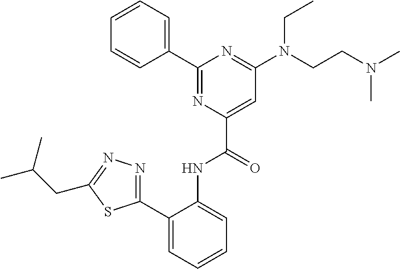 | B | B |
| 81 | 551 | 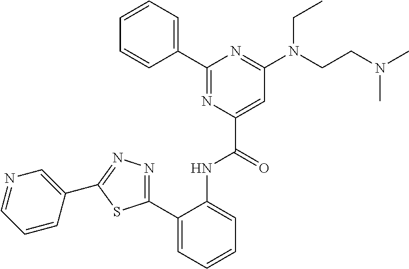 | A | A |
| 82 | 551 | 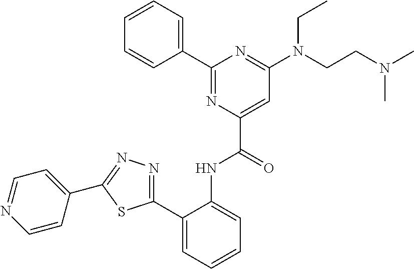 | A | A |
| 83 | 516 | 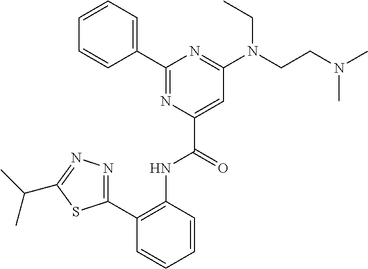 | A | A |
| 84 | 515 | 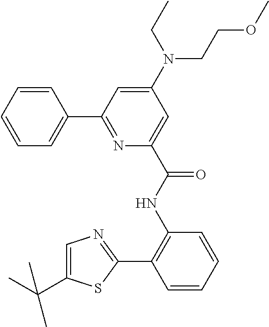 | A | B |
| 85 | 499 | 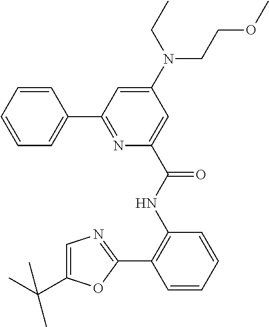 | A | A |
| 86 | 531 | 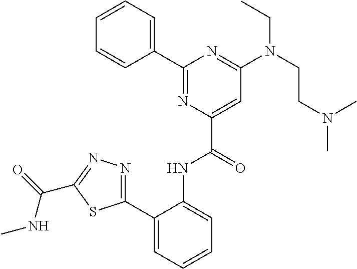 | A | B |
| 87 | 587 | 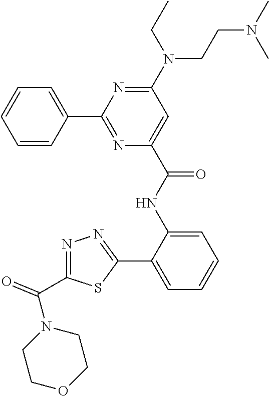 | A | A |
| 88 | 585 | 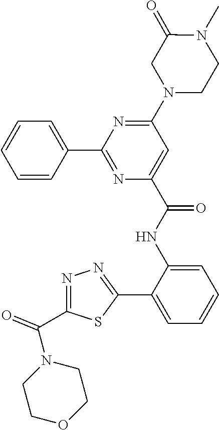 | A | A |
| 89 | 571 | 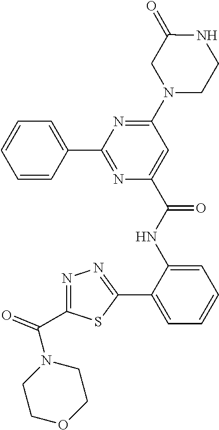 | A | A |
| 90 | 562 | 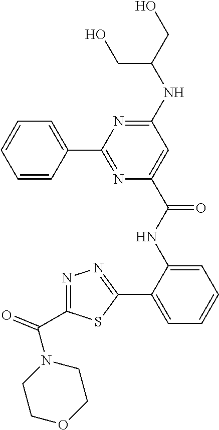 | A | A |
| 91 | 563 | 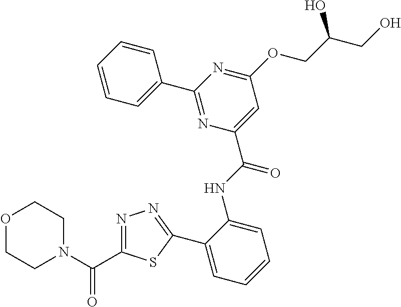 | A | B |
| 92 | 604 | 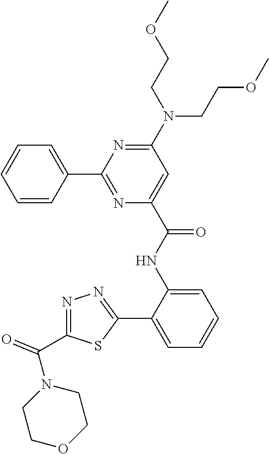 | A | A |
| 93 | 558 | 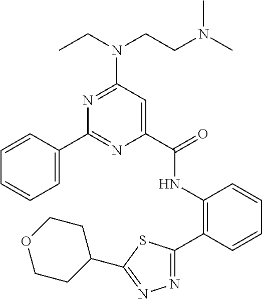 | A | A |
| 94 | 570 | 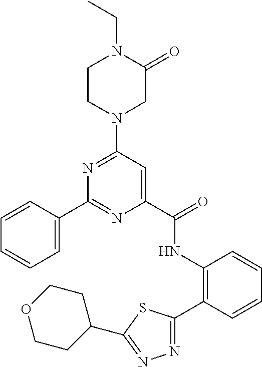 | A | B |
| 95 | 458 | 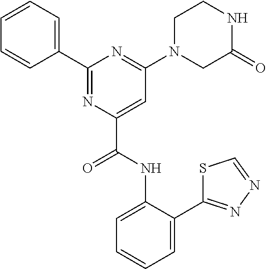 | A | A |
| 96 | 486 | 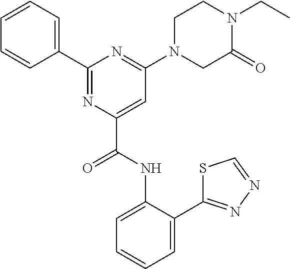 | A | B |
| 97 | 544 | 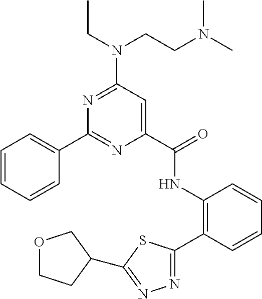 | A | A |
| 98 | 567 | 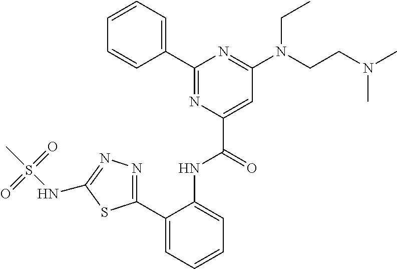 | A | A |
| 99 | 449 | 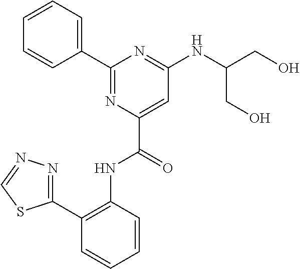 | A | A |
| 100 | 530 | 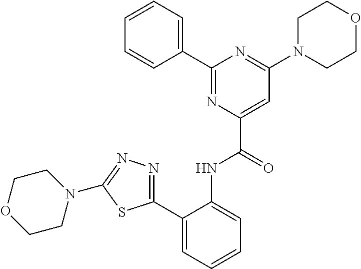 | A | B |
| 101 | 531 | 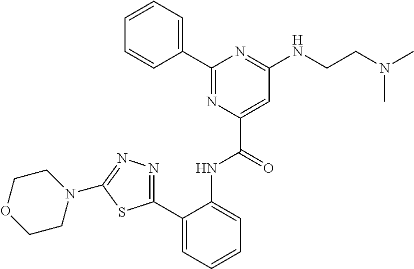 | A | A |
| 102 | 534 | 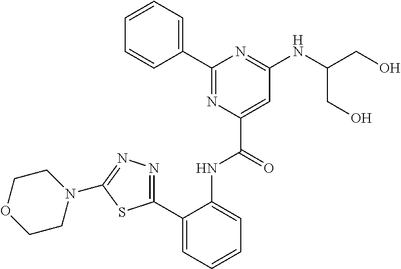 | A | A |
| 103 | 543 | 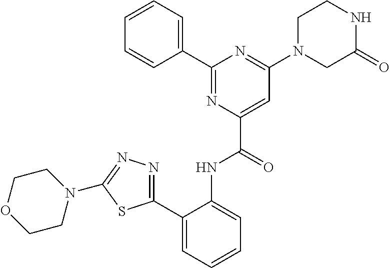 | A | A |
| 104 | 571 | 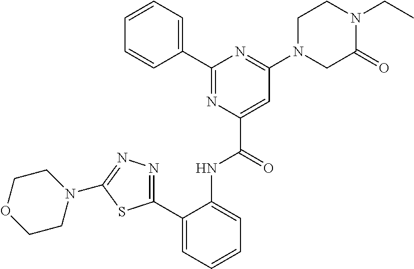 | A | B |
| 105 | 559 | 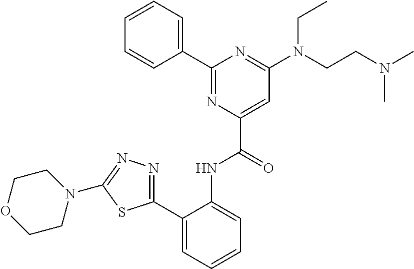 | A | A |
| 106 | 576 | 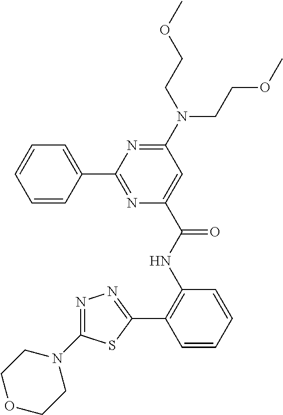 | A | B |
| 107 | 546 | 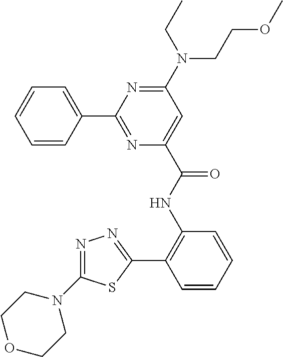 | A | B |
| 108 | 535 | 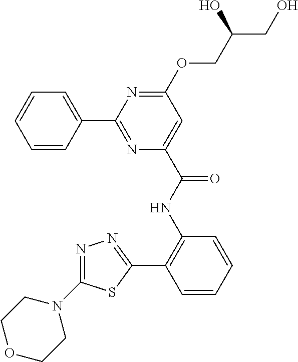 | A | A |
| 109 | 535 | 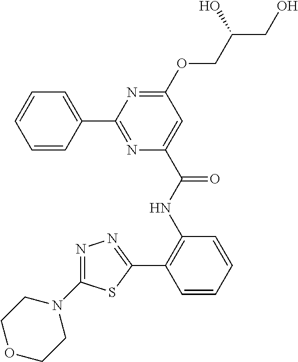 | A | A |
| 110 | 450 | 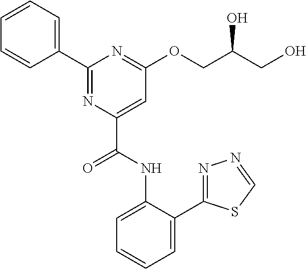 | A | A |
| 111 | 450 | 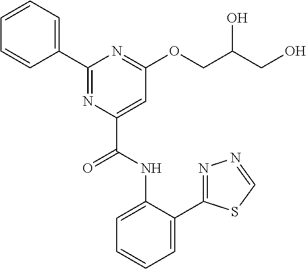 | A | A |
| 112 | 472 | 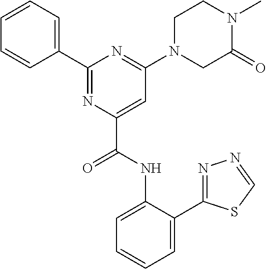 | A | A |
| 113 | 602 | 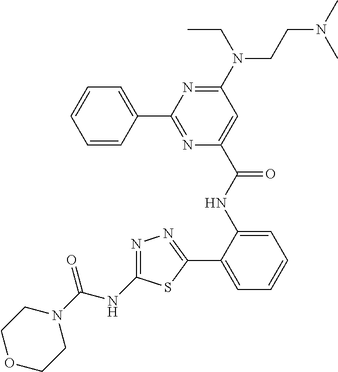 | A | A |
| 114 | 588 | 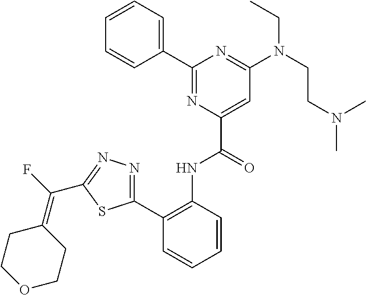 | A | A |
| 115 | 520 | 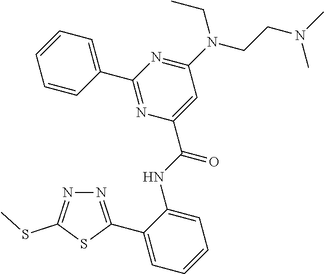 | A | A |
| 116 | 536 | 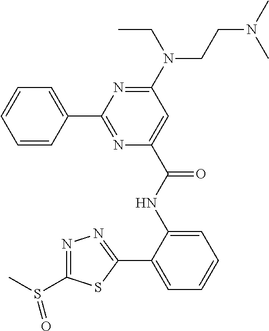 | A | A |
| 117 | 557 | 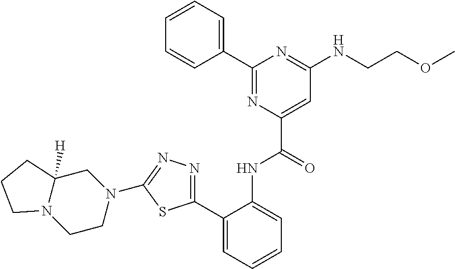 | A | B |
| 118 | 582 | 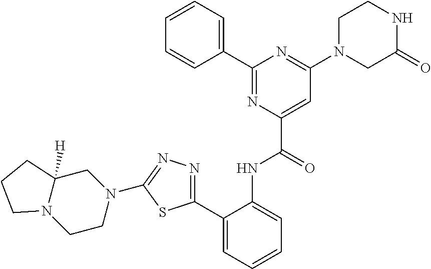 | A | B |
| 119 | 409 | 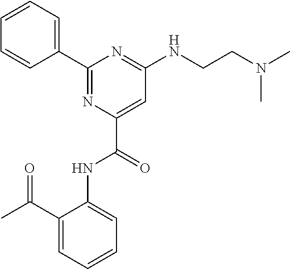 | A | A |
| 120 | 459 | 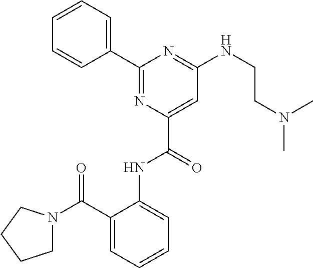 | C | A |
| 121 | 476 | 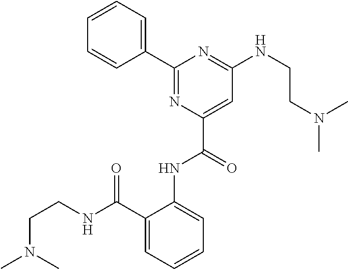 | B | B |
| 122 | 463 | 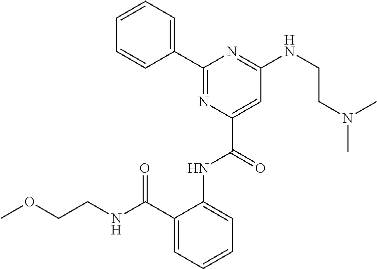 | C | A |
| 123 | 461 | 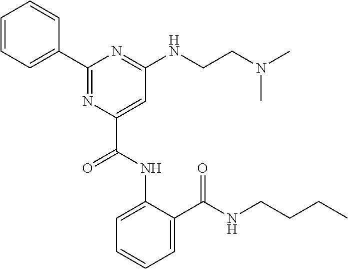 | C | C |
| 124 | 461 | 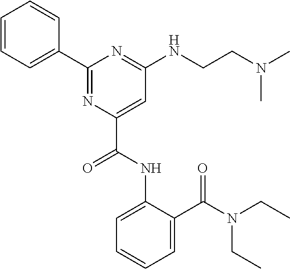 | B | B |
| 125 | 419 | 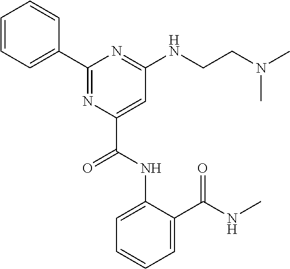 | B | A |
| 126 | 418 | 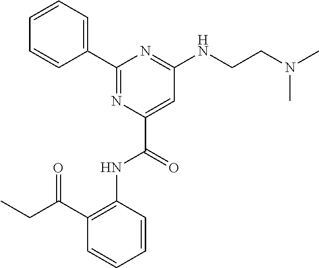 | B | B |
| 127 | 446 | 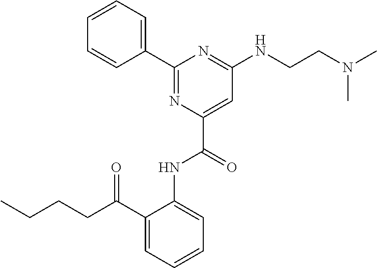 | B | B |
| 128 | 481 | 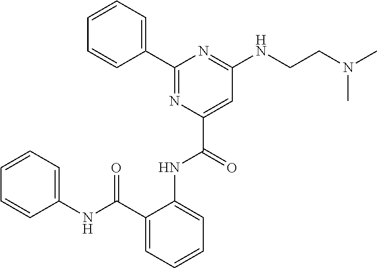 | B | B |
| 129 | 446 | 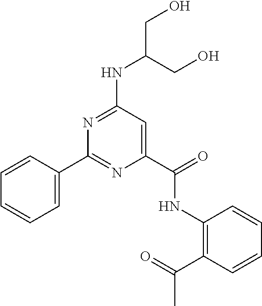 | A | A |
| 130 | 477 | 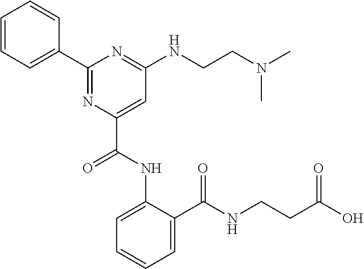 | B | B |
| 131 | 466 | 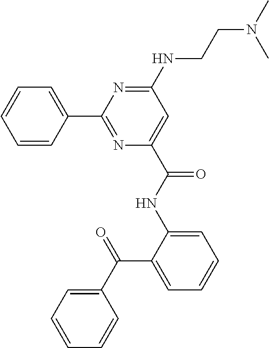 | B | B |
| 132 | 419 | 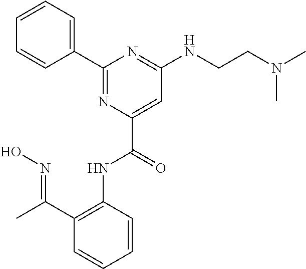 | B | B |
| 133 | 406 | 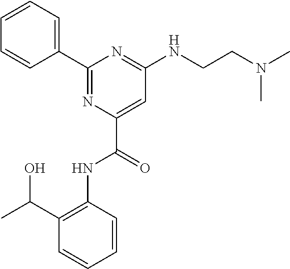 | C | B |
| 134 | 403 | 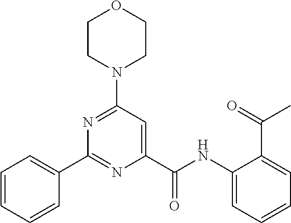 | A | B |
| 135 | 432 | 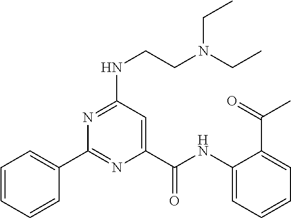 | B | A |
| 136 | 440 | 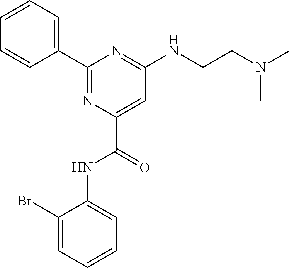 | B | B |
| 137 | 458 | 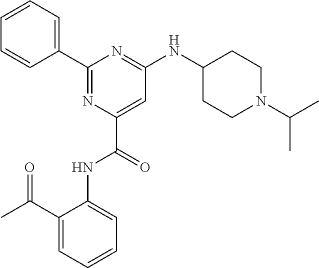 | A | A |
| 138 | 402 | 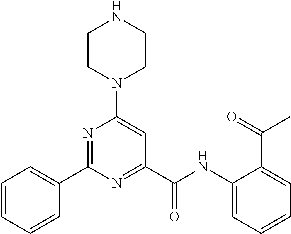 | B | A |
| 139 | 550 | 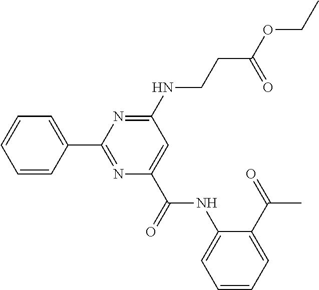 | B | B |
| 140 | 416 | 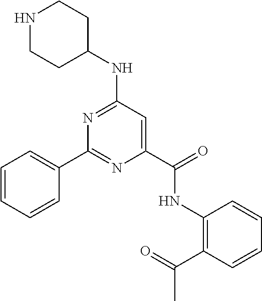 | A | A |
| 141 | 449 | 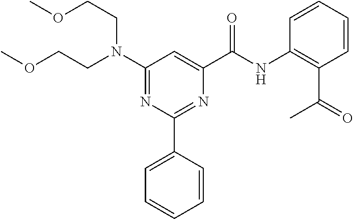 | A | A |
| 142 | 432 | 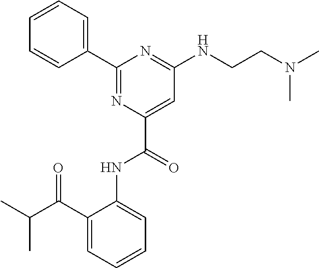 | B | B |
| 143 | 430 | 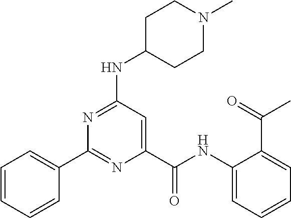 | A | A |
| 144 | 495 | 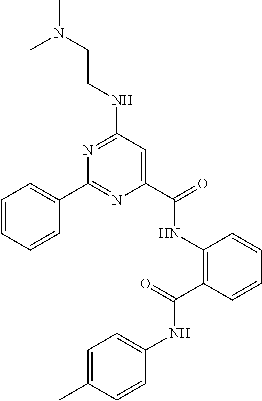 | A | B |
| 145 | 515 | 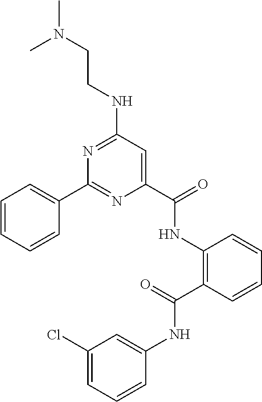 | A | A |
| 146 | 495 | 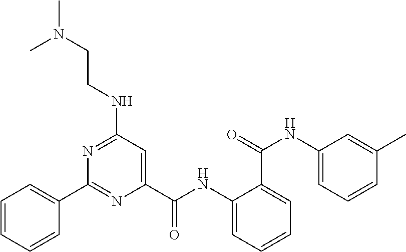 | B | A |
| 147 | 524 | 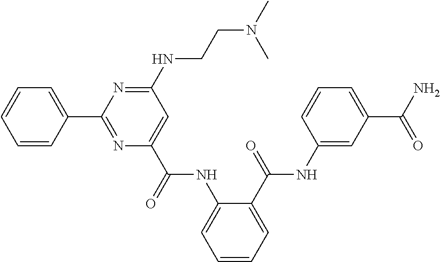 | A | A |
| 148 | 479 | 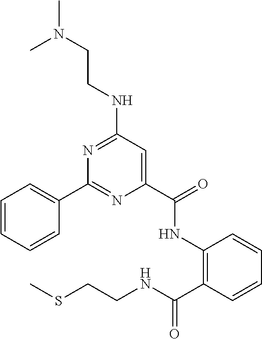 | B | A |
| 149 | 511 | 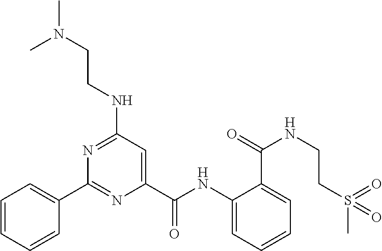 | B | B |
| 150 | 524 | 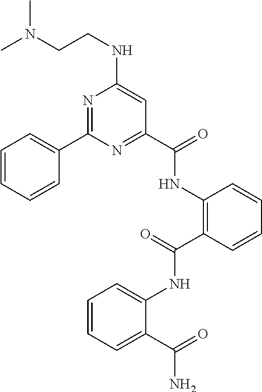 | A | A |
| 151 | 495 | 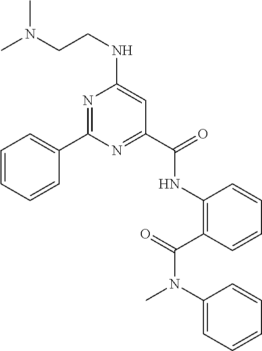 | C | B |
| 152 | 495 | 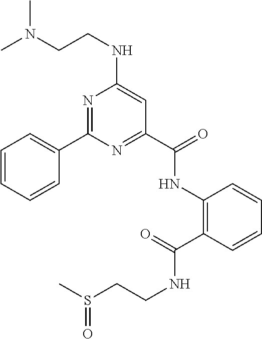 | B | B |
| 153 | 549 | 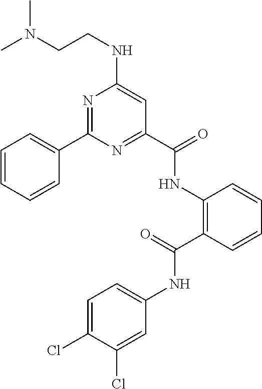 | C | B |
| 154 | 559 | 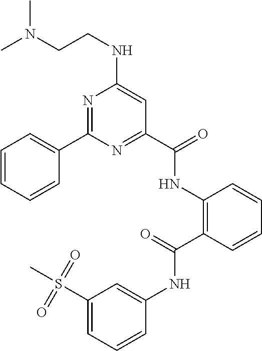 | A | A |
| 155 | 482 | 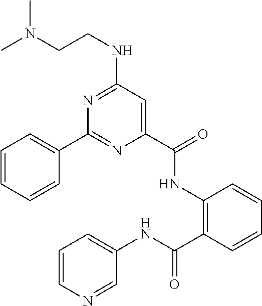 | A | A |
| 156 | 482 | 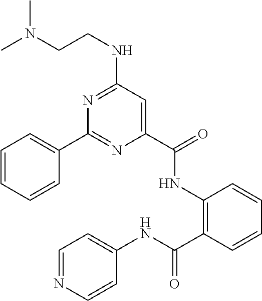 | A | A |
| 157 | 524 | 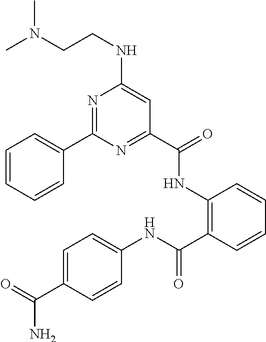 | A | B |
| 158 | 482 | 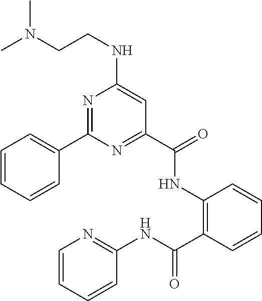 | A | B |
| 159 | 468 | 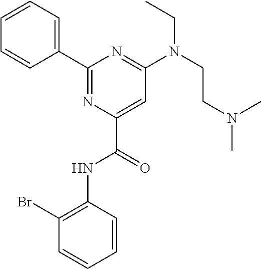 | B | B |
| 160 | 552 | 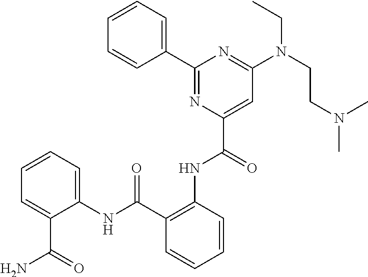 | A | A |
| 161 | 549 | 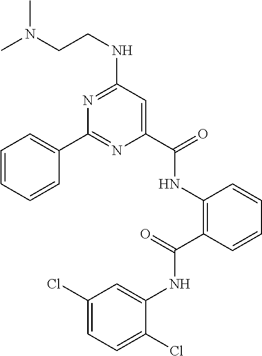 | B | B |
| 162 | 549 | 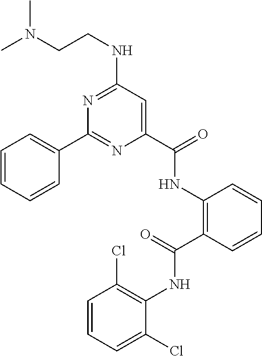 | A | A |
| 163 | 559 | 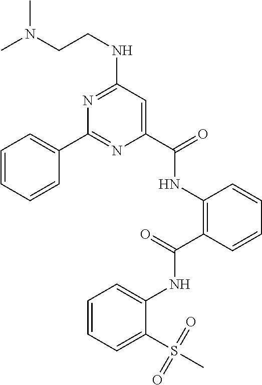 | B | B |
| 164 | 487 | 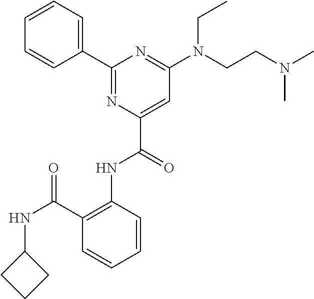 | A | A |
| 165 | 515 | 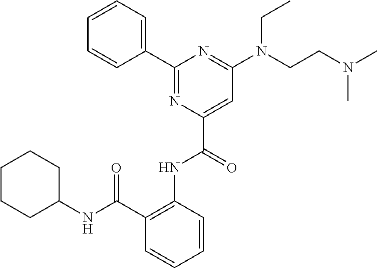 | A | B |
| 166 | 616 | 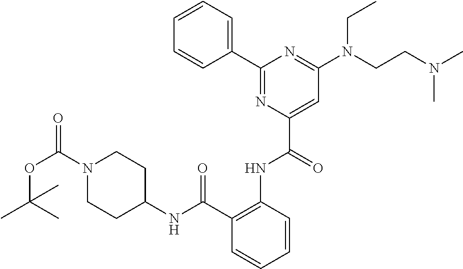 | B | B |
| 167 | 516 | 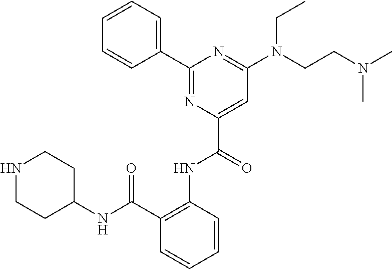 | A | A |
| 168 | 473 | 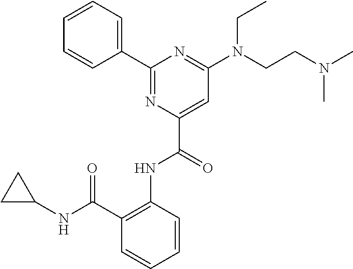 | A | B |
| 169 | 501 | 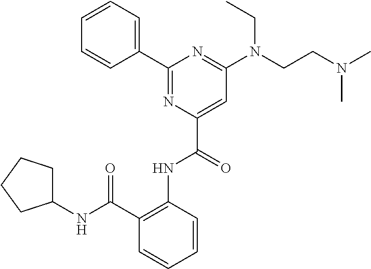 | B | A |
| 170 | 531 | 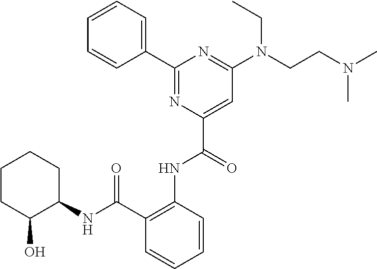 | A | A |
| 171 | 531 | 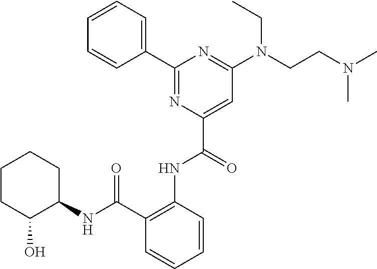 | A | A |
| 172 | 616 | 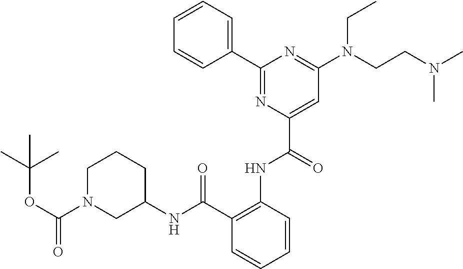 | B | B |
| 173 | 602 | 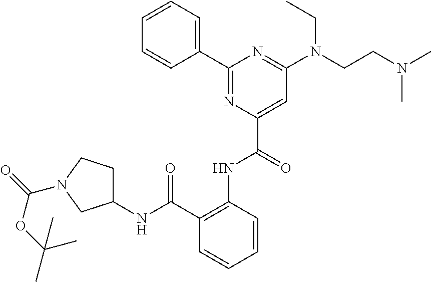 | B | A |
| 174 | 516 | 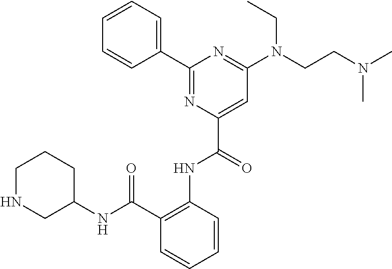 | B | A |
| 175 | 502 | 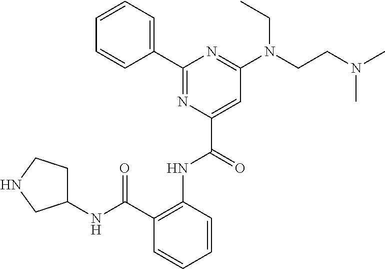 | A | A |
| 176 | 472 | 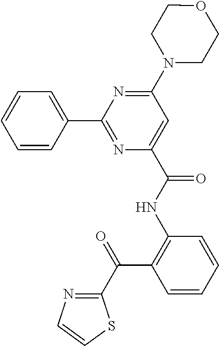 | A | B |
| 177 | 501 | 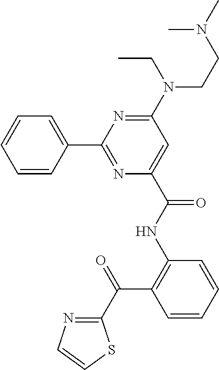 | A | A |
| 178 | 466 | 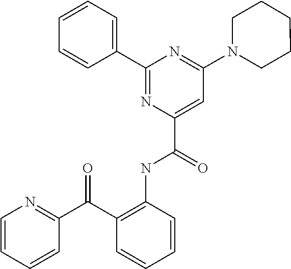 | A | B |
| 179 | 536 | 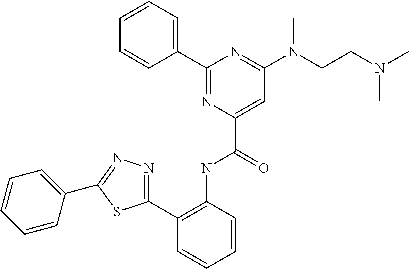 | C | B |

### EQUIVALENTS

The present invention provides among other things sirtuin-activating compounds and methods of use thereof. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### INCORPORATION BY REFERENCE

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

Also incorporated by reference are the following: PCT Publications WO 2005/002672; 2005/002555; and 2004/016726.

1. A compound represented by Structural Formula (I): 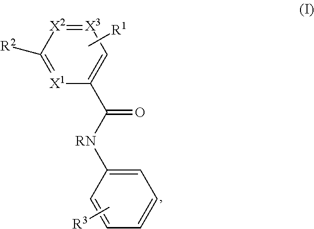
or a salt thereof, wherein:
two of X¹, X² and X³ are independently selected from —CH— and —N—;
the other of X¹, X² and X³ is —CH—;
R¹ is a solubilizing group;
R² is selected from phenyl, fluorophenyl and a 5- to 6-membered heterocycle containing an N heteroatom and, optionally, a second heteroatom selected from N, O or S, wherein said heterocycle is optionally substituted with methyl;
R is —H or —CH₃;
R³ is selected from H, —C(O)R⁴, —C(O)OR⁴, —OC(O)R⁴, —C(O)NR^{4a}R^{4b}, —NR^{4a}C(O)R^{4b}, —NR^{4a}R^{4b}, —C(═N—OH)R⁴, —OR⁴, —SR⁴, —CH₂R⁴, alkyl, alkenyl, alkynyl, cyano, monocyclyl and halo;
R⁴ in each occurrence is independently selected from hydrogen, lower alkyl and monocyclyl; and
R^{4a} and R^{4b} are independently selected from hydrogen, lower alkyl and monocyclyl; or R^{4a} and R^{4b} taken together with the atom to which they are connected form a heterocycle.

2. The compound of claim 1, wherein R³ is selected from H, —C(O)R⁴, —C(O)OR⁴, —OC(O)R⁴, —C(O)NR^{4a}R^{4b}, —NR^{4a}C(O)R^{4b}, —NR^{4a}R^{4b}, —OR⁴, —SR⁴, —CH₂R⁴, alkyl, alkenyl, alkynyl, cyano, monocyclyl and halo; and
R^{4a} and R^{4b} are independently selected from hydrogen, lower alkyl and monocyclyl,
wherein monocyclyl groups are optionally substituted with one or more substituents selected from halo, cyano, lower alkoxy, lower alkyl, hydroxyl, amino, lower alkylamino and lower dialkylamino.

3. The compound of claim 1, wherein X¹ is —N—.

4. The compound of claim 3, wherein X¹ and X² are —N—.

5. The compound of claim 1, wherein R² is selected from phenyl, fluorophenyl, methylthiazolyl, pyrimidinyl, pyridyl and pyrazolyl.

6. The compound of claim 5, wherein R² is phenyl.

7. The compound of claim 1, wherein R³ is selected from —C(O)R⁴, —C(O)OR⁴, —OC(O)R⁴, —C(O)NR^{4a}R^{4b}, —NR^{4a}C(O)R^{4b}, —OR⁴, —SR⁴, —CH₂R⁴, —NR^{4a}R^{4b}, alkyl, alkenyl, alkynyl, cyano, monocyclyl and halo.

8. The compound of claim 7, wherein R³ is —NR^{4a}R^{4b}; and
R^{4a} and R^{4b} are hydrogen or lower alkyl.

9. The compound of claim 7, wherein R³ is selected from alkyl, monocyclyl, —C(O)NR^{4a}R^{4b}, —NR^{4a}C(O)R^{4b}, —OC(O)R⁴, —C(O)OR⁴ or cyano.

10. The compound of claim 9, wherein R³ is selected from 5-7 membered heterocyclyl and 5-7 membered carbocyclyl.

11. The compound of claim 10, wherein R³ is a 5-7 membered heterocyclyl comprising at least one nitrogen.

12. The compound of claim 11, wherein R³ is selected from substituted or unsubstituted thiazolyl, oxazolyl, isoxazolyl, isothiozolyl, triazolyl, tetrazolyl, pyrazolyl, imidazolyl, pyridinyl, pyrrolyl, thiazinyl, oxazinyl, piperidinyl, piperazinyl, pyrimidinyl, morpholinyl, and thiomorpholinyl.

13. The compound of claim 11, wherein R³ is substituted or unsubstituted thiadiazolyl.

14. The compound of claim 13, wherein R³ is substituted or unsubstituted 2-(1,3,4-thiadiazolyl).

15. The compound of claim 14, wherein 2-(1,3,4-thiadiazolyl) is substituted with a solubilizing group.

16. The compound of claim 7, wherein R³ is selected from —C(O)OR⁴, —OC(O)R⁴, —C(O)NR^{4a}R^{4b} and —NR^{4a}C(O)R^{4b}.

17. The compound of claim 16, wherein R³ is selected from —C(O)OR⁴ and —C(O)NR^{4a}R^{4b}.

18. The compound of claim 17, wherein R⁴, R^{4a} and R^{4b} are selected from H and lower alkyl.

19. The compound of claim 1, wherein R³ is —CH₂R⁴; and
R⁴ is a nitrogen-containing heterocycle.

20. The compound of claim 1, wherein R¹ is —OR⁵, —SR⁵, —NHR⁵, or —NR⁷R⁸; and
R⁵ is lower alkyl.

21. The compound of claim 20, wherein R¹ is —NHR⁵; and
R⁵ is aminoalkyl, alkyl aminoalkyl, dialkyl aminoalkyl, acetyl aminoalkyl, lower alkyl carboxy lower alkyl, alkyloxycarbonyl alkyl, hydroxyalkyl, alkoxyalkyl, alkyl thioalkyl, monocyclyl, monocyclylalkyl, or alkyl sulfonylalkyl.

22. The compound of claim 20, wherein R¹ is —NR⁷R⁸ and R⁷ and R⁸ together with the nitrogen to which they are attached form a 5, 6 or 7-membered heterocycle.

23. The compound of claim 1, wherein R¹ is —CH₂R⁶; and
R⁶ is a nitrogen-containing heterocycle.

24. The compound of claim 1, wherein:
R² is selected from phenyl, 3-fluorophenyl and pyridyl; and
X¹ and X² are —N— and X³ is —CH—.

25. The compound of claim 24, wherein R³ is —NR^{4a}R^{4b}; and
R^{4a} and R^{4b} are hydrogen or lower alkyl.

26. The compound of claim 24, wherein R³ is —CH₂R⁴; and
R⁴ is a nitrogen-containing heterocycle.

27. The compound of claim 24, wherein R³ is selected from alkyl, monocyclyl, —C(O)NR^{4a}R^{4b}, —NR^{4a}C(O)R^{4b}, —OC(O)R⁴, —C(O)OR⁴ or cyano.

28. The compound of claim 27, wherein R³ is monocyclyl.

29. The compound of claim 24, wherein R¹ is —NHR⁵; and R⁵ is lower alkyl.

30. The compound of claim 24, wherein R¹ is —CH₂R⁶; and R⁶ is a nitrogen-containing heterocycle.

31. A pyrogen-free composition comprising a compound of claim 1, or a pharmaceutically acceptable salt thereof and a carrier.

32. A pharmaceutical composition comprising a compound of claim 1 and a pharmaceutically acceptable carrier.

33. The pharmaceutical composition of claim 32, further comprising an additional active agent.

34. A method for treating or preventing insulin resistance, a metabolic syndrome, diabetes, or complications thereof, or for increasing insulin sensitivity in a subject, comprising administering to the subject in need thereof a pharmaceutical composition of claim 32.
